SEQUENCE LISTING (2 sequences)

SEQ ID NO: 1 - protein, 214 aa, synthetic construct
DIQMTQSPSSVSASVGDRVTITCRASQGIGSWLAWYQQKPGKAPKLLIYAADRLQSGVPSRFSGSGSGTDFTLTISSLQPEDFATYYCQQYHTYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 453 aa, synthetic construct
QVQLVESGGGVVQPGRSLRLSCAASGFNFSDYGMHWVRQAPGKGLEWVAVIWYDGSISYYADSVKGRFTISRDNSKNTLYLQMNSLRAEDTAVYYCARGGTVEHGAVYGTDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2026/0027221 A1
(43) Pub. Date: Jan. 29, 2026

(54) Targeted Pyrrolobenzodiazapine Conjugates

(71) Applicant: BeOne Medicines I GmbH, Basel (CH)

(72) Inventors: Charng-Sheng Tsai, Beijing (CN); Mei-Hsuan Tsai, Shanghai (CN); Maomao He, Shanghai (CN); Zewei Wang, Shanghai (CN); Xiaokun Yang, Shanghai (CN); Lingyan Li, Shanghai (CN)

(73) Assignee: BeOne Medicines I GmbH, Basel (CH)

(21) Appl. No.: 19/352,620

(22) Filed: Oct. 8, 2025

Related U.S. Application Data
(63) Continuation of application No. PCT/CN24/87465, filed on Apr. 12, 2024.

(30) Foreign Application Priority Data
Apr. 13, 2023 (CN) CNPCT/CN23/88051

(51) Int. Cl. A61K 47/68; A61K 47/55

(52) U.S. Cl. CPC A61K 47/68035; A61K 47/55; A61K 47/6889

(57) ABSTRACT

Provided are compounds comprising pyrrolobenzodiazepine (PBD) conjugates and methods of using such conjugates. In some embodiments, a conjugate is of Formula (I):

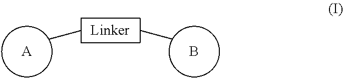

or a pharmaceutically acceptable salt, tautomer, solvate, or stereoisomer thereof, wherein each of ring A and ring B is, independently, one of the following formulas:

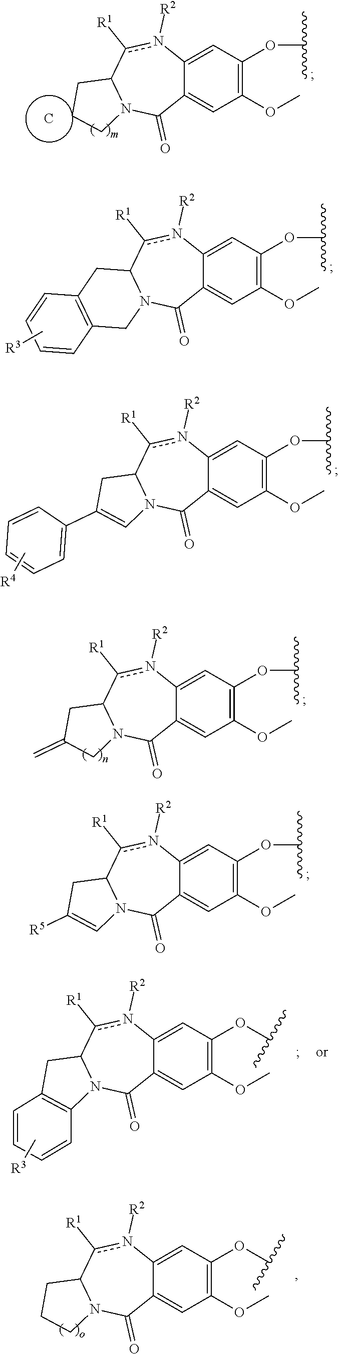

and values for the remaining variables (e.g., Linker, ring C, R¹, R², R³, R⁴, R⁵, m, n, o) are as described herein.

### 1. CROSS-REFERENCE TO RELATED APPLICATIONS

This application is a continuation of International Application No. PCT/CN2024/087465, filed Apr. 12, 2024, which claims priority to International Application No. PCT/CN2023/088051, filed Apr. 13, 2023, the contents of which are hereby incorporated by reference in their entirety.

### 2. FIELD

The present disclosure relates to targeted pyrrolobenzodiazepine (PBD) conjugates

### 3. SEQUENCE LISTING

The instant application contains a Sequence Listing, which has been submitted electronically in XML format and is hereby incorporated by reference in its entirety. Said XML copy, created on Oct. 7, 2025, is named “01368-0071-OOUS” and is 3,439 bytes in size.

### 4. BACKGROUND

Some pyrrolobenzodiazepines (PBDs) have the ability to recognize and bind to specific sequences of DNA; the preferred sequence is PuGPu. The first PBD antitumor antibiotic, anthramycin, was discovered in 1965 (Leimgruber, et al., J. Am. Chem. Soc., 87, 5793-5795 (1965); Leimgruber, et al., J. Am. Chem. Soc., 87, 5791-5793 (1965)). Since then, a number of naturally occurring PBDs have been reported, and more than ten synthetic routes have been developed for a variety of analogues (Thurston, et al., Chem. Rev. 1994, 433-465 (1994)). Family members include abbeymycin (Hochlowski, et al., J. Antibiotics, 40, 145-148 (1987)), chicamycin (Konishi, et al., J. Antibiotics, 37, 200-206 (1984)), DC-81 (Japanese Patent 58-180 487; Thurston, et al., Chem. Brit., 26, 767-772 (1990); Bose, et al., Tetrahedron, 48, 751-758 (1992)), mazethramycin (Kuminoto, et al., J. Antibiotics, 33, 665-667 (1980)), neothramycins A and B (Takeuchi, et al., J. Antibiotics, 29, 93-96 (1976)), porothramycin (Tsunakawa, et al., J. Antibiotics, 41, 1366-1373 (1988)), prothracarcin (Shimizu, et al, J. Antibiotics, 29, 2492-2503 (1982); Langley and Thurston, J. Org. Chem., 52, 91-97 (1987)), sibanomicin (DC-102) (Hara, et al., J. Antibiotics, 41, 702-704 (1988); Itoh, et al., J. Antibiotics, 41, 1281-1284 (1988)), sibiromycin (Leber, et al., J. Am. Chem. Soc., 110, 2992-2993 (1988)), and tomamycin (Arima, et al., J. Antibiotics, 25, 437-444 (1972)). PBDs have the following general structure:

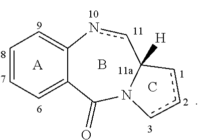

PBDs differ in the number, type, and position of substituents, in both their aromatic A rings and pyrrolo C rings, and in the degree of saturation of the C ring. In the B-ring there is an imine (N═C), a carbinolamine (NH—CH(OH)), or a carbinolamine methyl ether (NH—CH(OMe)) at the N10-C11 position, which is the electrophilic center responsible for alkylating DNA. All of the known natural products have an (S)-configuration at the chiral C11a position which provides them with a right-handed twist when viewed from the C ring towards the A ring. This gives them the appropriate three-dimensional shape for isohelicity with the minor groove of B-form DNA, leading to a snug fit at the binding site (Kohn, In Antibiotics III. Springer-Verlag, New York, pp. 3-11 (1975); Hurley and Needham-VanDevanter, Acc. Chem. Res., 19, 230-237 (1986)). The ability of PBDs to form an adduct in the minor groove enables them to interfere with DNA processing, hence their use as antitumor agents.

The biological activity of these molecules can be potentiated by joining two PBD units together through their C8/C′-hydroxyl functionalities via a flexible alkylene linker (Bose, D. S., et al., J. Am. Chem. Soc., 114, 4939-4941 (1992); Thurston, D. E., et al., J. Org. Chem., 61, 8141-8147 (1996)). The PBD dimers are thought to form sequence-selective DNA lesions such as the palindromic 5′-Pu-GATC-Py-3′ interstrand cross-link (Smellie, M., et al., Biochemistry, 42, 8232-8239 (2003); Martin, C., et al., Biochemistry, 44, 4135-4147) which is thought to be mainly responsible for their biological activity. One example of a PBD dimer is SG2000 (SJG-136):

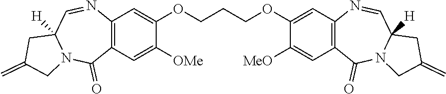

(Gregson, S., et al., J. Med. Chem., 44, 737-748 (2001); Alley, M. C., et al., Cancer Research, 64, 6700-6706 (2004); Hartley, J. A., et al., Cancer Research, 64, 6693-6699 (2004)).

Due to the manner in which these highly potent compounds act to cross-link DNA, previous PBD dimers have been made symmetrically, i.e., both monomers of the dimer are the same. This synthetic route provides for straightforward synthesis, either by constructing the PBD dimer moiety simultaneously having already formed the dimer linkage, or by reacting already constructed PBD monomer moieties with the dimer linking group. These synthetic approaches have limited the options for preparation of targeted conjugates containing PBDs. Due to the observed potency of PBD dimers, however, there exists a need for asymmetrical PBD dimers that are conjugatable to targeting agents for use in targeted therapy.

### 5. BRIEF SUMMARY

Provided herein are compounds of Formula (I):

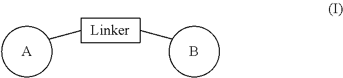

or a pharmaceutically acceptable salt, tautomer, solvate, or stereoisomer thereof,
   wherein each of ring A and ring B is, independently, one of the following formulas:

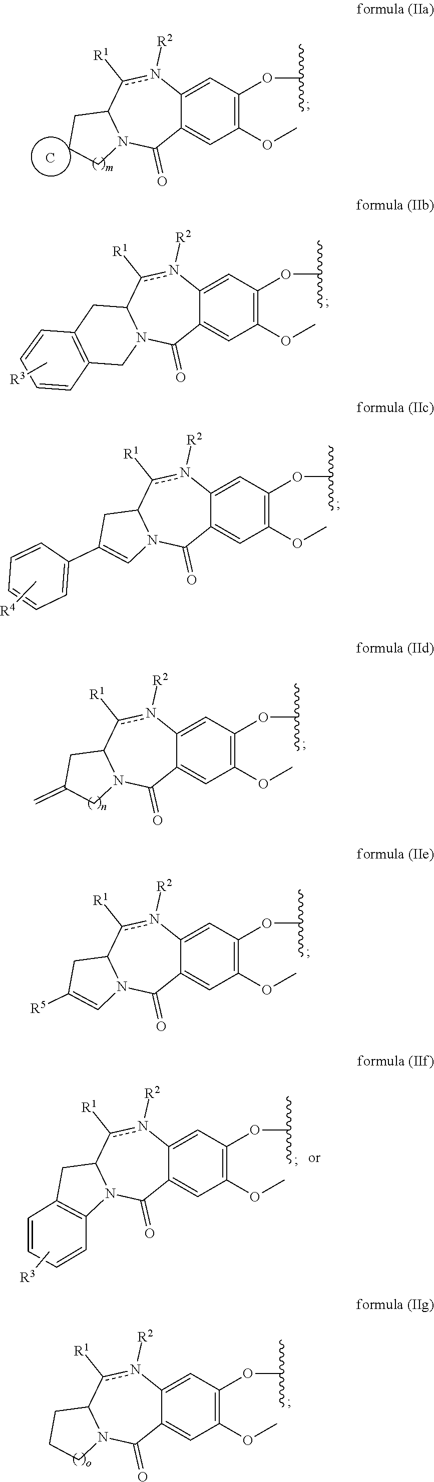

[symbol] indicates the point of attachment to Linker;
Linker is —(CH₂)ᵣ—, —(CH₂)ₚ—X—(CH₂)_{q}—, or —(CH₂)ₚ—CH═CH—(CH₂)_{q}—;
X is NR⁶, NHC(═O), C(═O)NH, O, SO₂, a substituted or unsubstituted aryl ring, substituted or unsubstituted heteroaryl ring, substituted or unsubstituted heterocyclic ring, or substituted or unsubstituted cyclic ring;
ring C is a cyclopropyl ring or a cyclobutyl ring;
each of the dotted bonds [symbol] between —C(R¹)— and —N(R²)— is, independently, a single bond or a double bond;
when the dotted bond is a single bond, each R¹ is, independently, H or OH, and each R² is H;
when the dotted bond is a double bond, each R¹ is H, and each R² is absent;
each of R³ and R⁴, independently, is H, NH₂, NR^{a}R^{b}, OH, C₁₋₄ alkyl, C₁₋₄ alkoxy, or aryl;
R^{a} and R^{b} are each independently H or C₁₋₄ alkyl;
R⁵ is H, C₁₋₄ alkyl, C₁₋₄ alkoxy, or aryl;
R⁶ is H, or C₁₋₄ alkyl;
each of m, n, and o is, independently, 1 or 2;
each of r, p, and q, is, independently, an integer from 1 to 8; and
the sum of p and q is an integer from 1 to 8.

Also provided herein are compounds of formula B(i) or B(ii):

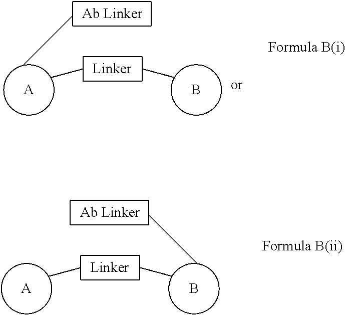

or a pharmaceutically acceptable salt, tautomer, solvate, or stereoisomer thereof, wherein:
each of ring A and ring B is, independently, one of the following formulas:

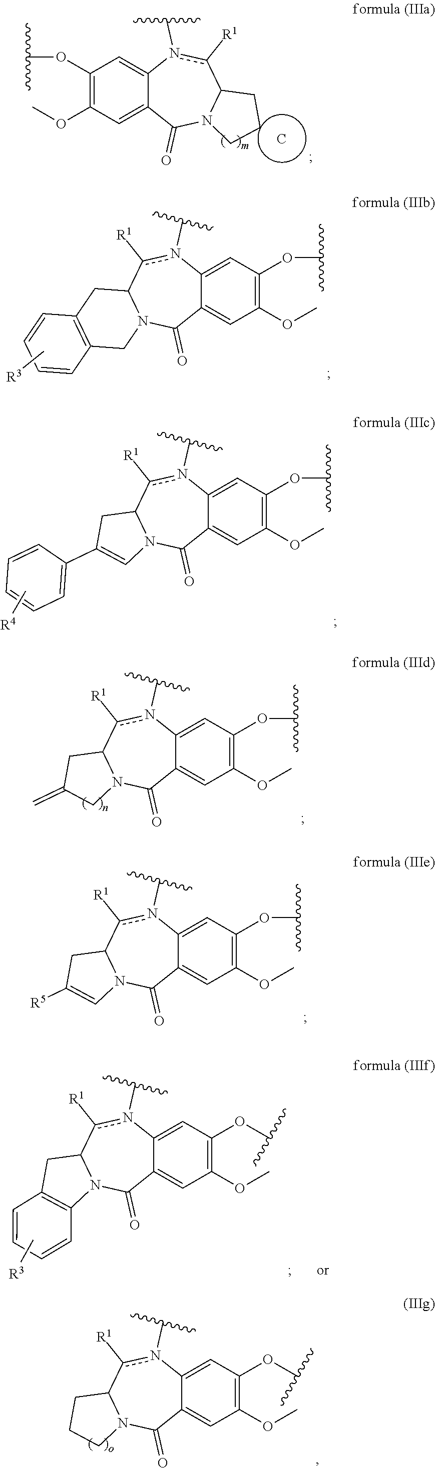

[symbol] indicates the point of attachment to Linker or Ab Linker;
Linker is —(CH₂)ᵣ—, —(CH₂)ₚ—X—(CH₂)_{q}—, or —(CH₂)ₚ—CH═CH—(CH₂)_{q}—;
X is NR⁶, NHC(═O), C(═O)NH, O, SO₂, a substituted or unsubstituted aryl ring, substituted or unsubstituted heteroaryl ring, substituted or unsubstituted heterocyclic ring, or substituted or unsubstituted cyclic ring;
ring C is a cyclopropyl ring or a cyclobutyl ring;
each of the dotted bonds [symbol] between —C(R¹)— and —N(R²)— is, independently, a single bond or a double bond;
when the dotted bond is a single bond, each R¹ is, independently, H or OH, and each R² is H;
when the dotted bond is a double bond, each R¹ is, independently, H, and each R² is absent;
each of R³ and R⁴ is, independently, H, NH₂, NaR^{b}, OH, C₁₋₄ alkyl, C₁₋₄ alkoxyl, or aryl;
R^{a} and R^{b} are each independently H or C₁₋₄ alkyl;
R⁵ is H, C₁₋₄ alkyl, C₁₋₄ alkoxyl, or aryl;
R⁶ is H or C₁₋₄ alkyl;
each of m, n, and o is, independently, 1 or 2;
each of r, p, and q, is, independently, an integer from 1 to 8;
the sum of p and q is an integer from 1 to 8; and
Ab Linker is a compound able to join ring A or ring B to a binding agent.

Also provided herein are conjugates of Formula A(i) or A(ii):

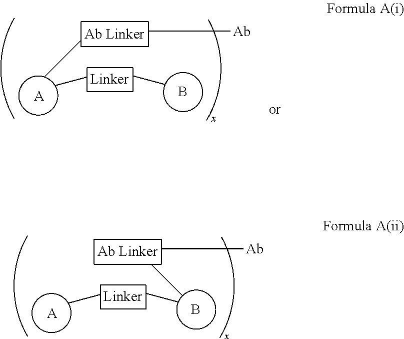

or a pharmaceutically acceptable salt, tautomer, solvate, or stereoisomer thereof, wherein:
each of ring A and ring B is, independently, one of the following formulas:

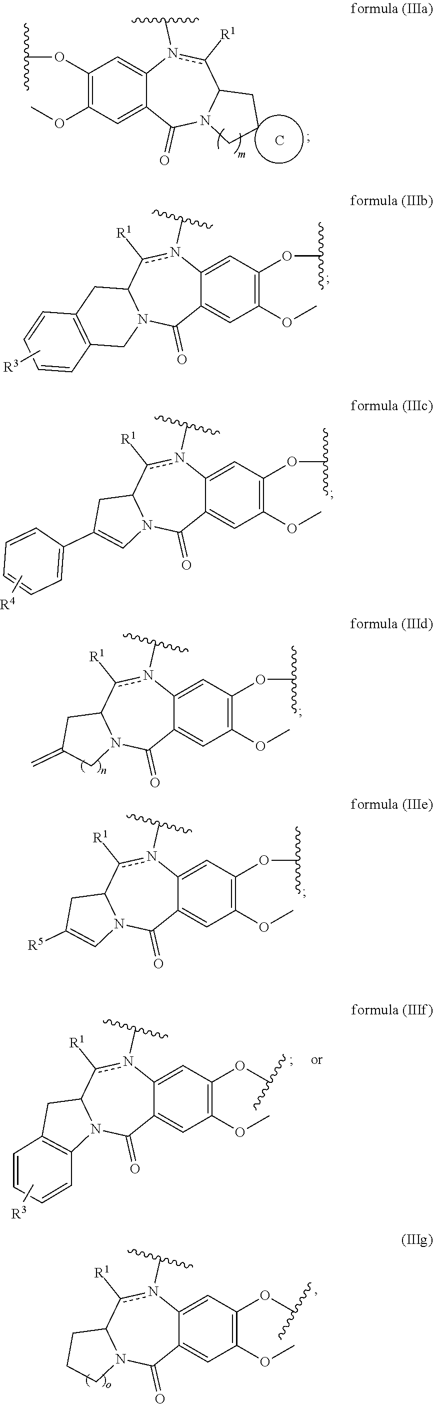

[symbol] indicates the point of attachment to Linker or Ab Linker;
Linker is —(CH₂)ᵣ—, —(CH₂)ₚ—X—(CH₂)_{q}—, or —(CH₂)ₚ—CH═CH—(CH₂)_{q}—;
X is NR⁶, NHC(═O), C(═O)NH, O, SO₂, a substituted or unsubstituted aryl ring, substituted or unsubstituted heteroaryl ring, substituted or unsubstituted heterocyclic ring, or substituted or unsubstituted cyclic ring;
ring C is a cyclopropyl ring or a cyclobutyl ring;
each of the dotted bonds between —C(R¹)— and —N(R²)— is, independently, a single bond or a double bond;
when the dotted bond is a single bond, each R¹ is, independently, H or OH, and each R² is, independently, H;
when the dotted bond is a double bond, each R¹ is, independently, H, and each R² is, independently, absent;
each of R³ and R⁴, independently, is H, NH₂, N^{a}R^{b}, OH, C₁₋₄ alkyl, C₁₋₄ alkoxyl, or aryl, and R^{a} and R^{b} are each independently H or C₁₋₄ alkyl;
R⁵ is H, C₁₋₄ alkyl, C₁₋₄ alkoxyl, or aryl;
R⁶ is H or C₁₋₄ alkyl;
each of m, n, and o is, independently, 1 or 2;
each of r, p, and q, is, independently, an integer from 1 to 8; and
the sum of p and q is an integer from 1 to 8;
Ab Linker is a compound that joins Ab to ring A or ring B;
Ab is a binding agent selected from a humanized, chimeric, or human antibody, or an antigen binding fragment thereof, and
subscript x is from 1 to 15.

### 6. BRIEF DESCRIPTION OF FIGURES

FIG. 1 is a line graph showing A375 cellular killing by compounds disclosed herein.

FIG. 2 is a line graph showing Calu-6 cellular killing by compounds disclosed herein.

FIG. 3 is a line graph showing A375 cellular killing by compounds disclosed herein.

FIG. 4 is a line graph showing Calu-6 cellular killing by compounds disclosed herein.

FIG. 5 is a line graph showing A375 cellular killing by compounds disclosed herein.

FIG. 6 is a line graph showing Calu-6 cellular killing by compounds disclosed herein.

FIG. 7 is a line graph showing A375 cellular killing by compounds disclosed herein.

FIG. 8 is a line graph showing Calu-6 cellular killing by compounds disclosed herein.

FIG. 9 is a line graph showing A375 cellular killing by compounds disclosed herein.

FIG. 10 is a line graph showing Calu-6 cellular killing by compounds disclosed herein.

FIG. 11 is a line graph showing A375 cellular killing by compounds disclosed herein.

FIG. 12 is a line graph showing Calu-6 cellular killing by compounds disclosed herein.

FIG. 13 is a line graph showing A375 cellular killing by compounds disclosed herein.

FIG. 14 is a line graph showing Calu-6 cellular killing by compounds disclosed herein.

### 7. DETAILED DESCRIPTION

Provided herein are pyrrolobenzodiazepine (PBD)-based compounds that include two PBDs, which may be the same or different, joined by a linker. The compounds may be used as the drug or payload portion of antibody drug conjugates (ADCs). The ADCs may be used to treat a disease or disorder, such as cancer, such as by providing a composition comprising an ADC.

### 7.1. Definitions

In the present disclosure, the following terms have the following meanings unless indicated otherwise. Unless defined otherwise, all technical and scientific terms used herein have the same meaning as is commonly understood by one of ordinary skill in the art to which this disclosure pertains. In the event that there is a plurality of definitions for a term provided herein, these Definitions prevail unless stated otherwise.

When a trade name is used herein, reference to the trade name also refers to the product formulation, the generic drug, and the active pharmaceutical ingredient(s) of the trade name product, unless otherwise indicated by context.

The term “antibody” herein is used in the broadest sense and specifically covers intact monoclonal antibodies, polyclonal antibodies, monospecific antibodies, multispecific antibodies (e.g., bispecific antibodies), and antibody fragments that exhibit the desired biological activity. An intact antibody has primarily two regions: a variable region and a constant region. The variable region binds to and interacts with a target antigen. The variable region includes a complementary determining region (CDR) that recognizes and binds to a specific binding site on a particular antigen. The constant region may be recognized by and interact with the immune system (see, e.g., Janeway et al., 2001, Immuno. Biology, 5th Ed., Garland Publishing, New York). An antibody can be of any type (e.g., IgG, IgE, IgM, IgD, and IgA), class (e.g., IgG1, IgG2, IgG3, IgG4, IgAQ1, and IgA2) or subclass. The antibody can be derived from any suitable species. In some embodiments, the antibody is of human or murine origin. An antibody can be, for example, human, humanized, or chimeric.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. The modifier “monoclonal” is not to be construed as requiring production of the antibody by any particular method.

An “intact antibody” is one that comprises an antigen-binding variable region as well as a light chain constant domain (CL) and heavy chain constant domains, CH1, CH2, CH3, and CH4, as appropriate for the antibody class. The constant domains may be native sequence constant domains (e.g., human native sequence constant domains) or amino acid sequence variant thereof.

An “antibody fragment” comprises a portion of an intact antibody, comprising the antigen-binding or variable region thereof. Examples of antibody fragments include Fab, Fab′, F(ab′)₂, and Fv fragments, diabodies, triabodies, tetrabodies, linear antibodies, single-chain antibody molecules, scFv, scFv-Fc, multispecific antibody fragments formed from antibody fragment(s), a fragment(s) produced by a Fab expression library, or an epitope-binding fragment of any of the above which immunospecifically binds to a target antigen (e.g., a cancer cell antigen, a viral antigen or a microbial antigen).

An “antigen” is an entity to which an antibody specifically binds.

The terms “specific binding” and “specifically binds” mean that the antibody or antibody derivative will bind, in a highly selective manner, to its corresponding target antigen and not with the multitude of other antigens. Typically, the antibody or antibody derivative binds with an affinity of at least about 1×10⁻⁷ M, 10⁻⁸ M, 10⁻⁹M, 10⁻¹⁰ M, 10⁻¹¹ M, or 10⁻¹² M and binds to the predetermined antigen with an affinity that is at least two-fold greater than its affinity for binding to a non-specific antigen (e.g., BSA, casein) other than the predetermined antigen or a closely related antigen.

The term “inhibit” or “inhibition of” means to reduce by a measurable amount, or to prevent entirely.

The term “therapeutically effective amount” refers to an amount of a drug effective to treat a disease or disorder in a mammal. In the case of cancer, the therapeutically effective amount of a drug may reduce the number of cancer cells; reduce the tumor size; inhibit (i.e., slow to some extent or stop) cancer cell infiltration into peripheral organs; inhibit (i.e., slow to some extent or stop) tumor metastasis; inhibit, to some extent, tumor growth; and/or relieve to some extent one or more of the symptoms associated with the cancer. To the extent the drug may inhibit growth and/or kill existing cancer cells, it may be cytostatic and/or cytotoxic. For cancer therapy, efficacy can, for example, be measured by assessing the time to disease progression (TTP) and/or determining the response rate (RR).

The term “substantial” or “substantially” refers to a majority, i.e. >50% of a population, of a mixture or a sample, preferably more than 50%, 55%, 60%, 65%, 70%, 75%, 80%, 85%, 90%, 91%, 92%, 93%, 94%, 95%, 96%, 97%, 98%, or 99% of a population.

The terms “intracellularly cleaved” and “intracellular cleavage” refer to a metabolic process or reaction inside a cell on a ligand drug conjugate (e.g., an antibody drug conjugate (ADC)), whereby the covalent attachment, e.g., the linker, between the drug moiety (D) and the ligand unit (e.g., an antibody (BA or Ab)) is broken, resulting in the free drug, or another metabolite of the conjugate dissociated from the antibody inside the cell. The cleaved moieties of the drug-linker-ligand conjugate are thus intracellular metabolites.

The terms “cancer” and “cancerous” refer to or describe the physiological condition or disorder in mammals that is typically characterized by unregulated cell growth. A “tumor” comprises one or more cancerous cells. Examples of cancer include, but are not limited to, carcinoma, lymphoma, blastoma, sarcoma, and leukemia or lymphoid malignancies. More particular examples of such cancers include squamous cell cancer (e.g., epithelial squamous cell cancer); lung cancer including small-cell lung cancer, non-small cell lung cancer (“NSCLC”), adenocarcinoma of the lung, and squamous carcinoma of the lung; cancer of the peritoneum; hepatocellular cancer; gastric or stomach cancer including gastrointestinal cancer; pancreatic cancer; glioblastoma; cervical cancer; ovarian cancer; liver cancer; bladder cancer; hepatoma; breast cancer; colon cancer; rectal cancer; colorectal cancer; endometrial or uterine carcinoma; salivary gland carcinoma; kidney or renal cancer; prostate cancer; vulval cancer; thyroid cancer; hepatic carcinoma; anal carcinoma; penile carcinoma; as well as head and neck cancer.

An “autoimmune disease” herein is a disease or disorder arising from and directed against an individual's own tissues or proteins.

Examples of a “patient” or “subject” include, but are not limited to, mammals such as a human, rat, mouse, guinea pig, monkey, pig, goat, cow, horse, dog, or cat, and birds or fowl. In an embodiment, the patient is a human.

The terms “treat” or “treatment,” unless otherwise indicated by context, refer to therapeutic treatment and prophylactic measures to prevent relapse, wherein the object is to inhibit or slow down (lessen) an undesired physiological change or disorder, such as the development or spread of cancer. For purposes of this disclosure, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder.

In the context of cancer, the term “treating” includes any or all of inhibiting growth of tumor cells, cancer cells, or of a tumor, inhibiting replication of tumor cells or cancer cells, lessening of overall tumor burden or decreasing the number of cancerous cells, and ameliorating one or more symptoms associated with the disease.

In the context of an autoimmune disease, the term “treating” includes any or all of inhibiting replication of cells associated with an autoimmune disease state including, but not limited to, cells that produce an autoimmune antibody, lessening the autoimmune-antibody burden, and ameliorating one or more symptoms of an autoimmune disease.

As used herein, and in the specification and the accompanying claims, the indefinite articles “a” and “an” and the definite article “the” include the plural as well as single referents, unless the context clearly indicates otherwise.

As used herein, and unless otherwise specified, the terms “about” and “approximately,” when used in connection with amounts, or weight percentage of ingredients of a composition, mean an amount or weight percent that is recognized by one of ordinary skill in the art to provide a pharmacological effect equivalent to that obtained from the specified amount or weight percent. In certain embodiments, the terms “about” and “approximately,” when used in this context, contemplate an amount or weight percent within 30%, within 20%, within 15%, within 10%, or within 5%, of the specified amount or weight percent.

As used herein, and unless otherwise specified, the terms “about” and “approximately,” when used in connection with a numeric value or range of values that is provided to characterize a particular solid form, e.g., a specific temperature or temperature range, such as, for example, that describes a melting, dehydration, desolvation, or glass transition temperature; a mass change, such as, for example, a mass change as a function of temperature or humidity; a solvent or water content, in terms of, for example, mass or a percentage; or a peak position, such as, for example, in analysis by, for example, IR or Raman spectroscopy or XRPD; indicate that the value or range of values may deviate to an extent deemed reasonable to one of ordinary skill in the art while still describing the solid form. Techniques for characterizing crystal forms and amorphous solids include, but are not limited to, thermal gravimetric analysis (TGA), differential scanning calorimetry (DSC), X-ray powder diffractometry (XRPD), single-crystal X-ray diffractometry, vibrational spectroscopy, e.g., infrared (TR) and Raman spectroscopy, solid-state and solution nuclear magnetic resonance (NMR) spectroscopy, optical microscopy, hot stage optical microscopy, scanning electron microscopy (SEM), electron crystallography and quantitative analysis, particle size analysis (PSA), surface area analysis, solubility studies, and dissolution studies. In certain embodiments, the terms “about” and “approximately,” when used in this context, indicate that the numeric value or range of values may vary within 30%, 20%, 15%, 10%, 9%, 8%, 7%, 6%, 5%, 4%, 3%, 2%, 1.5% I, %, 0.5%, or 0.25% of the recited value or range of values. For example, in some embodiments, the value of an XRPD peak position may vary by up to ±0.2° 20 while still describing the particular XRPD peak.

An “alkyl” group is a saturated, partially saturated, or unsaturated straight chain or branched non-cyclic hydrocarbon having from 1 to 10 carbon atoms, typically from 1 to 8 carbons or, in some embodiments, from 1 to 6, 1 to 4, or 2 to 6 carbon atoms. Representative alkyl groups include -methyl, -ethyl, -n-propyl, -n-butyl, -n-pentyl, and n-hexyl; saturated branched alkyls include -isopropyl, -sec-butyl, -isobutyl, -tert-butyl, -isopentyl, 2-methylpentyl, 3-methylpentyl, 4-methylpentyl, 2,3-dimethylbutyl and the like. Examples of unsaturated alkyl groups include, but are not limited to, vinyl, allyl, CH═CH(CH₃), —CH═C(CH₃)₂, —C(CH₃)=CH₂, —C(CH₃)═CH(CH₃), C(CH₂CH₃)=CH₂, C≡CH, —C≡C(CH₃), —C≡C(CH₂CH₃), —CH₂C≡CH, —CH₂C≡C(CH₃), and CH₂C≡C(CH₂CH₃), among others. An alkyl group can be substituted or unsubstituted. In certain embodiments, when the alkyl groups described herein are said to be “substituted,” they may be substituted with any substituent or substituents as those found in the compounds and embodiments disclosed herein, as well as halogen (chloro, iodo, bromo, or fluoro); hydroxyl; alkoxy; alkoxyalkyl; amino; alkylamino; carboxy; nitro; cyano; thiol; thioether; imine; imide; amidine; guanidine; enamine; aminocarbonyl; acylamino; phosphonato; phosphine; thiocarbonyl; sulfonyl; sulfone; sulfonamide; ketone; aldehyde; ester; urea; urethane; oxime; hydroxyl amine; alkoxyamine; aralkoxyamine; N-oxide; hydrazine; hydrazide; hydrazone; azide; isocyanate; isothiocyanate; cyanate; thiocyanate; B(OH)₂; or O(alkyl)aminocarbonyl.

An “alkenyl” group is a straight chain or branched non-cyclic hydrocarbon having from 2 to 10 carbon atoms, typically from 2 to 8 carbon atoms, and including at least one carbon-carbon double bond. Representative straight chain and branched (C₂-C₈)alkenyls include -vinyl, -allyl, -1-butenyl, -2-butenyl, -isobutylenyl, -1-pentenyl, -2-pentenyl, -3-methyl-1-butenyl, -2-methyl-2-butenyl, -2,3-dimethyl-2-butenyl, -1-hexenyl, 2-hexenyl, -3-hexenyl, -1-heptenyl, -2-heptenyl, -3-heptenyl, -1-octenyl, -2-octenyl, 3-octenyl and the like. The double bond of an alkenyl group can be unconjugated or conjugated to another unsaturated group. An alkenyl group can be unsubstituted or substituted.

A “cycloalkyl” group is a saturated or a partially saturated cyclic alkyl group of from 3 to 10 carbon atoms having a single cyclic ring or multiple condensed or bridged rings which can be optionally substituted with from 1 to 3 alkyl groups. In some embodiments, the cycloalkyl group has 3 to 8 ring members, whereas in other embodiments the number of ring carbon atoms ranges from 3 to 5, 3 to 6, or 3 to 7. Such cycloalkyl groups include, by way of example, single ring structures such as cyclopropyl, cyclobutyl, cyclopentyl, cyclohexyl, cycloheptyl, cyclooctyl, 1-methylcyclopropyl, 2-methylcyclopentyl, 2-methylcyclooctyl, and the like, or multiple or bridged ring structures such as adamantyl and the like. Examples of unsaturated cycloalkyl groups include cyclohexenyl, cyclopentenyl, cyclohexadienyl, butadienyl, pentadienyl, and hexadienyl, among others. A cycloalkyl group can be substituted or unsubstituted. Such substituted cycloalkyl groups include, by way of example, cyclohexanone and the like.

An “aryl” group is an aromatic carbocyclic group of from 6 to 14 carbon atoms having a single ring (e.g., phenyl) or multiple condensed rings (e.g., naphthyl or anthryl). In some embodiments, aryl groups contain 6 to 14 carbons, and in others from 6 to 12 or even 6 to 10 carbon atoms in the ring portions of the groups. Particular aryls include phenyl, biphenyl, naphthyl and the like. An aryl group can be substituted or unsubstituted. The phrase “aryl groups” also includes groups containing fused rings, such as fused aromatic-aliphatic ring systems (e.g., indanyl, tetrahydronaphthyl, and the like).

An “arylene” group is a bivalent aryl group as defined herein.

A “heteroaryl” group is an aryl ring system having one to four heteroatoms as ring atoms in a heteroaromatic ring system, wherein the remainder of the atoms are carbon atoms. In some embodiments, heteroaryl groups contain 5 to 6 ring atoms, and in others from 6 to 9 or 6 to 10 atoms in the ring portions of the groups. Suitable heteroatoms include oxygen, sulfur, and nitrogen. In certain embodiments, the heteroaryl ring system is monocyclic or bicyclic. Non-limiting examples include, but are not limited to, groups such as pyrrolyl, pyrazolyl, imidazolyl, triazolyl, tetrazolyl, oxazolyl, isoxazolyl, thiazolyl, pyrrolyl, pyridyl, pyridazinyl, pyrimidinyl, pyrazinyl, thiophenyl, benzothiophenyl, furanyl, benzofuranyl (for example, isobenzofuran-1,3-diimine), indolyl, azaindolyl (for example, pyrrolopyridyl or 1H-pyrrolo[2,3-b]pyridyl), indazolyl, benzimidazolyl (for example, 1H-benzo[d]imidazolyl), imidazopyridyl (for example, azabenzimidazolyl, 3H-imidazo[4,5-b]pyridyl or 1H-imidazo[4,5-b]pyridyl), pyrazolopyridyl, triazolopyridyl, benzotriazolyl, benzoxazolyl, benzothiazolyl, benzothiadiazolyl, isoxazolopyridyl, thianaphthalenyl, purinyl, xanthinyl, adeninyl, guaninyl, quinolinyl, isoquinolinyl, tetrahydroquinolinyl, quinoxalinyl, and quinazolinyl groups.

A “heteroarylene” group is a bivalent heteroaryl group as defined herein.

A “heterocyclyl” is an aromatic (also referred to as heteroaryl) or non-aromatic cycloalkyl in which one to four of the ring carbon atoms are independently replaced with a heteroatom from the group consisting of O, S and N. In some embodiments, heterocyclyl groups include 3 to 10 ring members, whereas other such groups have 3 to 5, 3 to 6, or 3 to 8 ring members. Heterocyclyls can also be bonded to other groups at any ring atom (i.e., at any carbon atom or heteroatom of the heterocyclic ring). A heterocyclyl group can be substituted or unsubstituted. Heterocyclyl groups encompass unsaturated, partially saturated, and saturated ring systems, such as, for example, imidazolyl, imidazolinyl, and imidazolidinyl groups. The term “heterocyclyl” includes fused ring species, including those comprising fused aromatic and non-aromatic groups, such as, for example, benzotriazolyl, 2,3-dihydrobenzo[1,4]dioxinyl, and benzo[1,3]dioxolyl. The term also includes bridged polycyclic ring systems containing a heteroatom such as, but not limited to, quinuclidyl. Representative examples of a heterocyclyl group include, but are not limited to, aziridinyl, azetidinyl, pyrrolidyl, imidazolidinyl, pyrazolidinyl, thiazolidinyl, tetrahydrothiophenyl, tetrahydrofuranyl, dioxolyl, furanyl, thiophenyl, pyrrolyl, pyrrolinyl, imidazolyl, imidazolinyl, pyrazolyl, pyrazolinyl, triazolyl, tetrazolyl, oxazolyl, isoxazolyl, thiazolyl, thiazolinyl, isothiazolyl, thiadiazolyl, oxadiazolyl, piperidyl, piperazinyl, morpholinyl, thiomorpholinyl, tetrahydropyranyl (for example, tetrahydro-2H-pyranyl), tetrahydrothiopyranyl, oxathiane, dioxyl, dithianyl, pyranyl, pyridyl, pyrimidinyl, pyridazinyl, pyrazinyl, triazinyl, dihydropyridyl, dihydrodithiinyl, dihydrodithionyl, homopiperazinyl, quinuclidyl, indolyl, indolinyl, isoindolyl, azaindolyl (pyrrolopyridyl), indazolyl, indolizinyl, benzotriazolyl, benzimidazolyl, benzofuranyl, benzothiophenyl, benzthiazolyl, benzoxadiazolyl, benzoxazinyl, benzodithiinyl, benzoxathiinyl, benzothiazinyl, benzoxazolyl, benzothiazolyl, benzothiadiazolyl, benzo[1,3]dioxolyl, pyrazolopyridyl, imidazopyridyl (azabenzimidazolyl; for example, 1H-imidazo[4,5-b]pyridyl, or 1H-imidazo[4,5-b]pyridin-2(3H)-onyl), triazolopyridyl, isoxazolopyridyl, purinyl, xanthinyl, adeninyl, guaninyl, quinolinyl, isoquinolinyl, quinolizinyl, quinoxalinyl, quinazolinyl, cinnolinyl, phthalazinyl, naphthyridinyl, pteridinyl, thianaphthalenyl, dihydrobenzothiazinyl, dihydrobenzofuranyl, dihydroindolyl, dihydrobenzodioxinyl, tetrahydroindolyl, tetrahydroindazolyl, tetrahydrobenzimidazolyl, tetrahydrobenzotriazolyl, tetrahydropyrrolopyridyl, tetrahydropyrazolopyridyl, tetrahydroimidazopyridyl, tetrahydrotriazolopyridyl, and tetrahydroquinolinyl groups. Representative substituted heterocyclyl groups may be mono-substituted or substituted more than once, such as, but not limited to, pyridyl or morpholinyl groups, which are 2-, 3-, 4-, 5-, or 6-substituted, or disubstituted with various substituents such as those listed below.

A “cycloalkylalkyl” group is a radical of the formula -alkyl-cycloalkyl, wherein alkyl and cycloalkyl are defined above. Substituted cycloalkylalkyl groups may be substituted at the alkyl, the cycloalkyl, or both the alkyl and the cycloalkyl portions of the group. Representative cycloalkylalkyl groups include but are not limited to cyclopentylmethyl, cyclopentylethyl, cyclohexylmethyl, cyclohexylethyl, and cyclohexylpropyl. Representative substituted cycloalkylalkyl groups may be mono-substituted or substituted more than once.

An “aralkyl” group is a radical of the formula -alkyl-aryl, wherein alkyl and aryl are defined above. Substituted aralkyl groups may be substituted at the alkyl, the aryl, or both the alkyl and the aryl portions of the group. Representative aralkyl groups include, but are not limited to, benzyl and phenethyl groups and fused (cycloalkylaryl)alkyl groups such as 4-ethyl-indanyl.

A “heterocyclylalkyl” group is a radical of the formula -alkyl-heterocyclyl, wherein alkyl and heterocyclyl are defined above. Substituted heterocyclylalkyl groups may be substituted at the alkyl, the heterocyclyl, or both the alkyl and the heterocyclyl portions of the group. Representative heterocyclylalkyl groups include, but are not limited to, 4-ethyl-morpholinyl, 4-propylmorpholinyl, furan-2-yl methyl, furan-3-yl methyl, pyrdine-3-yl methyl, (tetrahydro-2H-pyran-4-yl)methyl, (tetrahydro-2H-pyran-4-yl)ethyl, tetrahydrofuran-2-yl methyl, tetrahydrofuran-2-yl ethyl, and indol-2-yl propyl.

A “halogen” is chloro, iodo, bromo, or fluoro.

A “hydroxyalkyl” group is an alkyl group as described above substituted with one or more hydroxy groups.

An “alkoxy” group is O(alkyl), wherein alkyl is defined above.

An “alkoxyalkyl” group is (alkyl)O(alkyl), wherein alkyl is defined above.

As used herein, “alkynyl” refers to a monovalent hydrocarbon radical moiety containing at least two carbon atoms and one or more carbon-carbon triple bonds. Alkynyl is optionally substituted and can be linear, branched, or cyclic. Alkynyl includes, but is not limited to, those radicals having 2-20 carbon atoms, i.e., C₂₋₂₀ alkynyl; 2-12 carbon atoms, i.e., C₂₋₁₂ alkynyl; 2-8 carbon atoms, i.e., C₂₋₈ alkynyl; 2-6 carbon atoms, i.e., C₂₋₆ alkynyl; and 2-4 carbon atoms, i.e., C₂₋₄ alkynyl. Examples of alkynyl moieties include, but are not limited to ethynyl, propynyl, and butynyl.

As used herein, “haloalkyl” refers to alkyl, as defined above, wherein the alkyl includes at least one substituent selected from a halogen, for example, fluorine (F), chlorine (Cl), bromine (Br), or iodine (I). Examples of haloalkyl include, but are not limited to, —CF₃, —CH₂CF₃, —CCl₂F, and —CCl₃.

As used herein, “haloalkoxy” refers to alkoxy, as defined above, wherein the alkoxy includes at least one substituent selected from a halogen, e.g., F, Cl, Br, or I.

As used herein, “arylalkyl” refers to a monovalent moiety that is a radical of an alkyl compound, wherein the alkyl compound is substituted with an aromatic substituent, i.e., the aromatic compound includes a single bond to an alkyl group and wherein the radical is localized on the alkyl group. An arylalkyl group bonds to the illustrated chemical structure via the alkyl group. An arylalkyl can be represented by the structure, e.g., B—CH₂—, B—CH₂—CH₂—, B—CH₂—CH₂—CH₂—, B—CH₂—CH₂—CH₂—CH₂—, B—CH(CH₃)—CH₂—CH₂—, B—CH₂—CH(CH₃)—CH₂—, wherein B is an aromatic moiety, e.g., phenyl. Arylalkyl is optionally substituted, i.e., the aryl group and/or the alkyl group, can be substituted as disclosed herein. Examples of arylalkyl include, but are not limited to, benzyl.

As used herein, “alkylaryl” refers to a monovalent moiety that is a radical of an aryl compound, wherein the aryl compound is substituted with an alkyl substituent, i.e., the aryl compound includes a single bond to an alkyl group and wherein the radical is localized on the aryl group. An alkylaryl group bonds to the illustrated chemical structure via the aryl group. An alkylaryl can be represented by the structure, e.g., —B—CH₃, —B—CH₂—CH₃, —B—CH₂—CH₂—CH₃, —B—CH₂—CH₂—CH₂—CH₃, —B—CH(CH₃)—CH₂—CH₃, —B—CH₂—CH(CH₃)—CH₃, wherein B is an aromatic moiety, e.g., phenyl. Alkylaryl is optionally substituted, i.e., the aryl group and/or the alkyl group, can be substituted as disclosed herein. Examples of alkylaryl include, but are not limited to, toluyl.

As used herein, “aryloxy” refers to a monovalent moiety that is a radical of an aromatic compound wherein the ring atoms are carbon atoms and wherein the ring is substituted with an oxygen radical, i.e., the aromatic compound includes a single bond to an oxygen atom and wherein the radical is localized on the oxygen atom, e.g., C₆H₅—O—, for phenoxy. Aryloxy substituents bond to the compound which they substitute through this oxygen atom. Aryloxy is optionally substituted. Aryloxy includes, but is not limited to, those radicals having 6 to 20 ring carbon atoms, i.e., C₆₋₂₀ aryloxy; 6 to 15 ring carbon atoms, i.e., C₆₋₁₅ aryloxy, and 6 to 10 ring carbon atoms, i.e., C₆₋₁₀ aryloxy. Examples of aryloxy moieties include, but are not limited to phenoxy, naphthoxy, and anthroxy.

An “amino” group is a radical of the formula NH₂.

A “hydroxyl amine” group is a radical of the formula N(R^{#})OH or NHOH, wherein R^{#} is a substituted or unsubstituted alkyl, cycloalkyl, cycloalkylalkyl, aryl, aralkyl, heterocyclyl or heterocyclylalkyl group as defined herein.

An “alkoxyamine” group is a radical of the formula —N(R^{#})O-alkyl or —NHO-alkyl, wherein R^{#} is as defined above.

An “aralkoxyamine” group is a radical of the formula N(R^{#})O-aryl or NHOaryl, wherein R^{#} is as defined above.

An “alkylamine” group is a radical of the formula NHalkyl or N(alkyl)₂, wherein each alkyl is independently as defined above.

An “aminocarbonyl” group is a radical of the formula —C(═O)N(R^{#})₂, —C(═O)NH(R^{#}), or C(═O)NH₂, wherein each R^{#} is as defined above.

An “acylamino” group is a radical of the formula NHC(═O)(R^{#}) or N(alkyl)C(═O)(R^{#}), wherein each alkyl and R^{#} are independently as defined above.

An “O(alkyl)aminocarbonyl” group is a radical of the formula —O(alkyl)C(═O)N(R^{#})₂, —O(alkyl)C(═O)NH(R^{#}), or —O(alkyl)C(═O)NH₂, wherein each R^{#} is independently as defined above.

An “N-oxide” group is a radical of the formula —N⁺—O⁻.

A “carboxy” group is a radical of the formula C(═O)OH.

A “ketone” group is a radical of the formula C(═O)(R^{#}), wherein R^{#} is as defined above.

An “aldehyde” group is a radical of the formula —CH(═O).

An “ester” group is a radical of the formula C(═O)O(R^{#}) or OC(═O)(R^{#}), wherein R^{#} is as defined above.

A “urea” group is a radical of the formula —N(alkyl)C(═O)N(R⁴)₂, —N(alkyl)C(═O)NH(R^{#}), —N(alkyl)C(═O)NH₂, —NHC(═O)N(R^{#})₂, —NHC(═O)NH(R^{#}), or NHC(═O)NH₂^{#}, wherein each alkyl and R^{#} are independently as defined above.

An “imine” group is a radical of the formula —N═C(R^{#})₂ or —C(R^{#})═N(R^{#}), wherein each R^{#} is independently as defined above.

An “imide” group is a radical of the formula —C(═O)N(R^{#})C(═O)(R^{#}) or N((C═O)(R^{#}))₂, wherein each R^{#} is independently as defined above.

A “urethane” group is a radical of the formula —OC(═O)N(R^{#})₂, —OC(═O)NH(R^{#}), —N(R^{#})C(═O)O(R^{#}), or —NHC(═O)O(R^{#}), wherein each R^{#} is independently as defined above.

An “amidine” group is a radical of the formula —C(═N(R^{#}))N(R^{#})₂, —C(═N(R^{#}))NH(R^{#}), —C(═N(R^{#}))NH₂, —C(═NH)N(R^{#})₂, —C(═NH)NH(R^{#}), —C(═NH)NH₂, —N═C(R^{#})N(R^{#})₂, —N═C(R^{#})NH(R^{#}), —N═C(R^{#})NH₂, —N(R^{#})C(R^{#})═N(R^{#}), —NHC(R^{#})═N(R^{#}), —N(R)C(R)═NH, or —NHC(R^{#})=NH, wherein each R^{#} is independently as defined above.

A “guanidine” group is a radical of the formula —N(R^{#})C(═N(R^{#}))N(R^{#})₂, —NHC(═N(R^{#}))N(R^{#})₂, —N(R^{#})C(═NH)N(R^{#})₂, —N(R^{#})C(═N(R^{#}))NH(R^{#}), —N(R^{#})C(═N(R^{#}))NH₂, —NHC(═NH)N(R^{#})₂, —NHC(═N(R^{#}))NH(R^{#}), —NHC(═N(R^{#}))NH₂, —NHC(═NH)NH(R^{#}), —NHC(═NH)NH₂, —N═C(N(R^{#})₂)₂, —N═C(NH(R^{#}))₂, or —N═C(NH₂)₂, wherein each R^{#} is independently as defined above.

An “enamine” group is a radical of the formula —N(R^{#})C(R^{#})═C(R^{#})₂, —NHC(R^{#})═C(R^{#})₂, —C(N(R^{#})₂)═C(R^{#})₂, —C(NH(R^{#}))═C(R^{#})₂, —C(NH₂)═C(R⁴)₂, —C(R^{#})═C(R^{#})(N(R^{#})₂), C(R^{#})═C(R^{#})(NH(R^{#})) or —C(R^{#})═C(R^{#})(NH₂), wherein each R^{#} is independently as defined above.

An “oxime” group is a radical of the formula —C(═NO(R^{#}))(R^{#}), —C(═NOH)(R^{#}), —CH(═NO(R^{#})), or —CH(═NOH), wherein each R^{#} is independently as defined above.

A “hydrazide” group is a radical of the formula —C(═O)N(R^{#})N(R^{#})₂, —C(═O)NHN(R^{#})₂, —C(═O)N(R^{#})NH(R^{#}), —C(═O)N(R^{#})NH₂, —C(═O)NHNH(R^{#})₂, or —C(═O)NHNH₂, wherein each R^{#} is independently as defined above.

A “hydrazine” group is a radical of the formula —N(R)N(R^{#})₂, —NHN(R^{#})₂, —N(R^{#})NH(R^{#}), —N(R^{#})NH₂, —NHNH(R^{#})₂, or —NHNH₂, wherein each R is independently as defined above.

A “hydrazone” group is a radical of the formula —C(═N—N(R^{#})₂)(R^{#})₂, —C(═NNH(R^{#}))(R^{#})₂, —C(═N—NH₂)(R^{#})₂, —N(R^{#})(N═C(R^{#})₂), or —NH(N═C(R^{#})₂), wherein each R⁴ is independently as defined above.

An “azide” group is a radical of the formula —N₃.

An “isocyanate” group is a radical of the formula N═C═O.

An “isothiocyanate” group is a radical of the formula N═C═S.

A “cyanate” group is a radical of the formula OCN.

A “thiocyanate” group is a radical of the formula SCN.

A “thioether” group is a radical of the formula —S(R^{#}), wherein R^{#} is as defined above.

A “thiocarbonyl” group is a radical of the formula —C(═S)(R^{#}), wherein R^{#} is as defined above.

A “sulfinyl” group is a radical of the formula —S(═O)(R^{#}), wherein R^{#} is as defined above.

A “sulfone” group is a radical of the formula —S(═O)₂(R^{#}), wherein R^{#} is as defined above.

A “sulfonylamino” group is a radical of the formula —NHSO₂(R^{#}) or —N(alkyl)SO₂(R^{#}), wherein each alkyl and R^{#} are defined above.

A “sulfonamide” group is a radical of the formula —S(═O)₂N(R^{#})₂, or —S(═O)₂NH(R^{#}), or —S(═O)₂NH₂, wherein each R^{#} is independently as defined above.

A “phosphonate” group is a radical of the formula —P(═O)(O(R^{#}))₂, —P(═O)(OH)₂, —OP(═O)(O(R^{#}))(R^{#}), or —OP(═O)(OH)(R^{#}), wherein each R^{#} is independently as defined above.

A “phosphine” group is a radical of the formula —P(R^{#})₂, wherein each R^{#} is independently as defined above.

When the groups described herein, with the exception of alkyl groups, are said to be “substituted,” they may be substituted with any appropriate substituent or substituents. Illustrative examples of substituents are those found in the compounds and embodiments disclosed herein, as well as halogen (chloro, iodo, bromo, or fluoro); alkyl; hydroxyl; alkoxy; alkoxyalkyl; amino; alkylamino; carboxy; nitro; cyano; thiol; thioether; imine; imide; amidine; guanidine; enamine; aminocarbonyl; acylamino; phosphonate; phosphine; thiocarbonyl; sulfinyl; sulfone; sulfonamide; ketone; aldehyde; ester; urea; urethane; oxime; hydroxyl amine; alkoxyamine; aralkoxyamine; N-oxide; hydrazine; hydrazide; hydrazone; azide; isocyanate; isothiocyanate; cyanate; thiocyanate; oxygen (═O); B(OH)₂, O(alkyl)aminocarbonyl; cycloalkyl, which may be monocyclic or fused or non-fused polycyclic (e.g., cyclopropyl, cyclobutyl, cyclopentyl, or cyclohexyl), or a heterocyclyl, which may be monocyclic or fused or non-fused polycyclic (e.g., pyrrolidyl, piperidyl, piperazinyl, morpholinyl, or thiazinyl); monocyclic or fused or non-fused polycyclic aryl or heteroaryl (e.g., phenyl, naphthyl, pyrrolyl, indolyl, furanyl, thiophenyl, imidazolyl, oxazolyl, isoxazolyl, thiazolyl, triazolyl, tetrazolyl, pyrazolyl, pyridinyl, quinolinyl, isoquinolinyl, acridinyl, pyrazinyl, pyridazinyl, pyrimidinyl, benzimidazolyl, benzothiophenyl, or benzofuranyl) aryloxy; aralkyloxy; heterocyclyloxy; and heterocyclyl alkoxy.

As used herein, the term “pharmaceutically acceptable salt(s)” refers to a salt prepared from a pharmaceutically acceptable non-toxic acid or base including an inorganic acid or base and an organic acid or base.

As used herein and unless otherwise indicated, the term “solvate” means a compound, or a salt thereof, that further includes a stoichiometric or non-stoichiometric amount of a solvent bound by non-covalent intermolecular forces. In one embodiment, the solvate is a hydrate.

As used herein and unless otherwise indicated, the term “hydrate” means a compound, or a salt thereof, that further includes a stoichiometric or non-stoichiometric amount of water bound by non-covalent intermolecular forces.

As used herein and unless otherwise indicated, the term “prodrug” means a compound derivative that can hydrolyze, oxidize, or otherwise react under biological conditions (in vitro or in vivo) to provide an active compound. Examples of prodrugs include, but are not limited to, derivatives and metabolites of a compound that include biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. In certain embodiments, prodrugs of compounds with carboxyl functional groups are the lower alkyl esters of the carboxylic acid. The carboxylate esters may be formed by esterifying any of the carboxylic acid moieties present on the molecule. Prodrugs can typically be prepared using well-known methods, such as those described by Burger's Medicinal Chemistry andDrug Discovery 6^{th} ed. (Donald J. Abraham ed., 2001, Wiley) and Design and Application of Prodrugs (H. Bundgaard ed., 1985, Harwood Academic Publishers Gmfh).

As used herein and unless otherwise indicated, the term “stereoisomer” or “stereomerically pure” means one stereoisomer of a compound that is substantially free of other stereoisomers of that compound. For example, a stereomerically pure compound having one chiral center will be substantially free of the opposite enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, greater than about 90% by weight of one stereoisomer of the compound and less than about 10% by weight of the other stereoisomers of the compound, greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound. The compounds can have chiral centers and can occur as racemates, individual enantiomers or diastereomers, and mixtures thereof. All such isomeric forms are included within the embodiments disclosed herein, including mixtures thereof. The use of stereomerically pure forms of such compounds, as well as the use of mixtures of those forms, are encompassed by the embodiments disclosed herein. For example, mixtures comprising equal or unequal amounts of the enantiomers of a particular compound may be used in methods and compositions disclosed herein. These isomers may be asymmetrically synthesized or resolved using standard techniques such as chiral columns or chiral resolving agents. See, e.g., Jacques, J., et al., Enantiomers, Racemates and Resolutions (WileyInterscience, New York, 1981); Wilen, S. H., et al., Tetrahedron 33:2725 (1977); Eliel, E. L., Stereochemistry of Carbon Compounds (McGrawHill, NY, 1962); and Wilen, S. H., Tables of Resolving Agents and Optical Resolutions p. 268 (E. L. Eliel, Ed., Univ. of Notre Dame Press, Notre Dame, IN, 1972).

It should also be noted that the compounds can include E and Z isomers, or a mixture thereof, and cis and trans isomers, or a mixture thereof. In certain embodiments, the compounds are isolated as either the cis or trans isomer. In other embodiments, the compounds are a mixture of the cis and trans isomers.

“Tautomers” refers to isomeric forms of a compound that are in equilibrium with each other. The concentrations of the isomeric forms will depend on the environment the compound is found in and may be different depending upon, for example, whether the compound is a solid or is in an organic or aqueous solution. For example, in an aqueous solution, pyrazoles may exhibit the following isomeric forms, which are referred to as tautomers of each other:

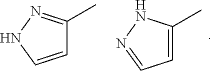

As readily understood by one skilled in the art, a wide variety of functional groups and other structures may exhibit tautomerism and all tautomers of the compounds are within the scope of the present disclosure.

It should also be noted the compounds can contain unnatural proportions of atomic isotopes at one or more of the atoms. For example, the compounds may be radiolabeled with radioactive isotopes, such as for example tritium (³H), iodine-125 (¹²⁵I), sulfur-35 (³⁵S), or carbon-14 (¹⁴C), or may be isotopically enriched, such as with deuterium (²H), carbon-13 (¹³C), or nitrogen-15 (¹⁵N). As used herein, an “isotopologue” is an isotopically enriched compound. The term “isotopically enriched” refers to an atom having an isotopic composition other than the natural isotopic composition of that atom. “Isotopically enriched” may also refer to a compound containing at least one atom having an isotopic composition other than the natural isotopic composition of that atom. The term “isotopic composition” refers to the amount of each isotope present for a given atom. Radiolabeled and isotopically enriched compounds are useful as therapeutic agents, e.g., cancer and inflammation therapeutic agents, research reagents, e.g., binding assay reagents, and diagnostic agents, e.g., in vivo imaging agents. All isotopic variations of the compounds as described herein, whether radioactive or not, are intended to be encompassed within the scope of the embodiments provided herein. In some embodiments, there are provided isotopologues of the compounds, for example, the isotopologues are deuterium, carbon-13, or nitrogen-15 enriched compounds.

It should be noted that if there is a discrepancy between a depicted structure and a name for that structure, the depicted structure is to be accorded more weight.

As used herein, the term “residue” refers to the chemical moiety within a compound that remains after a chemical reaction. For example, the term “amino acid residue” or “N-alkyl amino acid residue” refers to the product of an amide coupling or peptide coupling of an amino acid or a N-alkyl amino acid to a suitable coupling partner; wherein, for example, a water molecule is expelled after the amide or peptide coupling of the amino acid or the N-alkylamino acid, resulting in the product having the amino acid residue or N-alkyl amino acid residue incorporated therein.

As used herein, “sugar” or “sugar group” or “sugar residue” refers to a carbohydrate moiety which may comprise 3-carbon (those) units, 4-carbon (tetrose) units, 5-carbon (pentose) units, 6-carbon (hexose) units, 7-carbon (heptose) units, or combinations thereof, and may be a monosaccharide, a disaccharide, a trisaccharide, a tetrasaccharide, a pentasaccharide, an oligosaccharide, or any other polysaccharide. In some instances, a “sugar” or “sugar group” or “sugar residue” comprises furanoses (e.g., ribofuranose, fructofuranose) or pyranoses (e.g., glucopyranose, galactopyranose), or a combination thereof. In some instances, a “sugar” or “sugar group” or “sugar residue” comprises aldoses or ketoses, or a combination thereof. Non-limiting examples of monosaccharides include ribose, deoxyribose, xylose, arabinose, glucose, mannose, galactose, and fructose. Non-limiting examples of disaccharides include sucrose, maltose, lactose, lactulose, and trehalose. Other “sugars” or “sugar groups” or “sugar residues” include polysaccharides and/or oligosaccharides, including, but not limited to, amylose, amylopectin, glycogen, inulin, and cellulose. In some instances, a “sugar” or “sugar group” or “sugar residue” is an amino-sugar. In some instances, a “sugar” or “sugar group” or “sugar residue” is a glucamine residue (1-amino-1-deoxy-D-glucitol) linked to the rest of molecule via its amino group to form an amide linkage with the rest of the molecule (i.e., a glucamide).

Certain groups, moieties, substituents, and atoms are depicted with a wiggly line, e.g., that intersects a bond or bonds, to indicate the atom through which the groups, moieties, substituents, atoms are bonded. For example, a phenyl group that is substituted with a propyl group depicted as:

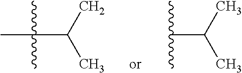

has the following structure:

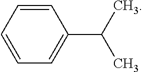

As used herein, “binding agent” refers to any molecule, e.g., antibody, capable of binding with specificity to a given binding partner, e.g., antigen.

As used herein, the term “amino acid” refers to an organic compound that contains amino (—NH₂) and carboxyl (—COOH) functional groups, along with a side chain (R group), which is specific to each amino acid. Amino acids may be proteinogenic or non-proteinogenic. By “proteinogenic” is meant that the amino acid is one of the twenty naturally occurring amino acids found in proteins. The proteinogenic amino acids include alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine. By “non-proteinogenic” is meant that either the amino acid is not found naturally in protein or is not directly produced by cellular machinery (e.g., is the product of post-translational modification). Non-limiting examples of non-proteinogenic amino acids include gamma-aminobutyric acid (GABA), taurine (2-aminoethanesulfonic acid), theanine (L-γ-glutamylethylamide), hydroxyproline, beta-alanine, ornithine, and citrulline.

As used herein “peptide”, in its various grammatical forms, is defined in its broadest sense to refer to a compound of two or more subunit amino acids, amino acid analogs, or other peptidomimetics. The subunits may be linked by peptide bonds or by other bonds, for example, ester, ether, and the like. As used herein, the term “amino acid” refers to either natural and/or unnatural, proteinogenic or non-proteinogenic, or synthetic amino acids, including glycine and both the D and L optical isomers, and amino acid analogs and peptidomimetics. If the peptide chain is short, e.g., two, three or more amino acids, it is commonly called an oligopeptide. If the peptide chain is longer, the peptide is typically called a polypeptide or a protein. Full-length proteins, analogs, mutants, and fragments thereof are encompassed by the definition. The terms also include post-expression modifications of the polypeptide, for example, glycosylation, acetylation, phosphorylation, and the like. Furthermore, as ionizable amino and carboxyl groups are present in the molecule, a particular peptide may be obtained as an acidic or basic salt, or in neutral form. A peptide may be obtained directly from the source organism or may be recombinantly or synthetically produced.

The amino acid sequence of an antibody can be numbered using any known numbering schemes, including those described by Kabat et al., (“Kabat” numbering scheme); Al-Lazikani et al., 1997, J. Mol. Biol., 273:927-948 (“Chothia” numbering scheme); MacCallum et al., 1996, J. Mol. Biol. 262:732-745 (“Contact” numbering scheme); Lefranc et al., Dev. Comp. Immunol., 2003, 27:55-77 (“IMGT” numbering scheme); and Honegge and Pluckthun, J. Mol. Biol., 2001, 309:657-70 (“AHo” numbering scheme). Unless otherwise specified, the numbering scheme used herein is the Kabat numbering scheme. However, selection of a numbering scheme is not intended to imply differences in sequences where they do not exist, and one of skill in the art can readily confirm a sequence position by examining the amino acid sequence of one or more antibodies. Unless stated otherwise, the “EU numbering scheme” is generally used when referring to a residue in an antibody heavy chain constant region (e.g., as reported in Kabat et al., supra).

As used herein, the term “cell-killing activity” refers to the activity that decreases or reduces the cell viability of the tested cell line.

In the claims that follow and in the preceding description, except where the context requires otherwise due to express language or necessary implication, the word “comprise” or variations such as “comprises” or “comprising” is used in an inclusive sense, i.e., to specify the presence of the stated features but not to preclude the presence or addition of further features in various embodiments.

### 7.2. PBD Compounds

Described herein are compounds of Formula (I):

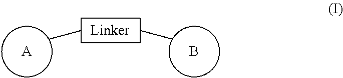

or a pharmaceutically acceptable salt, tautomer, solvate, or stereoisomer thereof, wherein: each of ring A and ring B is, independently, one of the following formulas:

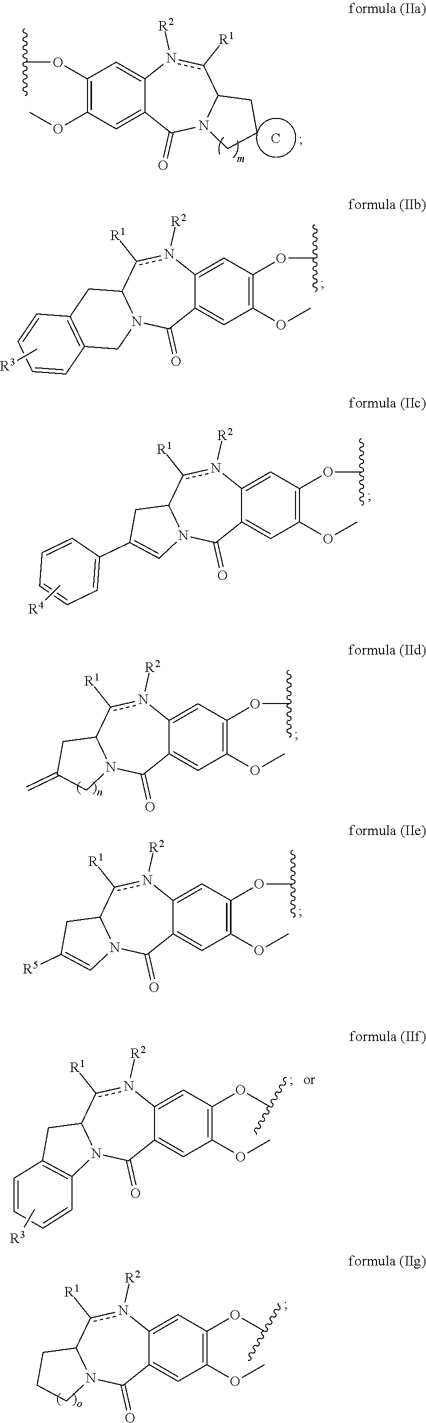

[symbol] indicates the point of attachment to Linker;
Linker is —(CH₂)ᵣ—, —(CH₂)ₚ—X—(CH₂)_{q}—, or —(CH₂)ₚ—CH═CH—(CH₂)_{q}—;
X is NR⁶, NHC(═O), C(═O)NH, O, SO₂, a substituted or unsubstituted divalent aryl ring, substituted or unsubstituted divalent heteroaryl ring, substituted or unsubstituted divalent heterocyclic ring, or substituted or unsubstituted divalent cyclic ring;
ring C is a cyclopropyl ring or a cyclobutyl ring;
each of the dotted bonds between —C(R¹)— and —N(R²)— is, independently, a single bond or a double bond;
when the dotted bond is a single bond, each R¹ is, independently, H or OH, and each R² is, independently, H;
when the dotted bond is a double bond, each R¹ is, independently, H, and each R² is, independently, absent;
each of R³ and R⁴, independently, is H, NH₂, NR^{a}R^{b}, OH, C₁₋₄ alkyl, C₁₋₄ alkoxyl, or aryl, and R^{a} and R^{b} are each independently H or C₁₋₄ alkyl;
R⁵ is H, C₁₋₄ alkyl, C₁₋₄ alkoxyl, or aryl;
R⁶ is H or C₁₋₄ alkyl;
each of m, n, and o is, independently, 1 or 2;
each of r, p, and q, is, independently, an integer from 1 to 8; and
the sum of p and q is an integer from 1 to 8.

In some embodiments, X is NR⁶, NHC(═O), O, a substituted or unsubstituted aryl ring, substituted or unsubstituted heteroaryl ring, substituted or unsubstituted heterocyclic ring, or substituted or unsubstituted cyclic ring.

In some embodiments, ring A and ring B are the same formula.

In some embodiments, ring A and ring B each have a different formula.

In some embodiments, when Linker is —(CH₂)ᵣ—, ring A and ring B are not the same formula.

In some embodiments, when Linker is —(CH₂)ᵣ—, ring A is formula (IIa) and ring B is formula (IIb).

In some embodiments, when Linker is —(CH₂)ₚ—X—(CH₂)_{q}— or —(CH₂)ₚ—CH═CH—(CH₂)_{q}—, ring A is formula (IIa), m is 2 in ring A, and ring B is one of formula (IIa), (IIb), (IIc), (IId), (IIe), (IIf), and (IIg). In some embodiments, when Linker is —(CH₂)ₚ—X—(CH₂)_{q}— or —(CH₂)ₚ—CH═CH—(CH₂)_{q}—, ring A is formula (IIa), m is 1 in ring A, and ring B is one of formula (IIa), (IIb), (IIc), (IId), (IIe), (IIf), and (IIg). In some embodiments, formula (IIc) is the following formula (IIc3) or formula (IIc4):

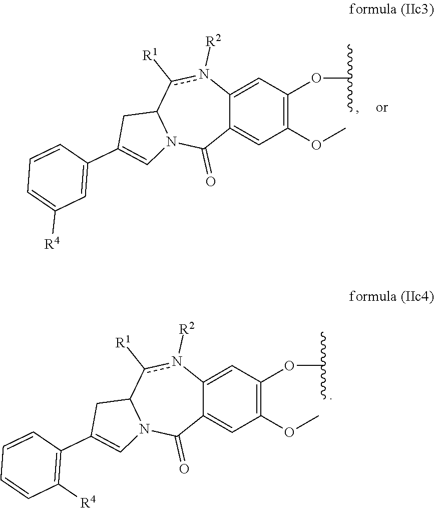

In some embodiments, when Linker is —(CH₂)ₚ—X—(CH₂)_{q}— or —(CH₂)ₚ—CH═CH—(CH₂)_{q}—, ring A is formula (Iha), and ring B is one of formula (IIa), (IIb), (IIc), (IId), (IIe), (IIf), or (IIg).

In some embodiments, Linker is —(CH₂)ᵣ—, —(CH₂)ₚ—X—(CH₂)_{q}—, or —(CH₂)ₚ—CH═CH—(CH₂)_{q}—; X is O, NR⁶, NHC(═O), -(m-C₆H₄)—,

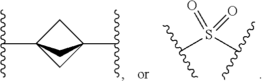 [SMILES: *[C@]12C[C@](*)(C1)C2 ; *S(*)(=O)=O]

In some embodiments, R⁶ is H or methyl.

In some embodiments, Linker is —(CH₂)ᵣ—.

In some embodiments, r is 3 or 5.

In some embodiments, Linker is —(CH₂)ₚ—O—(CH₂)_{q}— or —(CH₂)ₚ—NH—(CH₂)_{q}—.

In some embodiments, the sum of p and q is 4.

In some embodiments, Linker is

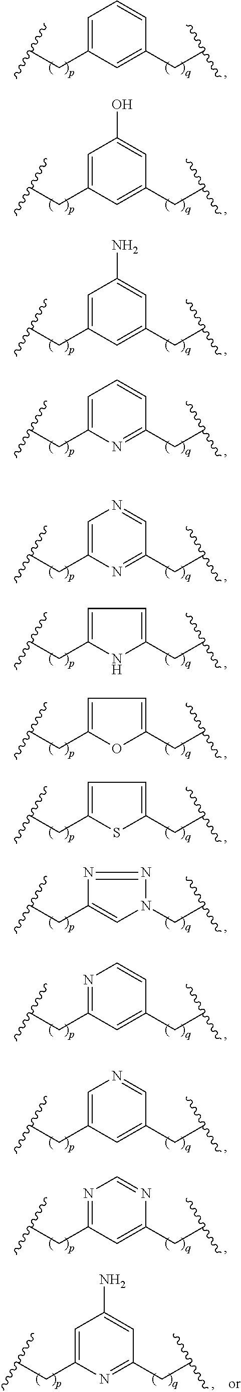

In some embodiments, the sum of p and q is 2.

In some embodiments, Linker is

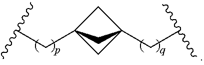

In some embodiments, the sum of p and q is 2.

In some embodiments, Linker is —(CH₂)ₚ—CH═CH—(CH₂)_{q}—.

In some embodiments, the sum of p and q is 3.

In some embodiments, ring B is formula (IIa).

In some embodiments, m is 1.

In some embodiments, ring C is a cyclopropyl ring.

In some embodiments, the dotted bond in ring B is a single bond, R¹ is H or OH, and R² is H.

In some embodiments, the dotted bond in ring B is a double bond, R¹ is H, and R² is absent.

In some embodiments, ring A is formula (IIb).

In some embodiments, ring A is formula (IIb2):

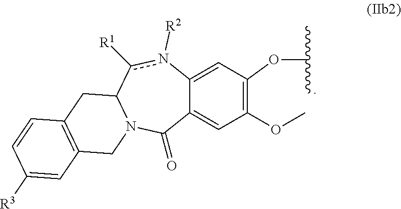

In some embodiments, R³ is H.

In some embodiments, the dotted bond in ring A is a single bond, R¹ is H or OH, and R² is H.

In some embodiments, the dotted bond in ring A is a double bond, R¹ is H, and R² is absent.

In some embodiments, the compound is

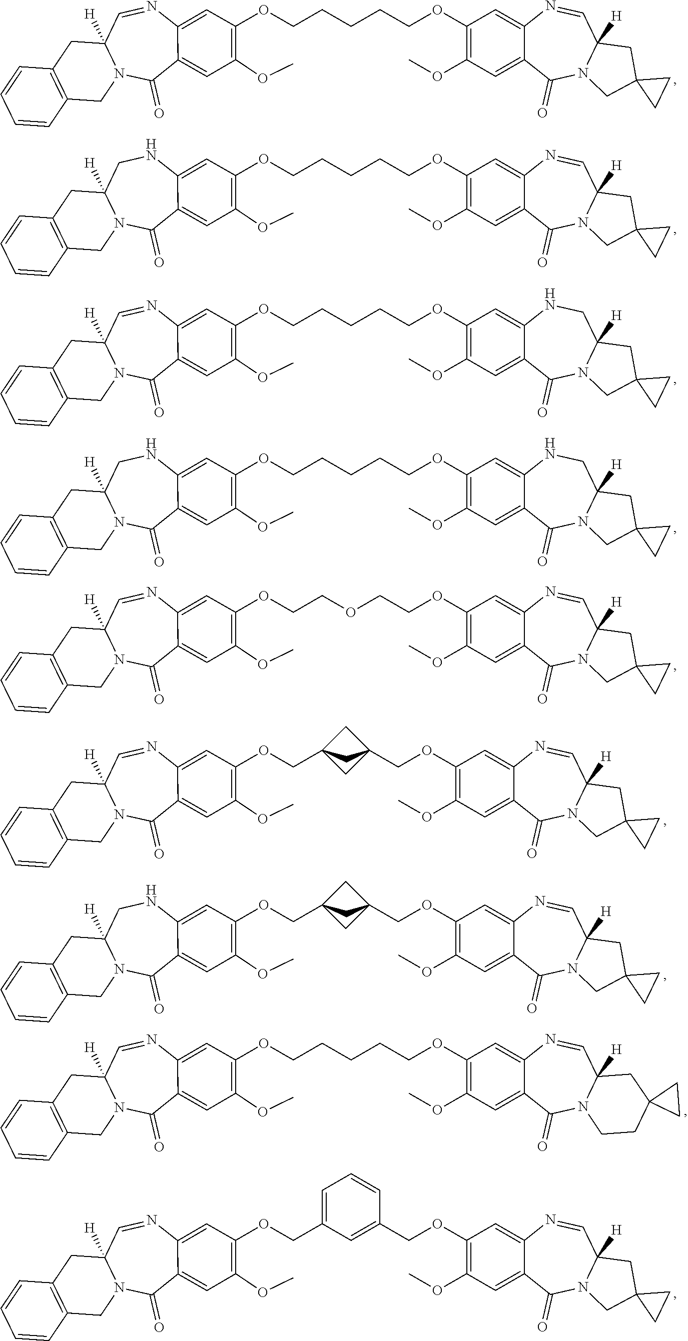

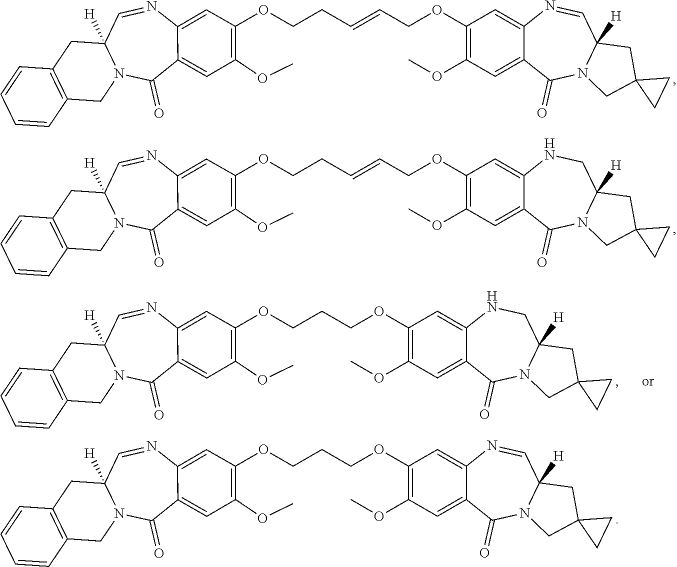

In some embodiments, ring A is formula (IIg).

In some embodiments, o is 2.

In some embodiments, the dotted bond in ring A is a single bond, R¹ is H or OH, and R² is H.

In some embodiments, the dotted bond in ring A is a double bond, R¹ is H, and R² is absent.

In some embodiments, the compound is

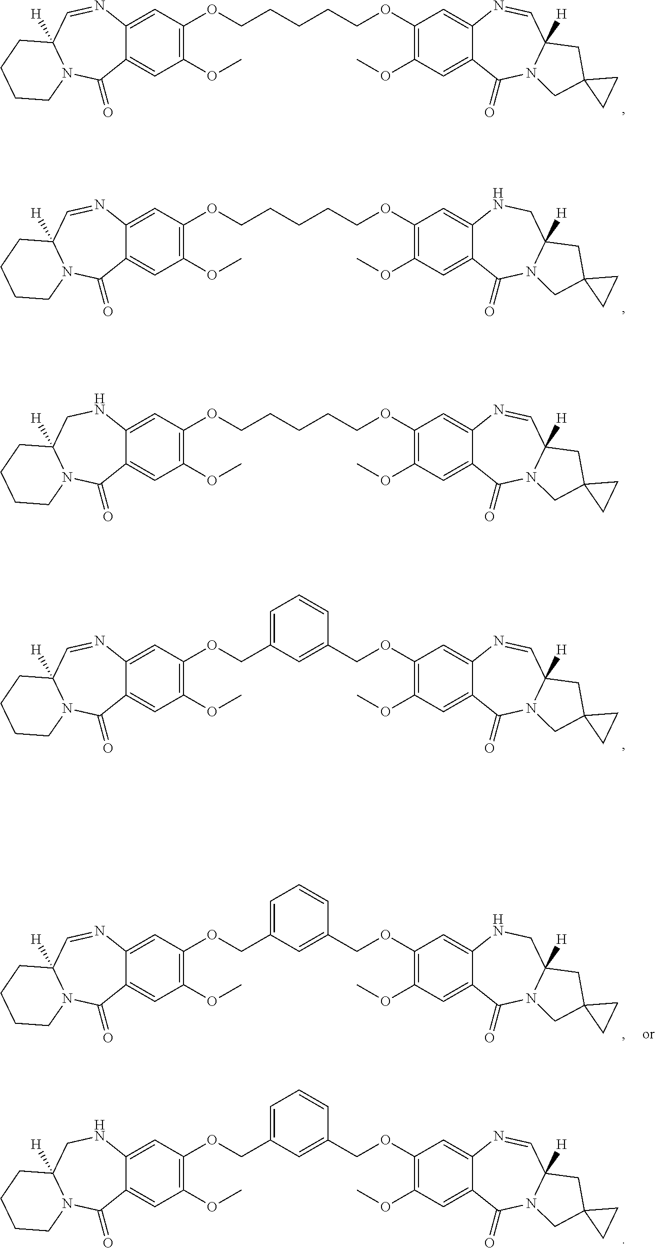

In some embodiments, ring A is formula (IId).

In some embodiments, n is 1.

In some embodiments, the dotted bond in ring A is a single bond, R¹ is H or OH, and R² is H.

In some embodiments, the dotted bond in ring A is a double bond, R¹ is H, and R² is absent.

In some embodiments, the compound is

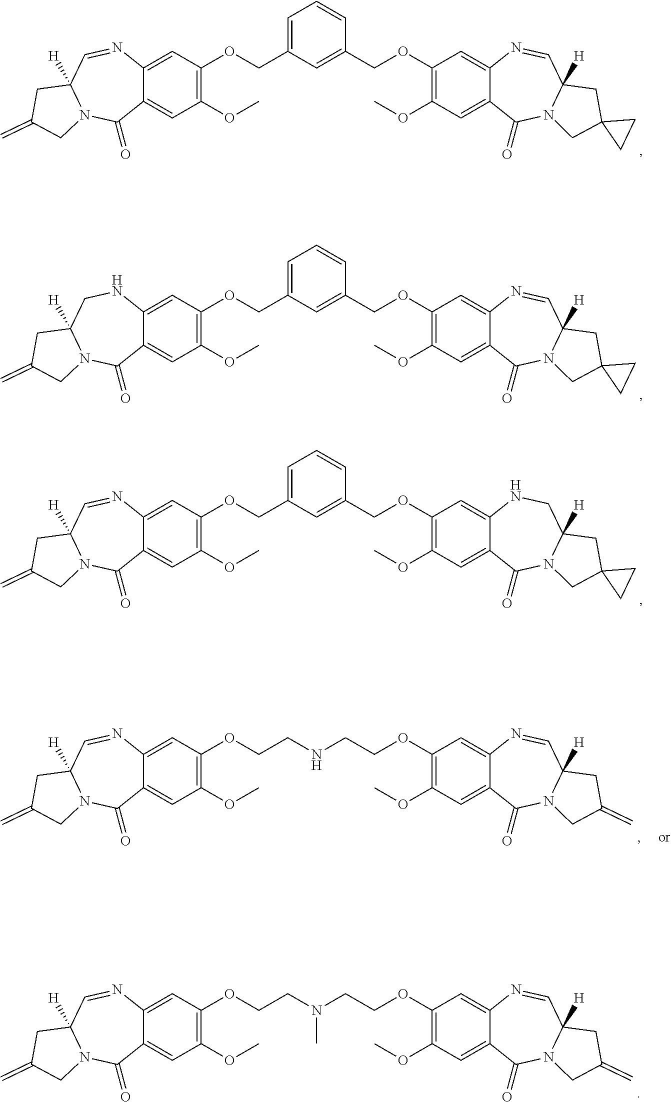

In some embodiments, ring A is formula (IIc).

In some embodiments, ring A is formula (IIc2):

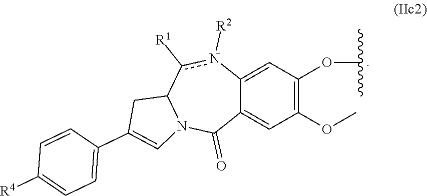

In some embodiments, R⁴ is CH₃O—.

In some embodiments, the dotted bond in ring A is a single bond, R¹ is H or OH, and R² is H.

In some embodiments, the dotted bond in ring A is a double bond, R¹ is H, and R² is absent.

In some embodiments, the compound is

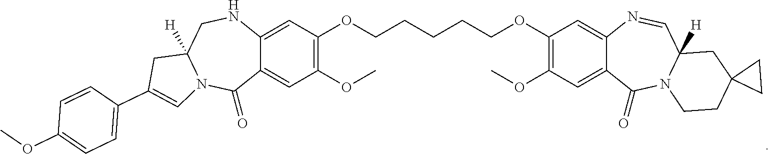 [SMILES: COc1ccc(C2=CN3C(=O)c4cc(OC)c(OCCCCCOc5cc6c(cc5OC)C(=O)N5CCC7(CC7)C[C@H]5C=N6)cc4NC[C@@H]3C2)cc1]

In some embodiments, ring A is formula (IId).

In some embodiments, n is 2.

In some embodiments, the dotted bond in ring A is a single bond, R¹ is H or OH, and R² is H.

In some embodiments, the dotted bond in ring A is a double bond, R¹ is H, and R² is absent.

In some embodiments, the compound is

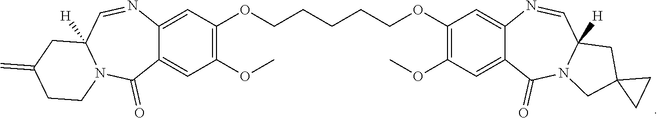 [SMILES: C=C1CCN2C(=O)c3cc(OC)c(OCCCCCOc4cc5c(cc4OC)C(=O)N4CC6(CC6)C[C@H]4C=N5)cc3N=C[C@@H]2C1]

In some embodiments, ring A is formula (IIe).

In some embodiments, R⁵ is methyl.

In some embodiments, the dotted bond in ring A is a single bond, R¹ is H or OH, and R² is H.

In some embodiments, the dotted bond in ring A is a double bond, R¹ is H, and R² is absent.

In some embodiments, the compound is

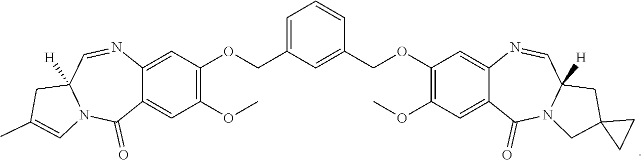 [SMILES: COc1cc2c(cc1OCc1cccc(COc3cc4c(cc3OC)C(=O)N3CC5(CC5)C[C@H]3C=N4)c1)N=C[C@@H]1CC(C)=CN1C2=O]

In some embodiments, each of ring A and ring B, independently, comprises formula (IIa).

In some embodiments, m is 1.

In some embodiments, ring C is a cyclopropyl ring.

In some embodiments, when the dotted bond in ring A is a single bond, R¹ is H or OH, and R² is H.

In some embodiments, when the dotted bond in ring A is a double bond, R¹ is H, and R² is absent.

In some embodiments, when the dotted bond in ring B is a single bond, R¹ is H or OH, and R² is H.

In some embodiments, when the dotted bond in ring B is a double bond, R¹ is H, and R² is absent.

In some embodiments, the compound is

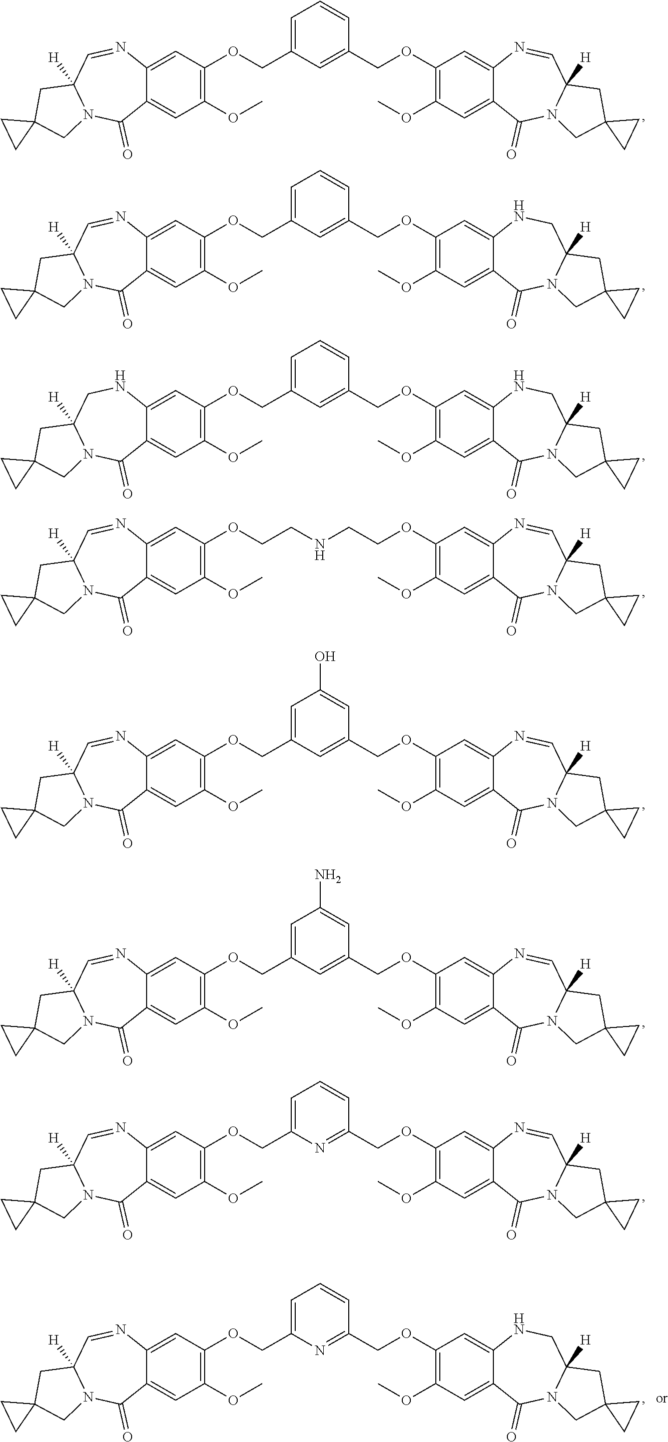

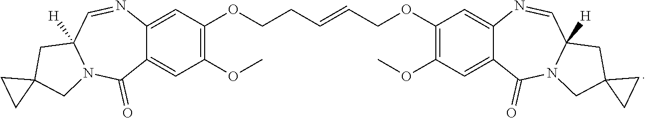 [SMILES: COc1cc2c(cc1OC/C=C/CCOc1cc3c(cc1OC)C(=O)N1CC4(CC4)C[C@H]1C=N3)N=C[C@@H]1CC3(CC3)CN1C2=O]

### 7.3. Linker-Payload Compounds

Also disclosed herein are compounds, or a pharmaceutically acceptable salt, tautomer, solvate, or stereoisomer thereof, including a covalent linker coupled to a residue of at least one PBD-based compound disclosed herein. The covalent linker (or “Ab Linker”) is capable of binding to a binding agent, such as to form a conjugate, as described below.

In some embodiments, the compounds have one of the following formulas:

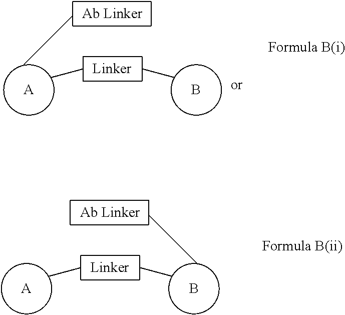

or a pharmaceutically acceptable salt, tautomer, solvate, or stereoisomer thereof, wherein:
each of ring A and ring B is, independently, one of the following formulas:

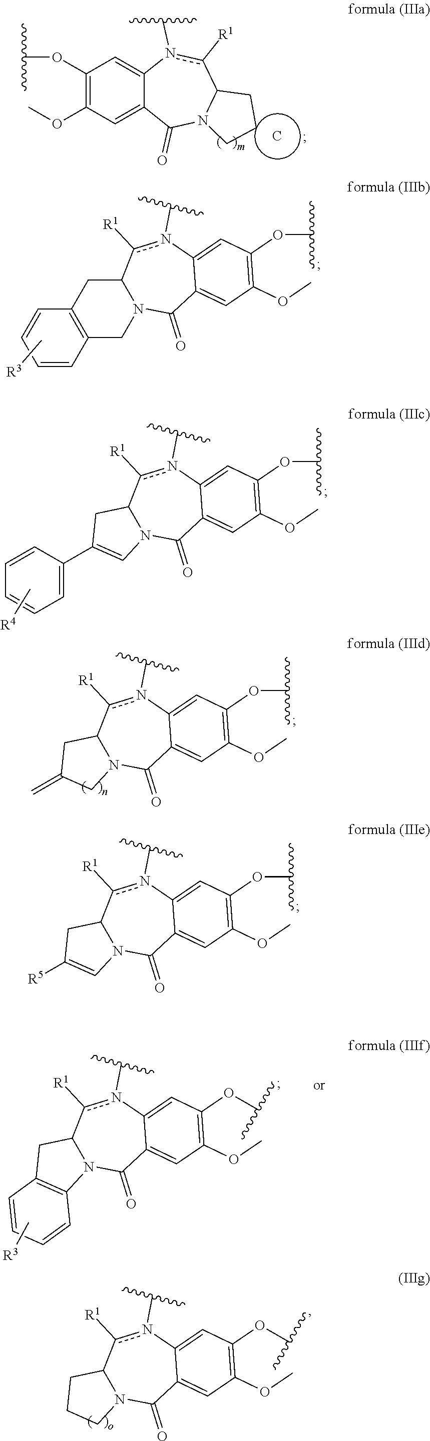

[symbol] indicates the point of attachment to Linker or Ab Linker;
Linker is —(CH₂)ᵣ—, —(CH₂)ₚ—X—(CH₂)_{q}—, or —(CH₂)ₚ—CH═CH—(CH₂)_{q}—;
X is NR⁶, NHC(═O), C(═O)NH, O, SO₂, a substituted or unsubstituted aryl ring, substituted or unsubstituted heteroaryl ring, substituted or unsubstituted heterocyclic ring, or substituted or unsubstituted cyclic ring;
ring C is a cyclopropyl ring or a cyclobutyl ring;
each of the dotted bonds [symbol] between —C(R¹)— and —N(R²)— is, independently, a single bond or a double bond;
when the dotted bond is a single bond, each R¹ is, independently, H or OH, and each R² is H;
when the dotted bond is a double bond, each R¹ is, independently, H, and each R² is absent;
each of R³ and R⁴ is, independently, H, NH₂, N^{a}R^{b}, OH, C₁₋₄ alkyl, C₁₋₄ alkoxyl, or aryl;
R^{a} and R^{b} are each independently H or C₁₋₄ alkyl;
R⁵ is H, C₁₋₄ alkyl, C₁₋₄ alkoxyl, or aryl;
R⁶ is H or C₁₋₄ alkyl;
each of m, n, and o is, independently, 1 or 2;
each of r, p, and q, is, independently, an integer from 1 to 8;
the sum of p and q is an integer from 1 to 8; and
Ab Linker is a compound able to join ring A or ring B to a binding agent.

In some embodiments, Ab Linker has the following formula:

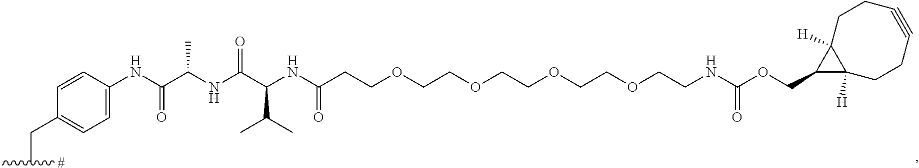

wherein [symbol] # indicates the point of attachment to ring A or ring B.

In some embodiments, the compound has the following formula:

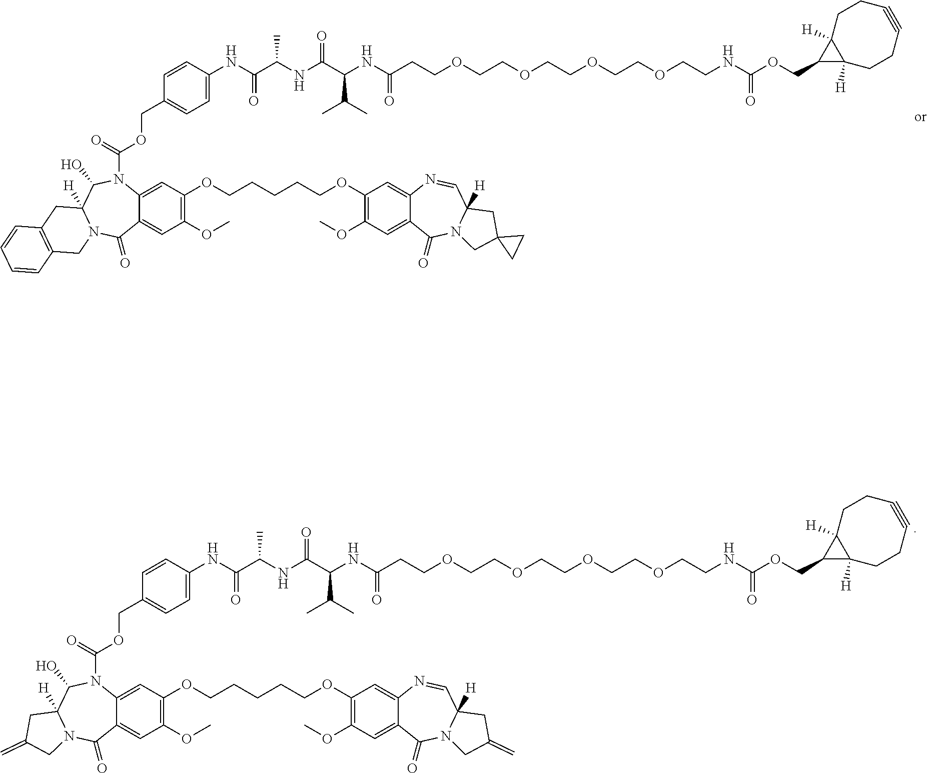 [SMILES: COc1cc2c(cc1OCCCCCOc1cc3c(cc1OC)C(=O)N1Cc4ccccc4C[C@H]1[C@H](O)N3C(=O)OCc1ccc(NC(=O)[C@H](C)NC(=O)[C@@H](NC(=O)CCOCCOCCOCCOCCNC(=O)OC[C@@H]3[C@@H]4CCC#CCC[C@@H]43)C(C)C)cc1)N=C[C@@H]1CC3(CC3)CN1C2=O ; C=C1C[C@H]2C=Nc3cc(OCCCCCOc4cc5c(cc4OC)C(=O)N4CC(=C)C[C@H]4[C@H](O)N5C(=O)OCc4ccc(NC(=O)[C@H](C)NC(=O)[C@@H](NC(=O)CCOCCOCCOCCOCCNC(=O)OC[C@@H]5[C@@H]6CCC#CCC[C@@H]65)C(C)C)cc4)c(OC)cc3C(=O)N2C1]

### 7.4. Conjugates

Also disclosed herein are conjugates, or a pharmaceutically acceptable salt, tautomer, solvate, or stereoisomer thereof, including a protein coupled to a residue of at least one PBD-based compound disclosed herein via a covalent linker. In some embodiments, the protein is a binding agent, such as an antibody or antigen binding fragment thereof. The conjugate may be an antibody drug conjugate (ADC).

In some embodiments, the protein is bonded directly to a covalent linker, such as an Ab Linker set forth herein. In such cases, the binding agent is one bond position away from the covalent linker. The covalent linker may also be bonded directly to a payload residue such that the covalent linker is one bond position away from a payload residue. The payload may be any PBD-based compound set forth herein.

In some embodiments, the conjugates have one of the following formulas:

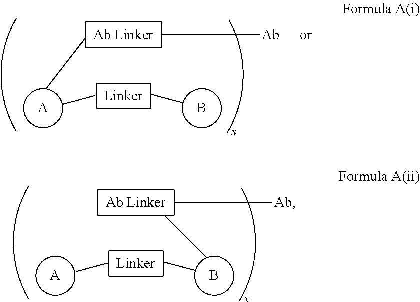

or a pharmaceutically acceptable salt, tautomer, solvate, or stereoisomer thereof, wherein:
each of ring A and ring B is, independently, one of the following formulas:

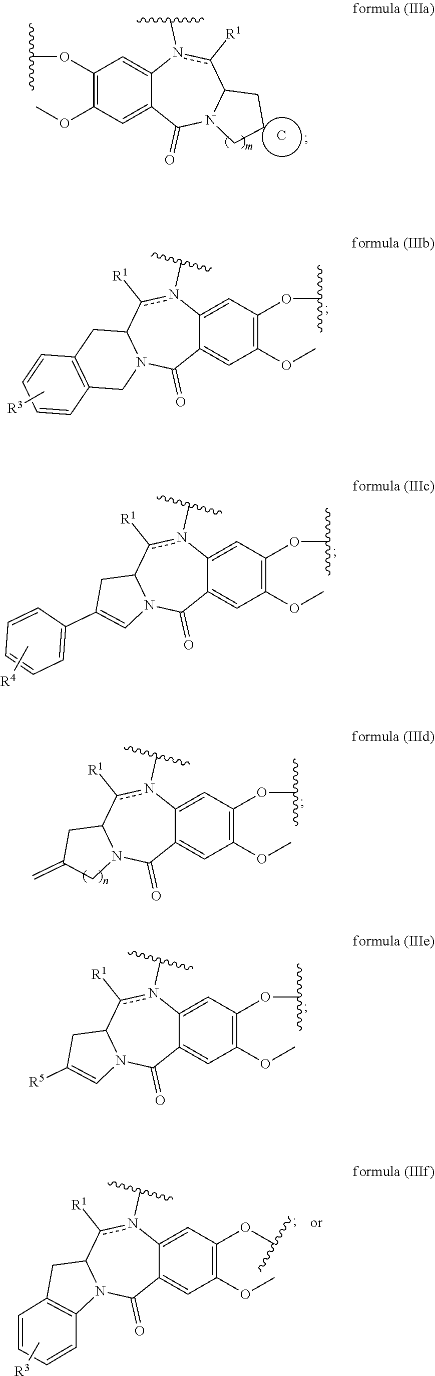

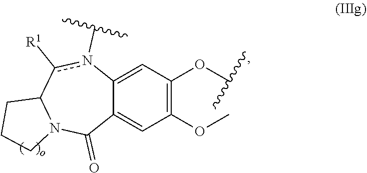

[symbol] indicates the point of attachment to Linker or Ab Linker;
Linker is —(CH₂)ᵣ—, —(CH₂)ₚ—X—(CH₂)_{q}—, or —(CH₂)ₚ—CH═CH—(CH₂)_{q}—;
X is NR⁶, NHC(═O), C(═O)NH, O, SO₂, a substituted or unsubstituted aryl ring, substituted or unsubstituted heteroaryl ring, substituted or unsubstituted heterocyclic ring, or substituted or unsubstituted cyclic ring;
ring C is a cyclopropyl ring or a cyclobutyl ring;
each of the dotted bonds between —C(R¹)— and —N(R²)— is, independently, a single bond or a double bond;
when the dotted bond is a single bond, each R¹ is, independently, H or OH, and each R² is, independently, H;
when the dotted bond is a double bond, each R¹ is, independently, H, and each R² is, independently, absent;
each of R³ and R⁴, independently, is H, NH₂, NR^{a}R^{b}, OH, C₁₋₄ alkyl, C₁₋₄ alkoxyl, or aryl, and R^{a} and R^{b} are each independently H or C₁₋₄ alkyl;
R⁵ is H, C₁₋₄ alkyl, C₁₋₄ alkoxyl, or aryl;
R⁶ is H or C₁₋₄ alkyl;
each of m, n, and o is, independently, 1 or 2;
each of r, p, and q, is, independently, an integer from 1 to 8; and the sum of p and q is an integer from 1 to 8.

In some embodiments, Ab Linker has the following formula:

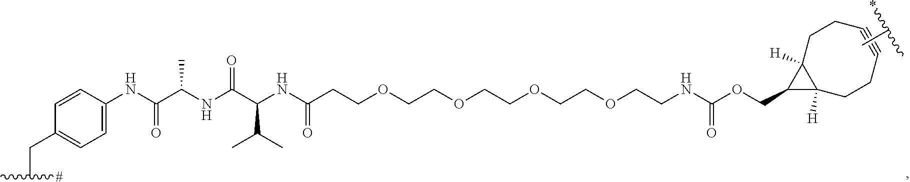

[symbol] * indicates a point of attachment to Ab, and [symbol] # indicates a point of attachment to ring A or ring B.

In some embodiments, Ab Linker has the following formula:

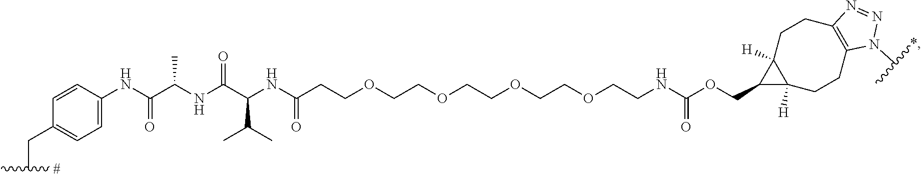

[symbol] * indicates a point of attachment to Ab, and [symbol] # in a point of attachment to ring A or ring B.

In some embodiments, the conjugate has the following formula:

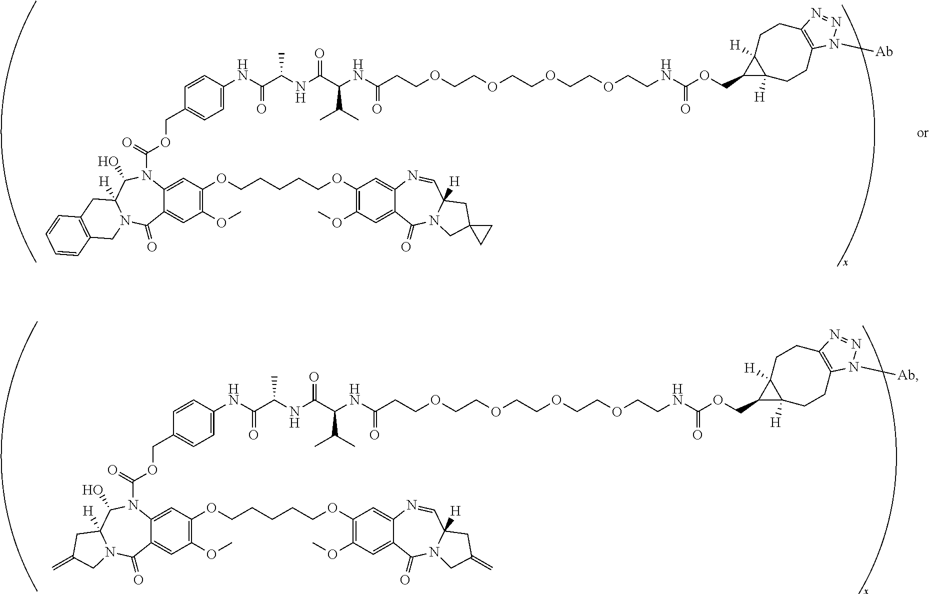

wherein Ab is a binding agent selected from a humanized, chimeric, or human antibody, or an antigen binding fragment thereof, and subscript x is from 1 to 15. In some embodiments, x is about 2.

In some embodiments, the ADC has the following formula:

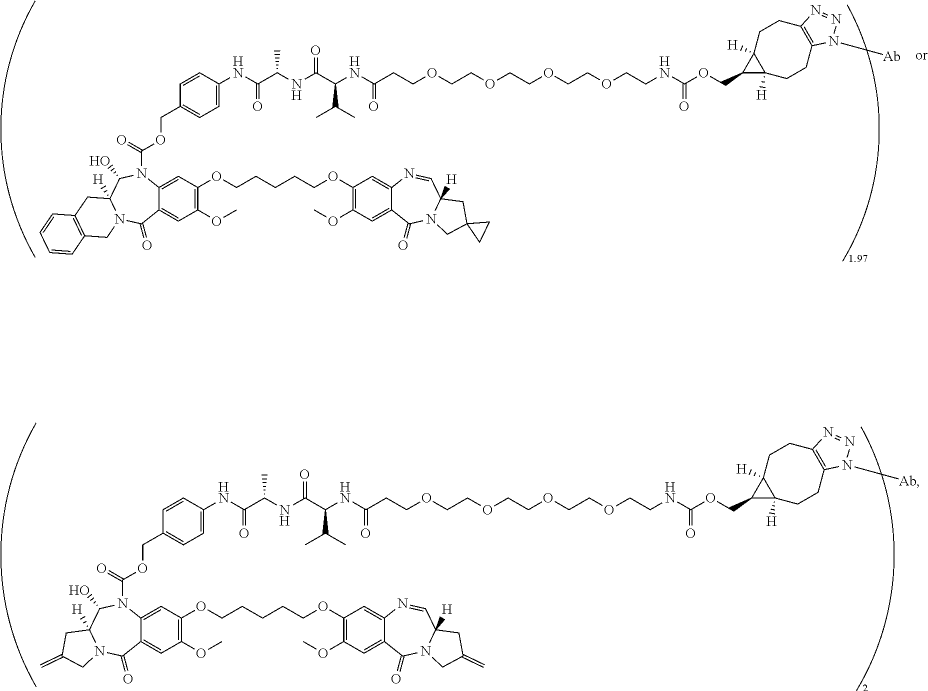

wherein Ab is a binding agent selected from a humanized, chimeric, or human antibody, or an antigen binding fragment thereof.

### 7.5. Methods or Processes of Making the Conjugates

Provided herein are methods of preparing a conjugate by contacting a binding agent (BA) with a linker-payload compound under conditions suitable for forming a bond between the binding agent and the linker-payload compound. The reaction conditions may be any suitable reaction conditions known in the art. The binding agent may be an antibody and the bond may form an antibody-drug conjugate

Examples of such reactions are provided in the Examples below.

In some embodiments, methods of making a conjugate including treating or contacting a compound with a binding agent under coupling conditions. The compound may include a reactive linker bonded to at least one payload. The compound may be any of the linker or platform compounds disclosed herein.

### 7.6. Pharmaceutical Compositions

Also provided herein are compositions, including pharmaceutical compositions, comprising an ADC set forth herein. In some embodiments, the compositions (e.g., pharmaceutical compositions) further comprise a pharmaceutically acceptable excipient.

Pharmaceutical compositions in accordance with the present disclosure can be prepared by mixing an antibody drug conjugate having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to, buffers such as phosphate, citrate, and other organic acids; antioxidants including ascorbic acid and methionine; preservatives (such as octadecyl dimethyl benzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in U.S. Pat. No. 7,871,607 and 2006/0104968. In one aspect, a sHASEGP is combined with one or more additional glycosaminoglycanases such as chondroitinases.

Exemplary lyophilized formulations are described in U.S. Pat. No. 6,267,958. Aqueous formulations include those described in U.S. Pat. No. 6,171,586 and WO2006/044908, the latter formulations including a histidine-acetate buffer.

### 7.7. Methods of Using

In some embodiments, set forth herein is a method of treating a disease or disorder (e.g., a cancer) in a subject (e.g., patient) in need thereof, comprising administering to the patient a therapeutically effective amount of a conjugate disclosed herein.

The conjugates disclosed herein can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g., by injection, such as intravenous or subcutaneous injection, depending in part on whether the administration is brief or chronic. Various dosing schedules, including but not limited to, single or multiple administrations over various time-points, bolus administration, and pulse infusion are contemplated herein.

Conjugates of the disclosure can be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

### 8. EXAMPLES

The examples below are intended to be exemplary and should not be considered limiting in any way. Unless otherwise specified, the experimental methods in the Examples described below are conventional methods. Unless otherwise specified, the reagents and materials are commercially available. All solvents and chemicals employed were of analytical grade or chemical purity. Solvents were redistilled before use. Anhydrous solvents were prepared according to standard methods or reference methods. Silica gel (100-200 meshes) for column chromatography and silica gel (GF254) for thin-layer chromatography (TLC) are commercially available from Tsingdao Haiyang Chemical Co., Ltd. or Yantai Chemical Co., Ltd. of China; all were eluted with petroleum ether (60-90° C.)/ethyl acetate (v/v) and visualized by iodine or the solution of molybdphosphoric acid in ethanol unless otherwise specified. All extraction solvents, unless otherwise specified, were dried over anhydrous Na₂SO₄. ¹H NMR spectra were recorded on Bruck-400, Varian 400MR nuclear magnetic resonance spectrometer with TMS (tetramethylsilane) as the internal standard. Coupling constants were given in hertz. Peaks were reported as singlet (s), doublet (d), triplet (t), quartet (q), quintet (p), sextet (h), septet (hept), multiplet (m), or a combination thereof; br stands for broad. LC/MS data was recorded by using Agilent1100, 1200 High Performance Liquid Chromatography-Ion Trap Mass Spectrometer (LC-MSD Trap) equipped with a diode array detector (DAD) detected at 214 nm and 254 nm, and an ion trap (ESI source). All compound names except the reagents were generated by ChemDraw® 18.0.

For the sake of conciseness, certain abbreviations are used herein. One example is the single letter abbreviation to represent an amino acid. The amino acids and their corresponding three letter and single letter abbreviations are as follows:

| | | |
|---|---|---|
| alanine | Ala | (A) |
| arginine | Arg | (R) |
| asparagine | Asn | (N) |
| aspartic acid | Asp | (D) |
| cysteine | Cys | (C) |
| glutamic acid | Glu | (E) |
| glutamine | Gln | (Q) |
| glycine | Gly | (G) |
| histidine | His | (H) |
| isoleucine | Ile | (I) |
| leucine | Leu | (L) |
| lysine | Lys | (K) |
| methionine | Met | (M) |
| phenylalanine | Phe | (F) |
| proline | Pro | (P) |
| serine | Ser | (S) |
| threonine | Thr | (T) |
| tryptophan | Trp | (W) |
| tyrosine | Tyr | (Y) |
| valine | Val | (V) |

In the following examples, the following abbreviations are used:

| | |
|---|---|
| TEA | Triethyl amine |
| THF | Tetrahydrofuran |
| MeOH | Methanol |
| DIPEA | N,N-Diisopropylethylamine |
| DMF | N,N-Dimethylformamide |
| NH₄Cl | Ammonium chloride |
| HCl | Hydrochloric acid |
| TFA | Trifluoroacetic acid |
| K₂CO₃ | Potassium carbonate |
| Na₂SO₄ | Sodium sulfate |
| EtOAc | Ethyl acetate |
| prep-HPLC | Preparative high performance liquid chromatography |
| TBSCl | t-Butyldimethylchlorosilane |
| Et₂NH | Diethylamine |
| r.t. | Room temperature |
| MS | Mass spectrometry |
| ESI | Electron spray ionization |
| FA | Formic acid |
| MTBE | Methyl tert-butyl ether |
| HPLC | High Performance Liquid Chromatography |
| DMSO | Dimethylsulfoxide |
| MeCN | Acetonitrile |
| TBAF | Tetrabutylammonium fluoride |
| Alloc-Cl | Allyloxycarbonyl chloride |
| Pd(PPh₃)₄ | Tetrakis(triphenylphosphine)palladium(0) |
| TBSOTf | tert-Butyldimethylsilyl trifluoromethanesulfonate |
| DMP | Dess-Martin periodinane |
| HATU | Hexafluorophosphate azabenzotriazole tetramethyl uronium |
| DIAD | Diisopropyl azodicarboxylate |
| EDCI | 1-Ethyl-3-(3-dimethylaminopropyl)carbodiimide |

### UPLC Analysis Methods

Method A: Mobile phase A: 0.1% FA in water, B: MeCN; Gradient: 10% B maintain 0.2 min, 10%-95% B, 5.8 min, 95% B maintain 0.5 min; Flow rate: 0.6 mL/min; Column: ACQUITY UPLC® BEH C18 1.7 μm.

Method B: Mobile phase A: 0.1% FA in water, B: MeCN; Gradient: 10% B maintain 0.5 min, 10%-90% B, 2.5 min, 90% B maintain 0.2 min; Flow rate: 0.6 mL/min; Column: ACQUITY UPLC® BEH C18 1.7 μm.

Method C: Mobile phase A: 0.1% FA in water, B: MeCN; Gradient: 10% B maintain 0.2 min, 10%-90% B, 1.3 min, 90% B maintain 0.3 min; Flow rate: 0.6 mL/min; Column: ACQUITY UPLC® BEH C18 1.7 μm.

### Example 1-1

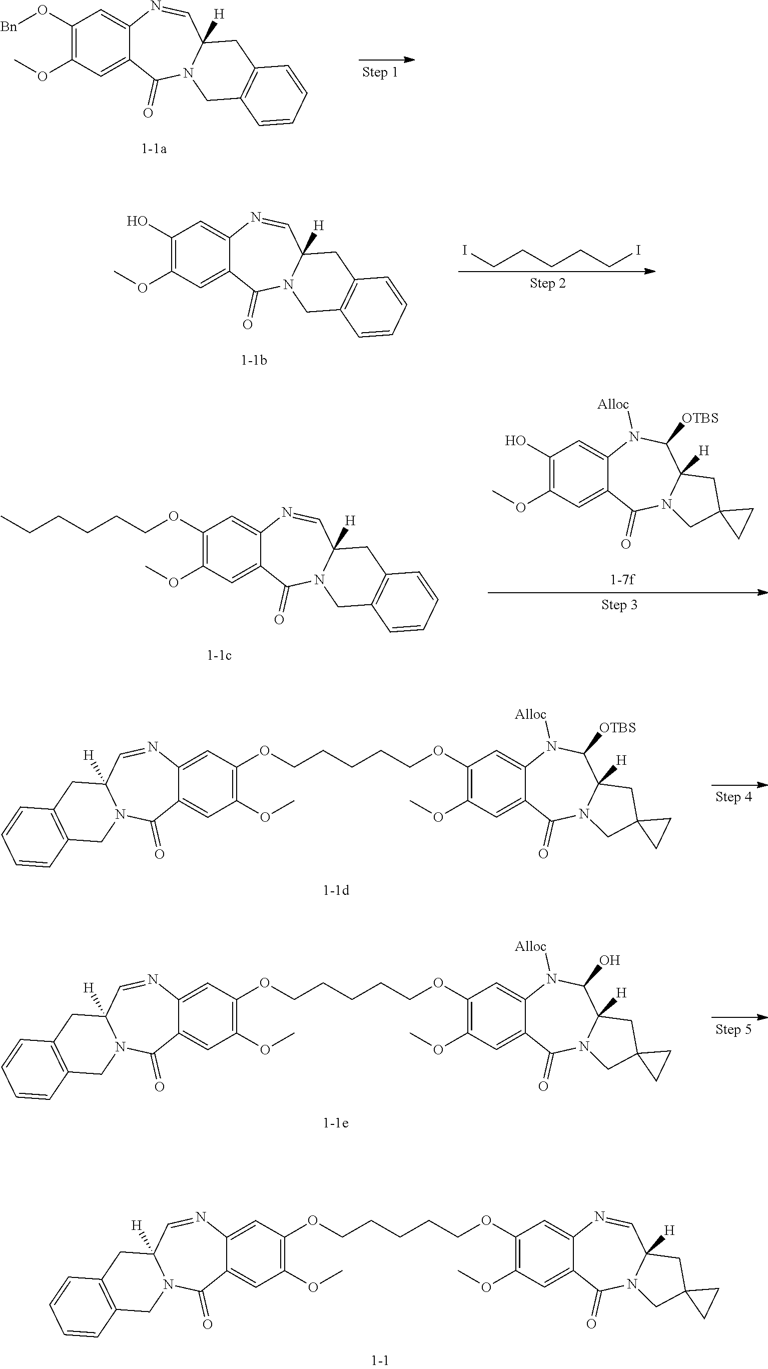

### Step 1: (S)-3-hydroxy-2-methoxy-7,12-dihydrobenzo[5,6][1,4]diazepino[1,2-b]isoquinolin-14(6aH)-one (1-1b)

Compound 1-1b was synthesized according to the procedure described in Bioorg Med Chem Lett. 2019 Sep. 1; 29(17):2455-2458.

### Step 2: (S)-3-((5-iodopentyl)oxy)-2-methoxy-7,12-dihydrobenzo[5,6][1,4]diazepino[1,2-b]isoquinolin-14(6aH)-one (1-1c)

1-1b (50 mg, 0.16 mmol) and 1,5-diiodopentane (0.12 mL, 0.81 mmol) were dissolved in dry DMF (0.5 mL). The solution was cooled to 0° C., and K₂CO₃ (45 mg, 0.32 mmol) was added in one portion. The mixture was warmed to r.t. and stirred at r.t. for 6 h. After that, EtOAc (5 mL) was added, and the diluted organic phase was washed with H₂O (10 mL) and brine (10 mL). The organic phase was dried over Na₂SO₄ and concentrated to give the crude product, which was purified by silica column gel chromatography (eluent: hexane/EtOAc=100/0 to 25/75) to give 1-1c as a light-yellow solid (49 mg, 60% yield). MS (ESI) m/z: 505.3 [M+H]⁺; ¹H NMR (400 MHz, CDCl₃) δ 7.53 (s, 1H), 7.49 (d, J=5.3 Hz, 1H), 7.39-7.34 (m, 2H), 7.31 (dd, J=3.8, 1.6 Hz, 2H), 6.80 (s, 1H), 5.01 (d, J=15.6 Hz, 1H), 4.56 (d, J=15.5 Hz, 1H), 4.16-4.01 (m, 3H), 3.95 (s, 3H), 3.33-3.09 (m, 4H), 1.95-1.84 (m, 4H), 1.59 (tt, J=9.8, 6.1 Hz, 2H).

### Step 3: allyl (11S,11aS)-11-((tert-butyldimethylsilyl)oxy)-7-methoxy-8-((5-(((S)-2-methoxy-14-oxo-6a,7,12,14-tetrahydrobenzo[5,6][1,4]diazepino[1,2-b]isoquinolin-3-yl)oxy)pentyl)oxy)-5-oxo-11,11a-dihydro-1H,3H-spiro[benzo[e]pyrrolo[1,2-a][1,4]diazepine-2,1′-cyclopropane]-10(5H)-carboxylate (1-1d)

K₂CO₃ (12.5 mg, 0.09 mmol) was added to a solution of 1-1c (50 mg, 0.098 mmol) and 1-7g (40 mg, 0.082 mmol) in 0.5 mL DMF. The mixture was stirred at r.t. for 3 h. LCMS indicated the full consumption of 1-7g. After that, EtOAc (5 mL) was added, and the diluted organic phase was washed with H₂O (10 mL) and brine (10 mL). The organic phase was dried over Na₂SO₄ and concentrated to give the crude product, which was purified by silica column gel chromatography (eluent: hexane/EtOAc=100/0 to 0/100) to give 1-1d as a white solid (60 mg, 70.6% yield). MS (ESI) m/z: 865.5 [M+H]⁺.

### Step 4: allyl (11S,11aS)-11-hydroxy-7-methoxy-8-((5-(((S)-2-methoxy-14-oxo-6a,7,12,14-tetrahydrobenzo[5,6][1,4]diazepino[1,2-b]isoquinolin-3-yl)oxy)pentyl)oxy)-5-oxo-11,11a-dihydro-1H,3H-spiro[benzo[e]pyrrolo[1,2-a][1,4]diazepine-2,1′-cyclopropane]-10(5H)-carboxylate (1-1e)

To a solution of 1-1d (60 mg, 0.07 mmol) in dry THE (1 mL) was added AcOH (24 L), followed by dropwise addition of TBAF (350 μL, 0.35 mmol, 1M in THF). The mixture was stirred at r.t. for 6 h. On completion of the reaction as checked by LCMS, the mixture was quenched with sat. NaHCO₃. The organic phase was extracted with EtOAc (5 mL*3), and washed with H₂O (10 mL) and brine (10 mL). The organic phase was dried over Na₂SO₄ and concentrated to give the crude product, which was purified by silica column gel chromatography (eluent: CH₂Cl₂/MeOH=100/0 to 5/95) to give 1-ie as a white solid (50 mg, 96% yield). MS (ESI) m/z: 751.5 [M+H]⁺.

### Step 5: (S)-2-methoxy-3-((5-(((S)-7-methoxy-5-oxo-5,11a-dihydro-1H,3H-spiro[benzo[e]pyrrolo[1,2-a][1,4]diazepine-2,1′-cyclopropan]-8-yl)oxy)pentyl)oxy)-7,12-dihydrobenzo[5,6][1,4]diazepino[1,2-b]isoquinolin-14(6aH)-one (1-1)

Pd(PPh₃)₄(2 mg, cat. amount) was added to a solution of 1-ie (50 mg, 0.067 mmol) in CH₂Cl₂ (1 mL) and pyrrolidine (14 μL, 0.17 mmol). The reaction mixture was stirred at r.t. under N₂ for 0.5 h. The reaction was diluted with CH₂Cl₂ (5 mL), and washed with sat. NH₄Cl and brine. The organic phase was dried over Na₂SO₄. The organic phase was concentrated and purified by silica column gel chromatography (eluent: CH₂Cl₂/MeOH=100/0 to 5/95) to give 1-1 as a white solid (43 mg, 99% yield). MS (ESI) m/z: 649.4 [M+H]⁺; ¹H NMR (400 MHz, CDCl₃) δ 7.79 (d, J=4.4 Hz, 1H), 7.52 (d, J=7.3 Hz, 2H), 7.48 (d, J=5.2 Hz, 1H), 7.40-7.29 (m, 4H), 6.80 (d, J=4.4 Hz, 2H), 5.01 (d, J=15.5 Hz, 1H), 4.56 (d, J=15.5 Hz, 1H), 4.19-4.02 (m, 4H), 3.94 (d, J=2.9 Hz, 7H), 3.87 (ddd, J=7.8, 4.5, 2.7 Hz, 1H), 3.68 (d, J=11.7 Hz, 1H), 3.55-3.47 (m, 1H), 3.26 (d, J=5.5 Hz, 1H), 3.16 (dd, J=15.4, 4.2 Hz, 1H), 2.52 (dd, J=13.0, 8.1 Hz, 1H), 2.08-1.90 (m, 6H), 1.68 (td, J=8.6, 5.9 Hz, 2H), 0.81-0.68 (m, 4H).

### Example 1-2

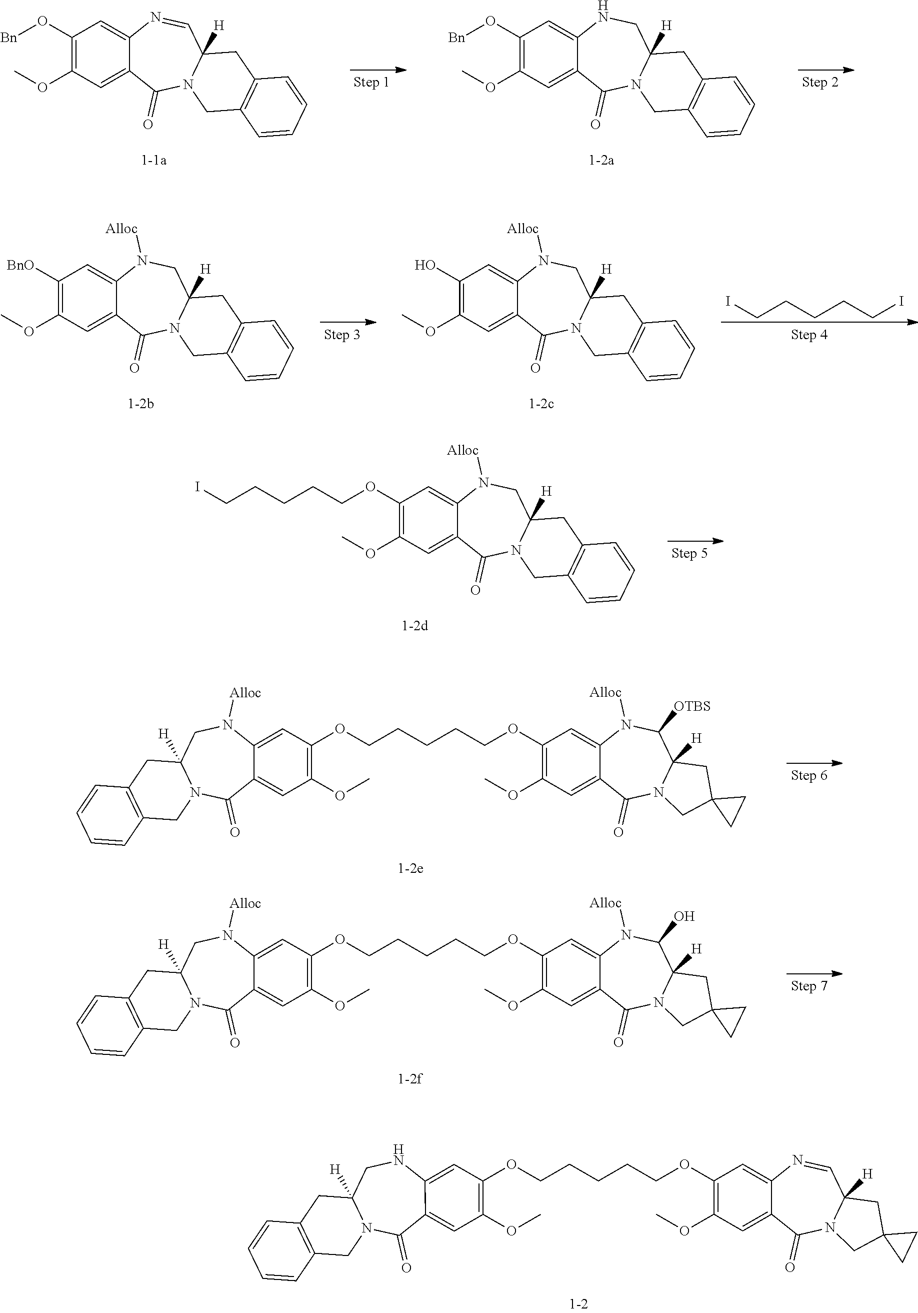

### Step 1: (S)-3-(benzyloxy)-2-methoxy-6,6a,7,12-tetrahydrobenzo[5,6][1,4]diazepino[1,2-b]isoquinolin-14(5H)-one (1-2a)

NaBH(OAc)₃ (531.9 mg, 2.5 mmol) was added to a solution of 1-1a (500 mg, 1.255 mmol) in CH₂Cl₂ (6 mL) at 0° C. The reaction mixture was then warmed to r.t. and stirred at r.t. under N₂ for 2 h. The reaction was then quenched with sat. NaHCO₃, washed with H₂O and brine, and dried over Na₂SO₄. The organic phase was filtrated and concentrated to give crude product 1-2a as a white solid (485 mg, 97% yield), which was used in the next step without further purification. MS (ESI) m/z: 401.3 [M+H]⁺.

### Step 2: allyl (S)-3-(benzyloxy)-2-methoxy-14-oxo-6,6a,7,12-tetrahydrobenzo[5,6][1,4]diazepino[1,2-b]isoquinoline-5(14H)-carboxylate (1-2b)

Alloc-Cl (0.14 mL, 1.33 mmol) was added dropwise to a solution of 1-2a (485 mg, 1.2 mmol) and pyridine (0.36 mL, 2.9 mmol) in CH₂Cl₂ (2 mL) at 0° C. The reaction mixture was stirred at 0° C. under N₂ for 15 min. The reaction was diluted with 10 mL CH₂Cl₂, washed with 0.1 N citric acid (10 mL), H₂O (10 mL) and brine (10 mL), and dried over Na₂SO₄. The organic phase was filtered and the filtrate was concentrated. The crude product was purified by silica column gel chromatography (eluent: hexane/EtOAc=100/0 to 50/50) to give 1-2b as a white solid (480 mg, 82% yield). MS (ESI) m/z: 485.5 [M+H]⁺.

### Step 3: allyl (S)-3-hydroxy-2-methoxy-14-oxo-6,6a,7,12-tetrahydrobenzo[5,6][1,4]diazepino[1,2-b]isoquinoline-5(14H)-carboxylate (1-2c)

MeSO₃H (0.64 mL, 9.9 mmol) was added dropwise to a solution of 1-2b (484.5 mg, 0.99 mmol) in CH₂Cl₂ (3 mL) at 0° C. The reaction mixture was stirred at 0° C. for 10 min and then warmed to r.t. and stirred under N₂ for 2 h. the mixture was quenched with sat. NaHCO₃. The organic phase was extracted with CH₂Cl₂ (5 mL*3), and washed with H₂O (10 mL) and brine (10 mL). The organic phase was dried over Na₂SO₄, filtrated, and the filtrate was concentrated to give the crude product, which was purified by silica column gel chromatography (eluent: hexane/EtOAc=100/0 to 25/75) to give 1-2c as a white solid (332 mg, 84.3% yield). MS (ESI) m/z: 395.4 [M+H]⁺.

¹H NMR (400 MHz, CDCl₃) δ 7.28 (d, J=3.3 Hz, 3H), 7.21 (s, 2H), 6.75 (s, 1H), 5.88 (s, 1H), 5.76 (ddd, J=17.3, 10.5, 5.2 Hz, 1H), 5.10 (dd, J=10.3, 1.4 Hz, 2H), 4.79-4.65 (m, 2H), 4.57 (dd, J=13.9, 5.2 Hz, 1H), 4.40 (d, J=13.3 Hz, 1H), 4.05-3.95 (m, 2H), 3.94 (s, 3H), 3.37 (d, J=11.4 Hz, 1H), 3.12 (dd, J=15.2, 5.6 Hz, 1H), 2.77 (dd, J=15.2, 4.1 Hz, 1H).

### Step 4: allyl (S)-3-((5-iodopentyl)oxy)-2-methoxy-14-oxo-6,6a,7,12-tetrahydrobenzo[5,6][1,4]diazepino[1,2-b]isoquinoline-5(14H)-carboxylate (1-2d)

1-2d was prepared according to the procedure described in Step 2 of Example 1-1 to obtain a white solid (138 mg, yield 92.2%). MS (ESI) m/z: 591.3 [M+H]⁺.

¹H NMR (400 MHz, CDCl₃) δ 7.35 (d, J=3.5 Hz, 3H), 7.28 (d, J=1.9 Hz, 2H), 6.72 (s, 1H), 5.84 (ddt, J=16.3, 10.8, 5.2 Hz, 1H), 5.25-5.08 (m, 2H), 4.80 (q, J=15.9 Hz, 1H), 4.67 (dd, J=13.7, 5.3 Hz, 1H), 4.48-4.40 (m, 1H), 4.14-4.00 (m, 4H), 3.98 (s, 3H), 3.46 (d, J=10.7 Hz, 1H), 3.29 (t, J=7.0 Hz, 2H), 3.20 (dd, J=15.2, 5.4 Hz, 1H), 2.85 (d, J=15.2 Hz, 1H), 1.95 (dq, J=13.4, 7.0 Hz, 4H), 1.66 (q, J=8.0 Hz, 4H).

### Step 5: allyl (S)-3-((5-(((11S,11aS)-10-((allyloxy)carbonyl)-11-((tert-butyldimethylsilyl)oxy)-7-methoxy-5-oxo-5,10,11,11a-tetrahydro-1H,3H-spiro[benzo[e]pyrrolo[1,2-a][1,4]diazepine-2,1′-cyclopropan]-8-yl)oxy)pentyl)oxy)-2-methoxy-14-oxo-6,6a,7,12-tetrahydrobenzo[5,6][1,4]diazepino[1,2-b]isoquinoline-5(14H)-carboxylate (1-2e)

1-2e was prepared according to the procedure described in Step 3 of Example 1-1 to obtain a colorless oil (70 mg, yield 85.3%). MS (ESI) m/z: 973.6 [M+Na]⁺.

### Step 6: allyl (S)-3-((5-(((11S,11aS)-10-((allyloxy)carbonyl)-11-hydroxy-7-methoxy-5-oxo-5,10,11,11a-tetrahydro-1H,3H-spiro[benzo[e]pyrrolo[1,2-a][1,4]diazepine-2,1′-cyclopropan]-8-yl)oxy)pentyl)oxy)-2-methoxy-14-oxo-6,6a,7,12-tetrahydrobenzo[5,6][1,4]diazepino[1,2-b]isoquinoline-5(14H)-carboxylate (1-2f)

1-2f was prepared according to the procedure described in Step 4 of Example 1-1 to obtain a white solid (54 mg, yield 87.7%). MS (ESI) m/z: 837.5 [M+H]⁺.

### Step 7: (S)-2-methoxy-3-((5-(((S)-7-methoxy-5-oxo-5,11a-dihydro-1H,3H-spiro[benzo[e]pyrrolo[1,2-a][1,4]diazepine-2,1′-cyclopropan]-8-yl)oxy)pentyl)oxy)-6,6a,7,12-tetrahydrobenzo[5,6][1,4]diazepino[1,2-b]isoquinolin-14(5H)-one (1-2)

1-2 was prepared according to the procedure described in Step 5 of Example 1-1 to obtain a white solid (40 mg, yield 95.2%). MS (ESI) m/z: 651.4 [M+H]⁺.

¹H NMR (400 MHz, CDCl₃) δ 7.79 (d, J=4.4 Hz, 1H), 7.49 (s, 1H), 7.35 (s, 1H), 7.27 (d, J=6.9 Hz, 4H), 6.79 (s, 1H), 6.27 (s, 1H), 4.87 (d, J=15.7 Hz, 1H), 4.73 (d, J=15.7 Hz, 1H), 4.23-3.97 (m, 5H), 3.95 (s, 3H), 3.85 (s, 3H), 3.67 (d, J=11.7 Hz, 1H), 3.53-3.41 (m, 2H), 3.22 (dd, J=12.1, 9.5 Hz, 1H), 3.11 (dd, J=15.1, 5.8 Hz, 1H), 2.81 (dd, J=15.2, 5.3 Hz, 1H), 2.51 (dd, J=13.1, 8.1 Hz, 1H), 1.96 (dq, J=24.2, 9.3, 8.1 Hz, 6H), 1.72-1.62 (m, 2H).

### Examples 1-3 and 1-4

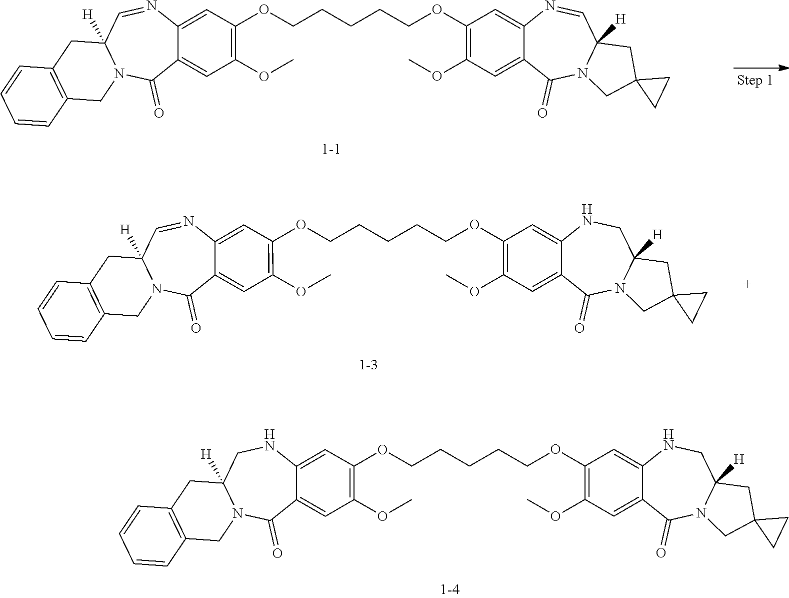 [SMILES: COc1cc2c(cc1OCCCCCOc1cc3c(cc1OC)C(=O)N1CC4(CC4)C[C@H]1C=N3)N=C[C@@H]1Cc3ccccc3CN1C2=O ; COc1cc2c(cc1OCCCCCOc1cc3c(cc1OC)C(=O)N1CC4(CC4)C[C@H]1CN3)N=C[C@@H]1Cc3ccccc3CN1C2=O ; COc1cc2c(cc1OCCCCCOc1cc3c(cc1OC)C(=O)N1CC4(CC4)C[C@H]1CN3)NC[C@@H]1Cc3ccccc3CN1C2=O]

### Step 1: (S)-2-methoxy-3-((5-(((S)-7-methoxy-5-oxo-5,10,11,11a-tetrahydro-1H,3H-spiro[benzo[e]pyrrolo[1,2-a][1,4]diazepine-2,1′-cyclopropan]-8-yl)oxy)pentyl)oxy)-7,12-dihydrobenzo[5,6][1,4]diazepino[1,2-b]isoquinolin-14(6aH)-one (1-3) and(S)-2-methoxy-3-((5-(((S)-7-methoxy-5-oxo-5,10,11,11a-tetrahydro-1H,3H-spiro[benzo[e]pyrrolo[1,2-a][1,4]diazepine-2,1′-cyclopropan]-8-yl)oxy)pentyl)oxy)-6,6a,7,12-tetrahydrobenzo[5,6][1,4]diazepino[1,2-b]isoquinolin-14(5H)-one (1-4)

NaBH(OAc)₃ (3.4 mg, 1.05 mmol) was added to a solution of 1-1 (10 mg, 0.015 mmol) in CH₂Cl₂ (0.5 mL) at 0° C. The reaction mixture was then warmed to r.t. and stirred at r.t. under N₂ for 0.5 h. The reaction was quenched with sat. NaHCO₃, washed with H₂O and brine, and dried over Na₂SO₄. The organic phase was filtrated and concentrated to give crude product, which was purified by prep-HPLC (0.01% FA in H₂O) to give 1-3 (3 mg, 30% yield) as a white solid, retention time=4.9 min, and 1-4 (2.7 mg, 27% yield) as a white solid, retention time=5.12 min.

1-3 MS (ESI) m/z: 651.4 [M+H]⁺.

¹H NMR (400 MHz, CDCl₃) δ 7.71-7.59 (m, 1H), 7.55-7.43 (m, 3H), 7.39-7.27 (m, 4H), 6.76 (s, 1H), 6.18 (s, 1H), 4.98 (d, J=15.5 Hz, 1H), 4.53 (d, J=15.5 Hz, 1H), 4.17-3.95 (m, 5H), 3.92 (d, J=5.1 Hz, 3H), 3.82 (s, 3H), 3.65 (d, J=12.0 Hz, 1H), 3.58-3.39 (m, 3H), 3.25 (dd, J=15.4, 5.5 Hz, 1H), 3.14 (dd, J=15.4, 4.3 Hz, 1H), 2.02 (dd, J=12.7, 7.3 Hz, 1H), 1.91 (h, J=7.3 Hz, 4H), 1.68 (dq, J=31.0, 7.8, 7.0 Hz, 4H), 0.75-0.50 (m, 4H).

1-4 MS (ESI) m/z: 653.6 [M+H]⁺.

¹H NMR (400 MHz, CDCl₃) δ 7.56 (s, 1H), 7.37 (s, 1H), 7.32-7.27 (m, 5H), 7.22-7.13 (m, 1H), 6.21 (s, 1H), 6.08 (s, 1H), 4.88 (d, J=15.7 Hz, 1H), 4.75 (d, J=15.7 Hz, 1H), 4.14 (d, J=6.2 Hz, 1H), 4.00 (q, J=6.1 Hz, 5H), 3.89-3.79 (m, 6H), 3.71 (d, J=12.0 Hz, 1H), 3.60-3.51 (m, 2H), 3.50-3.39 (m, 2H), 3.23 (t, J=10.8 Hz, 1H), 3.12 (dd, J=15.2, 5.8 Hz, 1H), 2.83 (dd, J=15.2, 5.4 Hz, 1H), 2.02 (t, J=10.3 Hz, 1H), 1.91 (p, J=6.9 Hz, 4H), 1.78 (dd, J=12.7, 6.9 Hz, 1H), 1.66 (q, J=7.8 Hz, 2H), 0.78-0.52 (m, 4H).

### Example 1-5

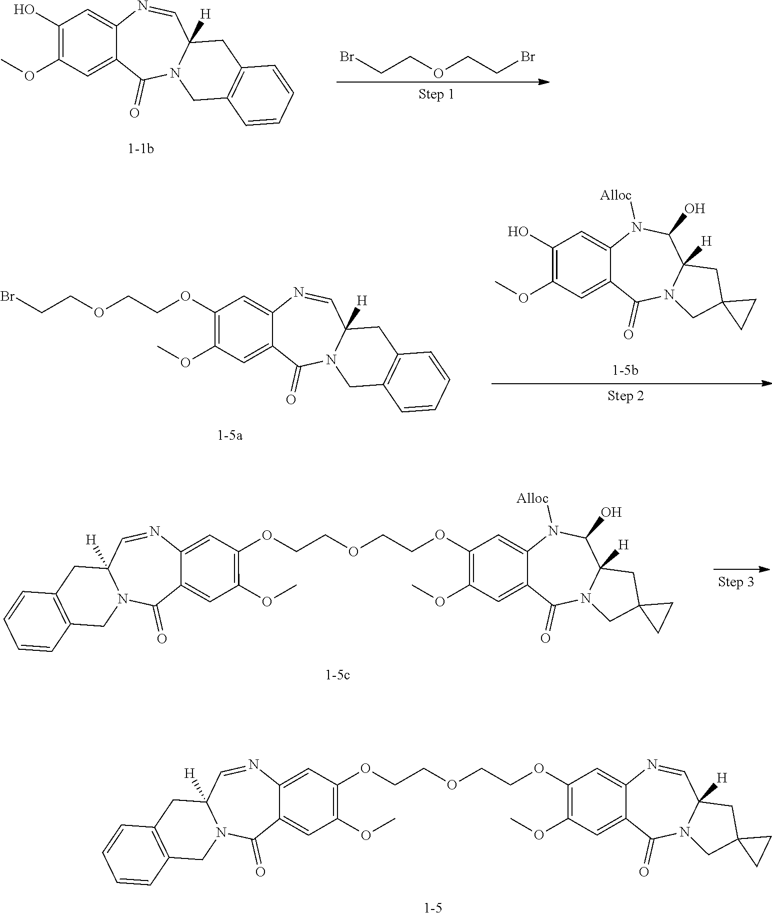

### Step 1: (S)-3-(2-(2-bromoethoxy)ethoxy)-2-methoxy-7,12-dihydrobenzo[5,6][1,4]diazepino[1,2-b]isoquinolin-14(6aH)-one (1-5a)

1-5a was prepared according to the procedure described in Step 2 of Example 1-1 to obtain a white solid (50 mg, yield 67.1%).

MS (ESI) m/z: 461.2 [M+H]⁺.

### Step 2: allyl (11S,11aS)-11-hydroxy-7-methoxy-8-(2-(2-(((S)-2-methoxy-14-oxo-6a,7,12,14-tetrahydrobenzo[5,6][1,4]diazepino[1,2-b]isoquinolin-3-yl)oxy)ethoxy)ethoxy)-5-oxo-11,11a-dihydro-1H,3H-spiro[benzo[e]pyrrolo[1,2-a][1,4]diazepine-2,1′-cyclopropane]-10(5H)-carboxylate (1-5c)

1-5c was prepared according to the procedure described in Step 3 of Example 1-1 to obtain a white solid (51 mg, yield 60.3%).

MS (ESI) m/z: 753.5 [M+H]⁺.

### Step 3: (S)-2-methoxy-3-(2-(2-(((S)-7-methoxy-5-oxo-5,11a-dihydro-1H,3H-spiro[benzo[e]pyrrolo[1,2-a][1,4]diazepine-2,1′-cyclopropan]-8-yl)oxy)ethoxy)ethoxy)-7,12-dihydrobenzo[5,6][1,4]diazepino[1,2-b]isoquinolin-14(6aH)-one (1-5)

1-5 was prepared according to the procedure described in Step 4 of Example 1-1 to obtain a white solid (25.5 mg, yield 59%).

MS (ESI) m/z: 651.4 [M+H]⁺.

¹H NMR (400 MHz, CDCl₃) δ 7.78 (d, J=4.5 Hz, 1H), 7.51 (d, J=7.4 Hz, 2H), 7.47 (dd, J=5.3, 3.3 Hz, 1H), 7.40-7.30 (m, 4H), 6.83 (dd, J=7.3, 4.5 Hz, 2H), 5.00 (d, J=15.5 Hz, 1H), 4.56 (d, J=15.5 Hz, 1H), 4.37-4.16 (m, 4H), 4.01 (t, J=5.0 Hz, 4H), 3.96-3.90 (m, 6H), 3.88-3.81 (m, 2H), 3.67 (d, J=11.7 Hz, 1H), 3.49 (d, J=11.7 Hz, 1H), 3.27 (dd, J=15.4, 5.5 Hz, 1H), 3.15 (dd, J=15.4, 4.2 Hz, 1H), 2.51 (dd, J=13.1, 8.1 Hz, 1H), 1.99 (dd, J=13.2, 2.8 Hz, 1H), 0.81-0.66 (m, 4H).

### Example 1-6

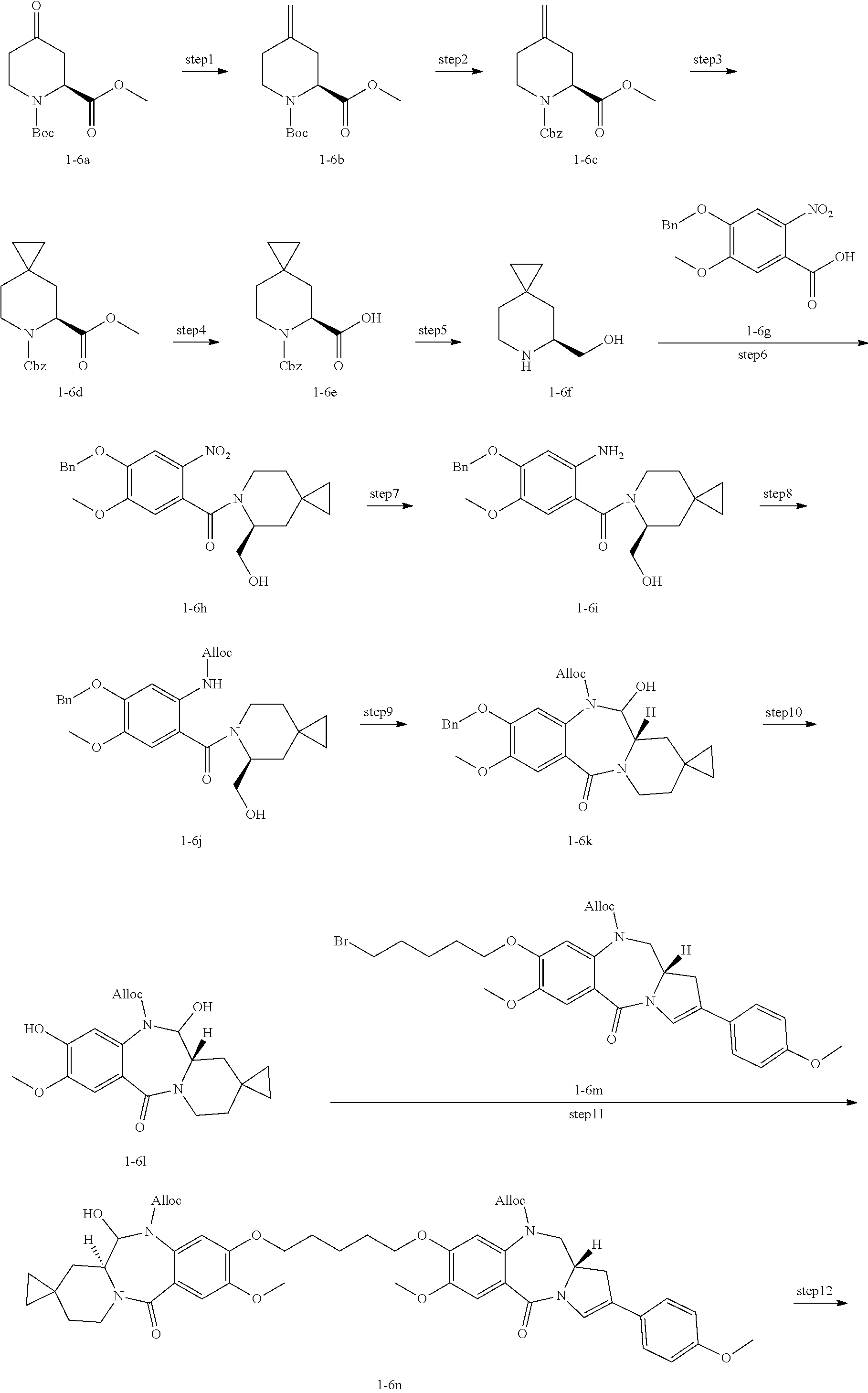

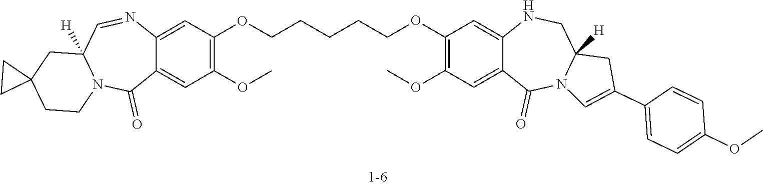 [SMILES: COc1ccc(C2=CN3C(=O)c4cc(OC)c(OCCCCCOc5cc6c(cc5OC)C(=O)N5CCC7(CC7)C[C@H]5C=N6)cc4NC[C@@H]3C2)cc1]

### Step 1: 1-(tert-butyl) 2-methyl (S)-4-methylenepiperidine-1,2-dicarboxylate (1-6b)

To a solution of MePh₃PBr (1.56 g, 4.27 mmol) in dry THE (20 mL) was added KO^{t}Bu (potassium tert-butoxide) in dry THE (lM, 4.7 mL, 4.7 mmol) under N₂ atmosphere at 0° C., and stirred at 0° C. for 1 h. To the reaction solution was added the solution of 1-6a (1.0 g, 3.89 mmol) in dry THE (20 mL) under N₂ atmosphere at 0° C., and stirred for 1 h. Water (5 mL) and sat. NH₄Cl (10 mL) were added to the solution. The organic phase was separated and the water phase was extracted with EtOAc (20 mL*3). The combined organic phase was concentrated and purified by flash column chromatography (petroleum ether/EtOAc=90/10) to afford the title compound 1-6b as a colorless oil (588 mg, 59.3% yield). MS (ESI) m/z: 156.1 [M+H-Boc]⁺.

¹H NMR (400 MHz, CDCl₃) δ 5.08-4.97 (m, 0.5H), 4.87-4.78 (m, 0.5H), 4.79 (s, 2H), 4.20-3.97 (m, 1H), 3.71 (s, 3H), 3.12-2.90 (m, 1H), 2.81-2.67 (m, 1H), 2.49-2.37 (m, 1H), 2218-2.10 (m, 2H), 1.47 (s, 9H).

### Step 2: benzyl 2-methyl (S)-4-methylenepiperidine-1,2-dicarboxylate (1-6c)

To the solution of 1-6b (585 mg, 2.29 mmol) in dry MeOH (3 mL) was added 3N HCl in MeOH (7 mL) under N₂ atmosphere at 0° C., and stirred at r.t. for 2 h. The solution was concentrated and dissolved in CH₂Cl₂ (10 mL). 4N HCl in EtOAc (10 mL) was added, and stirred for 20 min. The solution was concentrated and the residue was dissolved in CH₂Cl₂ (10 mL). To the solution was added Cbz Cl (benzyl chloroformate, 0.40 mL, 2.75 mmol) and TEA (0.65 mL, 4.58 mmol) at 0° C., and stirred at r.t. for 30 min. The solution was added into 0.5 N HCl (3 mL) and water (3 mL), and extracted with CH₂Cl₂ (5 mL*3). The organic phase was concentrated and purified by flash column chromatography (petroleum ether/EtOAc=90/10) to give the title compound 1-6c as a colorless oil (310 mg, 46.8% yield). MS (ESI) m/z: 312.2 [M+Na]⁺.

¹H NMR (400 MHz, CDCl₃) δ 7.45-7.24 (m, 5H), 5.17 (s, 2H), 5.14-4.90 (m, 1H), 4.81 (s, 2H), 4.28-4.08 (m, 1H), 3.69 (d, J=18.8 Hz, 3H), 3.23-3.01 (m, 1H), 2.77 (t, J=15.7 Hz, 1H), 2.50-2.39 (m, 1H), 2.30-2.12 (m, 2H).

### Step 3: 6-benzyl 5-methyl (S)-6-azaspiro[2.5]octane-5,6-dicarboxylate (1-6d)

To dry CH₂Cl₂ (5 mL) was added ZnEt₂ (2M in hexane, 2.1 mL, 4.2 mmol) under N₂ atmosphere at 0° C., stirred at 0° C. for 10 min. Dry TFA (0.33 mL, 4.2 mmol) was added slowly, and stirred at 0° C. for 1 h. Diiodomethane (0.35 mL, 4.2 mmol) was added slowly, and stirred at 0° C. for 1 h. The solution of 1-6c (305 mg, 1.05 mmol) in dry CH₂Cl₂ (3 mL*2) was added slowly, stirred at 0° C. for 30 min. and then stirred at r.t. for 18 h. The solution was filtered through celite. The filtration was washed with sat. NH₄Cl (5 mL) and water (5 mL). The water phase was extracted with CH₂Cl₂ (5 mL*3). The organic phase was concentrated and purified by flash column chromatography (petroleum ether/EtOAc=90/10) to give the title compound 1-6d as a light-yellow oil (195 mg, 61% yield). MS (ESI) m/z: 326.3 [M+Na]*.

¹H NMR (400 MHz, CDCl₃) δ 7.45-7.25 (m, 5H), 5.24-5.08 (m, 2H), 5.04-4.84 (m, 1H), 4.21-4.02 (m, 1H), 3.72 (d, J=6 Hz, 3H), 3.36-3.14 (m, 1H), 2.24-2.12 (m, 1H), 2.00-1.85 (m, 1H), 1.62-1.54 (m, 1H), 0.91-0.72 (m, 1H), 0.42-0.22 (m, 4H).

### Step 4: benzyl (S)-5-(hydroxymethyl)-6-azaspiro[2.5]octane-6-carboxylate (1-6e)

To the solution of 1-6d (195 mg, 0.64 mmol) in dry THE (4 mL) was added LiBHEt₃ (1M in THF, 1.3 mL, 1.3 mmol) under N₂ atmosphere at 0° C., and stirred at 0° C. for 1 h. To the solution was added water (0.5 mL), washed with brine (5 mL), and extracted with EtOAc (5 mL*3). The organic phase was concentrated and purified by flash column chromatography (petroleum ether/EtOAc=50/50) to give the title compound 1-6e as a colorless oil (155 mg, 87.6% yield). MS (ESI) m/z: 276.3 [M+H]⁺.

¹H NMR (400 MHz, CDCl₃) δ 7.40-7.29 (m, 5H), 5.22-5.09 (m, 2H), 4.53-4.39 (m, 1H), 4.20-4.06 (m, 1H), 4.02 (dd, J=11.0, 9.5 Hz, 1H), 3.67 (dd, J=11.1, 5.6 Hz, 1H), 3.19-3.05 (m, 1H), 2.08-1.97 (m, 1H), 1.95-1.81 (m, 1H), 1.08-0.97 (m, 1H), 0.91-0.79 (m, 2H), 0.47-0.38 (m, 1H), 0.35-0.23 (m, 3H).

### Step 5: (S)-(6-azaspiro[2.5]octan-5-yl)methanol (1-6f)

To the solution of 1-6e (155 mg, 0.56 mmol) in MeOH (2 mL) were added 7M NH₃ in MeOH (0.2 mL) and 10% wet Pd/C (16 mg) under N₂ atmosphere, and stirred at H₂ atmosphere for 3 h. The solution was filtered and concentrated to give the title compound 1-6f as a pale yellow oil (82 mg, quant.) which was used directly for the next step without further purification. MS (ESI) m/z: 142.1 [M+H]⁺.

### Step 6: (S)-(4-(benzyloxy)-5-methoxy-2-nitrophenyl)(5-(hydroxymethyl)-6-azaspiro[2.5]octan-6-yl)methanone (1-6 h)

To the solution of 1-6g (160 mg, 0.53 mmol) in CH₂Cl₂ (4 mL) was added oxalyl chloride (0.14 mL, 1.58 mmol) under N₂ atmosphere at 0° C., followed by addition of 1 drop of DMF, stirred for 20 min. The solution became clear and no gas eluted. The solution was concentrated to remove the excess amount of oxalyl chloride. The residue was dissolved in dry CH₂Cl₂ (2 mL) and added to the solution of 1-6f (80 mg, 0.53 mmol) and DIPEA (0.38 mL, 2.1 mmol) in dry CH₂Cl₂ (2 mL) at 0° C., stirred for 20 min. The solution was added into water (5 mL), extracted with CH₂Cl₂ (5 mL*3). The organic phase was concentrated and purified by flash column chromatography (petroleum ether/EtOAc=30/70) to give the title compound 1-6 h as an off-white solid (178 mg, 79% yield). MS (ESI) m/z: 427.4 [M+H]⁺.

### Step 7: (S)-(2-amino-4-(benzyloxy)-5-methoxyphenyl)(5-(hydroxymethyl)-6-azaspiro[2.5]octan-6-yl)methanone (1-6i)

To the solution of 1-6 h (175 mg, 0.41 mmol) and NH₄Cl (336 mg, 6.16 mmol) in MeOH/H₂O (3/1 mL) was added Fe powder (117 mg, 2.05 mmol) under N₂ atmosphere, refluxed for 3 h. The solution was filtered through elite. The filtration was washed with brine (5 mL), extracted with EtOAc (5 mL*3), dried over anhydrous Na₂SO₄, filtered, and concentrated to give the title compound 1-6i (162 mg, quant.) which was used directly for the next step without further purification. MS (ESI) m/z: 397.3 [M+H]⁺.

### Step 8: allyl (S)-(5-(benzyloxy)-2-(5-(hydroxymethyl)-6-azaspiro[2.5]octane-6-carbonyl)-4-methoxyphenyl)carbamate (1-6j)

To the solution of 1-6i (162 mg, 0.41 mmol) and pyridine (67 μL, 0.82 mmol) in dry CH₂Cl₂ (4 mL) was added AllocCl (53 μL, 0.49 mmol) under N₂ atmosphere at −10° C., stirred for 20 min. The solution was added into water (2 mL) and 0.5 N HCl (2 mL), extracted with CH₂Cl₂ (5 mL*3). The organic phase was concentrated and purified by flash column chromatography (petroleum ether/EtOAc=25/75) to give the title compound 1-6j as an off-white solid (157 mg, 80% yield). MS (ESI) m/z: 481.4 [M+H]⁺.

### Step 9: allyl (6aS)-3-(benzyloxy)-6-hydroxy-2-methoxy-12-oxo-6a,7,9,10-tetrahydro-6H-spiro[benzo[e]pyrido[1,2-a][1,4]diazepine-8,1′-cyclopropane]-5(12H)-carboxylate (1-6k)

To the solution of 1-6j (155 mg, 0.32 mmol) in CH₂Cl₂ (3 mL) was added DMP (209 mg, 0.48 mmol) at 0° C., stirred at r.t. for 30 min. To the solution was added sat. Na₂S₂O₃ (2 mL) and sat. NaHCO₃ (2 mL), extracted with CH₂Cl₂ (5 mL*3). The organic phase was concentrated and purified by flash column chromatography (petroleum ether/EtOAc=50/50) to give the title compound 1-6k as a white solid (130 mg, 84% yield). MS (ESI) m/z: 479.4 [M+H]⁺.

### Step 10: allyl (6aS)-3,6-dihydroxy-2-methoxy-12-oxo-6a,7,9,10-tetrahydro-6H-spiro[benzo[e]pyrido[1,2-a][1,4]diazepine-8,1′-cyclopropane]-5(12H)-carboxylate (1-61)

To the solution of 1-6k (75 mg, 0.16 mmol) in CH₂Cl₂ (2.5 mL) was added MsOH (104 μL, 1.56 mmol) under N₂ atmosphere at 0° C., stirred at r.t. for 5 h. The solution was added into brine (3 mL), extracted with CH₂Cl₂/MeOH (10:1, 5.5 mL*3). The organic phase was concentrated and purified by flash column chromatography (petroleum ether/EtOAc=50/50) to give the title compound 1-61 as a white solid (20 mg, 33% yield). MS (ESI) m/z: 389.3 [M+H]⁺.

¹H NMR (400 MHz, CDCl₃) δ 7.18 (s, 1H), 6.73 (s, 1H), 6.11 (d, J=10.3 Hz, 1H), 5.91 (s, 1H), 5.90-5.75 (m, 1H), 5.28-5.10 (m, 2H), 4.66 (dd, J=13.0, 5.1 Hz, 1H), 4.57-4.46 (m, 1H), 4.42 (dt, J=13.4, 4.5 Hz, 1H), 3.96 (s, 3H), 3.61-3.52 (m, 1H), 3.45 (brs, 1H), 3.23 (ddd, J=13.5, 11.5, 4.1 Hz, 1H), 2.02 (dd, J=15.5, 7.4 Hz, 1H), 1.91-1.80 (m, 1H), 1.58 (d, J=14.3 Hz, 1H), 1.35-1.27 (m, 1H), 0.62-0.46 (m, 2H), 0.45-0.33 (m, 2H).

### Step 11: allyl (6aS)-3-((5-(((S)-10-((allyloxy)carbonyl)-7-methoxy-2-(4-methoxyphenyl)-5-oxo-5,10,11,11a-tetrahydro-1H-benzo[e]pyrrolo[1,2-a][1,4]diazepin-8-yl)oxy)pentyl)oxy)-6-hydroxy-2-methoxy-12-oxo-6a,7,9,10-tetrahydro-6H-spiro[benzo[e]pyrido[1,2-a][1,4]diazepine-8,1′-cyclopropane]-5(12H)-carboxylate (1-6n)

To a solution of 1-61 (20 mg, 0.05 mmol) and 1-6m (31 mg, 0.05 mmol, synthesized according to the procedure described in US20200261594A1) in dry DMF (1 mL) was added K₂CO₃ (8.7 mg, 0.06 mmol) under N₂ atmosphere, stirred at r.t. for 4 days. The solution was added into water (12 mL), and extracted with CH₂Cl₂ (5 mL*4). The organic phase was concentrated and purified by flash column chromatography (petroleum ether/EtOAc=25/75) to give the title compound 1-6n as an off-white solid (40 mg, 87% yield). MS (ESI) m/z: 893.6 [M+H]⁺.

### Step 12: (S)-2-methoxy-3-((5-(((S)-7-methoxy-2-(4-methoxyphenyl)-5-oxo-5,10,11,11a-tetrahydro-1H-benzo[e]pyrrolo[1,2-a][1,4]diazepin-8-yl)oxy)pentyl)oxy)-6a,7,9,10-tetrahydro-12H-spiro[benzo[e]pyrido[1,2-a][1,4]diazepine-8,1′-cyclopropan]-12-one (1-6)

To a solution of 1-6n (35 mg, 0.04 mmol) and Pd(PPh₃)₄(1.2 mg, 0.001 mmol) in dry CH₂Cl₂ (1 mL) was added pyrrolidine (4 μL, 0.04 mmol) under N₂ atmosphere, stirred at r.t. for 30 min. The solution was concentrated and purified by flash column chromatography (petroleum ether/EtOAc=0/100) to give the title compound 1-6 as an off-white solid (16 mg, 57.8% yield). MS (ESI) m/z: 707.5 [M+H]⁺.

¹H NMR (400 MHz, CDCl₃) δ 8.10-8.01 (m, 1H), 7.54-7.47 (m, 2H), 7.44 (d, J=6.2 Hz, 1H), 7.31 (d, J=8.7 Hz, 2H), 6.88 (d, J=8.8 Hz, 2H), 6.77 (d, J=8.2 Hz, 1H), 6.23-6.05 (m, 1H), 4.37-4.21 (m, 2H), 4.19-4.04 (m, 2H), 4.00 (t, J=6.6 Hz, 2H), 3.95 (d, J=9.6 Hz, 3H), 3.85 (d, J=3.2 Hz, 3H), 3.82 (s, 3H), 3.64-3.50 (m, 2H), 3.49-3.29 (m, 2H), 2.73 (dd, J=16.1, 3.5 Hz, 1H), 2.23 (dd, J=14.5, 5.9 Hz, 1H), 2.00-1.85 (m, 5H), 1.76-1.59 (m, 4H), 1.49-1.38 (m, 2H), 0.72-0.57 (m, 2H), 0.55-0.43 (m, 2H).

### Example 1-7

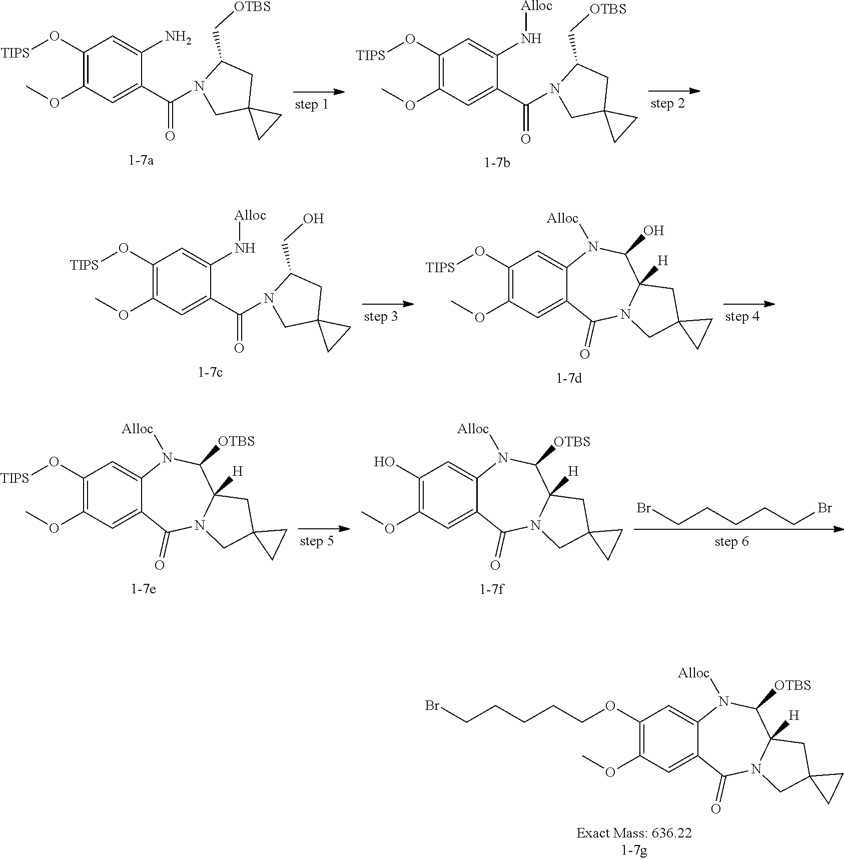

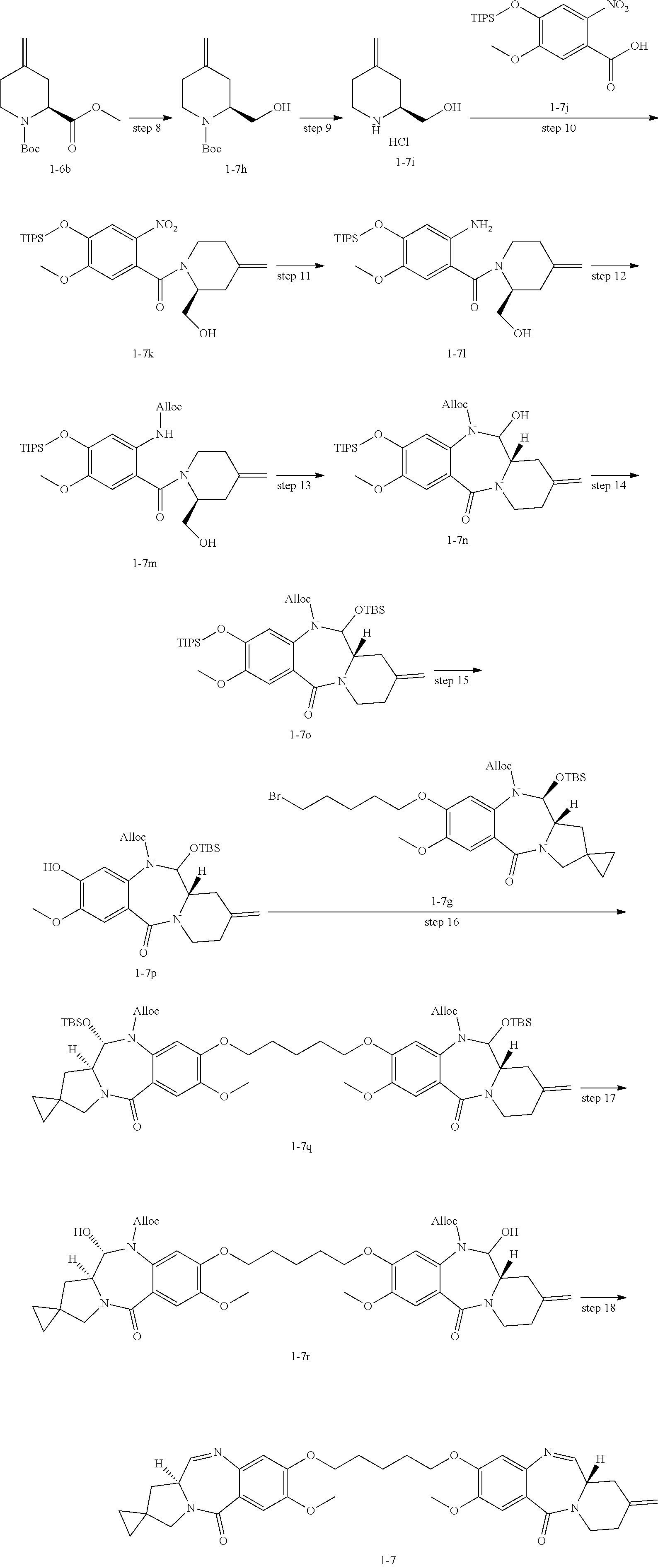

### Step 1: allyl (S)-(2-(6-(((tert-butyldimethylsilyl)oxy)methyl)-5-azaspiro[2.4]heptane-5-carbonyl)-4-methoxy-5-((triisopropylsilyl)oxy)phenyl)carbamate (1-7b)

Pyridine (433 μL) was added to a solution of 1-7a (1370 mg, 2.44 mmol) in dry CH₂Cl₂ at −5° C. Then AllocCl (322 μL) was added to the mixture at −5° C. and the mixture was stirred at −5° C. for 1 h. Reaction completion was observed by TLC (petroleum ether/EtOAc=5:1). The mixture was diluted with CH₂Cl₂, washed with 5% citric acid, sat. NaHCO₃, brine, and dried with Na₂SO₄. The organic phase was concentrated to give the crude product which was used directly for the next step. MS (ESI) m/z: 647.4 [M+H]⁺.

### Step 2: allyl (S)-(2-(6-(hydroxymethyl)-5-azaspiro[2.4]heptane-5-carbonyl)-4-methoxy-5-((triisopropylsilyl)oxy)phenyl)carbamate (1-7c)

para-Toluenesulfonic acid hydrate (282 mg, 1.49 mmol) was added to a solution of 1-7b (1.60 g, 2.48 mmol) in THE (20 mL) and water (1 mL). The reaction mixture was allowed to stir for 1 h at 22° C. Completed reaction was observed by TLC (petroleum ether/EtOAc=5:1, 1:1), diluted with EtOAc (60 ml), washed with water and brine. The organic phase was concentrated and purified by flash column chromatography to give 1.09 g 1-7c (83% yield). MS (ESI) m/z: 533.3 [M+H]⁺.

### Step 3: allyl (11S,11aS)-11-hydroxy-7-methoxy-5-oxo-8-((triisopropylsilyl)oxy)-11,11a-dihydro-1H,3H-spiro[benzo[e]pyrrolo[1,2-a][1,4]diazepine-2,1′-cyclopropane]-10(5H)-carboxylate (1-7d)

Anhydrous DMSO (436 μL, 6.14 mmol) was added dropwise to a solution of oxalyl chloride (260 μL, 3.07 mmol) in dry CH₂Cl₂ (20 mL) at −70° C. After 30 min, a solution of 1-7c (1.09 g, 2.05 mmol) in dry CH₂Cl₂ (10 mL) was added slowly while maintaining the temperature at −70° C. After 40 min, triethylamine (1423 μL, dried over 4 Å molecular sieves) was added dropwise and the temperature was allowed to reach −50° C. for 1 h. The reaction mixture was allowed to warm to r.t. and stirred for 1 h. Completed reaction was observed by TLC (petroleum ether/EtOAc=1:1, CH₂Cl₂/EtOAc=10:1). The reaction mixture was washed with 5% aqueous citric acid (10 V) to pH=3. The organic phase was washed with saturated aqueous NaHCO₃ and water and dried over sodium sulfate. The organic phase was concentrated to give the crude product which was purified by flash column chromatography (CH₂Cl₂/EtOAc=95/5) to give 1-7d (416 mg, 38% yield). MS (ESI) m/z: 531.3 [M+H]⁺.

### Step 4: allyl (11S,11aS)-11-((tert-butyldimethylsilyl)oxy)-7-methoxy-5-oxo-8-((triisopropylsilyl)oxy)-11,11a-dihydro-1H,3H-spiro[benzo[e]pyrrolo[1,2-a][1,4]diazepine-2,1′-cyclopropane]-10(5H)-carboxylate (1-7e)

TBSOTf (0.54 mL, 2.35 mmol) was added to a mixture of 1-7d (416 mg, 0.78 mmol) and 2,6-lutidine (0.37 mL, 3.14 mmol) in dry CH₂Cl₂ (10 mL) at 0° C. The reaction mixture was allowed to stir at 5° C. for 30 min, followed by 1 h at 25° C. Completion was observed by LCMS. The reaction mixture was washed with saturated aqueous NaHCO₃ and brine, dried with Na₂SO₄, and concentrated to give crude product. The crude product was purified by flash column chromatography (petroleum ether/EtOAc=80/20) to give 1-7e (486 mg, 96% yield). MS (ESI) m/z: 645.5 [M+H]⁺.

### Step 5: allyl (11S,11aS)-11-((tert-butyldimethylsilyl)oxy)-8-hydroxy-7-methoxy-5-oxo-11,11a-dihydro-1H,3H-spiro[benzo[e]pyrrolo[1,2-a][1,4]diazepine-2,1′-cyclopropane]-10(5H)-carboxylate (1-7f)

Lithium acetate (50 mg, 0.73 mmol) was added to a solution of 1-7e (468 mg, 0.73 mmol) in wet DMF (10 mL, 49/1 DMF/water). The reaction was allowed to proceed for 2 h at 25° C. Completion was observed by TLC (petroleum ether/EtOAc=2:1, 1:1). The mixture was diluted with EtOAc, washed with 5% aqueous citric acid and brine. The organic phase was dried with Na₂SO₄ and concentrated to give the crude product which was purified by flash column chromatography to give 1-7f (303 mg, 85% yield). MS (ESI) m/z: 489.4 [M+H]⁺.

### Step 6: allyl (11S,11aS)-8-((5-bromopentyl)oxy)-11-((tert-butyldimethylsilyl)oxy)-7-methoxy-5-oxo-11,11a-dihydro-1H,3H-spiro[benzo[e]pyrrolo[1,2-a][1,4]diazepine-2,1′-cyclopropane]-10(5H)-carboxylate (1-7g)

To a solution of 1-7f (100 mg, 0.20 mmol) and 1,5-dibromopentane (0.42 mL, 3.07 mmol) in 2 mL DMF was added K₂CO₃ (34 mg, 0.25 mmol) at r.t. The mixture was stirred at r.t. for 2 h. LCMS showed the starting material was consumed completely. The mixture was diluted with EtOAc, washed with H₂O and brine. The organic phase was concentrated and purified by flash column chromatography (petroleum ether/EtOAc) to give 1-7g (110 mg, 84% yield). MS (ESI) m/z: 637.4.3 [M+H]⁺.

### Step 7: tert-butyl (S)-2-(hydroxymethyl)-4-methylenepiperidine-1-carboxylate (1-7 h)

LiCl (180 mg, 4.23 mmol) in water (0.67 mL) was added to 1-6b (830 mg, 3.25 mmol) in dry THE (10 mL) at r.t. Then NaBH₄ (160 mg, 4.23 mmol) was added to the mixture. The reaction mixture was allowed to stir at r.t. overnight. 2 N HCl (5 V) was added to the mixture at 0° C., then sat. NaHCO₃ was added to the mixture until the pH=7-8. The reaction mixture was extracted with EtOAc, washed with brine, dried with Na₂SO₄ and concentrated to give crude product. The crude product was purified by flash column chromatography (petroleum ether/EtOAc=75/25) to give 1-7 h (495 mg, 71% yield).

¹H NMR (400 MHz, CDCl₃) δ 4.79 (d, J=23.7 Hz, 2H), 4.41 (d, J=6.1 Hz, 1H), 4.06 (s, 1H), 3.67 (dd, J=11.1, 9.0 Hz, 1H), 3.57 (dd, J=11.2, 5.9 Hz, 1H), 2.91 (s, 1H), 2.37 (dd, J=14.1, 6.1 Hz, 1H), 2.27-2.15 (m, 3H), 1.48 (s, 9H).

### Step 8: (S)-2-(hydroxymethyl)-4-methylenepiperidin-1-ium chloride (1-7i)

4 M HCl in MeOH (6 mL) was added to 1-7 h (525 mg, 2.31 mmol) in MeOH (5 mL) at 0° C. The reaction mixture was allowed to stir at r.t. for 2 h. Completion was observed by TLC (petroleum ether/EtOAc=2:1). The reaction mixture was washed with saturated aqueous NaHCO3 and water, dried with Na₂SO₄ and concentrated to give crude product. The crude product was purified by flash column chromatography to give 1-7i (425 mg). MS (ESI) m/z: 128.1 [M+H]⁺.

### Step 9: (S)-(2-(hydroxymethyl)-4-methylenepiperidin-1-yl)(5-methoxy-2-nitro-4-((triisopropylsilyl)oxy)phenyl)methanone (1-7k)

Oxalyl chloride (603 μL, 7.07 mmol) was added dropwise to a stirred solution of 1-7i (870 mg, 2.36 mmol) in dry CH₂Cl₂ (8 mL) and THE (8 mL) and DMF (4 μL) at 0° C. under N₂. The reaction was warmed to r.t. and was stirred for 1 h at r.t. Completion was observed by TLC (petroleum ether/EtOAc=1:1). The mixture was concentrated to give the crude product which was used directly for the next step. The crude product and 1-7j (423 mg, 2.59 mmol) were dissolved in CH₂Cl₂ (8 mL). The reaction mixture was cooled to 0° C. and triethylamine (983 μL, 7.07 mmol) was dropwise under N₂. The mixture was then warmed to r.t. and stirred for 3 h. The solution was concentrated, and the crude product was purified by flash column chromatography to give 1-7k (788 mg, 79% yield). MS (ESI) m/z: 479.4 [M+H]⁺.

### Step 10: (S)-(2-amino-5-methoxy-4-((triisopropylsilyl)oxy)phenyl)(2-(hydroxymethyl)-4-methylenepiperidin-1-yl)methanone (1-71)

Zinc powder (1.77 g, 27.08 mmol) was added to a mixture of ethanol (4 mL), water (0.25 mL), and AcOH (0.25 mL) at 0° C. The reaction mixture was stirred at 5° C. for 30 min. A solution of 1-7k (0.35 g, 0.73 mmol) in ethanol (2 mL) was added dropwise at 5° C. The reaction was allowed to proceed at 5° C. for 30 min. The solids were removed by filtration. The filtrate was diluted with ethyl acetate and washed with water, saturated aqueous NaHCO₃ and brine. The organic phase was dried over sodium sulfate and filtered, the solvent removed by rotary evaporation under reduced pressure to afford the product as a brown oil (268 mg, 82% yield) which was used directly in the next step. MS (ESI) m/z: 449.3 [M+H]⁺.

### Step 11: allyl (S)-(2-(2-(hydroxymethyl)-4-methylenepiperidine-1-carbonyl)-4-methoxy-5-((triisopropylsilyl)oxy)phenyl)carbamate (1-7m)

Pyridine (104 μL) was added to a solution of 1-71 (268 mg, 0.60 mmol) in dry CH₂Cl₂ at −5° C. Then AllocCl (64 μL, 72.04 mmol) was added to the mixture at −5° C. and the mixture was stirred at −5° C. for 0.5 h. Reaction completion was observed by LCMS. The mixture was diluted with CH₂Cl₂, washed with 5% citric acid, sat. NaHCO₃, brine and dried with Na₂SO₄. The organic phase was concentrated to give the crude product which was purified by flash column chromatography (petroleum ether/EtOAc=65/35) to give 1-7m (236 mg, 74% yield). MS (ESI) m/z: 533.4 [M+H]⁺.

### Step 12: allyl (6S,6aS)-6-hydroxy-2-methoxy-8-methylene-12-oxo-3-((triisopropylsilyl)oxy)-6,6a,7,8,9,10-hexahydrobenzo[e]pyrido[1,2-a][1,4]diazepine-5(12H)-carboxylate (1-7n)

DMP (172 mg, 0.41 mmol) was added slowly, portion-wise to a solution of 1-7m (206 mg, 0.39 mmol) in dry CH₂Cl₂ (5 mL) at 0° C. The reaction was then warmed to r.t. and stirred for 2 h. 0.5 eq. DMP was added to the reaction in batches. After 9 h, the starting material was consumed. The mixture was quenched with sat. Na₂S₂O₃, followed by addition of sat. NaHCO₃ and water. The layers were separated, and the organic layer was washed with sat. Na₂S₂O₃, sat. NaHCO₃ and brine, dried over Na₂SO₄. The crude product was purified by flash column chromatography (CH₂Cl₂/EtOAc=93/7) to give 1-7n (157 mg, 85% yield). MS (ESI) m/z: 531.3 [M+H]⁺.

### Step 13: allyl (6S,6aS)-6-((tert-butyldimethylsilyl)oxy)-2-methoxy-8-methylene-12-oxo-3-((triisopropylsilyl)oxy)-6,6a,7,8,9,10-hexahydrobenzo[e]pyrido[1,2-a][1,4]diazepine-5(12H)-carboxylate (1-70)

TBSOTf (0.23 mL, 1.0 mmol) was added to a mixture of 1-7n (177 mg, 0.33 mmol) and 2,6-lutidine (0.16 mL) in dry CH₂Cl₂ (5 mL) at 0° C. The reaction mixture was allowed to stir at 5° C. for 30 min, followed by 1 h at 25° C. The reaction mixture was washed with saturated aqueous NaHCO₃ and brine, dried with Na₂SO₄ and concentrated to give crude product. The crude product was purified by flash column chromatography (CH₂Cl₂/EtOAc=98/2) to give 1-7o (135 mg, 63% yield). MS (ESI) m/z: 645.4 [M+H]⁺.

### Step 14: allyl (6S,6aS)-6-((tert-butyldimethylsilyl)oxy)-3-hydroxy-2-methoxy-8-methylene-12-oxo-6,6a,7,8,9,10-hexahydrobenzo[e]pyrido[1,2-a][1,4]diazepine-5(12H)-carboxylate (1-7p)

Lithium acetate (14 mg, 0.21 mmol) was added to a solution of 1-7o (135 mg, 0.21 mmol) in wet dimethylformamide (3 mL, 49/1 DMF/water). The reaction was allowed to proceed for 2 h at 25° C. Completion was observed by TLC (petroleum ether/EtOAc=1:1). The mixture was diluted with EtOAc, washed with 5% aqueous citric acid, sat. NaHCO₃ and brine. The organic phase was dried with Na₂SO₄ and concentrated to give the crude product which was purified by flash column chromatography to give 1-7p (100 mg, 98% yield). MS (ESI) m/z: 489.3 [M+H]⁺.

### Step 15: allyl (11S,11aS)-8-((5-(((6S,6aS)-5-((allyloxy)carbonyl)-6-((tert-butyldimethylsilyl)oxy)-2-methoxy-8-methylene-12-oxo-5,6,6a,7,8,9,10,12-octahydrobenzo[e]pyrido[1,2-a][1,4]diazepin-3-yl)oxy)pentyl)oxy)-11-((tert-butyldimethylsilyl)oxy)-7-methoxy-5-oxo-11,11a-dihydro-1H,3H-spiro[benzo[e]pyrrolo[1,2-a][1,4]diazepine-2,1′-cyclopropane]-10(5H)-carboxylate (1-7q)

1-7p (28 mg, 0.06 mmol) and K₂CO₃ (10 mg, 0.07 mmol) were added to a solution of 1-7g (38 mg, 0.06 mmol) in 1 mL DMF. The mixture was stirred at r.t. overnight. The product (petroleum ether/EtOAc=1:2) was detected by LCMS. The mixture was diluted with EtOAc, washed with water, brine. The organic phase was concentrated and purified by flash column chromatography (petroleum ether/EtOAc=27/73) to give 1-7q (48 mg, 84% yield). MS (ESI) m/z: 1045.7 [M+H]⁺.

### Step 16: allyl (11S,11aS)-8-((5-(((6S,6aS)-5-((allyloxy)carbonyl)-6-hydroxy-2-methoxy-8-methylene-12-oxo-5,6,6a,7,8,9,10,12-octahydrobenzo[e]pyrido[1,2-a][1,4]diazepin-3-yl)oxy)pentyl)oxy)-11-hydroxy-7-methoxy-5-oxo-11,11a-dihydro-1H,3H-spiro[benzo[e]pyrrolo[1,2-a][1,4]diazepine-2,1′-cyclopropane]-10(5H)-carboxylate (1-7r)

1 M TBAF (230 μL, 0.23 mmol) was added to the solution of 1-7q (48 mg, 0.05 mmol) in dry 3 mL THE and AcOH (16 μL). The mixture was stirred at r.t. for 2 h. On the completion of the reaction by LCMS, the mixture was diluted with EtOAc, washed with sat. NaHCO₃ and brine. The organic phase was concentrated and purified by flash column chromatography to give 1-7r (21 mg, 56% yield). MS (ESI) m/z: 817.5 [M+H]⁺.

### Step 17: (S)-7-methoxy-8-((5-(((S)-2-methoxy-8-methylene-12-oxo-6a,7,8,9,10,12-hexahydrobenzo[e]pyrido[1,2-a][1,4]diazepin-3-yl)oxy)pentyl)oxy)-1,11a-dihydro-3H,5H-spiro[benzo[e]pyrrolo[1,2-a][1,4]diazepine-2,1′-cyclopropan]-5-one (1-7)

Pd(PPh₃)₄(2.2 mg, 0.002 mmol) was added to a solution of 1-7r (15 mg, 0.02 mmol) in CH₂Cl₂ (0.5 mL) and pyrrolidine (8 μL, 0.09 mmol). The reaction mixture was allowed to stir for 0.5 h at r.t. The reaction was diluted with CH₂Cl₂ (10 mL), washed with sat. NH₄Cl and brine. The organic phase was concentrated and purified by prep-HPLC (0.01% FA in H₂O) to give 1-7 (9 mg, 80% yield) as a white solid. MS (ESI) m/z: 613.4 [M+H]⁺.

¹H NMR (400 MHz, CDCl₃) δ 7.82 (d, J=5.1 Hz, 1H), 7.78 (d, J=5.1 Hz, 1H), 7.51 (s, 1H), 7.47 s, 1H), 6.81 (d, J=7.2 Hz, 2H), 5.09 (d, J=28.2 Hz, 2H), 4.21-4.02 (m, 4H), 4.03-3.74 (m, 11H), 3.68 (d, J=11.7 Hz, 1H), 3.49 (d, J=11.7 Hz, 1H), 2.87-2.75 (m, 1H), 2.70-2.64 (m, 2H), 2.53 (dd, J=12.9, 8.1 Hz, 2H), 1.97 (m, 6H), 0.84-0.66 (m, 4H).

### Example 1-8

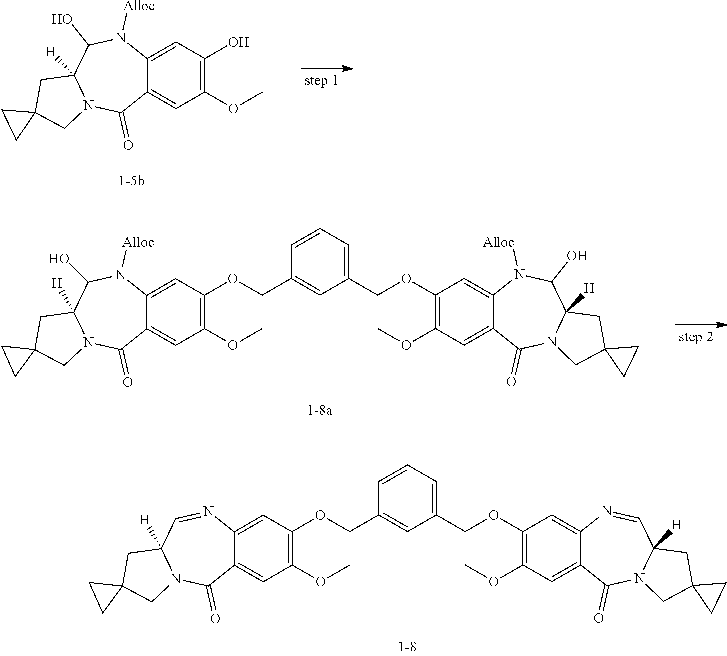

### Step 1: diallyl 8,8″-((1,3-phenylenebis(methylene))bis(oxy))(11aS,11a″S)-bis(11-hydroxy-7-methoxy-5-oxo-11,11a-dihydro-1H,3H-spiro[benzo[e]pyrrolo[1,2-a][1,4]diazepine-2,1′-cyclopropane]-10(5H)-carboxylate) (1-8a)

To a solution of 1-5b (50 mg, 0.13 mmol) and 1,3-bis(bromomethyl)benzene (17.5 mg, 0.07 mmol) in DMF (2 mL) was added K₂CO₃ (36.94 mg, 0.27 mmol) at r.t. The mixture was stirred at 20° C. for 3 h. The reaction mixture was diluted with EtOAc (10 mL) and washed with brine (8 mL*3). The organic layer was dried over Na₂SO₄ and concentrated to give a residue which was purified by silica column gel chromatography (eluent: petroleum ether/EtOAc=100/0 to 10/90) to afford 1-8a (50 mg, 78.2% yield) as a white solid.

MS (ESI) m/z: 851.3 [M+H]⁺.

### Step 2: (11aS,11a″S)-8,8″-((1,3-phenylenebis(methylene))bis(oxy))bis(7-methoxy-1,11a-dihydro-3H,5H-spiro[benzo[e]pyrrolo[1,2-a][1,4]diazepine-2,1′-cyclopropan]-5-one) (1-8)

To a solution of 1-8a (50 mg, 0.06 mmol) and CH₂Cl₂ (2 mL) was added Pd(PPh₃)₄ (6.79 mg, 0.01 mmol) and pyrrolidine (12 μL, 0.15 mmol) at r.t. The mixture was stirred at 20° C. for 30 min. Solvent was evaporated and the residue was purified by silica column gel chromatography (eluent: CH₂Cl₂/MeOH=100/0 to 1/99) and prep-HPLC (0.01% FA) to give product 1-8 (33.2 mg, 78.63% yield) as a white solid.

MS (ESI) m/z: 647.3 [M+H]⁺.

¹H NMR (400 MHz, CDCl₃) δ 7.80-7.75 (2H, m), 7.54 (2H, s), 7.51 (1H, s), 7.40 (3H, d, J=4.9), 6.85 (2H, d, J 1.4), 5.25-5.14 (4H, m), 3.96 (6H, s), 3.85 (2H, dd, J=7.9, 3.2), 3.67 (2H, d, J=11.7), 3.52-3.45 (2H, m), 2.51 (2H, dd, J=13.0, 8.1), 1.99 (2H, d, J=12.8), 0.74 (10H, dd, J=10.6, 6.6).

### Examples 1-9 & 1-10

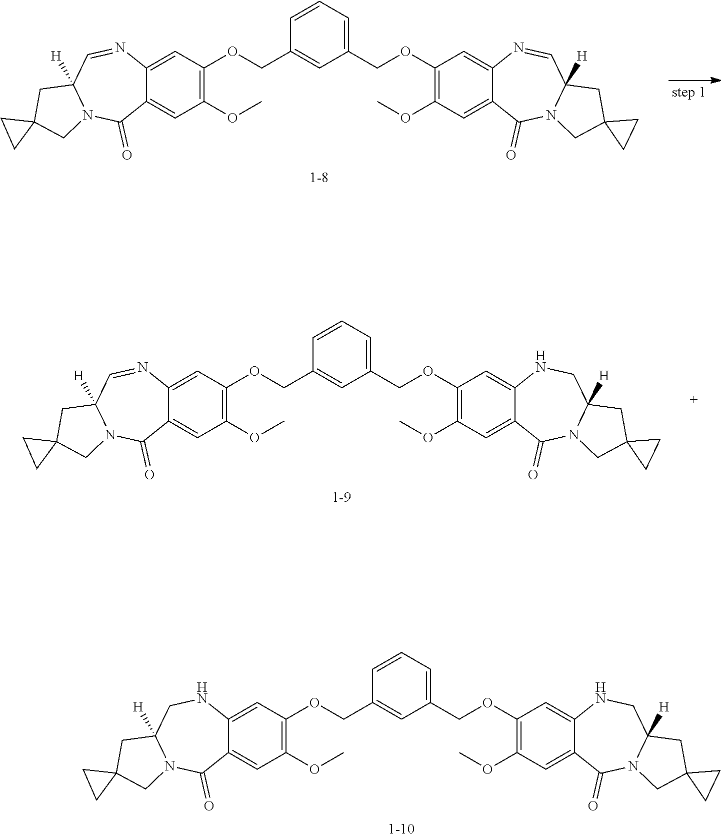 [SMILES: COc1cc2c(cc1OCc1cccc(COc3cc4c(cc3OC)C(=O)N3CC5(CC5)C[C@H]3C=N4)c1)N=C[C@@H]1CC3(CC3)CN1C2=O ; COc1cc2c(cc1OCc1cccc(COc3cc4c(cc3OC)C(=O)N3CC5(CC5)C[C@H]3CN4)c1)N=C[C@@H]1CC3(CC3)CN1C2=O ; COc1cc2c(cc1OCc1cccc(COc3cc4c(cc3OC)C(=O)N3CC5(CC5)C[C@H]3CN4)c1)NC[C@@H]1CC3(CC3)CN1C2=O]

### Step 1: (S)-7-methoxy-8-((3-((((S)-7-methoxy-5-oxo-5,11a-dihydro-1H,3H-spiro[benzo[e]pyrrolo[1,2-a][1,4]diazepine-2,1′-cyclopropan]-8-yl)oxy)methyl)benzyl)oxy)-1,10,11,11a-tetrahydro-3H,5H-spiro[benzo[e]pyrrolo[1,2-a][1,4]diazepine-2,1′-cyclopropan]-5-one (1-9) and (S)-7-methoxy-8-((3-((((S)-7-methoxy-5-oxo-5,11a-dihydro-1H,3H-spiro[benzo[e]pyrrolo[1,2-a][1,4]diazepine-2,1′-cyclopropan]-8-yl)oxy)methyl)benzyl)oxy)-1,10,11,11a-tetrahydro-3H,5H-spiro[benzo[e]pyrrolo[1,2-a][1,4]diazepine-2,1′-cyclopropan]-5-one (1-10)

To a solution of 1-8 (30 mg, 0.05 mmol) in CH₂Cl₂ (3 mL) was added NaBH(OAc)₃ (14.76 mg, 0.07 mmol) at 0° C. The mixture was stirred at 20° C. for 30 min. The reaction was quenched with water (10 mL) and extracted with CH₂Cl₂ (10 mL*3). The combined organic layer was dried over Na₂SO₄ and filtered. Solvent was evaporated and the residue was purified by prep-HPLC (0.01% FA) to give product 1-9 (8 mg, 25.25% yield) as a white solid and 1-10 (12 mg, 38.96%) as a white solid.

1-9 MS (ESI) m/z: 649.3 [M+H]⁺.

1-10 MS (ESI) m/z: 651.3 [M+H]⁺.

1-10 ¹H NMR (400 MHz, CDCl₃) δ 7.57 (2H, s), 7.56-7.48 (1H, m), 7.37 (2H, d, J=9.8), 7.34 (3H, s), 6.08 (2H, s), 5.13-5.02 (4H, m), 3.96 (2H, t, J=7.1), 3.86 (6H, s), 3.68 (2H, d, J=12.0), 3.54 (2H, d, J=12.0), 3.50-3.46 (2H, m), 3.38 (2H, dd, J=12.4, 8.9), 2.06-1.94 (2H, m), 1.75 (2H, dd, J=12.6, 6.7), 0.74-0.51 (8H, m).

### Example 1-11

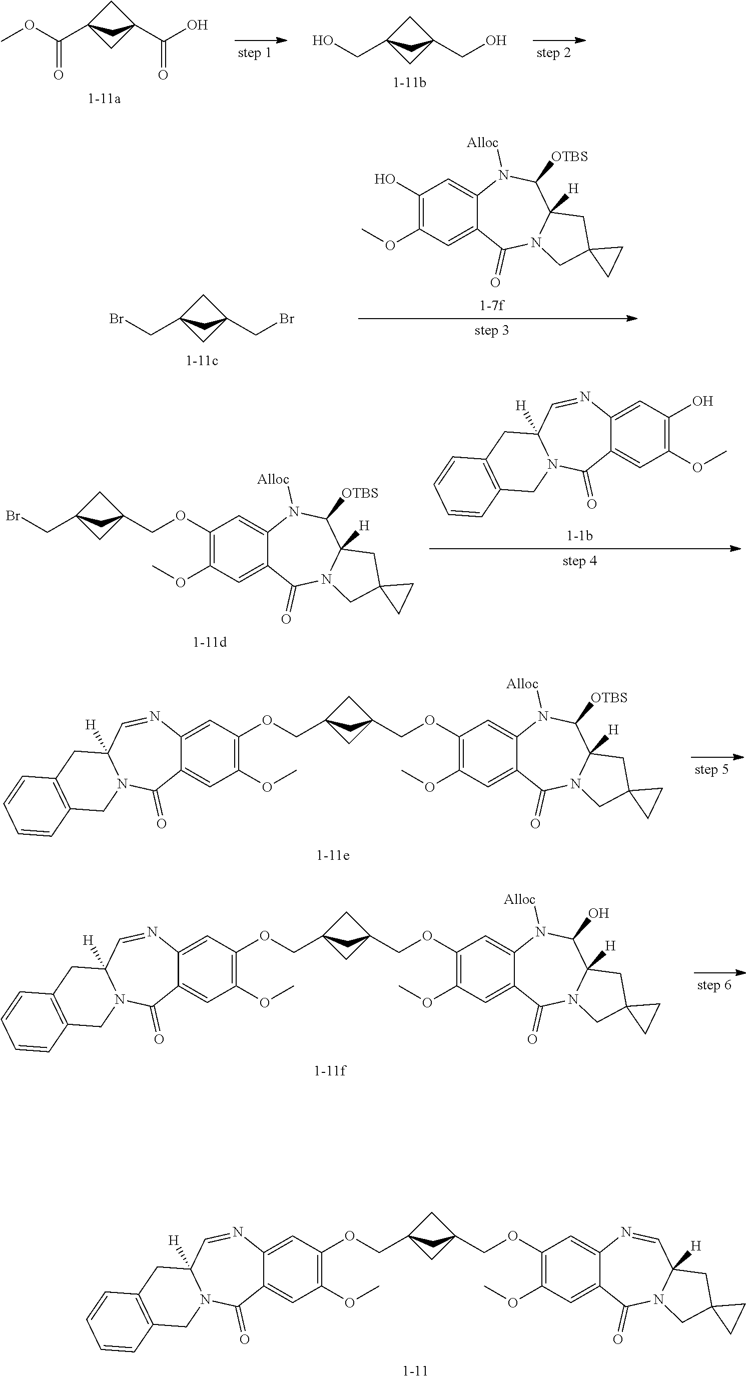

### Step 1: bicyclo[1.1.1]pentane-1,3-diyldimethanol (1-11b)

LiAlH₄ (2.1 g, 52.9 mmol) was added to a solution of 1-11a (3 g, 17.64 mmol) in dry THE (80 ml) in batches at 0° C., then the mixture was warmed to r.t. and stirred overnight. After the reaction was completed, the reaction mixture was quenched with sodium sulfate decahydrate for 1 h, filtered and the filtrate was concentrated under reduced pressure to obtain oil product 1-lib (2.2 g, 97% yield).

¹H NMR (400 MHz, d6-DMSO) δ 4.39 (t, J=5.6 Hz, 2H), 3.34 (t, J=4.9 Hz, 4H), 1.45 (s, 6H).

### Step 2: 1,3-bis(bromomethyl)bicyclo[1.1.1]pentane (1-11c)

To a solution of triphenylphosphine (2.05 g, 7.81 mmol) in MeCN (30 mL) was added dropwise a solution of liquid bromine (0.4 mL, 7.81 mmol) in 5 mL MeCN at 0° C., then 1-11b (500 mg, 3.90 mmol) was added. The reaction was heated to 80° C. and refluxed overnight. The solvent was removed under vacuum and purified by flash column chromatography to give 1-11c (770 mg, 78% yield).

¹H NMR (400 MHz, CDCl₃) δ 3.47 (s, 4H), 1.73 (s, 6H).

### Step 3: allyl (11S,11As)-8-((3-(bromomethyl)bicyclo[1.1.1]pentan-1-yl)methoxy)-11-((tert-butyldimethylsilyl)oxy)-7-methoxy-5-oxo-11,11a-dihydro-1H,3H-spiro[benzo[e]pyrrolo[1,2-a][1,4]diazepine-2,1′-cyclopropane]-10(5H)-carboxylate (1-11d)

To a solution of 1-7f (30 mg, 0.06 mmol) and 1-11c (154 mg, 0.61 mmol) in 2 mL DMF was added K₂CO₃ (12 mg, 0.07 mmol) at r.t. The mixture was stirred at r.t. for 3 h. The mixture was diluted with EtOAc, washed with H₂O and brine. The organic phase was concentrated and purified by flash column chromatography to give 1-11d (40 mg, 99% yield). MS (ESI) m/z: 661.3 [M+H]⁺.

### Step 4: allyl (11S,11aS)-11-((tert-butyldimethylsilyl)oxy)-7-methoxy-8-((3-((((S)-2-methoxy-14-oxo-6a,7,12,14-tetrahydrobenzo[5,6][1,4]diazepino[1,2-b]isoquinolin-3-yl)oxy)methyl)bicyclo[1.1.1]pentan-1-yl)methoxy)-5-oxo-11,11a-dihydro-1H,3H-spiro[benzo[e]pyrrolo[1,2-a][1,4]diazepine-2,1′-cyclopropane]-10(5H)-carboxylate (1-11e)

1-1b (19 mg, 0.06 mmol) and K₂CO₃ (11 mg, 0.07 mmol) were added to a solution of 1-11d (43 mg, 0.06 mmol) in 1 mL DMF. The reaction was warmed to 40° C. for 36 h. The mixture was diluted with EtOAc, washed with water and brine. The organic phase was concentrated and purified by flash column chromatography (CH₂Cl₂/MeOH=94/6) to give 1-11e (28 mg, 51% yield). MS (ESI) m/z: 889.6 [M+H].

### Step 5: allyl (11S,11aS)-11-hydroxy-7-methoxy-8-((3-((((S)-2-methoxy-14-oxo-6a,7,12,14-tetrahydrobenzo[5,6][1,4]diazepino[1,2-b]isoquinolin-3-yl)oxy)methyl)bicyclo[1.1.1]pentan-1-yl)methoxy)-5-oxo-11,11a-dihydro-1H,3H-spiro[benzo[e]pyrrolo[1,2-a][1,4]diazepine-2,1′-cyclopropane]-10(5H)-carboxylate (1-11f)

A buffer of 1 M TBAF (158 μL, 0.16 mmol) and AcOH (11 μL, 0.19 mmol) was added to a solution of 1-11e (28 mg, 0.03 mmol) in dry 5 mL THF. The mixture was stirred at r.t. for 2 h. On the completion of the reaction as determined by LCMS, the mixture was diluted with EtOAc, washed with H₂O, 5% citric acid, and brine. The organic phase was concentrated and purified by flash column chromatography to give 1-11f (24 mg, 98% yield). MS (ESI) m/z: 775.4 [M+H]⁺.

### Step 6: (S)-2-methoxy-3-((3-((((S)-7-methoxy-5-oxo-5,11a-dihydro-1H,3H-spiro[benzo[e]pyrrolo[1,2-a][1,4]diazepine-2,1′-cyclopropan]-8-yl)oxy)methyl)bicyclo[1.1.1]pentan-1-yl)methoxy)-7,12-dihydrobenzo[5,6][1,4]diazepino[1,2-b]isoquinolin-14(6aH)-one (1-11)

Pd(PPh₃)₄(3.6 mg, 0.003 mmol) was added to a solution of 1-11f (24 mg, 0.03 mmol) in CH₂Cl₂ (0.5 mL) and pyrrolidine (11 μL, 0.15 mmol). The reaction mixture was allowed to stir for 0.5 h at r.t. The reaction was concentrated and purified by prep-HPLC (0.01% FA in H₂O) to give 1-11 (10.1 mg, 53% yield) as a white solid. MS (ESI) m/z: 673.3 [M+H]⁺.

¹H NMR (400 MHz, CDCl₃) δ 7.78 (d, J=4.1 Hz, 1H), 7.51 (d, J=6.8 Hz, 2H), 7.46 (d, J=5.0 Hz, 1H), 7.42-7.29 (m, 4H), 6.79 (d, J=5.2 Hz, 2H), 5.00 (d, J=15.5 Hz, 1H), 4.56 (d, J=15.4 Hz, 1H), 4.25-4.07 (m, 4H), 3.96-3.94 (m, 1H), 3.94 (d, J=2.0 Hz, 6H), 3.85 (d, J=5.4 Hz, 1H), 3.67 (d, J=11.6 Hz, 1H), 3.49 (d, J=11.6 Hz, 1H), 3.27 (dd, J=15.2, 5.3 Hz, 1H), 3.16 (dd, J=15.4, 3.9 Hz, 1H), 2.52 (dd, J=13.0, 7.9 Hz, 1H), 2.07-1.97 (m, 1H), 1.90 (s, 6H), 0.80-0.68 (m, 4H).

### Example 1-12

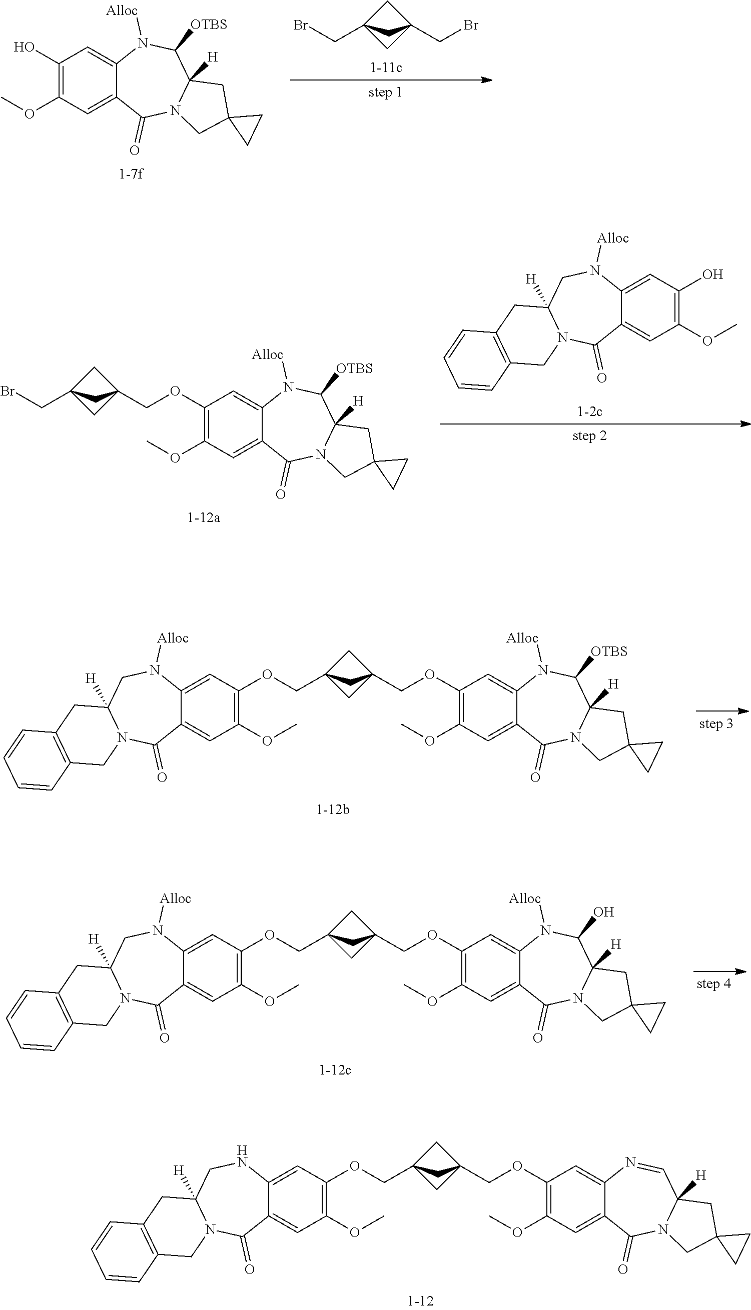

### Step 1: allyl (11S,11aS)-8-((3-(bromomethyl)bicyclo[1.1.1]pentan-1-yl)methoxy)-11-((tert-butyldimethylsilyl)oxy)-7-methoxy-5-oxo-11,11a-dihydro-1H,3H-spiro[benzo[e]pyrrolo[1,2-a][1,4]diazepine-2,1′-cyclopropane]-10(5H)-carboxylate (1-12a)

To a solution of 1-7f (35 mg, 0.07 mmol) and 1-11c (180 mg, 0.72 mmol) in 2 mL DMF was added K₂CO₃ (12 mg, 0.09 mmol) at r.t. The mixture was stirred at r.t. for 5 h. The mixture was diluted with EtOAc, washed with H₂O and brine. The organic phase was concentrated and purified by flash column chromatography to give 1-12a (41 mg, 86% yield). MS (ESI) m/z: 661.3 [M+H]⁺.

### Step 2: allyl (S)-3-((3-((((11S,11aS)-10-((allyloxy)carbonyl)-11-((tert-butyldimethylsilyl)oxy)-7-methoxy-5-oxo-5,10,11,11a-tetrahydro-1H,3H-spiro[benzo[e]pyrrolo[1,2-a][1,4]diazepine-2,1′-cyclopropan]-8-yl)oxy)methyl)bicyclo[1.1.1]pentan-1-yl)methoxy)-2-methoxy-14-oxo-6,6a,7,12-tetrahydrobenzo[5,6][1,4]diazepino[1,2-b]isoquinoline-5(14H)-carboxylate (1-12b)

1-2c (23 mg, 0.06 mmol) and K₂CO₃ (11 mg, 0.08 mmol) were added to a solution of 1-12a (41 mg, 0.06 mmol) in 1 mL DMF. The mixture was stirred at r.t. overnight. The product (petroleum ether/EtOAc=1:2) was detected by LCMS. The mixture was diluted with EtOAc, washed with water, brine. The organic phase was concentrated and purified by flash column chromatography (petroleum ether/EtOAc=27/73) to give 1-12b (48 mg, 84% yield). MS (ESI) m/z: 4974.6 [M+H]⁺.

### Step 3: allyl (S)-3-((3-((((11S,11aS)-10-((allyloxy)carbonyl)-11-hydroxy-7-methoxy-5-oxo-5,10,11,11a-tetrahydro-1H,3H-spiro[benzo[e]pyrrolo[1,2-a][1,4]diazepine-2,1′-cyclopropan]-8-yl)oxy)methyl)bicyclo[1.1.1]pentan-1-yl)methoxy)-2-methoxy-14-oxo-6,6a,7,12-tetrahydrobenzo[5,6][1,4]diazepino[1,2-b]isoquinoline-5(14H)-carboxylate (1-12c)

A mixed solution of 1 M TBAF (248 μL, 0.25 mmol) and AcOH (18 μL, 0.30 mmol) was added to the solution of 1-12b (48 mg, 0.05 mmol) in dry 5 mL THF. The mixture was stirred at r.t. for 2 h. On the completion of the reaction by LCMS, the mixture was diluted with EtOAc, washed with H₂O, 5% citric acid, brine. The organic phase was concentrated and purified by flash column chromatography to give 1-12c (30 mg, 78% yield). MS (ESI) m/z: 861.5 [M+H]⁺.

### Step 4: (S)-2-methoxy-3-((3-((((S)-7-methoxy-5-oxo-5,11a-dihydro-1H,3H-spiro[benzo[e]pyrrolo[1,2-a][1,4]diazepine-2,1′-cyclopropan]-8-yl)oxy)methyl)bicyclo[1.1.1]pentan-1-yl)methoxy)-6,6a,7,12-tetrahydrobenzo[5,6][1,4]diazepino[1,2-b]isoquinolin-14(5H)-one (1-12)

Pd(PPh₃)₄(4.0 mg, 0.003 mmol) was added to a solution of 1-12c (30 mg, 0.03 mmol) in CH₂Cl₂ (0.5 mL) and pyrrolidine (13 μL, 0.17 mmol). The reaction mixture was allowed to stir for 0.5 h at r.t. The reaction was concentrated and purified by prep-HPLC (0.01% FA in H₂O) to give 1-12 (18 mg, 77% yield) as a white solid. MS (ESI) m/z: 675.4 [M+H]⁺.

¹H NMR (400 MHz, CDCl₃) δ 7.78 (d, J=4.4 Hz, 1H), 7.71-7.63 (m, 2H), 7.57-7.53 (m, 1H), 7.51-7.43 (m, 3H), 7.34-7.32 (m, 1H), 7.21-7.15 (m, 1H), 6.80 (s, 1H), 4.87 (d, J=15.7 Hz, 1H), 4.71 (d, J=15.7 Hz, 1H), 4.21-4.02 (m, 6H), 3.94 (s, 3H), 3.84-3.82 (s, 3H), 3.67 (d, J=11.7 Hz, 1H), 3.53-3.39 (m, 2H), 3.21 (t, J=10.9 Hz, 1H), 3.10 (dd, J=15.2, 5.7 Hz, 1H), 2.80 (dd, J=15.2, 5.0 Hz, 1H), 2.52 (dd, J=13.0, 8.1 Hz, 1H), 2.00 (dd, J=13.1, 2.6 Hz, 1H), 1.89 (s, 6H), 0.74 (m, 4H).

### Example 1-13

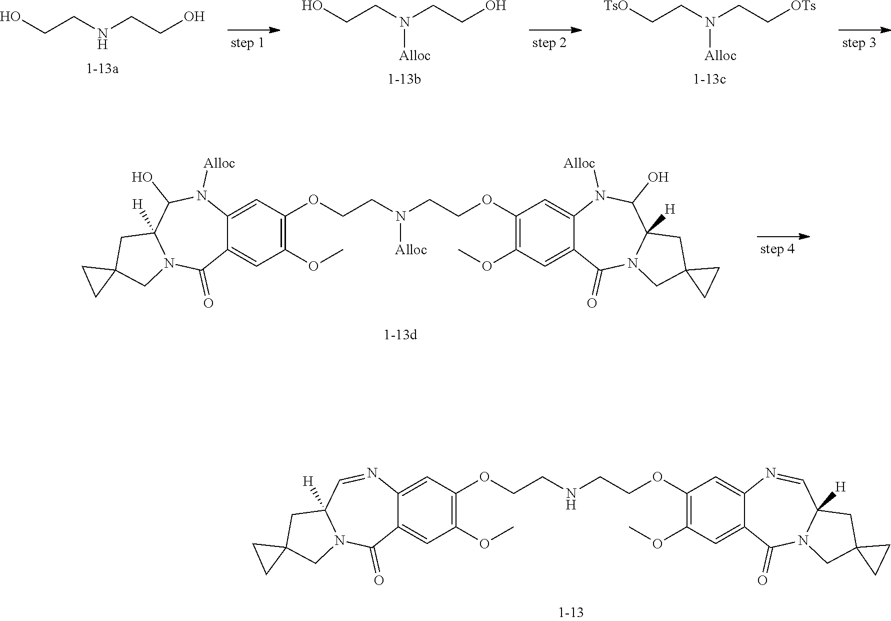

### Step 1: allyl bis(2-hydroxyethyl)carbamate (1-13b)

To a solution of 1-13a (423 mg, 3 mmol) in THE (3.1 mL) and water (5.7 mL) were added AllocCl (361.45 mg, 3 mmol) and K₂CO₃ (1036 mg, 7.5 mmol) at 0° C. The mixture was stirred at 20° C. for 16 h. TLC (petroleum ether:EtOAc=1:1, v/v) showed the reaction was completed. The reaction mixture was poured into water (10 mL) and extracted with EtOAc (20 mL*3). The combined organic layer was dried over Na₂SO₄ and filtered. Solvent was evaporated and the crude product 1-13b (567 mg) was used in the next step without further workup or purification.

### Step 2: (((allyloxy)carbonyl)azanediyl)bis(ethane-2,1-diyl)bis(4-methylbenzenesulfonate) (1-13c)

To a solution of 1-13b (567 mg, 3 mmol) in CH₂Cl₂ (7 mL) was added TsC1 (1.7 g, 9 mmol) and triethylamine (1.67 mL, 12 mmol) at 0° C. The mixture was stirred at 20° C. for 16 h. TLC (petroluem ether:EtOAc=3:1, v/v) showed the reaction was completed. The reaction was poured into water (20 mL) and extracted with EtOAc (30 mL*3). The combined organic layer was dried over Na₂SO₄ and filtered. Solvent was evaporated and the residue was purified by silica column gel chromatography (eluent: petroleum ether/EtOAc=100/0 to 30/70) to afford 1-13c (1.2 g, 73.08% yield) as a colorless oil.

### Step 3: diallyl 8,8″-(((((allyloxy)carbonyl)azanediyl)bis(ethane-2,1-diyl))bis(oxy))(11aS,11a″S)-bis(11-hydroxy-7-methoxy-5-oxo-11,11a-dihydro-1H,3H-spiro[benzo[e]pyrrolo[1,2-a][1,4]diazepine-2,1′-cyclopropane]-10(5H)-carboxylate) (1-13d)

To a solution of 1-13c (50 mg, 0.10 mmol) and 1-5b (75.25 mg, 0.20 mmol) in DMSO (2 mL) was added K₂CO₃ (41.70 mg, 0.30 mmol) at r.t. The mixture was stirred at 50° C. for 16 h. LCMS showed the reaction was completed. The reaction mixture was diluted with EtOAc (10 mL) and washed with brine (8 mL*3). The organic layer was dried over Na₂SO₄ and concentrated to give the residue which was purified by silica column gel chromatography (eluent: petroleum ether/EtOAc=100/0 to 10/90) to afford 1-13d (38 mg, 49.64% yield) as a colorless oil.

MS (ESI) m/z: 902.3 [M+H]⁺.

### Step 4: (11aS,11a″S)-8,8″-((azanediylbis(ethane-2,1-diyl))bis(oxy))bis(7-methoxy-1,11a-dihydro-3H,5H-spiro[benzo[e]pyrrolo[1,2-a][1,4]diazepine-2,1′-cyclopropan]-5-one) (1-13)

To a solution of 1-13d (45 mg, 0.05 mmol) and CH₂Cl₂ (3 mL) were added Pd(PPh₃)₄ (5.77 mg, 0.005 mmol) and pyrrolidine (12 μL, 0.12 mmol) at r.t. The mixture was stirred at 20° C. for 30 min. LCMS showed the reaction was completed. Solvent was evaporated and the residue was purified by prep-HPLC (0.01% FA) to give product 1-13 (12 mg, 35.27% yield) as a white solid.

MS (ESI) m/z: 614.3 [M+H]⁺.

¹H NMR (400 MHz, CDCl₃) δ 7.79 (2H, d, J=4.4), 7.51 (2H, s), 6.84 (2H, d, J=2.2), 4.30-4.13 (4H, m), 3.93 (6H, d, J=0.9), 3.87-3.81 (2H, m), 3.67 (2H, d, J=11.7), 3.49 (2H, d, J=11.7), 3.26-3.17 (4H, m), 2.52 (2H, dd, J=13.0, 8.1), 2.00 (2H, dd, J=13.0, 2.6), 0.73 (8H, ddd, J=11.3, 8.3, 4.5).

### Example 1-14

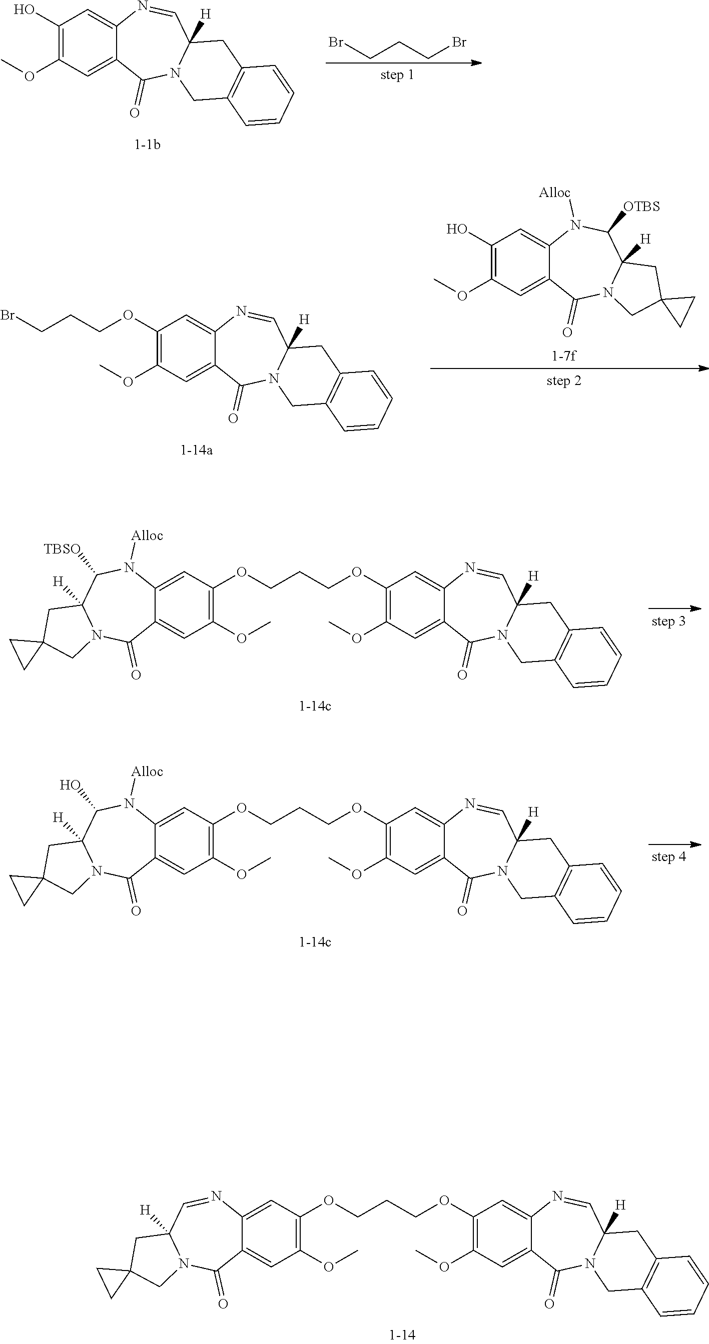

### Step 1: (S)-3-(3-bromopropoxy)-2-methoxy-7,12-dihydrobenzo[5,6][1,4]diazepino[1,2-b]isoquinolin-14(6aH)-one (1-14a)

To a solution of 1-1b (83 mg, 0.27 mmol) and 1,3-dibromopropane (0.42 mL, 4.04 mmol) in 2 mL DMF was added K₂CO₃ (45 mg, 0.32 mmol) at r.t. The mixture was stirred at r.t. for 2 h. The mixture was diluted with EtOAc, washed with H₂O and brine. The organic phase was dried over Na₂SO₄ and concentrated. The residue was purified by silica gel column chromatography (CH₂Cl₂/MeOH=96/4) to give 1-14a (98 mg, 85% yield).

MS (ESI) m/z: 429.1 [M+H]⁺.

### Step 2: allyl (11S,11aS)-11-((tert-butyldimethylsilyl)oxy)-7-methoxy-8-(3-(((S)-2-methoxy-14-oxo-6a,7,12,14-tetrahydrobenzo[5,6][1,4]diazepino[1,2-b]isoquinolin-3-yl)oxy)propoxy)-5-oxo-11,11a-dihydro-1H,3H-spiro[benzo[e]pyrrolo[1,2-a][1,4]diazepine-2,1′-cyclopropane]-10(5H)-carboxylate (1-14b)

1-7f (25 mg, 0.06 mmol) and K₂CO₃ (10.5 mg, 0.08 mmol) was added to a solution of 1-14a (43 mg, 0.09 mmol) in 1 mL DMF. The mixture was stirred at 40° C. for 36 h. The mixture was diluted with EtOAc, washed with water, brine. The organic phase was dried over Na₂SO₄ and concentrated. The residue was purified by silica gel column chromatography (CH₂Cl₂/MeOH=96/4) to give 1-14b (40 mg, 82% yield).

MS (ESI) m/z: 837.5 [M+H]⁺.

### Step 3: allyl (11S,11aS)-11-hydroxy-7-methoxy-8-(3-(((S)-2-methoxy-14-oxo-6a,7,12,14-tetrahydrobenzo[5,6][1,4]diazepino[1,2-b]isoquinolin-3-yl)oxy)propoxy)-5-oxo-11,11a-dihydro-1H,3H-spiro[benzo[e]pyrrolo[1,2-a][1,4]diazepine-2,1′-cyclopropane]-10(5H)-carboxylate (1-14c)

1 M TBAF (240 μL, 0.24 mmol) was added to a solution of 1-14b (40 mg, 0.05 mmol) in dry 1.5 mL THE and AcOH (16 μL, 0.29 mmol). The mixture was stirred at r.t. for 2 h. The mixture was diluted with EtOAc, washed with sat. NaHCO₃ and brine. The organic phase was dried over Na₂SO₄ and concentrated. The residue was purified by silica gel column chromatography (CH₂Cl₂/MeOH=96/4) to give 1-14c (30 mg, 77% yield).

MS (ESI) m/z: 723.4 [M+H]⁺.

### Step 4: (S)-2-methoxy-3-(3-(((S)-7-methoxy-5-oxo-5,11a-dihydro-1H,3H-spiro[benzo[e]pyrrolo[1,2-a][1,4]diazepine-2,1′-cyclopropan]-8-yl)oxy)propoxy)-7,12-dihydrobenzo[5,6][1,4]diazepino[1,2-b]isoquinolin-14(6aH)-one (1-14)

Pd(PPh₃)₄(3.84 mg, 0.003 mmol) was added to a solution of 1-14c (24 mg, 0.033 mmol) in CH₂Cl₂ (1 mL) and pyrrolidine 6.82 μL, 0.083 mmol). The reaction mixture was allowed to stir for 20 min at r.t. The reaction was concentrated and purified by prep-HPLC (Method: column: XBridge Prep C18 OBD 5 μm 19*150 mm; Mobile phase: A-water (no formic acid): B-acetonitrile; flow rate: 20 mL/min). The fraction was lyophilized to give 1-14 (6.5 mg, 32% yield) as a white solid.

MS (ESI) m/z: 621.4 [M+H]⁺.

### Example 1-15

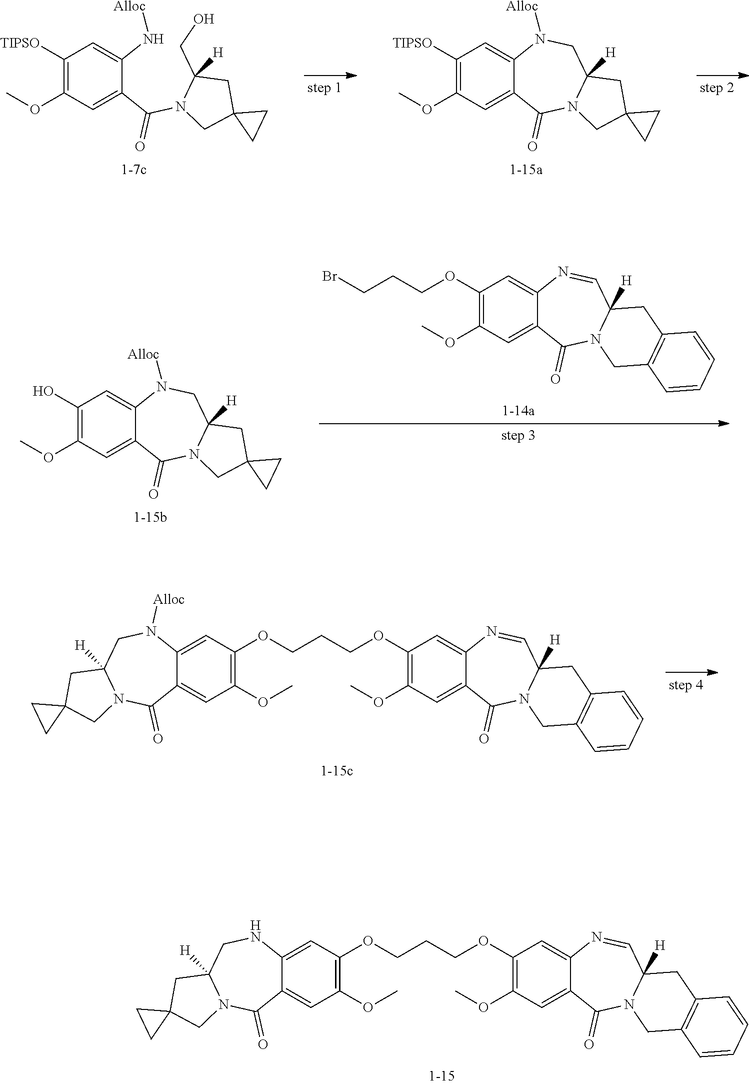

### Step 1: allyl (S)-7-methoxy-5-oxo-8-((triisopropylsilyl)oxy)-11,11a-dihydro-1H,3H-spiro[benzo[e]pyrrolo[1,2-a][1,4]diazepine-2,1′-cyclopropane]-10(5H)-carboxylate (1-15a)

DIAD (1.14 mL, 5.75 mmol) was added to a solution of 1-7c (1.53 g, 2.87 mmol) and PPh₃ (2.26 g, 8.62 mmol) in THE (30 mL). The reaction mixture was stirred for 1 h at 40° C. under N₂. The solvent was removed under vacuum, and the residue was purified by silica gel column chromatography (petroleum ether/EtOAc=30/70) to give 1-15a (940 mg, 64% yield).

MS (ESI) m/z: 515.4 [M+H]⁺.

### Step 2: allyl (S)-8-hydroxy-7-methoxy-5-oxo-11,11a-dihydro-1H,3H-spiro[benzo[e]pyrrolo[1,2-a][1,4]diazepine-2,1′-cyclopropane]-10(5H)-carboxylate (1-15b)

Lithium acetate (121 mg, 1.83 mmol) was added to a solution of 1-15a (940 mg, 1.83 mmol) in wet DMF (15 mL, DMF/water=49/1). The reaction was stirred for 2 h at 25° C. The mixture was diluted with EtOAc, washed with H₂O and brine twice. The organic phase was dried over Na₂SO₄ and concentrated. The residue was purified by silica gel column chromatography (CH₂Cl₂/MeOH=96/4) to give 1-15b (605 mg, 92% yield).

MS (ESI) m/z: 359.2 [M+H]⁺.

### Step 3: allyl (S)-7-methoxy-8-(3-(((S)-2-methoxy-14-oxo-6a,7,12,14-tetrahydrobenzo[5,6][1,4]diazepino[1,2-b]isoquinolin-3-yl)oxy)propoxy)-5-oxo-11,11a-dihydro-1H,3H-spiro[benzo[e]pyrrolo[1,2-a][1,4]diazepine-2,1′-cyclopropane]-10(5H)-carboxylate (1-15c)

1-15b (26 mg, 0.06 mmol) and K₂CO₃ (10.03 mg, 0.07 mmol) was added to a solution of 1-14a (20 mg, 0.06 mmol) in 1 mL DMF. The mixture was stirred at r.t. overnight. The mixture was diluted with EtOAc, washed with water and brine. The organic phase was dried over Na₂SO₄ and concentrated. The residue was purified by silica gel column chromatography (CH₂Cl₂/MeOH=96/4) to give 1-15c (33 mg, 84% yield).

MS (ESI) m/z: 707.4 [M+H]⁺.

### Step 4: (S)-2-methoxy-3-(3-(((S)-7-methoxy-5-oxo-5,10,11,11a-tetrahydro-1H,3H-spiro[benzo[e]pyrrolo[1,2-a][1,4]diazepine-2,1′-cyclopropan]-8-yl)oxy)propoxy)-7,12-dihydrobenzo[5,6][1,4]diazepino[1,2-b]isoquinolin-14(6aH)-one (1-15)

Pd(PPh₃)₄(5.4 mg, 0.005 mmol) was added to a solution of 1-15c (33 mg, 0.047 mmol) in CH₂Cl₂ (1 mL) and pyrrolidine (9.6 μL, 0.117 mmol). The reaction mixture was stirred for 0.5 h at r.t. under N₂. The reaction was concentrated and purified by prep-HPLC (Method: column: XBridge Prep C18 OBD 5 μm 19*150 mm; mobile phase: A-water (no formic acid): B-acetonitrile; flow rate: 20 mL/min). The fraction was lyophilized to give 1-15 (5.1 mg, 17% yield) as a white solid.

MS (ESI) m/z: 623.4 [M+H]⁺.

### Example 1-16

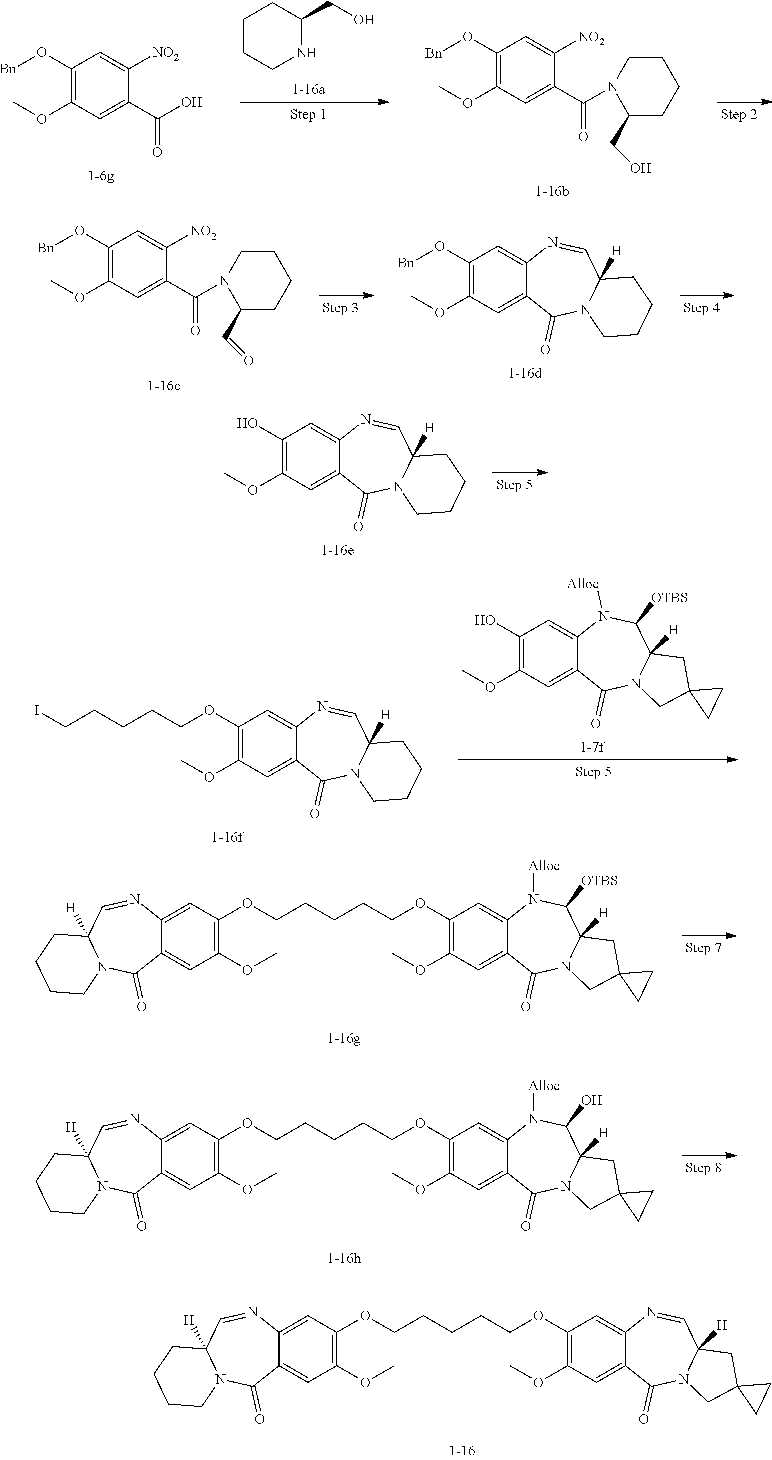

### Step 1: (S)-(4-(benzyloxy)-5-methoxy-2-nitrophenyl)(2-(hydroxymethyl)piperidin-1-yl)methanone (1-16b)

Oxalyl chloride (0.43 mL, 4.94 mmol) was added dropwise to a stirred solution of 1-6g (600 mg, 1.97 mmol) in CH₂Cl₂ (5.1 mL), THE (0.51 mL) and DMF (2.4 μL, 0.031 mmol) at 0° C. under N₂. The reaction mixture was warmed to r.t. and stirred for 1 h. The reaction mixture was concentrated to obtain a pale yellow solid and used for the next step without purification.

The obtained solid and 1-16a (250.6 mg, 2.18 mmol) were dissolved in CH₂Cl₂ (5.6 mL), then the reaction mixture was cooled to 0° C. and Et₃N (0.4 mL, 2.96 mmol) was added dropwise under N₂. The reaction mixture was then warmed to r.t. and stirred for 2 h. The reaction mixture was concentrated to give crude product, which was purified by silica gel chromatography (eluent: EtOAc/hexanes=0% to 80%) to obtain 1-16b (720 mg, 91% yield) as a yellow solid.

MS (ESI) m/z: 401.16 [M+H]⁺.

### Step 2: (S)-1-(4-(benzyloxy)-5-methoxy-2-nitrobenzoyl) piperidine-2-carbaldehyde (1-16c)

DMP (805 mg, 1.89 mmol) was added slowly, portion-wise at 0° C. to a solution of 1-16b (700 mg, 1.75 mmol) in CH₂Cl₂ (7 mL), the reaction was then warmed to r.t. and stirred for 3 h. The reaction mixture was filtered, and the filtrate was washed with sat. aq. sodium thiosulfate solution (10 mL), followed by a slow addition of sat. aq. NaHCO₃ (10 mL) and H₂O (10 mL). The mixture was extracted with CH₂Cl₂ (10 mL*3), and the organic layer was washed with brine (5 mL), dried over Na₂SO₄, filtered, and concentrated to obtain the title compound 1-16c (730 mg, crude) as a yellow solid, which was used for the next step without purification.

MS (ESI) m/z: 399.15 [M+H]⁺.

### Step 3: (S)-3-(benzyloxy)-2-methoxy-7,8,9,10-tetrahydrobenzo[e]pyrido[1,2-a][1,4]diazepin-12(6aH)-one(1-16d)

Compound 1-16c (730 mg, 1.83 mmol) was dissolved in a mixed solvent of THF (0.9 mL), methanol (4.5 mL) and water (0.9 mL), then NH₄Cl (980.06 mg, 18.32 mmol) was added, followed by iron powder (511.6 mg, 9.16 mmol). The reaction was then heated to 50° C. under N₂ and stirred for 16 h. The reaction mixture was filtered through celite. The filtrate was diluted with water (5 mL), extracted with EtOAc (10 mL*3). The organic layer was washed with brine, dried over Na₂SO₄, filtered, and concentrated to give crude product, which was purified by silica gel chromatography (eluent: EtOAc/hexane=0% to 60%) to obtain 1-16d (500 mg, 77.8% yield) as a yellow solid.

MS (ESI) m/z: 351.2 [M+H]⁺.

### Step 4: (S)-3-hydroxy-2-methoxy-7,8,9,10-tetrahydrobenzo[e]pyrido[1,2-a][1,4]diazepin-12(6aH)-one (1-16e)

MeSO₃H (0.37 mL, 5.7 mmol) was added dropwise to a solution of 1-16d (200 mg, 0.57 mmol) in CH₂Cl₂ (2.5 mL) at 0° C. The reaction mixture was stirred at 0° C. for 10 min, then warmed to r.t. and stirred for 2 h. The mixture was quenched with sat. NaHCO₃ (10 mL), then extracted with CH₂Cl₂ (5 mL*3), the organic layer was washed with brine, dried Na₂SO₄, filtered and concentrated to give the crude product, which was purified by silica column gel chromatography (eluent: hexane/EtOAc=100/0 to 20/80) to give 1-16e (120 mg, 80.7% yield) as a white solid.

MS (ESI) m/z: 261.2 [M+H]⁺.

### Step 5: (S)-3-((5-iodopentyl)oxy)-2-methoxy-7,8,9,10-tetrahydrobenzo[e]pyrido[1,2-a][1,4]diazepin-12(6aH)-one (1-16f)

1,5-diiodopentane (0.34 mL, 2.31 mmol) was added to a solution of 1-16e (120 mg, 0.46 mmol) in dry DMF (2 mL). The solution was then cooled to 0° C., K₂CO₃ (127 mg, 0.92 mmol) was added in one portion. The reaction was stirred at r.t. for 6 h. EtOAc (20 mL) and H₂O (10 mL) was added, the organic layer was washed with brine (5 mL), dried over Na₂SO₄, filtered and concentrated to give the crude product, which was purified by silica column gel chromatography (eluent: hexane/EtOAc=100/0 to 50/50) to give 1-16f (130 mg, 61.7% yield) as a yellow solid.

MS (ESI) m/z: 457.1 [M+H]⁺.

### Step 6: allyl(11S,11aS)-11-((tert-butyldimethylsilyl)oxy)-7-methoxy-8-((5-(((S)-2-methoxy-12-oxo-6a,7,8,9,10,12-hexahydrobenzo[e]pyrido[1,2-a][1,4]diazepin-3-yl)oxy)pentyl)oxy)-5-oxo-11,11a-dihydro-1H,3H-spiro[benzo[e]pyrrolo[1,2-a][1,4]diazepine-2,1′-cyclopropane]-10(5H)-carboxylate (1-16g)

A solution of 1-7f (50 mg, 0.102 mmol) and 1-16f (51.3 mg, 0.11 mmol) in DMF (0.5 mL) was added K₂CO₃ (16.9 mg, 0.12 mmol). The mixture was stirred at r.t. for 3 h. EtOAc (15 mL) and H₂O (10 mL) was added, the organic layer was washed with brine (5 mL), dried over Na₂SO₄, filtered and concentrated to give the crude product, which was purified by silica column gel chromatography (eluent: hexane/EtOAc=100/0 to 50/50) to give 1-16g (60 mg, 71.7% yield) as a white solid.

MS (ESI) m/z: 817.5 [M+H]⁺.

### Step 7: allyl(11S,11aS)-11-hydroxy-7-methoxy-8-((5-(((S)-2-methoxy-12-oxo-6a,7,8,9,10,12-hexahydrobenzo[e]pyrido[1,2-a][1,4]diazepin-3-yl)oxy)pentyl)oxy)-5-oxo-11,11a-dihydro-1H,3H-spiro[benzo[e]pyrrolo[1,2-a][1,4]diazepine-2,1′-cyclopropane]-10(5H)-carboxylate (1-16 h)

To a solution of 1-16g (60 mg, 0.07 mmol) in dry THE (1 mL) was added AcOH (24 μL), followed by dropwise addition of TBAF (350 μL, 0.35 mmol, 1M in THF). The mixture was stirred at r.t. for 6 h. The mixture was quenched with sat. NaHCO₃ and extracted with EtOAc (5 mL*3), the organic layer was washed brine (5 mL), dried over Na₂SO₄, filtered, and concentrated to give the crude product, which was purified by silica column gel chromatography (eluent: CH₂Cl₂/MeOH=100/0 to 5/95) to give 1-16 h (40 mg, 77.5% yield) as a white solid.

MS (ESI) m/z: 703.4 [M+H]⁺.

### Step 8: (S)-7-methoxy-8-((5-(((S)-2-methoxy-12-oxo-6a,7,8,9,10,12-hexahydrobenzo[e]pyrido[1,2-a][1,4]diazepin-3-yl)oxy)pentyl)oxy)-1,11a-dihydro-3H,5H-spiro[benzo[e]pyrrolo[1,2-a][1,4]diazepine-2,1′-cyclopropan]-5-one (1-16)

To a solution of 1-16 h (40 mg, 0.056 mmol) in CH₂Cl₂ (1 mL) was added Pd(PPh₃)₄ (2.0 mg, cat. amount) and pyrrolidine (11 μL, 0.14 mmol). The reaction mixture was stirred at r.t. under N₂ for 15 min. The reaction was neutralized with AcOH and concentrated to give the residue, which was purified by prep-HPLC (Method: column: XBridge Prep C18 OBD 5 μm 19*150 mm; mobile phase: A-water (0.01% formic acid): B-acetonitrile; flow rate: 20 mL/min) to give 1-16 (9.6 mg, 28.1% yield) as a white solid.

MS (ESI) m/z: 601.1 [M+H]⁺.

¹H NMR (400 MHz, CDCl₃) δ 7.92 (s, 1H), 7.81 (s, 1H), 7.52 (s, 1H), 7.44 (s, 1H), 6.79 (d, J=14.2 Hz, 2H), 4.24 (s, 1H), 4.10 (d, J=22.7 Hz, 4H), 3.94 (d, J=3.5 Hz, 6H), 3.87 (s, 1H), 3.79 (s, 1H), 3.69 (d, J=11.3 Hz, 1H), 3.51 (d, J=12.2 Hz, 1H), 3.25 (s, 1H), 2.62-2.47 (m, 1H), 2.19-1.77 (m, 11H), 1.68 (s, 4H), 0.83-0.64 (m, 4H).

### Example 1-17

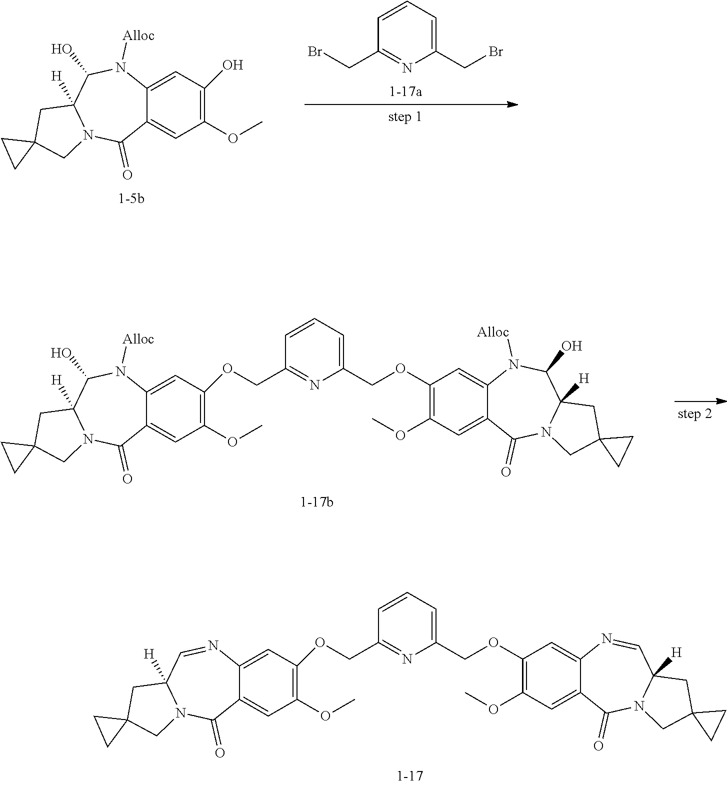

### Step 1: Diallyl 8,8″-((pyridine-2,6-diylbis(methylene))bis(oxy))(11S,11aS,11″S,11a″S)-bis(11-hydroxy-7-methoxy-5-oxo-11,11a-dihydro-1H,3H-spiro[benzo[e]pyrrolo[1,2-a][1,4]diazepine-2,1′-cyclopropane]-10(5H)-carboxylate) (1-17b)

To a solution of 1-5b (84 mg, 0.23 mmol) and 1-17a (30 mg, 0.11 mmol) in 3 mL DMF was added potassium carbonate (84 mg, 0.77 mmol), and the mixture was stirred at r.t. for 2 h. The mixture was diluted with water (10 mL) and extracted with ethyl acetate (10 mL*2). The combined organic phase was washed with water, brine and dried over anhydrous sodium sulfate. After that, the organic phase was concentrated under vacuum to give crude product, which was used in the next step directly without further purification.

MS (ESI) m/z: 852.9 [M+H]⁺.

### Step 2: (11aS,11a″S)-8,8″-((pyridine-2,6-diylbis(methylene))bis(oxy))bis(7-methoxy-1,11a-dihydro-3H,5H-spiro[benzo[e]pyrrolo[1,2-a][1,4]diazepine-2,1′-cyclopropan]-5-one) (1-17)

To a solution of 1-17b (84 mg, 0.23 mmol) in THF/CH₂Cl₂ (1 mL/1 mL) was added Pd(PPh₃)₄ (12 mg, 0.01 mmol) and 1,3-dimedone (32 mg, 0.23 mmol), and the mixture was stirred at r.t. for 1 h. the solution was purified by prep-HPLC (Method: column:XBridge Prep C18 OBD 5 μm 19*150 mm; mobile phase: A-water (0.01% formic acid): B-acetonitrile; flow rate: 20 mL/min) to give 1-17 (36 mg, 50% yield) as a white solid.

¹H NMR (400 MHz, CDCl₃) δ 7.81 (m, 3H), 7.61 (s, 2H), 7.53 (d, J=7.7 Hz, 2H), 6.92 (s, 2H), 5.85-5.03 (m, 4H), 4.27-3.96 (m, 6H), 3.96 (m, 2H), 3.73 (d, J=11.7 Hz, 2H), 3.55 (d, J=11.7 Hz, 2H), 2.57 (d J=12.9, 2H), 2.05 (d, J=12.8 Hz, 2H), 0.93-0.71 (m, 8H).

MS (ESI) m/z: 648.7 [M+H]⁺.

### Example 1-18

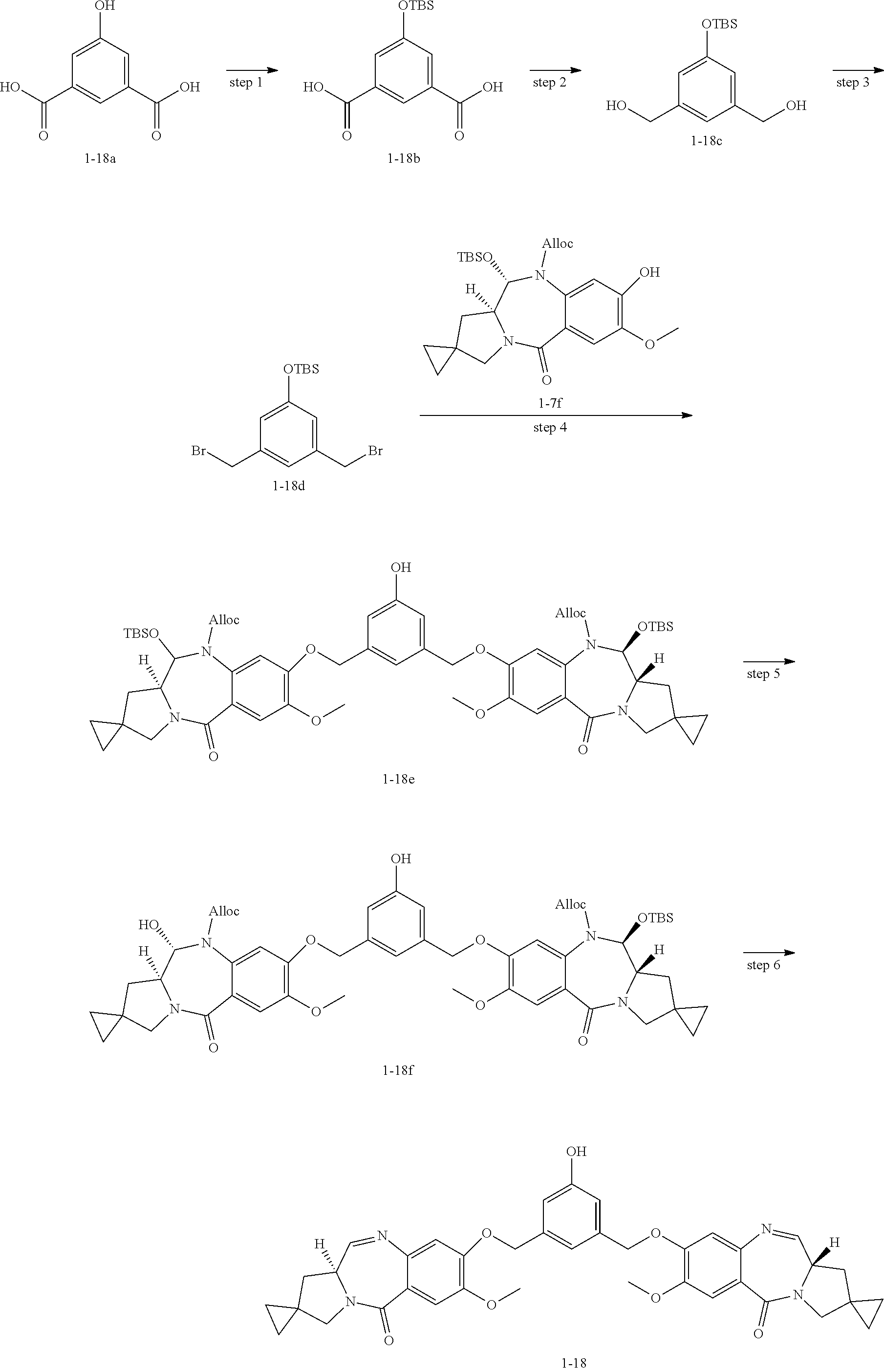

Step 1: 5-((tert-butyldimethylsilyl)oxy)isophthalic acid (1-18b)

To a solution of 1-18a (0.5 g, 2.74 mmol) in 10 mL DMF was added TBS-Cl (2 g, 13.7 mmol) and imidazole (1.12 g, 16.4 mmol), and the mixture was stirred at 50° C. for 4 h. The mixture was acidified with 1 N HCl to pH=3, diluted with water (10 mL) and extracted with ethyl acetate (10 mL*2). the combined organic phase was washed with water, brine and dried over anhydrous sodium sulfate. After that, the organic phase was concentrated under vacuum to give the residue, and the residue was purified by silica gel column chromatography (petroleum ether/ethyl acetate=50/50) to give 1-18b (170 mng, 21% yield) as a white solid.

MS (ESI) m/z: 297.4 [M+H]⁺.

### Step 2: (5-((tert-butyldimethylsilyl)oxy)-1,3-phenylene)dimethanol (1-18c)

To a solution of 1-18b (50 mg, 0.17 mmol) in 2 mL THE was added LiAlH₄ (1 M, 0.34 mL, 0.34 mmol), and the mixture was stirred at 40° C. for 2 h. The mixture was quenched with H₂O (36 μL), 10% aq. NaOH (40 μL) and H₂O (200 μL) the mixture was stirred for 1 h at r.t., the solution was filtered, and filtrate was concentrated under vacuum to give a residue. The residue was purified by prep-HPLC (Method: column:XBridge Prep C18 OBD 5 μm 19*150 mm; mobile phase: A-water (0.1% formic acid): B-acetonitrile; flow rate: 20 mL/min) to give 1-18c (27 mg, 27.1% yield) as a white solid.

MS (ESI) m/z: 269.4 [M+H]⁺.

### Step 3: (3,5-bis(bromomethyl)phenoxy)(tert-butyl)dimethylsilane (1-18d)

A solution of 1-18c (21 mg, 0.08 mmol) in 2 mL CH₃CN was cooled to 0° C., then PPh₃ (62 mg, 0.24 mmol) and CBr₄ (78 mg, 0.24 mmol) were added, and the mixture was stirred at r.t. for 1 h. The solution was diluted with water (10 mL) and extracted with ethyl acetate (10 mL*2). The combined organic phase was washed with water, brine and dried over anhydrous sodium sulfate. After that, the organic phase was concentrated under vacuum to give the residue. The residue was purified by silica gel column chromatography (petroleum ether/ethyl acetate=90/10) to give 1-18d (30 mg, 96% yield) as a colorless oil.

MS (ESI) m/z: 395.2 [M+H]⁺.

### Step 4: allyl (11S,11aS)-8-((3-((((1 1aS)-10-((allyloxy)carbonyl)-11-((tert-butyldimethylsilyl)oxy)-7-methoxy-5-oxo-5,10,11,11a-tetrahydro-1H,3H-spiro[benzo[e]pyrrolo[1,2-a][1,4]diazepine-2,1′-cyclopropan]-8-yl)oxy)methyl)-5-hydroxybenzyl)oxy)-11-((tert-butyldimethylsilyl)oxy)-7-methoxy-5-oxo-11,11a-dihydro-1H,3H-spiro[benzo[e]pyrrolo[1,2-a][1,4]diazepine-2,1′-cyclopropane]-10(5H)-carboxylate (1-18e)

To a solution of 1-18d (30 mg, 0.08 mmol) and 1-7f (79 mg, 0.16 mmol) in 3 mL DMF was added potassium carbonate (23 mg, 0.16 mmol), and the mixture was stirred at r.t. for 4 h. The mixture was diluted with water (10 mL) and extracted with ethyl acetate (10 mL*2). The combined organic phase was washed with water, brine and dried over anhydrous sodium sulfate. The solution was concentrated to give a residue that was purified by silica gel column chrornatography (petroleurn ether/ethyl acetate===90/10) to give 1-18e (58 mg, 63% yield) as a white solid.

MS (ESI) m/z: 1096.5 [M+H]⁺.

### Step 5: diallyl 8,8″-(((5-hydroxy-1,3-phenylene)bis(methylene))bis(oxy))(11S,11aS,11″S,11a″S)-bis(11-hydroxy-7-methoxy-5-oxo-11,11a-dihydro-1H,3H-spiro[benzo[e]pyrrolo[1,2-a][1,4]diazepine-2,1′-cyclopropane]-10(5H)-carboxylate) (1-18f)

To a solution of 1-18e (58 mg, 0.05 mmol) in 3 mL THE was added dropwise TBAF (159 μL, 0.16 mmol) and AcOH (15 μL, 0.27 mmol) and the mixture was stirred at r.t. for 1 h. The mixture was diluted with water (10 mL) and extracted with ethyl acetate (10 mL*2). The combined organic phase was washed with water, brine and dried over anhydrous sodium sulfate. The solution was concentrated to give a residue that was purified by sil ica gel colunm chromatography (CH₂Cl₂/MeOH=90/10) to give 1-18f (42 mg, 91.3% yield) as a white solid.

MS (ESI) m/z: 867.9 [M+H]⁺.

### Step 6: (11aS,11a″S)-8,8″-(((5-hydroxy-1,3-phenylene)bis(methylene))bis(oxy))bis(7-methoxy-1,11a-dihydro-3H,5H-spiro[benzo[e]pyrrolo[1,2-a][1,4]diazepine-2,1′-cyclopropan]-5-one) (1-18)

To a solution of 1-18f (42 mg, 0.05 mmol) in CH₂Cl₂ (2 mL) was added Pd(PPh₃)₄(3 mg, 0.003 mmol) and pyrrolidine (21 μL, 0.25 mmol), and the mixture was stirred at r.t. for 1 h. The solution was purified by prep-HPLC (Method: column:XBridge Prep C18 OBD 5 μm 19*150 mm; mobile phase: A-water (0.01% formic acid): B-acetonitrile; flow rate: 20 mL/min) to give 1-18 (18 mg, 55% yield) as a white solid.

¹H NMR (400 MHz, CDCl₃) δ 7.75 (s, 2H), 7.48 (s, 2H), 6.90 (m, 5H), 5.08 (m, 4H), 4.01-3.79 (m, 8H), 3.66 (d, J=11.8 Hz, 2H), 3.47 (d, J=11.7 Hz, 2H), 2.69-2.37 (m, 2H), 2.02-1.89 (m, 2H), 0.78 (dm, 8H).

MS (ESI) m/z: 663.7 [M+H]⁺.

### Example 1-19

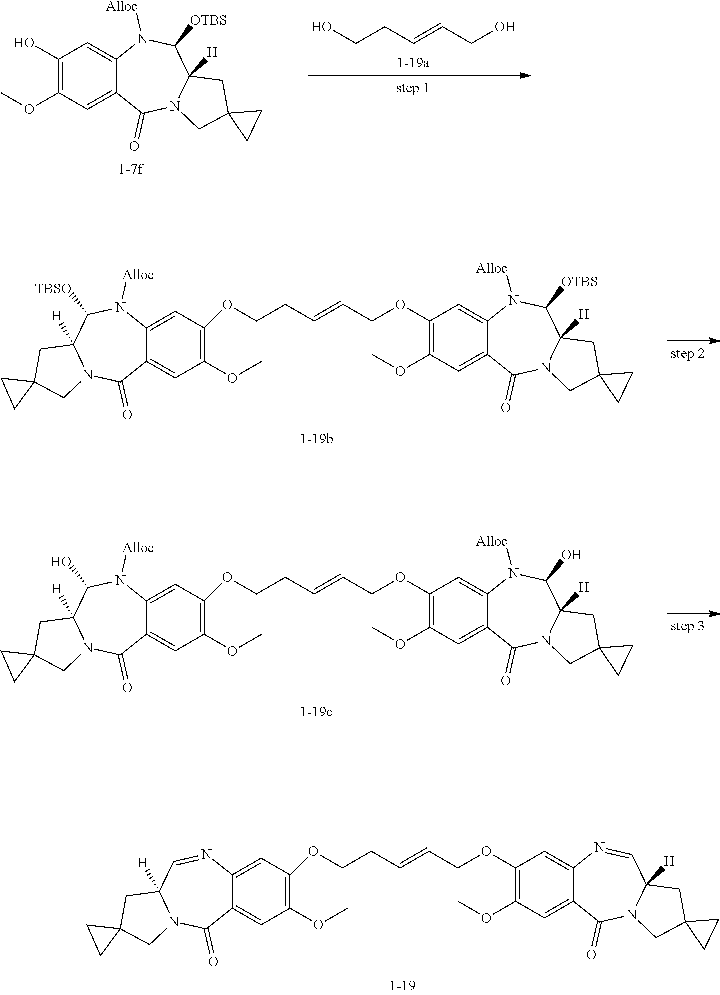

### Step 1: diallyl 8,8″-(((E)-pent-2-ene-1,5-diyl)bis(oxy))(11S,11aS,11″S,11a″S)-bis(11-((tert-butyldimethylsilyl)oxy)-7-methoxy-5-oxo-11,11a-dihydro-1H,3H-spiro[benzo[e]pyrrolo[1,2-a][1,4]diazepine-2,1′-cyclopropane]-10(5H)-carboxylate) (1-19b)

PPh₃ (96 mg, 0.37 mmol) and DIAD (74 μL, 0.37 mmol) were dissolved in THE (3 mL) and stirred at r.t. for 2 h. Then 1-7 f (107 mg, 0.22 mmol) and 1-19a (8 mg, 0.07 mmol) were added, and the mixture was attired for another 1 h at r.t. The solution was concentrated, and the crude was purified by silica gel column chromatography (CH₂Cl₂ EtOAc=50/50) to give 1-19b (33 mg, 15% yield) as a white solid.

MS (ESI) m/z: 1044.4 [M+H]⁺.

### Step 2: allyl (11S,11aS)-8-(((E)-5-(((11S,11aS)-10-((allyloxy)carbonyl)-11-((tert-butyldimethylsilyl)oxy)-7-methoxy-5-oxo-5,10,11,11a-tetrahydro-1H,3H-spiro[benzo[e]pyrrolo[1,2-a][1,4]diazepine-2,1′-cyclopropan]-8-yl)oxy)pent-2-en-1-yl)oxy)-11-hydroxy-7-methoxy-5-oxo-11,11a-dihydro-1H,3H-spiro[benzo[e]pyrrolo[1,2-a][1,4]diazepine-2,1′-cyclopropane]-10(5H)-carboxylate (1-19c)

To a solution of 1-19b (33 mg, 0.03 mmol) in 3 mL THE were added dropwise TBAF (106 μL, 0.11 mmol) and AcOH (10 μL, 0.15 mmol) the mixture was stirred at r.t. for 1 h. The mixture was diluted with water (10 mL) and extracted with ethyl acetate (10 mL*2). The combined organic phase was washed with water, brine and dried over anhydrous sodium sulfate. The solution was concentrated to give a residue that was purified by si ica gel column chromatography (CH₂Cl₂/MeOH=90/10) to give 1-19c (21 mg, 81% yield) as a white solid.

MS (ESI) m/z: 815.9 [M+H]⁺.

### Step 3: (11aS,11a″S)-8,8″-(((E)-pent-2-ene-1,5-diyl)bis(oxy))bis(7-methoxy-1,11a-dihydro-3H,5H-spiro[benzo[e]pyrrolo[1,2-a][1,4]diazepine-2,1′-cyclopropan]-5-one) (1-19)

To a solution of 1-19c (21 mg, 0.025 mmol) in CH₂Cl₂ (2 mL) was added Pd(PPh₃)₄ (2 mg, 0.0013 mmol) and pyrrolidine (11 μL, 0.13 mmol), and the mixture was stirred at r.t. for 1 h. The solution was purified by prep-HPLC (Method: column:XBridge Prep C18 OBD 5 μm 19*150 mm; mobile phase: A-water (0.01% formic acid): B-acetonitrile; flow rate: 20 mL/min) to give 1-19 (4 mg, 26% yield) as a white solid.

MS (ESI) m/z: 611.7 [M+H]⁺.

### Example 1-20

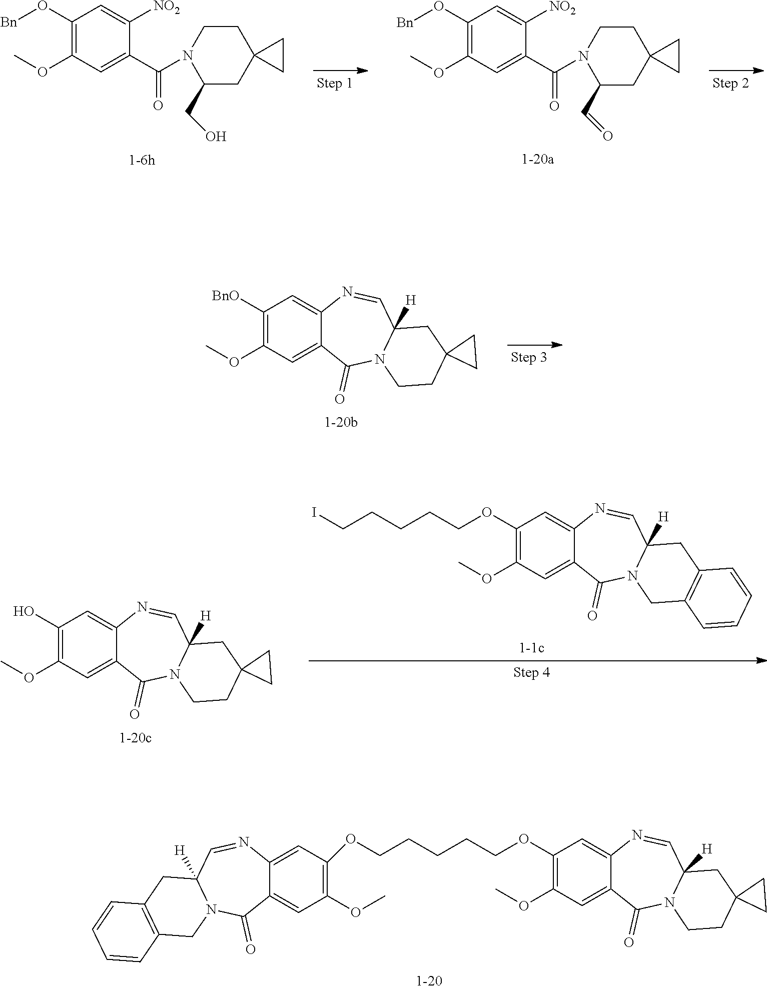 [SMILES: COc1cc(C(=O)N2CCC3(CC3)C[C@H]2CO)c([N+](=O)[O-])cc1OCc1ccccc1 ; COc1cc(C(=O)N2CCC3(CC3)C[C@H]2C=O)c([N+](=O)[O-])cc1OCc1ccccc1 ; COc1cc2c(cc1OCc1ccccc1)N=C[C@@H]1CC3(CCN1C2=O)CC3 ; COc1cc2c(cc1O)N=C[C@@H]1CC3(CCN1C2=O)CC3 ; COc1cc2c(cc1OCCCCCI)N=C[C@@H]1Cc3ccccc3CN1C2=O ; COc1cc2c(cc1OCCCCCOc1cc3c(cc1OC)C(=O)N1Cc4ccccc4C[C@H]1C=N3)N=C[C@@H]1CC3(CCN1C2=O)CC3]

### Step 1: (S)-6-(4-(benzyloxy)-5-methoxy-2-nitrobenzoyl)-6-azaspiro[2.5]octane-5-carbaldehyde (1-20a)

To a solution of 1-6 h (80 mg, 0.16 mmol) in CH₂Cl₂ (3 mL) was added DMP (105 mg, 0.24 mmol) at 0° C., then warmed to r.t. and stirred for 1 h. To the solution was added sat. Na₂S₂O₃ (5 mL) and sat. NaHCO₃ (5 mL), then extracted with CH₂Cl₂ (5 mL*3). The organic phase was dried over anhydrous Na₂SO₄, filtered and concentrated to give the residue, which was purified by flash column chromatography (eluent: hexane/EtOAc=100/0 to 50/50) to give 1-20a (70 mg, 90% yield) as a white solid.

MS (ESI) m/z: 425.1 [M+H]⁺.

### Step 2: (S)-3-(benzyloxy)-2-methoxy-6a,7,9,10-tetrahydro-12H-spiro[benzo[e]pyrido[1,2-a][1,4]diazepine-8,1′-cyclopropan]-12-one (1-20b)

To a solution of 1-20a (70 mg, 0.16 mmol) in MeOH (3 mL) and H₂O (1 mL), NH₄Cl (134 mg, 2.46 mmol) was added, followed by iron powder (46.5 mg, 0.81 mmol). The reaction was refluxed for 3 h under N₂. The reaction mixture was filtered through celite. The filtrate was diluted with water (5 mL), and extracted with EtOAc (5 mL*3). The organic layer was washed with brine, dried over Na₂SO₄, filtered and concentrated to give 1-20b (54 mg, crude), which was used directly for the next step without purification.

MS (ESI) m/z: 377.2 [M+H]⁺.

### Step 3: (S)-3-hydroxy-2-methoxy-6a,7,9,10-tetrahydro-12H-spiro[benzo[e]pyrido[1,2-a][1,4]diazepine-8,1′-cyclopropan]-12-one (1-20c)

To the solution of 1-20b (50 mg, 0.08 mmol) in CH₂Cl₂ (2.5 mL) was added MsOH (55 μL, 0.8 mmol) at 0° C., and the mixture was stirred at 0° C. for 5 h. The mixture was quenched with sat. NaHCO₃ and extracted with CH₂Cl₂ (5 mL*3). The organic layer was washed with brine (5 mL), dried over Na₂SO₄, and concentrated to give the crude product, which was purified by silica column gel chromatography (eluent: hexane/EtOAc=100/0 to 25/75) to give 1-20c (25 mg, 65.7% yield) as a yellow solid.

MS (ESI) m/z: 287.1 [M+H]⁺.

### Step 4: (S)-2-methoxy-3-((5-(((S)-2-methoxy-12-oxo-6a,9,10,12-tetrahydro-7H-spiro[benzo[e]pyrido[1,2-a][1,4]diazepine-8,1′-cyclopropan]-3-yl)oxy)pentyl)oxy)-7,12-dihydrobenzo[5,6][1,4]diazepino[1,2-b]isoquinolin-14(6aH)-one (1-20)

A solution of 1-20c (25 mg, 0.087 mmol) and 1-1c (48.4 mg, 0.096 mmol) in DMF (0.5 mL) was added K₂CO₃ (14.5 mg, 0.1 mmol). The reaction was stirred at r.t. for 3 h. The reaction was quenched with H₂O (15 mL), extracted with EtOAc (10 mL*3). The organic layer was washed with brine (5 mL), dried over Na₂SO₄, filtered and concentrated to give the crude product, which was purified by prep-HPLC (Method: column: XBridge Prep C18 OBD 5 μm 19*150 mm; mobile phase: A-water (0.01% formic acid): B-acetonitrile; flow rate: 20 mL/min) to give 1-20 (8.9 mg, 15.6% yield) as a white solid.

MS (ESI) m/z: 663.7 [M+H]⁺.

### Example 1-21

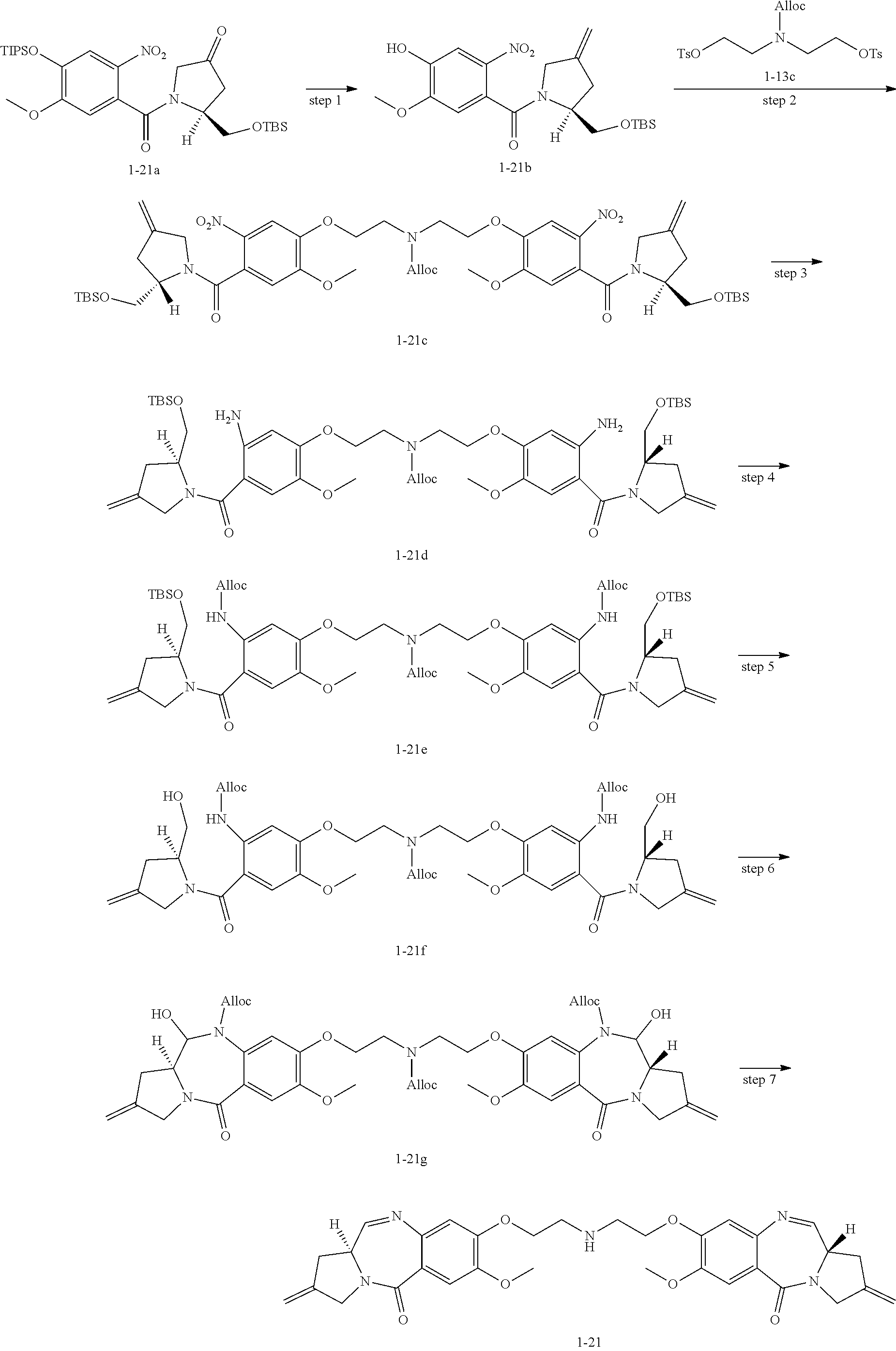

### Step 1: (S)-(2-(((tert-butyldimethylsilyl)oxy)methyl)-4-methylenepyrrolidin-1-yl)(4-hydroxy-5-methoxy-2-nitrophenyl)methanone (1-21b)

A mixture of MePh₃PBr (24.6 g, 68.86 mmol) in THE (100 mL) was added t-BuOK (6.95 g, 61.98 mmol) at 0° C. under N₂. The mixture was stirred at 0° C. for 2 h, then a solution of 1-21a (4 g, 6.89 mmol) in THE (30 mL) was added dropwise to the mixture and stirred at 0° C. for 16 h. The mixture was neutralized with citric acid and extracted with EtOAc (100 mL*3). The organic phase was dried over Na₂SO₄, filtered, and concentrated to give crude product, which was purified by silica column gel chromatography (eluent: hexane/EtOAc=100/0 to 25/75) to give 1-21b (1.1 g, 37.8% yield) as a yellow solid.

MS (ESI) m/z: 423.2 [M+H]⁺.

### Step 2: Allylbis(2-(4-((S)-2-(((tert-butyldimethylsilyl)oxy)methyl)-4-methylenepyrrolidine-1-carbonyl)-2-methoxy-5-nitrophenoxy)ethyl)carbamate (1-21c)

To a solution of 1-13c (100 mg, 0.2 mmol) and 1-21b (186.83 mg, 0.44 mmol) in DMSO (3 mL) was added K₂CO₃ (55.5 mg, 0.4 mmol). The mixture was stirred at 50° C. for 16 h. The reaction was quenched with H₂O and extracted with EtOAc (30 mL). The organic phase was dried over Na₂SO₄, filtered and concentrated to give crude product, which was purified by silica column gel chromatography (eluent: hexane/EtOAc=100/0 to 0/100) to give 1-21c (108 mg, 53.83% yield) as a yellow solid.

MS (ESI) m/z: 998.5 [M+H]⁺.

### Step 3: Allylbis(2-(5-amino-4-((S)-2-(((tert-butyldimethylsilyl)oxy)methyl)-4-methylenepyrrolidine-1-carbonyl)-2-methoxyphenoxy)ethyl)carbamate (1-21d)

A mixture of Zn powder (28.24 mg, 4.08 mmol) in EtOH (3 mL), AcOH (0.2 mL) and H₂O (0.2 mL) was stirred at r.t. for 10 min, then a solution of 1-21c (108 mg, 0.11 mmol) in EtOH (2 mL) was added and stirred at r.t. for 1 h. The reaction was filtered through celite, and the filtrate was concentrated to give crude product, which was purified by silica gel chromatography (eluent: CH₂Cl₂/MeOH=20/1) to obtain 1-21d (73 mg, 72.2% yield) as a yellow oil.

MS (ESI) m/z: 938.6 [M+H]⁺.

### Step 4: Allylbis(2-(5-(((allyloxy)carbonyl)amino)-4-((S)-2-(((tert-butyldimethylsilyl)oxy)methyl)-4-methylenepyrrolidine-1-carbonyl)-2-methoxyphenoxy)ethyl)carbamate (1-21e)

To a solution of 1-21d (73 mg, 0.077 mmol) in dry CH₂Cl₂ (2 mL) was added Alloc-Cl (53 μL, 0.49 mmol) and pyridine (24.73 μL, 0.311 mmol) under N₂ atmosphere at −10° C. The mixture was stirred at −10° C. for 1 h. The solution was added into water (10 mL), and extracted with CH₂Cl₂ (5 mL*3). The organic phase was dried over Na₂SO₄, filtered and concentrated to give crude product, which was purified by flash column chromatography (eluent: petroleum ether/EtOAc=1/3) to give 1-21e (75 mg, 87.13% yield) as an off-white solid.

MS (ESI) m/z: 1106.6 [M+H]⁺.

### Step 5: allylbis(2-(5-(((allyloxy)carbonyl)amino)-4-((S)-2-(hydroxymethyl)-4-methylenepyrrolidine-1-carbonyl)-2-methoxyphenoxy)ethyl)carbamate (1-21f)

Para-Toluenesulfonic acid hydrate (25.79 mg, 0.13 mmol) was added to a solution of 1-21e (75 mg, 0.067 mmol) in THE (2 mL) and water (0.1 mL). The reaction mixture was stirred at r.t. for 1 h. The mixture was directly concentrated and purified by flash column chromatography (eluent: CH₂Cl₂/MeOH=20/1) to give 1-21f (52 mg, 87.38% yield) as a yellow solid.

MS (ESI) m/z: 878.4 [M+H]⁺.

### Step 6: diallyl8,8′-(((((allyloxy)carbonyl)azanediyl)bis(ethane-2,1-diyl))bis(oxy))(11 aS,11a'S)-bis(11-hydroxy-7-methoxy-2-methylene-5-oxo-2,3,11,11a-tetrahydro-1H-benzo[e]pyrrolo[1,2-a][1,4]diazepine-10(5H)-carboxylate)1H,3H-spiro[benzo[e]pyrrolo[1,2-a][1,4]diazepine-2,1′-cyclopropane]-10(5H)-carboxylate (1-21g)

DMP (55.27 mg, 0.13 mmol) was added at 0° C. to a solution of 1-21f (52 mg, 0.059 mmol) in CH₂Cl₂ (2 mL), the reaction was then warmed to r.t. and stirred for 4 h. The reaction mixture was filtered, and the filtrate was quenched with sat. aq. sodium thiosulfate solution (5 mL), followed by a slow addition of sat. aq. NaHCO₃ (5 mL) and H₂O (10 mL). The mixture was extracted with dichloromethane (5 mL*3), and the organic layer was washed with brine (5 mL), dried over Na₂SO₄, filtered, and concentrated to give crude product, which was purified by flash column chromatography (eluent: CH₂Cl₂/MeOH=20/1) to give 1-21g (32 mg 61.82% yield) as a white solid.

MS (ESI) m/z: 874.4 [M+H]⁺.

### Step 7: (11aS,11a'S)-8,8′-((azanediylbis(ethane-2,1-diyl))bis(oxy))bis(7-methoxy-2-methylene-1,2,3,11a-tetrahydro-5H-benzo[e]pyrrolo[1,2-a][1,4]diazepin-5-one) (1-21)

To a solution of 1-21g (32 mg, 0.036 mmol) and CH₂Cl₂ (2 mL) was added Pd(PPh₃)₄ (2.12 mg, 0.0018 mmol) and pyrrolidine (6 μL, 0.073 mmol) and stirred at r.t. for 15 min under N₂. The reaction was neutralized with AcOH and concentrated to give the crude product, which was purified by prep-HPLC (column: XBridge Prep C18 OBD 5 μm 19*150 mm); mobile phase: A-water (0.01% formic acid): B-acetonitrile; flow rate: 20 mL/min) to give 1-21 (9.7 mg, 45.23% yield) as a white solid.

MS (ESI) m/z: 586.3 [M+H]⁺.

### Example 1-22

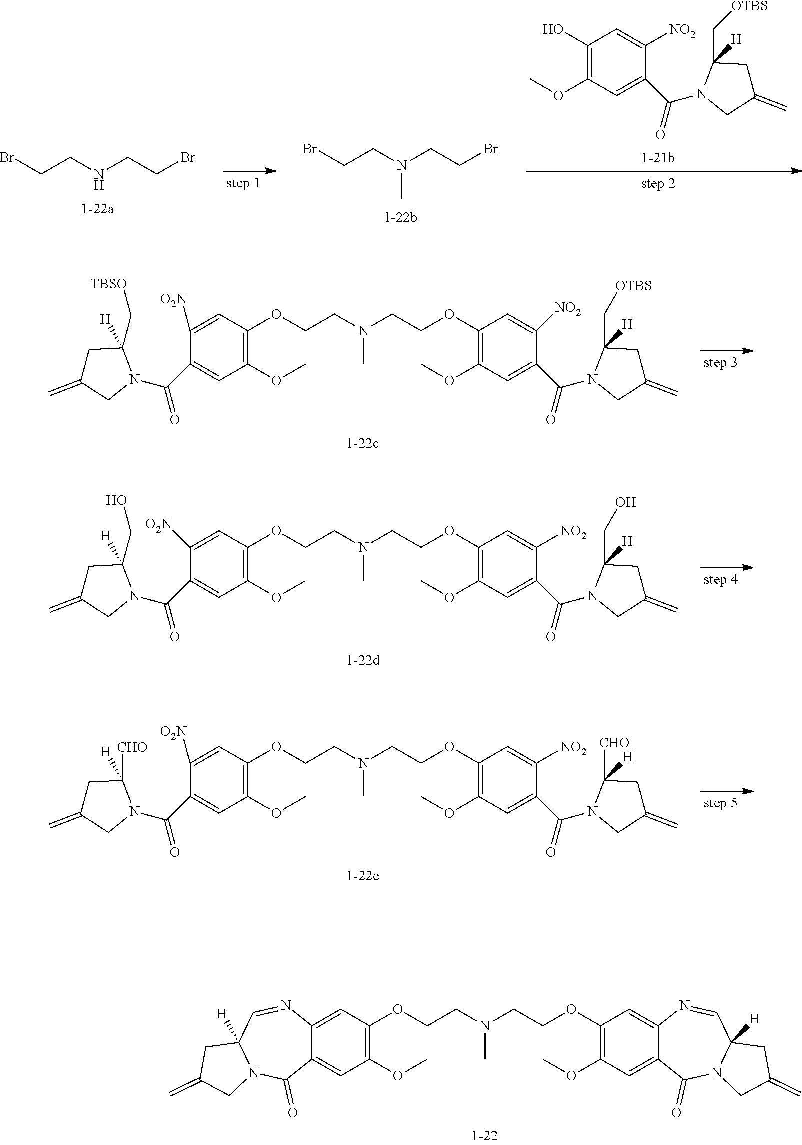

### Step 1: 2-bromo-N-(2-bromoethyl)-N-methylethan-1-amine (1-22b)

To a solution of 37% formaldehyde (0.66 mL) and 98% formic acid (0.26 mL) was added 1-22a (1.0 g) and the solution was heated at reflux for 2 h. The mixture was concentrated in vacuo to give a colorless oil, which was crystallized with MeOH to give 1-22b (700 mg, 65% yield) as a white solid.

¹H NMR (400 MHz, DMSO-d6) δ 9.66 (s, 1H), 3.79-3.72 (m, 4H), 3.65-3.58 (m, 4H), 2.85 (s, 3H).

### Step 2: ((((methylazanediyl)bis(ethane-2,1-diyl))bis(oxy))bis(5-methoxy-2-nitro-4,1-phenylene))bis(((S)-2-(((tert-butyldimethylsilyl)oxy)methyl)-4-methylenepyrrolidin-1-yl)methanone) (1-22c)

To a solution of 1-22b (100 mg, 0.408 mmol) and 1-21b (379.50 mg, 0.898 mmol) in DMSO (3 mL) was added K₂CO₃ (112.84 mg, 0.816 mmol). The mixture was stirred at 50° C. for 16 h. The reaction was quenched with H₂O and extracted with EtOAc (30 mL). The organic phase was dried over Na₂SO₄, filtered and concentrated to give crude product, which was purified by silica column gel chromatography (eluent: hexane/EtOAc=100/0 to 0/100) to give 1-22c (200 mg, 52.78% yield) as a yellow solid.

MS (ESI) m/z: 928.6 [M+H]⁺.

### Step 3: ((((methylazanediyl)bis(ethane-2,1-diyl))bis(oxy))bis(5-methoxy-2-nitro-4,1-phenylene))bis(((S)-2-(hydroxymethyl)-4-methylenepyrrolidin-1-yl)methanone) (1-22d)

Para-Toluenesulfonic acid hydrate (90.16 mg, 0.474 mmol) was added to a solution of 1-22c (200 mg, 0.215 mmol) in THE (2 mL) and water (0.1 mL). The reaction mixture was allowed to stir at r.t. for 2 h. The mixture was concentrated and purified by silica gel chromatography (eluent: CH₂Cl₂/MeOH=20/1) to give 1-22d (110 mg 72.96% yield) as a white solid.

MS (ESI) m/z: 700.4 [M+H]⁺.

### Step 4: (2S,2′S)-1,1′-(4,4′-(((methylazanediyl)bis(ethane-2,1-diyl))bis(oxy))bis(5-methoxy-2-nitrobenzoyl))bis(4-methylenepyrrolidine-2-carbaldehyde) (1-22e)

DMP (146.69 mg, 0.345 mmol) was added to a solution of 1-22d (110 mg, 0.157 mmol) in dichloromethane (2 mL), THE (2 mL) and DMF (1 mL) at 0° C. The reaction was then warmed to r.t. and stirred at r.t. for 4 h. The reaction mixture was filtered, and the filtrate was directly concentrated to give crude product, which was purified by silica gel chromatography (eluent: CH₂Cl₂/MeOH=20/1) to give 1-22e (60 mg 55.83% yield) as a yellow solid.

MS (ESI) m/z: 696.3 [M+H]⁺.

### Step 5: (11aS,11a′S)-8,8′-(((methylazanediyl)bis(ethane-2,1-diyl))bis(oxy))bis(7-methoxy-2-methylene-1,2,3,11a-tetrahydro-5H-benzo[e]pyrrolo[1,2-a][1,4]diazepin-5-one) (1-22)

To a solution of 1-22e (60 mg, 0.086 mmol) in THE (0.896 mL), methanol (4.48 mL) and water (0.896 mL) was added NH₄Cl (92.26 mg, 1.72 mmol), followed by iron powder (48.16 mg, 0.862 mmol). The mixture was then heated at 50° C. under N₂ for 16 h. The reaction mixture was cooled to r.t. and filtered through celite. The filtrate was directly concentrated to give crude product, which was purified by prep-HPLC (column: XBridge Prep C18 OBD 5 μm 19*150 mm; method: mobile phase: A-water (0.01% formic acid): B-acetonitrile; flow rate: 20 mL/min) to give 1-22 (4.8 mg, 9.28% yield) as a white solid.

MS (ESI) m/z: 600.3 [M+H]⁺.

¹H NMR (400 MHz, CDCl₃) δ 7.68 (d, J=4.4 Hz, 2H), 7.49 (s, 2H), 6.83 (s, 2H), 5.19 (d, J=11.2 Hz, 4H), 4.38-4.17 (m, 8H), 3.91 (s, 6H), 3.89-3.84 (m, 2H), 3.12-3.08 (m, 6H), 2.97-2.93 (m, 2H), 2.56 (s, 3H).

### Example 2-1

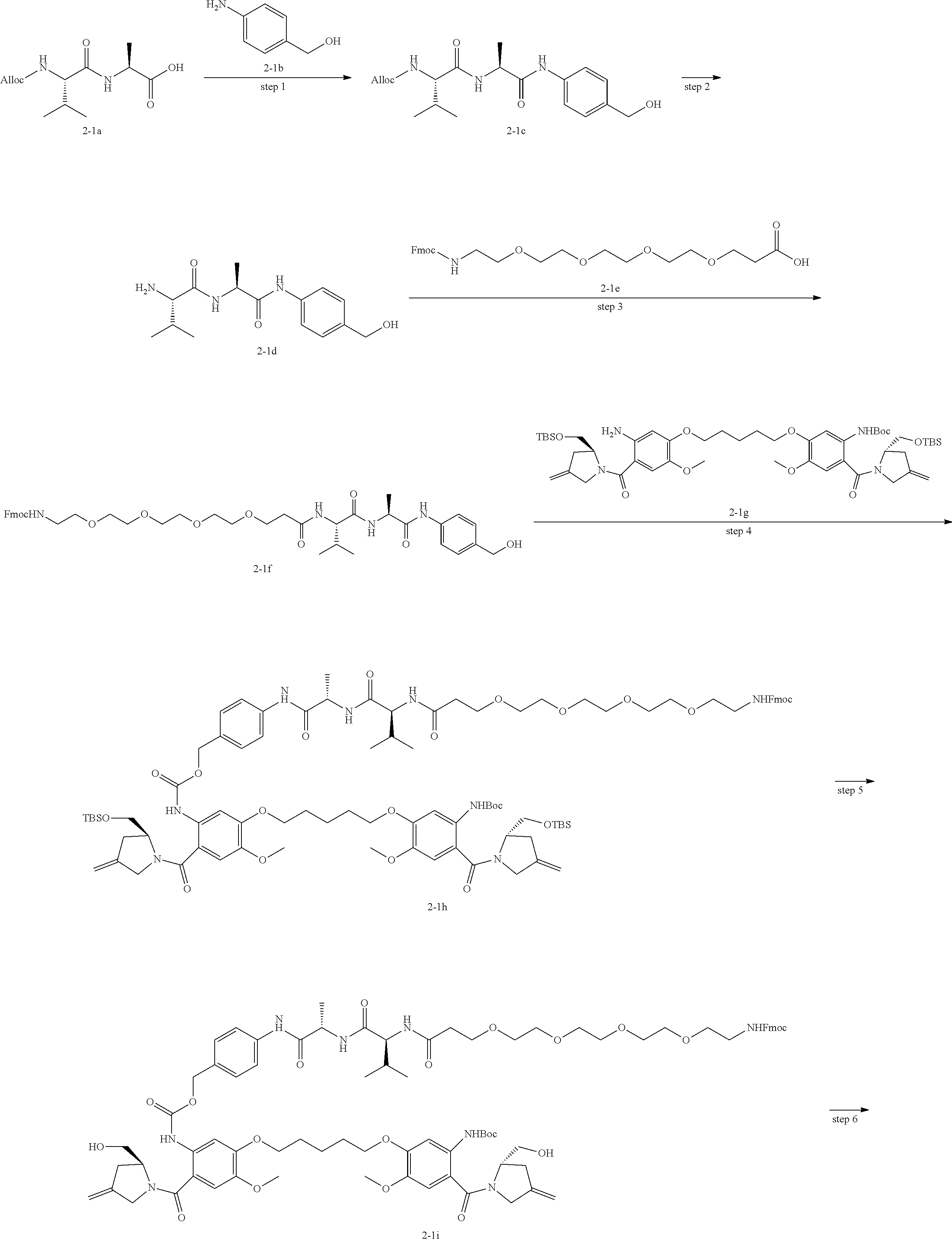

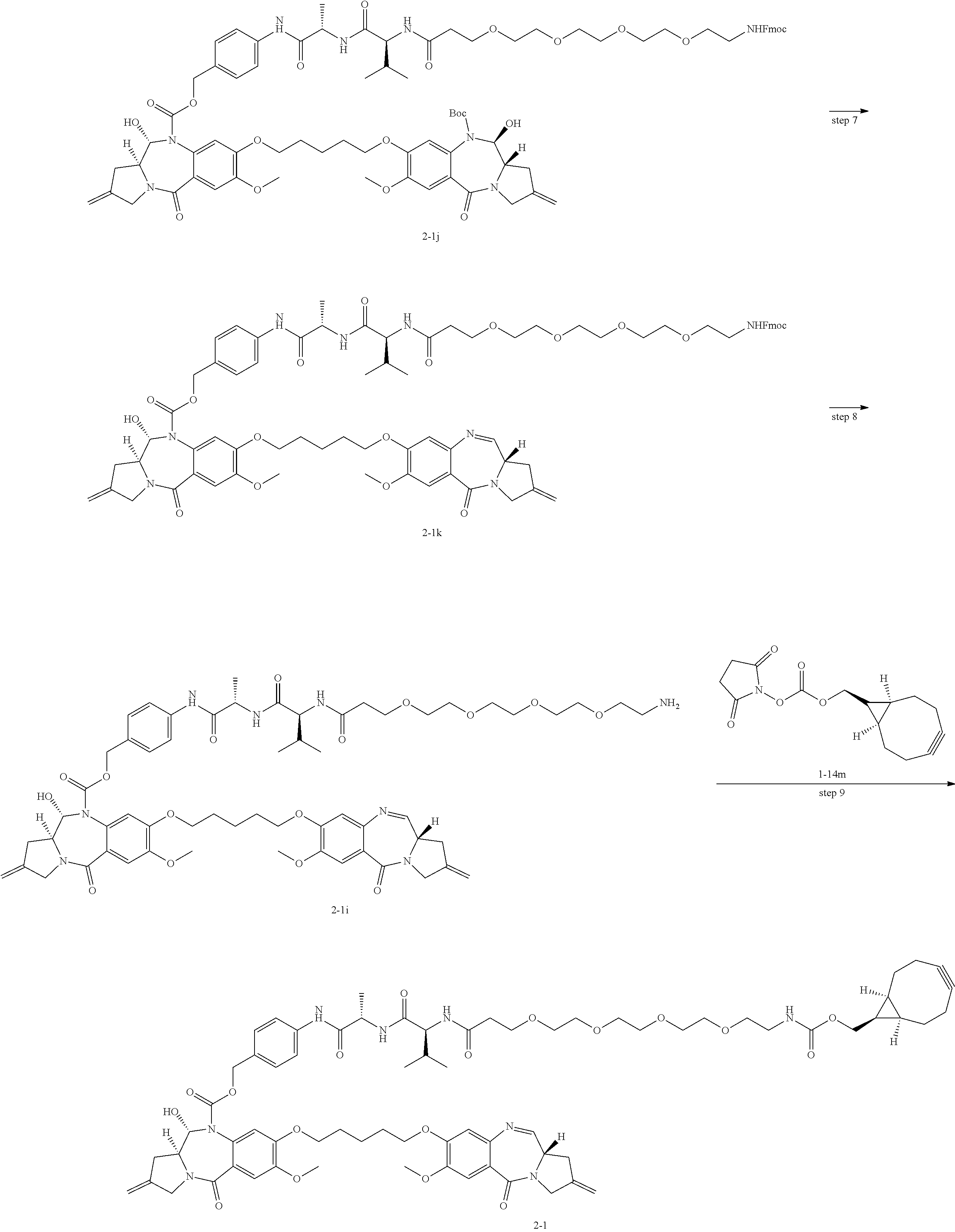

### Step 1: allyl ((S)-1-(((S)-1-((4-(hydroxymethyl)phenyl)amino)-1-oxopropan-2-yl)amino)-3-methyl-1-oxobutan-2-yl)carbamate (2-1c)

EEDQ (2-ethoxy-1-ethoxycarbonyl-1,2-dihydroquinoline, 4.77 g, 19.3 mmol) was added to a solution of 2-1a (5 g, 18.4 mmol) and 2-1b (2.37 g, 19.3 mmol) in 100 mL dry THF. The mixture was stirred at r.t. for 40 h. Upon reaction completion as determined by LCMS/TLC (CH₂Cl₂/MeOH=20:1), the mixture was concentrated. The residue was slurried with MTBE (30 V) and stirred for 2 h. The solid was isolated by filtration under vacuum for 3 h to give 2-1c (5.16 g, 74% yield). MS (ESI) m/z: 378.4 [M+H]⁺.

¹H NMR (400 MHz, d6-DMSO) δ 9.90 (s, 1H), 8.14 (d, J=7.0 Hz, 1H), 7.53 (d, J=8.5 Hz, 2H), 7.25 (t, J=9.0 Hz, 3H), 5.91 (ddd, J=22.3, 10.5, 5.3 Hz, 1H), 5.30 (dd, J=17.2, 1.5 Hz, 1H), 5.17 (d, J=10.5 Hz, 1H), 5.10 (t, J=5.7 Hz, 1H), 4.52-4.45 (m, 2H), 4.43 (d, J=5.6 Hz, 3H), 3.89 (dd, J=8.5, 7.1 Hz, 1H), 1.98 (dq, J=13.5, 6.7 Hz, 1H), 1.28 (t, J=11.8 Hz, 3H), 0.88 (d, J=6.8 Hz, 3H), 0.84 (d, J=6.7 Hz, 3H).

### Step 2: (S)-2-amino-N—((S)-1-((4-(hydroxymethyl)phenyl)amino)-1-oxopropan-2-yl)-3-methylbutanamide (2-1d)

Pd(PPh₃)₄(76.6 mg, 0.066 mmol) was added to a solution of 2-1c (500 mg, 1.33 mmol) in CH₂Cl₂ (10 mL) and pyrrolidine (270.8 μL, 3.31 mmol) at r.t. under N₂. The reaction mixture was allowed to stir for 0.5 h at r.t. The reaction was concentrated and purified by silica column gel chromatography (CH₂Cl₂/MeOH=90:10) to give the product 2-1d (370 mg, 95% yield) as a white solid.

MS (ESI) m/z: 294.3 [M+H]⁺.

¹H NMR (400 MHz, d6-DMSO) δ 9.99 (s, 1H), 8.20 (d, J=6.3 Hz, 1H), 7.53 (d, J=8.4 Hz, 2H), 7.24 (d, J=8.4 Hz, 2H), 5.10 (s, 1H), 4.53-4.45 (m, 1H), 4.43 (s, 2H), 3.05 (d, J=4.9 Hz, 1H), 1.93 (dd, J=12.0, 6.8 Hz, 1H), 1.30 (d, J=7.0 Hz, 3H), 0.89 (d, J=6.9 Hz, 3H), 0.80 (d, J=6.8 Hz, 3H).

### Step 3: (9H-fluoren-9-yl)methyl ((17S,20S)-21-((4-(hydroxymethyl)phenyl)amino)-17-isopropyl-20-methyl-15,18,21-trioxo-3,6,9,12-tetraoxa-16,19-diazahenicosyl)carbamate (2-1f)

DIPEA (326 mg, 2.52 mmol) was added to a solution of 2-1e (370 mg, 1.26 mmol) and HATU (575.8 mg, 1.51 mmol) in 4 mL dry DMF. The mixture was stirred at r.t. for 10 min. Then a solution of 2-1d (645 mg, 1.33 mmol) in DMF was added to the mixture. The reaction was stirred for 1 h. Upon reaction completion as determined by LCMS/TLC (CH₂Cl₂/MeOH=20:1), the mixture was concentrated and purified by silica column gel chromatography (CH₂Cl₂/MeOH=95:5) to give the product 2-1e (680 mg, 71% yield) as a light brown solid. MS (ESI) m/z: 763.5 [M+H]⁺.

### Step 4: tert-butyl (5-((5-(5-((((4-((21S,24S)-1-(9H-fluoren-9-yl)-21-isopropyl-24-methyl-3,19,22-trioxo-2,7,10,13,16-pentaoxa-4,20,23-triazapentacosan-25-amido)benzyl)oxy)carbonyl)amino)-4-((S)-2-(((tert-butyldimethylsilyl)oxy)methyl)-4-methylenepyrrolidine-1-carbonyl)-2-methoxyphenoxy)pentyl)oxy)-2-((S)-2-(((tert-butyldimethylsilyl)oxy)methyl)-4-methylenepyrrolidine-1-carbonyl)-4-methoxyphenyl)carbamate (2-1 h)

To a solution of 2-1g (200 mg, 0.121 mmol) and 200 mg 4 Å MS in THE (2.5 mL) was added triphosgene (24.9 mg, 0.084 mmol), then TEA (64 μL, 0.462 mmol) was added at 0° C. under N₂. The mixture was stirred at 0° C. for 10 min under N₂. Formation of the isocyanate was monitored by LCMS analysis by quenching with methanol. A solution of 2-1f (176 mg, 0.231 mmol), dibutyltin dilaurate (13.3 mg, 0.021 mmol), TEA (43.7 μL, 0.315 mmol) in THE (2.5 mL) was added to the mixture. The mixture was stirred for 3 h at r.t. The mixture was filtered and the filter was concentrated. The residue was purified by silica column gel chromatography (CH₂Cl₂/MeOH=96/4) to give the product 2-lh (302 mg, 83% yield) as a white solid. MS (ESI) m/z: 1472.3 [M+H]⁺.

### Step 5: tert-butyl (5-((5-(5-((((4-((21S,24S)-1-(9H-fluoren-9-yl)-21-isopropyl-24-methyl-3,19,22-trioxo-2,7,10,13,16-pentaoxa-4,20,23-triazapentacosan-25-amido)benzyl)oxy)carbonyl)amino)-4-((S)-2-(hydroxymethyl)-4-methylenepyrrolidine-1-carbonyl)-2-methoxyphenoxy)pentyl)oxy)-2-((S)-2-(hydroxymethyl)-4-methylenepyrrolidine-1-carbonyl)-4-methoxyphenyl)carbamate (2-1i)

para-Toluenesulfonic acid hydrate (40 mg, 0.21 mmol) was added to a solution of 2-1 h (304 mg, 0.17 mmol) in THE (3 mL) and water (0.15 mL). The reaction mixture was allowed to stir for 4 h at 22° C. Upon complete reaction as determined by TLC (CH₂Cl₂/MeOH=20:1), the mixture was diluted with EtOAc (20 mL), washed with water, sat. NaHCO₃ and brine. The organic phase was concentrated and purified by silica column gel chromatography (CH₂Cl₂/MeOH=95:5) to give the product 2-1i (213 mg, 81% yield). MS (ESI) m/z: 1514.1 [M+H]⁺.

### Step 6: 4-((21S,24S)-1-(9H-fluoren-9-yl)-21-isopropyl-24-methyl-3,19,22-trioxo-2,7,10,13,16-pentaoxa-4,20,23-triazapentacosan-25-amido)benzyl (11S,11aS)-8-((5-(((11S,11aS)-10-(tert-butoxycarbonyl)-11-hydroxy-7-methoxy-2-methylene-5-oxo-2,3,5,10,11,11a-hexahydro-1H-benzo[e]pyrrolo[1,2-a][1,4]diazepin-8-yl)oxy)pentyl)oxy)-11-hydroxy-7-methoxy-2-methylene-5-oxo-2,3,11,11a-tetrahydro-1H-benzo[e]pyrrolo[1,2-a][1,4]diazepine-10(5H)-carboxylate (2-1j)

DMP (58.9 mg, 0.139 mmol) was added slowly, portion-wise to a solution of 2-li (100 mg) in dry CH₂Cl₂ (2 mL) at 0° C. The reaction was then warmed to r.t. and stirred overnight. The reaction was quenched with sat. Na₂S₂O₃, followed by addition of sat. NaHCO₃ and water. The layers were separated, and the organic layer was washed with sat. Na₂S₂O₃, sat. NaHCO₃ and brine, dried over Na₂SO₄. The crude product was purified by silica column gel chromatography (CH₂Cl₂/MeOH=95/5) to give the product 2-1j (80 mg, 80% yield). MS (ESI) m/z: 1510.3 [M+H]⁺.

### Step 7: 4-((21S,24S)-1-(9H-fluoren-9-yl)-21-isopropyl-24-methyl-3,19,22-trioxo-2,7,10,13,16-pentaoxa-4,20,23-triazapentacosan-25-amido)benzyl (11S,11aS)-11-hydroxy-7-methoxy-8-((5-(((S)-7-methoxy-2-methylene-5-oxo-2,3, 5,11a-tetrahydro-1H-benzo[e]pyrrolo[1,2-a][1,4]diazepin-8-yl)oxy)pentyl)oxy)-2-methylene-5-oxo-2,3,11,11a-tetrahydro-1H-benzo[e]pyrrolo[1,2-a][1,4]diazepine-10(5H)-carboxylate (2-1k)

2-1j (60 mg, 0.040 mmol) was cooled to −3° C. Separately, a solution of 95% TFA in H₂O (1.5 mL) was cooled to −3° C. before adding to the 2-1j. The reaction mixture was stirred at -3° C. for 40 min before pouring into a 1:1 solution of CHCl₃/NaHCO₃ (40 mL) at 0° C. The organic layer was separated, dried on Na₂SO₄, filtered and the solvent was removed in vacuo. The crude material 2-1k was used directly in the next step.

MS (ESI) m/z: 1392.6 [M+H]⁺.

### Step 8: 4-((17S,20S)-1-amino-17-isopropyl-20-methyl-15,18-dioxo-3,6,9,12-tetraoxa-16,19-diazahenicosan-21-amido)benzyl (11S,11As)-11-hydroxy-7-methoxy-8-((5-(((S)-7-methoxy-2-methylene-5-oxo-2,3, 5,11a-tetrahydro-1H-benzo[e]pyrrolo[1,2-a][1,4]diazepin-8-yl)oxy)pentyl)oxy)-2-methylene-5-oxo-2,3,11,11a-tetrahydro-1H-benzo[e]pyrrolo[1,2-a][1,4]diazepine-10(5H)-carboxylate (2-11)

To a solution of crude 2-1k in 0.5 mL DMF was added Et₂NH (61 μL, 0.593 mmol). The mixture was stirred at r.t. for 0.5 h. After the reaction was completed, the mixture was purified by prep-HPLC (0.1% FA in H₂O) to give the product 2-11 (14.3 mg, 31% yield, two steps).

MS (ESI) m/z: 1170.3 [M+H]⁺.

### Step 9: 4-((21S,24S)-1-((1R,8S,9s)-bicyclo[6.1.0]non-4-yn-9-yl)-21-isopropyl-24-methyl-3,19,22-trioxo-2,7,10,13,16-pentaoxa-4,20,23-triazapentacosan-25-amido)benzyl (11S,11As)-11-hydroxy-7-methoxy-8-((5-(((S)-7-methoxy-2-methylene-5-oxo-2,3,5,11a-tetrahydro-1H-benzo[e]pyrrolo[1,2-a][1,4]diazepin-8-yl)oxy)pentyl)oxy)-2-methylene-5-oxo-2,3,11,11a-tetrahydro-1H-benzo[e]pyrrolo[1,2-a][1,4]diazepine-10(5H)-carboxylate (2-1)

DIPEA (8.6 μL, 0.05 mmol) was added to a solution of 2-11 (14.5 mg, 0.012 mmol) and 2-1m (10.8 mg, 0.037 mmol) in 1 mL DMF. The mixture was stirred at r.t. for 20 min. The mixture was purified by prep-HPLC (no additive in mobile phase) to give the product 2-1 (7.7 mg, 46% yield) as a light gray solid.

MS (ESI) m/z: 1346.4 [M+H]⁺.

After linker cleavage, the payload released from Linker-Payload 2-1 undergoes a dehydration reaction and forms a compound, SG-2057, which has similar potency to Payload Ref-1-1 (also known as SG-3199).

### Example 2-2

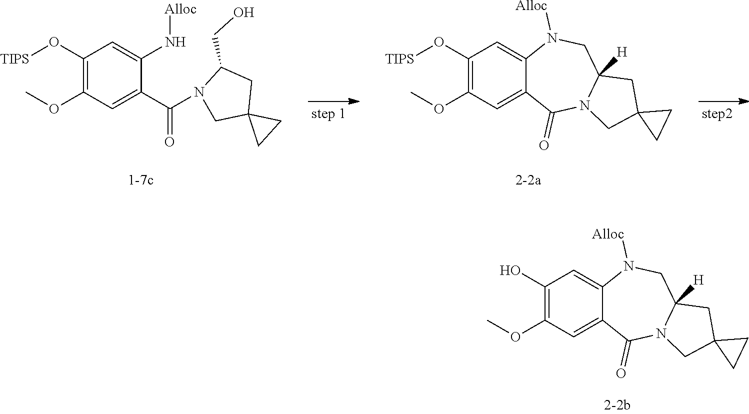

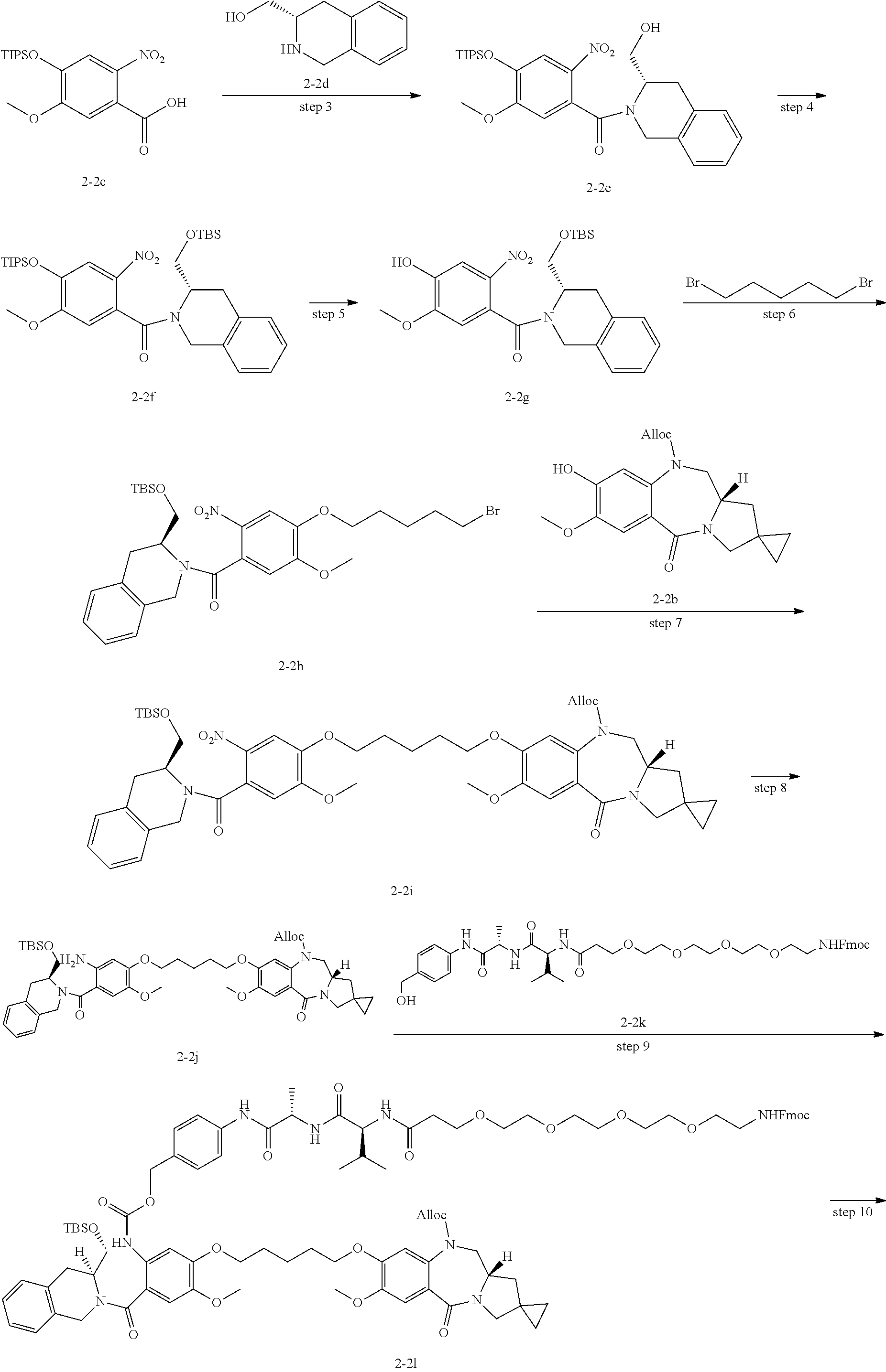

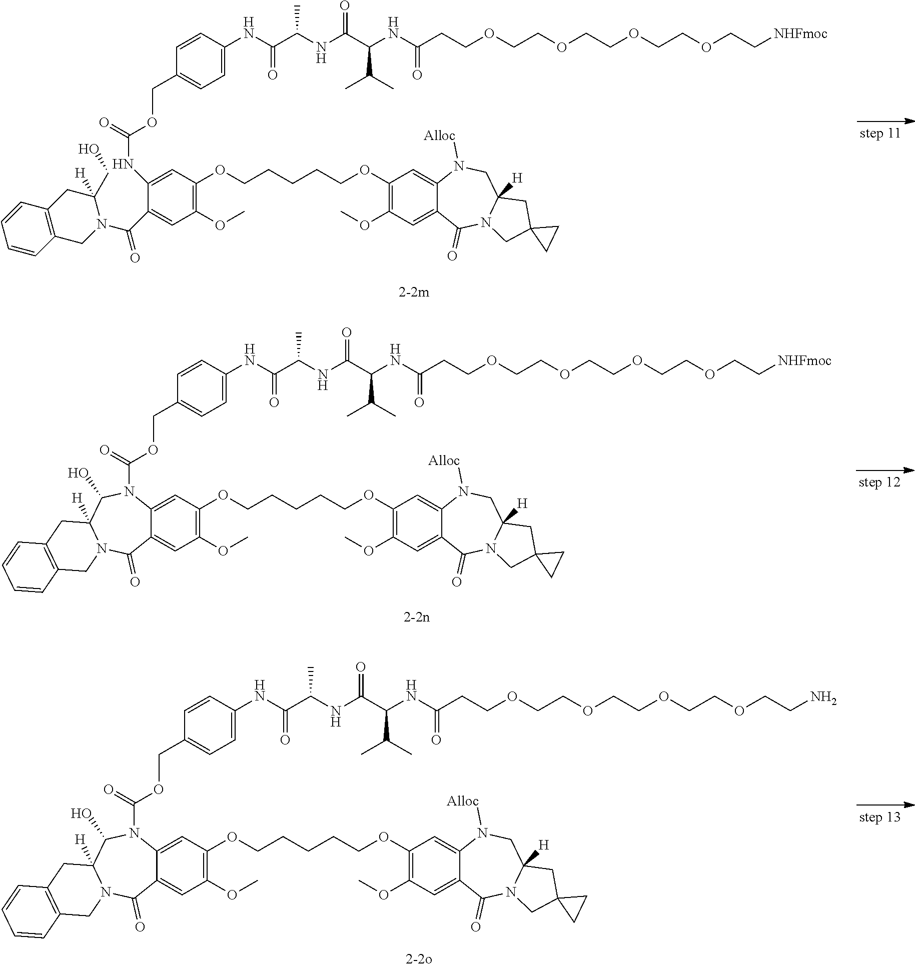

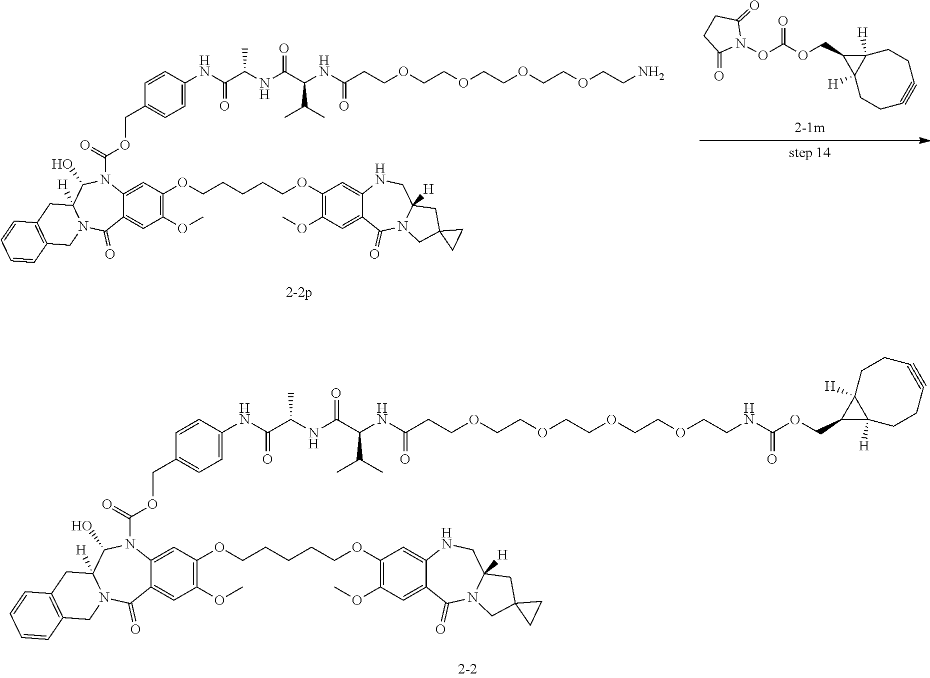 [SMILES: COc1cc2c(cc1OCCCCCOc1cc3c(cc1OC)C(=O)N1Cc4ccccc4C[C@H]1[C@H](O)N3C(=O)OCc1ccc(NC(=O)[C@H](C)NC(=O)[C@@H](NC(=O)CCOCCOCCOCCOCCN)C(C)C)cc1)NC[C@@H]1CC3(CC3)CN1C2=O ; O=C(OC[C@@H]1[C@@H]2CCC#CCC[C@@H]21)ON1C(=O)CCC1=O ; COc1cc2c(cc1OCCCCCOc1cc3c(cc1OC)C(=O)N1Cc4ccccc4C[C@H]1[C@H](O)N3C(=O)OCc1ccc(NC(=O)[C@H](C)NC(=O)[C@@H](NC(=O)CCOCCOCCOCCOCCNC(=O)OC[C@@H]3[C@@H]4CCC#CCC[C@@H]43)C(C)C)cc1)NC[C@@H]1CC3(CC3)CN1C2=O]

### Step 1: allyl (S)-7-methoxy-5-oxo-8-((triisopropylsilyl)oxy)-11,11a-dihydro-1H,3H-spiro[benzo[e]pyrrolo[1,2-a][1,4]diazepine-2,1′-cyclopropane]-10(5H)-carboxylate (2-2a)

DIAD (1.14 mL, 5.75 mmol) was added to a solution of 1-7c (1.53 g, 2.87 mmol) and PPh₃ (2.26 g, 8.62 mmol) in THE (30 mL). The reaction mixture was allowed to stir for 1 h at 40° C. under N₂. Upon completion of reaction by TLC (petroleum ether/EtOAc=1:2), the solvent was removed under vacuum, and the residue was purified by silica column gel chromatography to give the product (petroleum ether/EtOAc=40:60) 2-2a (940 mg, 64% yield). MS (ESI) m/z: 515.5 [M+H]⁺.

¹H NMR (400 MHz, CDCl₃) δ 7.19 (s, 1H), 6.70 (s, 1H), 5.84-5.68 (m, 1H), 5.12 (t, J=12.7 Hz, 2H), 4.58 (dd, J=12.9, 5.3 Hz, 1H), 4.45 (t, J=12.5 Hz, 2H), 3.89-3.86 (m, 1H), 3.85 (s, 3H), 3.74 (d, J=11.8 Hz, 1H), 3.48 (dd, J=12.2, 4.2 Hz, 1H), 3.37 (d, J=11.8 Hz, 1H), 2.37 (dd, J=12.8, 8.4 Hz, 1H), 1.42 (d, J=12.8 Hz, 1H), 1.25 (ddd, J=19.0, 9.0, 4.5 Hz, 3H), 1.08 (dd, J=7.3, 3.7 Hz, 18H), 0.86-0.57 (m, 4H).

### Step 2: allyl (S)-8-hydroxy-7-methoxy-5-oxo-11,11a-dihydro-1H,3H-spiro[benzo[e]pyrrolo[1,2-a][1,4]diazepine-2,1′-cyclopropane]-10(5H)-carboxylate (2-2b)

Lithium acetate (121 mg, 1.83 mmol) was added to a solution of 2-2a (940 mg, 1.83 mmol) in wet DMF (15 mL 49/1 DMF/water). The reaction was allowed to proceed for 2 h at 25° C. The mixture was diluted with EtOAc, washed with H₂O and brine twice. The organic phase was dried over Na₂SO₄, concentrated and purified by silica column gel chromatography to give the product 2-2b (605 mg, 92% yield). MS (ESI) m/z: 359.4 [M+H]⁺.

### Step 3: (S)-(3-(hydroxymethyl)-3,4-dihydroisoquinolin-2(1H)-yl)(5-methoxy-2-nitro-4-((triisopropylsilyl)oxy)phenyl)methanone (2-2e)

EDCI (935 mg, 4.88 mmol) was added to a solution of 2-2c (1.5 g, 4.06 mmol) and 2-pyridinol 1-oxide (HOPO, 497 mg, 4.47 mmol) in CH₂Cl₂ (15 mL) at 0° C. The reaction was allowed to proceed for 1 h at 15° C., at which time a solution of 2-2d (729 mg, 4.47 mmol) and triethylamine (0.71 mL, 5.08 mmol) in CH₂Cl₂ (15 mL) was added at −10° C. The reaction mixture was allowed to stir at r.t. for 3 h. The reaction mixture was washed with water (˜30 mL), followed by cold aqueous HCl (0.5 M) until the pH was adjusted to 4-5. The organic phase was then washed with saturated aqueous NaHCO₃ (˜30 mL), then water (˜30 mL). The solvents were removed under vacuum to give crude product which was purified by silica column gel chromatography to give product 2-2e (1.34 g, 57% yield). MS (ESI) m/z: 515.4 [M+H]⁺.

### Step 4: (S)-(3-(((tert-butyldimethylsilyl)oxy)methyl)-3,4-dihydroisoquinolin-2(1H)-yl)(5-methoxy-2-nitro-4-((triisopropylsilyl)oxy)phenyl)methanone (2-2f)

Imidazole (355 mg, 5.21 mmol) was added to a solution of 2-2e (1.34 g, 2.61 mmol) in 25 mL CH₂Cl₂. Then TBSCI (589 mg, 3.91 mmol) was added to the mixture at r.t. The mixture was stirred at r.t. overnight. The reaction was filtered and the filtrate was concentrated. The crude product was purified by silica column gel chromatography to give the product 2-2f (1.38 g, 84% yield). MS (ESI) m/z: 629.5 [M+H]⁺.

### Step 5: (S)-(3-(((tert-butyldimethylsilyl)oxy)methyl)-3,4-dihydroisoquinolin-2(1H)-yl)(4-hydroxy-5-methoxy-2-nitrophenyl)methanone (2-2g)

Lithium acetate (145 mg, 2.2 mmol) was added to a solution of 2-2f (1.38 g, 2.2 mmol) in wet DMF (15 mL, 49/1 DMF/water). The reaction was allowed to proceed for 2 h at 25° C. The mixture was diluted with EtOAc, washed with H₂O and brine twice. The organic phase was concentrated and purified by silica column gel chromatography to give the product 2-2g (930 mg, 90% yield). MS (ESI) m/z: 473.3 [M+H]⁺.

### Step 6: (S)-(4-((5-bromopentyl)oxy)-5-methoxy-2-nitrophenyl)(3-(((tert-butyldimethylsilyl)oxy)methyl)-3,4-dihydroisoquinolin-2(1H)-yl)methanone (2-2 h)

To a solution of 2-2g (930 mg, 1.97 mmol) and 1,5-dibromopentane (4.0 mL, 29.5 mmol) in 18 mL DMF was added K₂CO₃ (330 mg, 2.36 mmol) at r.t. The mixture was stirred at r.t. for 2 h. The mixture was diluted with EtOAc, washed with H₂O and brine. The organic phase was concentrated and purified by silica column gel chromatography (petroleum ether/EtOAc=75/25) to give product 2-2 h (818 mg, 67% yield). MS (ESI) m/z: 621.4 [M+H]⁺.

### Step 7: allyl (S)-8-((5-(4-((S)-3-(((tert-butyldimethylsilyl)oxy)methyl)-1,2,3,4-tetrahydroisoquinoline-2-carbonyl)-2-methoxy-5-nitrophenoxy)pentyl)oxy)-7-methoxy-5-oxo-11,11a-dihydro-1H,3H-spiro[benzo[e]pyrrolo[1,2-a][1,4]diazepine-2,1′-cyclopropane]-10(5H)-carboxylate (2-2i)

2-2b (450 mg, 1.26 mmol) and K₂CO₃ (226 mg, 1.63 mmol) was added to a solution of 2-2 h (818 mg, 1.32 mmol) in DMF (1 mL). The mixture was stirred at r.t. for 24 h. The product (petroleum ether/EtOAc=1:2) was detected by LCMS. The mixture was diluted with EtOAc, washed with water, brine. The organic phase was concentrated and purified by silica column gel chromatography (petroleum ether/EtOAc=33:67) to give the product 2-2i (930 mg, 82% yield). MS (ESI) m/z: 899.6 [M+H]⁺.

### Step 8: allyl (S)-8-((5-(5-amino-4-((S)-3-(((tert-butyldimethylsilyl)oxy)methyl)-1,2,3,4-tetrahydroisoquinoline-2-carbonyl)-2-methoxyphenoxy)pentyl)oxy)-7-methoxy-5-oxo-11,11a-dihydro-1H,3H-spiro[benzo[e]pyrrolo[1,2-a][1,4]diazepine-2,1′-cyclopropane]-10(5H)-carboxylate (2-2j)

Zinc powder (2.65 g, 38.3 mmol) was added to a mixture of ethanol (10 mL), water (0.625 mL), and AcOH (0.625 mL) at 0° C. The reaction mixture was stirred at 5° C. for 30 min. A solution of 2-2i (930 mg, 1.04 mmol) in ethanol (6 mL) was added dropwise at 5° C. The reaction was allowed to proceed at 5° C. for 50 min. The solids were removed by filtration. The filtrate was diluted with EtOAc and washed with water, saturated aqueous NaHCO₃, and brine. The organic phase was dried over sodium sulfate and filtered, the solvent removed by rotary evaporation under reduced pressure to afford the crude product which was purified by silica column gel chromatography (petroleum ether/EtOAc=33:67) to give the product 2-2j (806 mg, 90% yield) as a yellow solid. MS (ESI) m/z: 868.7 [M+H]⁺.

### Step 9: allyl (S)-8-((5-(5-((((4-((21S,24S)-1-(9H-fluoren-9-yl)-21-isopropyl-24-methyl-3,19,22-trioxo-2,7,10,13,16-pentaoxa-4,20,23-triazapentacosan-25-amido)benzyl)oxy)carbonyl)amino)-4-((S)-3-(((tert-butyldimethylsilyl)oxy)methyl)-1,2,3,4-tetrahydroisoquinoline-2-carbonyl)-2-methoxyphenoxy)pentyl)oxy)-7-methoxy-5-oxo-11,11a-dihydro-1H,3H-spiro[benzo[e]pyrrolo[1,2-a][1,4]diazepine-2,1′-cyclopropane]-10(5H)-carboxylate (2-21)

2-21 was prepared according to the procedure described in Step 4 of Example 2-1 to obtain a white solid (260 mg, yield 91%). MS (ESI) m/z: 1658.4 [M+H]⁺.

### Step 10: allyl (S)-8-((5-(5-((((4-((21S,24S)-1-(9H-fluoren-9-yl)-21-isopropyl-24-methyl-3,19,22-trioxo-2,7,10,13,16-pentaoxa-4,20,23-triazapentacosan-25-amido)benzyl)oxy)carbonyl)amino)-4-((S)-3-(hydroxymethyl)-1,2,3,4-tetrahydroisoquinoline-2-carbonyl)-2-methoxyphenoxy)pentyl)oxy)-7-methoxy-5-oxo-11,11a-dihydro-1H,3H-spiro[benzo[e]pyrrolo[1,2-a][1,4]diazepine-2,1′-cyclopropane]-10(5H)-carboxylate (2-2m)

para-Toluenesulfonic acid hydrate (29.8 mg, 0.16 mmol) was added to a solution of 2-21 (260 mg, 0.16 mmol) in THE (6 mL) and water (0.3 mL). The reaction mixture was allowed to stir overnight at 22° C. The mixture was diluted with EtOAc (20 mL), washed with water and brine. The organic phase was concentrated and purified by silica column gel chromatography to give the product 2-2m (172 mg, 71% yield). MS (ESI) m/z: 1544.3 [M+H]⁺.

### Step 11: 4-((21S,24S)-1-(9H-fluoren-9-yl)-21-isopropyl-24-methyl-3,19,22-trioxo-2,7,10,13,16-pentaoxa-4,20,23-triazapentacosan-25-amido)benzyl (6S,6As)-3-((5-(((S)-10-((allyloxy)carbonyl)-7-methoxy-5-oxo-5,10,11,11a-tetrahydro-1H,3H-spiro[benzo[e]pyrrolo[1,2-a][1,4]diazepine-2,1′-cyclopropan]-8-yl)oxy)pentyl)oxy)-6-hydroxy-2-methoxy-14-oxo-6,6a,7,12-tetrahydrobenzo[5,6][1,4]diazepino[1,2-b]isoquinoline-5(14H)-carboxylate (2-2n)

DMP (52 mg, 0.123 mmol) was added slowly, portion-wise to a solution of 2-2m (172 mg) in dry CH₂Cl₂ (2.5 mL) at 0° C. The reaction was then warmed to r.t. and stirred for 9 h. After 9 h, the reaction was quenched with sat. Na₂S₂O₃, followed by addition of sat. NaHCO₃ and water. The layers were separated, and the organic layer was washed with sat. Na₂S₂O₃, sat. NaHCO₃ and brine, dried over Na₂SO₄. The crude product was purified by silica column gel chromatography (CH₂Cl₂/MeOH=95/5) to give the product 2-2n (130 mg, 76% yield). MS (ESI) m/z: 1542.1 [M+H]⁺.

### Step 12: 4-((17S,20S)-1-amino-17-isopropyl-20-methyl-15,18-dioxo-3,6,9,12-tetraoxa-16,19-diazahenicosan-21-amido)benzyl (6S,6As)-3-((5-(((S)-10-((allyloxy)carbonyl)-7-methoxy-5-oxo-5,10,11,11a-tetrahydro-1H,3H-spiro[benzo[e]pyrrolo[1,2-a][1,4]diazepine-2,1′-cyclopropan]-8-yl)oxy)pentyl)oxy)-6-hydroxy-2-methoxy-14-oxo-6,6a,7,12-tetrahydrobenzo[5,6][1,4]diazepino[1,2-b]isoquinoline-5(14H)-carboxylate (2-20)

To a solution of 2-2n (50 mg, 0.04 mmol) in DMF (2 mL) was added Et₂NH (38 μL, 0.37 mmol). The mixture was stirred at r.t. for 1 h. After the reaction was completed, the mixture was concentrated to give crude 2-2o which was used directly for the next step. MS (ESI) m/z: 1320.2 [M+H]⁺.

### Step 13: 4-((17S,20S)-1-amino-17-isopropyl-20-methyl-15,18-dioxo-3,6,9,12-tetraoxa-16,19-diazahenicosan-21-amido)benzyl (6S,6aS)-6-hydroxy-2-methoxy-3-((5-(((S)-7-methoxy-5-oxo-5,10,11,11a-tetrahydro-1H,3H-spiro[benzo[e]pyrrolo[1,2-a][1,4]diazepine-2,1′-cyclopropan]-8-yl)oxy)pentyl)oxy)-14-oxo-6,6a,7,12-tetrahydrobenzo[5,6][1,4]diazepino[1,2-b]isoquinoline-5(14H)-carboxylate (2-2p)

Pd(PPh₃)₄(2.15 mg, 0.002 mmol) was added to a solution of crude 2-2o in THF/MeOH (2 mL/0.2 mL) and dimedone (10.42 mg, 0.074 mmol) at r.t. The reaction mixture was allowed to stir for 1 h at r.t. The reaction was concentrated. The residue was purified by prep-HPLC (0.1% FA in H₂O) to give the product 2-2p (16 mg, 61% yield) as a white solid. MS (ESI) m/z: 1236.1 [M+H]⁺.

### Step 14: 4-((21S,24S)-1-((1R,8S,9s)-bicyclo[6.1.0]non-4-yn-9-yl)-21-isopropyl-24-methyl-3,19,22-trioxo-2,7,10,13,16-pentaoxa-4,20,23-triazapentacosan-25-amido)benzyl (6S,6As)-6-hydroxy-2-methoxy-3-((5-(((S)-7-methoxy-5-oxo-5,10,11,11a-tetrahydro-1H,3H-spiro[benzo[e]pyrrolo[1,2-a][1,4]diazepine-2,1′-cyclopropan]-8-yl)oxy)pentyl)oxy)-14-oxo-6,6a,7,12-tetrahydrobenzo[5,6][1,4]diazepino[1,2-b]isoquinoline-5(14H)-carboxylate (2-2)

DIPEA (10.9 μL, 0.063 mmol) was added to the solution of 2-2p (19.3 mg, 0.016 mmol) and 2-1m (13.6 mg, 0.047 mmol) in DMF (1 mL). The mixture was stirred at r.t. for 20 min. The mixture was purified by prep-HPLC (0.1% FA in H₂O) to give the product 2-2 (9.8 mg, 44% yield) as a white solid. MS (ESI) m/z: 1412.4 [M+H]⁺.

After linker cleavage, the payload released from Linker-Payload 2-2 undergoes a dehydration reaction and forms the compound of Example 1-3.

The compounds disclosed here may also be synthesized based on the synthetic methods provided herein in combination with the common knowledge in the art.

TABLE 1
Payload Structures

| Example | |
|---|---|
| No. | Payload structure |
| Ref-1-1 | 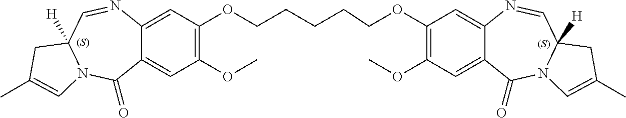 |
| 1-1 | 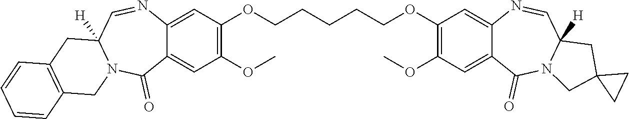 [SMILES: COc1cc2c(cc1OCCCCCOc1cc3c(cc1OC)C(=O)N1CC4(CC4)C[C@H]1C=N3)N=C[C@@H]1Cc3ccccc3CN1C2=O] |
| 1-2 | 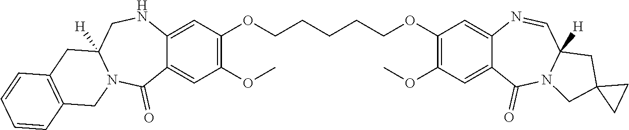 [SMILES: COc1cc2c(cc1OCCCCCOc1cc3c(cc1OC)C(=O)N1Cc4ccccc4C[C@H]1CN3)N=C[C@@H]1CC3(CC3)CN1C2=O] |
| 1-3 | 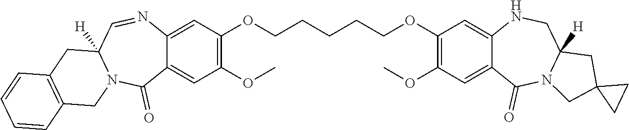 [SMILES: COc1cc2c(cc1OCCCCCOc1cc3c(cc1OC)C(=O)N1CC4(CC4)C[C@H]1CN3)N=C[C@@H]1Cc3ccccc3CN1C2=O] |
| 1-4 | 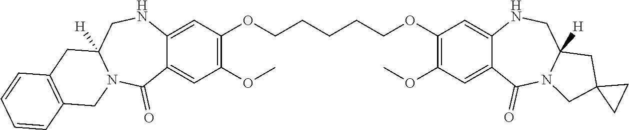 [SMILES: COc1cc2c(cc1OCCCCCOc1cc3c(cc1OC)C(=O)N1CC4(CC4)C[C@H]1CN3)NC[C@@H]1Cc3ccccc3CN1C2=O] |
| 1-5 | 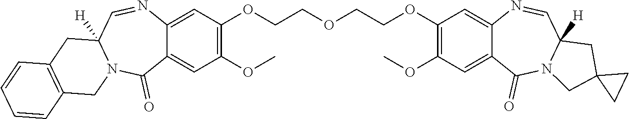 [SMILES: COc1cc2c(cc1OCCOCCOc1cc3c(cc1OC)C(=O)N1CC4(CC4)C[C@H]1C=N3)N=C[C@@H]1Cc3ccccc3CN1C2=O] |
| 1-6 | 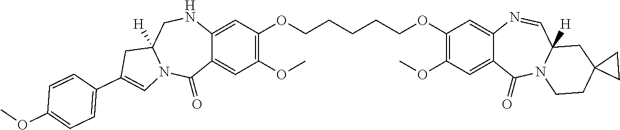 [SMILES: COc1ccc(C2=CN3C(=O)c4cc(OC)c(OCCCCCOc5cc6c(cc5OC)C(=O)N5CCC7(CC7)C[C@H]5C=N6)cc4NC[C@@H]3C2)cc1] |
| 1-7 | 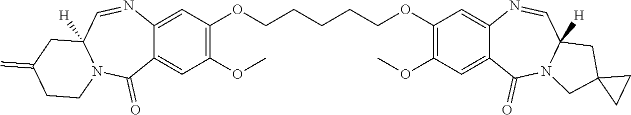 [SMILES: C=C1CCN2C(=O)c3cc(OC)c(OCCCCCOc4cc5c(cc4OC)C(=O)N4CC6(CC6)C[C@H]4C=N5)cc3N=C[C@@H]2C1] |
| 1-8 | 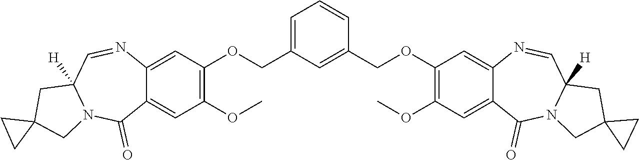 [SMILES: COc1cc2c(cc1OCc1cccc(COc3cc4c(cc3OC)C(=O)N3CC5(CC5)C[C@H]3C=N4)c1)N=C[C@@H]1CC3(CC3)CN1C2=O] |
| 1-9 | 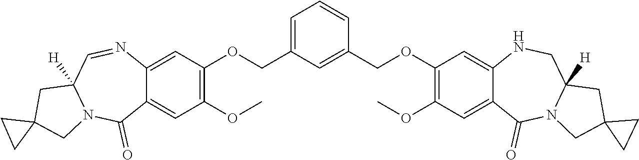 [SMILES: COc1cc2c(cc1OCc1cccc(COc3cc4c(cc3OC)C(=O)N3CC5(CC5)C[C@H]3CN4)c1)N=C[C@@H]1CC3(CC3)CN1C2=O] |
| 1-10 | 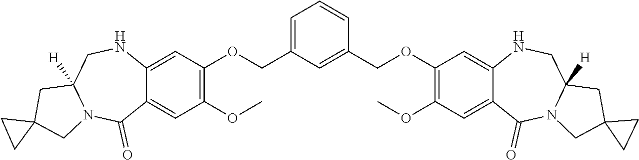 [SMILES: COc1cc2c(cc1OCc1cccc(COc3cc4c(cc3OC)C(=O)N3CC5(CC5)C[C@H]3CN4)c1)NC[C@@H]1CC3(CC3)CN1C2=O] |
| 1-11 | 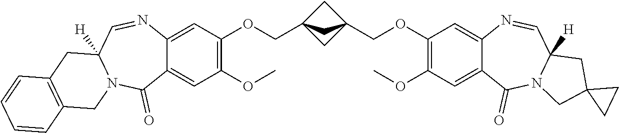 [SMILES: COc1cc2c(cc1OC[C@]13C[C@](COc4cc5c(cc4OC)C(=O)N4CC6(CC6)C[C@H]4C=N5)(C1)C3)N=C[C@@H]1Cc3ccccc3CN1C2=O] |
| 1-12 | 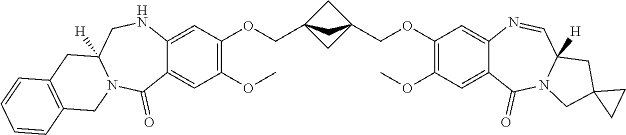 [SMILES: COc1cc2c(cc1OC[C@]13C[C@](COc4cc5c(cc4OC)C(=O)N4Cc6ccccc6C[C@H]4CN5)(C1)C3)N=C[C@@H]1CC3(CC3)CN1C2=O] |
| 1-13 | 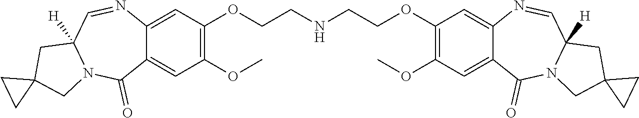 [SMILES: COc1cc2c(cc1OCCNCCOc1cc3c(cc1OC)C(=O)N1CC4(CC4)C[C@H]1C=N3)N=C[C@@H]1CC3(CC3)CN1C2=O] |
| 1-14 | 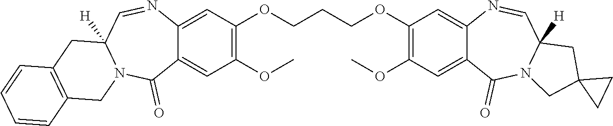 [SMILES: COc1cc2c(cc1OCCCOc1cc3c(cc1OC)C(=O)N1CC4(CC4)C[C@H]1C=N3)N=C[C@@H]1Cc3ccccc3CN1C2=O] |
| 1-15 | 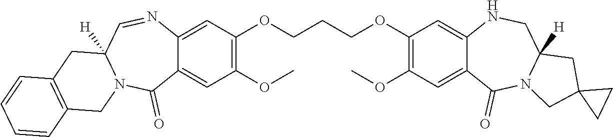 [SMILES: COc1cc2c(cc1OCCCOc1cc3c(cc1OC)C(=O)N1CC4(CC4)C[C@H]1CN3)N=C[C@@H]1Cc3ccccc3CN1C2=O] |
| 1-16 | 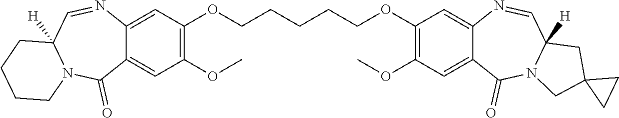 [SMILES: COc1cc2c(cc1OCCCCCOc1cc3c(cc1OC)C(=O)N1CC4(CC4)C[C@H]1C=N3)N=C[C@@H]1CCCCN1C2=O] |
| 1-17 | 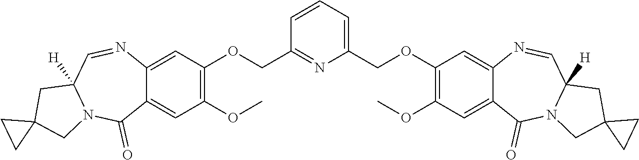 [SMILES: COc1cc2c(cc1OCc1cccc(COc3cc4c(cc3OC)C(=O)N3CC5(CC5)C[C@H]3C=N4)n1)N=C[C@@H]1CC3(CC3)CN1C2=O] |
| 1-18 | 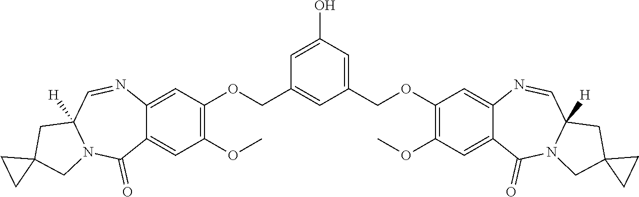 [SMILES: COc1cc2c(cc1OCc1cc(O)cc(COc3cc4c(cc3OC)C(=O)N3CC5(CC5)C[C@H]3C=N4)c1)N=C[C@@H]1CC3(CC3)CN1C2=O] |
| 1-19 | 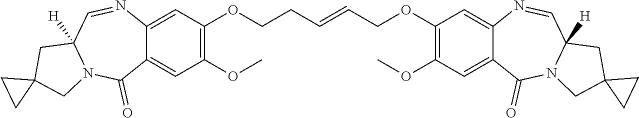 [SMILES: COc1cc2c(cc1OC/C=C/CCOc1cc3c(cc1OC)C(=O)N1CC4(CC4)C[C@H]1C=N3)N=C[C@@H]1CC3(CC3)CN1C2=O] |
| 1-20 | 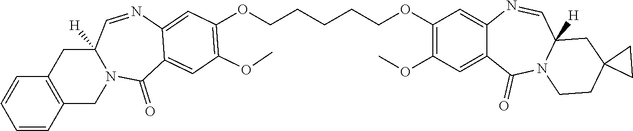 [SMILES: COc1cc2c(cc1OCCCCCOc1cc3c(cc1OC)C(=O)N1Cc4ccccc4C[C@H]1C=N3)N=C[C@@H]1CC3(CCN1C2=O)CC3] |
| 1-21 | 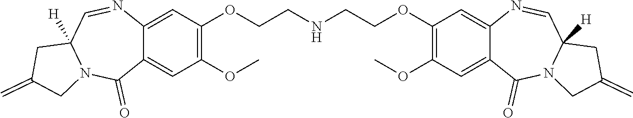 |
| 1-22 | 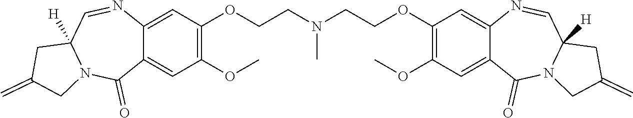 |

TABLE 2
Linker-Payload Structures

| Example | |
|---|---|
| No. | Linker-payload structure |
| 2-1 | 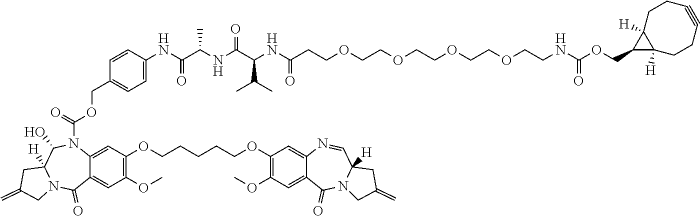 [SMILES: C=C1C[C@H]2C=Nc3cc(OCCCCCOc4cc5c(cc4OC)C(=O)N4CC(=C)C[C@H]4[C@H](O)N5C(=O)OCc4ccc(NC(=O)[C@H](C)NC(=O)[C@@H](NC(=O)CCOCCOCCOCCOCCNC(=O)OC[C@@H]5[C@@H]6CCC#CCC[C@@H]65)C(C)C)cc4)c(OC)cc3C(=O)N2C1] |
| 2-2 | 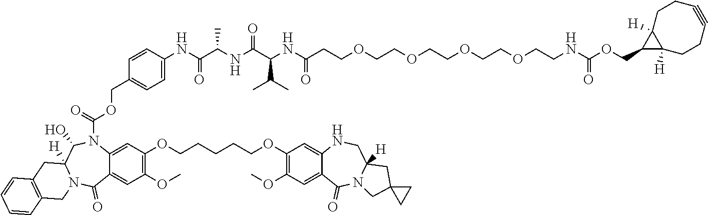 [SMILES: COc1cc2c(cc1OCCCCCOc1cc3c(cc1OC)C(=O)N1Cc4ccccc4C[C@H]1[C@H](O)N3C(=O)OCc1ccc(NC(=O)[C@H](C)NC(=O)[C@@H](NC(=O)CCOCCOCCOCCOCCNC(=O)OC[C@@H]3[C@@H]4CCC#CCC[C@@H]43)C(C)C)cc1)NC[C@@H]1CC3(CC3)CN1C2=O] |

### ADC Preparation and Characterization

DAR2 antibody drug conjugate preparation. Anti-CD74 antibody mAbI in reaction buffer (with concentration 0.5-25 mg/mL, 50 mM Tris-HCl buffer pH 7.0-8.5) was incubated with 1/2000-1/500 w/w (EndoS2/mAb weight ratio) endoS2 under reaction temperature (0-40° C.) for 1-24 h. 2-40 eq. UDP-GalNAz (20 mM) and 0.1 w/w %-10 w/w % (GalT/mAb weight ratio) β1,4-GalT were added into the reaction mixture and incubated in reaction buffer (50 mM Tris-HCl buffer pH 7.0-8.5, 20 mM MnCl₂) for 8-24 hours at reaction temperature (0-40° C.). The reaction mixture was purified with protein A resin to give the mAb 1-GaINAz.

Organic solvent (e.g., DMS0, DMIF, DMA, PG, acetonitrile, 0-25% o v/v) and linker-payload stock (10-25 eq., 10 mM stock in organic solvent) were added stepwise in reaction buffer (PBS buffer pH 7.0-8.5) with mAbl-GalNAz (1-20 mg/mL) at 0-25° C. for 0.5-24 h. The solution was submitted to buffer exchange (spin desalting column, ultrafiltration, and dialysis) into storage buffer (for example, pH 5.5-6.5 histidine acetate buffer, with optional additive such as sucrose, trehalose, tween 20, 60, 80).

ADC characterization. ADCs were characterized via the following analytical methods. SEC purity of all ADCs were >95% purity.

Drug to Antibody Ratio (DAR) Determination by LCMS or HIC Methods LCMS method: LC-MS analysis was carried out under the following measurement conditions:

LC-MS system: Vanquish Flex UHPLC and Orbitrap Exploris 240 Mass Spectrometer

Column: MAbPac™ RP, 2.1*50 mm, 4 μm, 1,500 Å, Thermo Scientific™

Column temperature: 80° C.

Mobile phase A: 0.1% formic acid (FA) aqueous solution

Mobile phase B: Acetonitrile solution caontaining 0.1% formic acid (FA)

Gradient program: 25% B-25% B (0 min-2 min), 25% B-50% B (2 min-18 min), 50% B-90% B (18 min-18.1 min), 90% B-90% B (18.1 min-20 min), 90% B-25% B (20 min-20.1 min), 25% B-25% B (20.1 min-25 min)

Injected sample amount: 1 μg

MS parameters: Intact and denaturing MS data were acquired in HMR mode at setting of R=15k and deconvolved using the ReSpect™ algorithm and Sliding Window integration in Thermo Scientific™ BioPharma Finder™ 4.0 software.

HIC method: HPLC analysis was carried out under the following measurement conditions:

HPLC system: Waters ACQUITY ARC HPLC System

Detector: measurement wavelength: 280 nm

Column: Tosoh Bioscience 4.6 μm IDx3.5 cm, 2.5 μm butyl-nonporous resin column

Column temperature: 25° C.

Mobile phase A: 1.5 M ammonium sulfate, 50 mM phosphate buffer, pH 7.0

Mobile phase B: 50 mM phosphate buffer, 25% (V/V) isopropanol, pH 7.0

Gradient program: 0% B-0% B (0 min-2 min), 0% B-100% B (2 min-15 min), 100% B-100% B (15 min-16 min), 100% B-0% B (16 min-17 min), 0% B-0% B (17 min-20 min)

Injected sample amount: 20 μg

### SEC Method to Determine ADC Purity

HPLC analysis was carried out under the following measurement conditions:

HPLC system: Waters H-Class UPLC System

Detector: measurement wavelength: 280 nm

Column: ACQUITY UPLC BEH200 SEC 1.7 μm 4.6×mm, Waters

Column temperature: room temperature

Mobile phase A: 200 mM phosphate buffer, 250 mM potassium chloride, 15% isopropyl alcohol, pH 7.0

Gradient program: under 10 min isocratic elutions with a flow rate of 0.3 mL/min

Injected sample amount: 20 μg

ADC hydrophobicity evaluation by HIC method: An ADC with a higher hydrophobic property would appear with a later retention time from HIC (hydrophobicity interaction column) chromatography. The DAR2 peak was used as a reference.

HPLC analysis was carried out under the following measurement conditions:

### Method 1

HPLC system: Waters ACQUITY ARC HPLC System

Detector: measurement wavelength: 280 nm

Column: Tosoh Bioscience 4.6 μm IDx3.5 cm, 2.5 μm butyl-nonporous resin column

Column temperature: 25° C.

Mobile phase A: 1.5 M ammonium sulfate, 50 mM phosphate buffer, pH 7.0

Mobile phase B: 50 mM Phosphate buffer, 25% (V/V) isopropanol, pH 7.0

Gradient program: 0% B-0% B (0 min-2 min), 0% B-100% B (2 min-15 min), 100% B-100% B (15 min-16 min), 100% B-0% B (16 min-17 min), 0% B-0% B (17 min-20 min)

Injected sample amount: 20 μg

### Method 2

HPLC system: Waters ACQUITY ARC HPLC System

Detector: measurement wavelength: 280 nm

Column: MABPac HIC-10, 5 μm, 4.6×10 mm (Thermo)

Column temperature: 25° C.

Mobile phase A: 1.5 M ammonium sulfate, 50 mM sodium phosphate, pH 7.0

Mobile phase B: 50 mM sodium phosphate, pH 7.0

Gradient program: 20% B-20% B (0 min-1 min), 0% B-0% B (1 min-35 min), 20% B-20% B (35 min-40 min)

Flow rate: 0.5 mL/min

Sample preparation: The sample was diluted with initial mobile phase to 0.5 mg/mL.

TABLE 3
ADC Structures

| | Anti- | |
|---|---|---|
| ADC | body | |
| No. | (mAb) | ADC structure |
| ADC 3-1 | Anti- CD74 anti- body mAb1 | 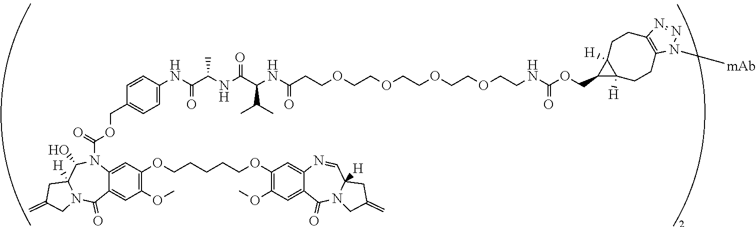 |
| ADC 3-2 | Anti- CD74 anti- body mAb1 | 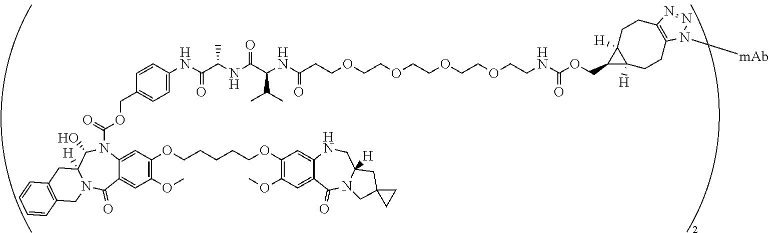 |

Anti-CD74 antibody mAb1
Light Chain Sequence
(SEQ ID NO: 1)
DIQMTQSPSSVSASVGDRVTITCRASQGIGSWLAWYQQKPGKAPK
LLIYAADRLQSGVPSRFSGSGSGTDFTLTISSLQPEDFATYYCQQ
YHTYPLTFGGGTKVEIKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSFNRGEC
Heavy Chain Sequence
(SEQ ID NO: 2)
QVQLVESGGGVVQPGRSLRLSCAASGFNFSDYGMHWVRQAPGKGL
EWVAVIWYDGSISYYADSVKGRFTISRDNSKNTLYLQMNSLRAED
TAVYYCARGGTVEHGAVYGTDVWGQGTTVTVSSASTKGPSVFPLA
PSSKSTSGGTAALGCLVKDYFPEPVTVSWNSGALTSGVHTFPAVL
QSSGLYSLSSVVTVPSSSLGTQTYICNVNHKPSNTKVDKKVEPKS
CDKTHTCPPCPAPELLGGPSVFLFPPKPKDTLMISRTPEVTCVVV
DVSHEDPEVKFNWYVDGVEVHNAKTKPREEQYNSTYRVVSVLTVL
HQDWLNGKEYKCKVSNKALPAPIEKTISKAKGQPREPQVYTLPPS
REEMTKNQVSLTCLVKGFYPSDIAVEWESNGQPENNYKTTPPVLD
SDGSFFLYSKLTVDKSRWQQGNVFSCSVMHEALHNHYTQKSLSLS
PGK

### Cell Lines

A375 (ATCC, CRL-1619). A-375 is a cell line exhibiting epithelial morphology that was isolated from the skin of a 54-year-old, female patient with malignant melanoma, and A375 was purchased from ATCC. The base medium for A375 is DMEM, high glucose, GlutaMAX™ Supplement (Gibco, 10566024). To make the complete growth medium, fetal bovine serum was added to the base mediumat a final concentration of 10% (Gibco, 10099-141C). The cell line was grown in a humidified 5% CO₂ atmosphere at 37° C., and was regularly tested for the presence of mycoplasma with MycoAlert™ PLUS Mycoplasma Detection Kit (Lonza, LT07-710).

Calu-6 (ATCC, HTB-56). Calu-6 is a cell line exhibiting epithelial morphology that was isolated from white female patient with anaplastic carcinoma, and Calu-6 was purchased from ATCC. The base medium for Calu-6 is Eagle's Minimum Essential Medium (ATCC, 30-2003). To make the complete growth medium, fetal bovine serum was added to the base mediumat a final concentration of 10% (Gibco, 10099-141C). The cell line was grown in a humidified 5% CO₂ atmosphere at 37° C., and was regularly tested for the presence of mycoplasma with MycoAlert™ PLUS Mycoplasma Detection Kit (Lonza, LT07-710).

### Compounds Cellular Killing in A375 and Calu-6 Cancer Lines

Payload direct killing by compounds 1-1 to 1-22 was assessed in A375 and Calu-6 cancer cell lines. Cells were seeded at 1E3/well (A375) 2E3/well (Calu-6) into 96-well plates (Greiner: 655090), 100 μL/well, and incubated at 37° C., 5% CO₂, overnight. Fresh growth medium was added containing varying concentrations of compounds, 50 μL/well, and incubated at 37° C., 5% CO₂, for 6 days. The cell viability detected by Cell Titer-Glo (Promega, G7573), 70 μL/well. The plates were allowed to incubate at room temperature for 10 minutes to stabilize the luminescent signal. The plates were analyzed with a microplate reader.

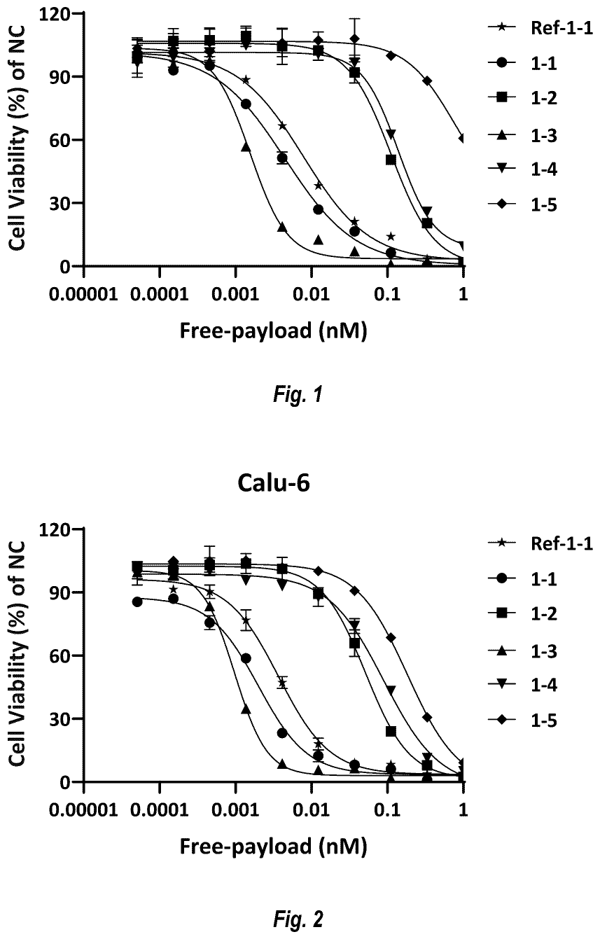

The cellular killing data of compounds 1-1 to 1-5 are presented in FIGS. 1 and 2 and in the following table:

| Compound | A375 | | Calu-6 | |
|---|---|---|---|---|
| No. | Emax (%) | EC50 (nM) | Emax (%) | EC50 (nM) |
| Ref-1-1 | 99.8 | 0.009 | 99.5 | 0.004 |
| 1-1 | 99.8 | 0.005 | 99.5 | 0.002 |
| 1-2 | 98.0 | 0.118 | 97.6 | 0.052 |
| 1-3 | 99.9 | 0.002 | 99.3 | 0.001 |
| 1-4 | 90.6 | 0.140 | 94.9 | 0.077 |
| 1-5 | 39.2 | >1 nM | 90.8 | 0.165 |

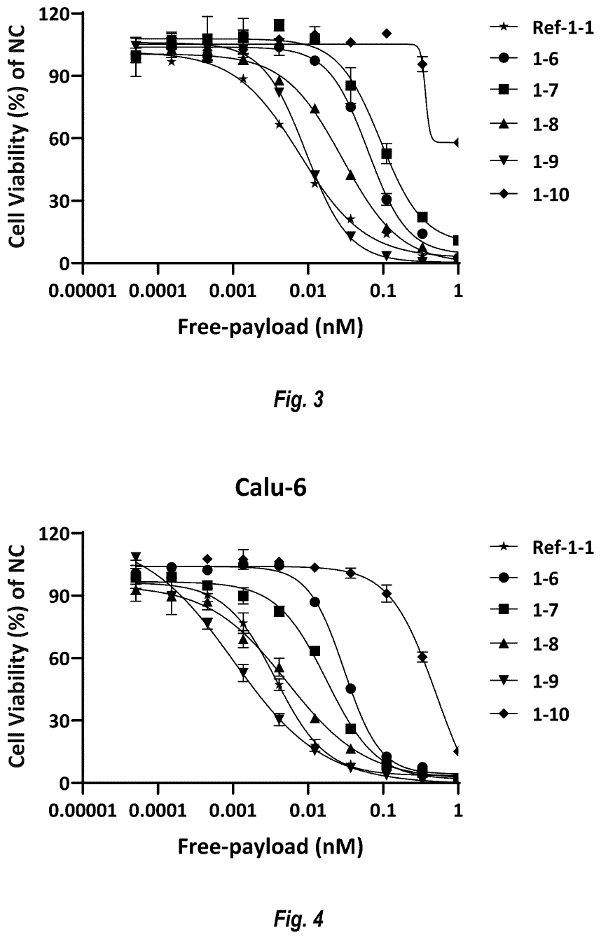

The cellular killing data of compounds 1-6 to 1-10 are presented in FIGS. 3 and 4 and in the following table:

| Compound | A375 | | Calu-6 | |
|---|---|---|---|---|
| No. | Emax (%) | EC50 (nM) | Emax (%) | EC50 (nM) |
| Ref-1-1 | 99.8 | 0.009 | 99.5 | 0.004 |
| 1-6 | 97.5 | 0.067 | 97.6 | 0.033 |
| 1-7 | 89.0 | 0.102 | 97.7 | 0.017 |
| 1-8 | 99.2 | 0.029 | 98.4 | 0.005 |
| 1-9 | 99.9 | 0.010 | 99.7 | 0.002 |
| 1-10 | 42.0 | >1 nM | 84.7 | 0.358 |

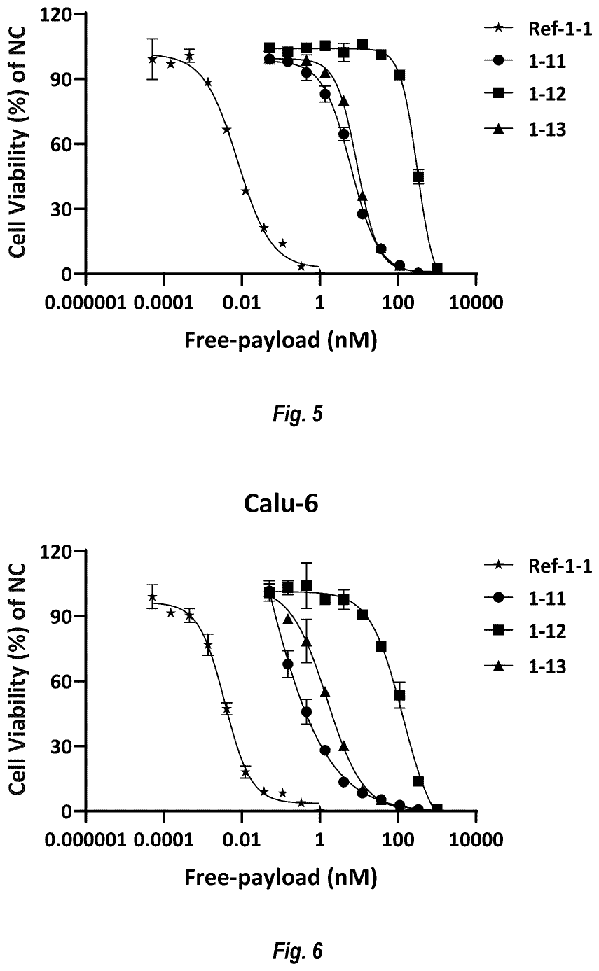

The cellular killing data of compounds 1-11 to 1-13 are presented in FIGS. 5 and 6 and in the following table:

| Compound | A375 | | Calu-6 | |
|---|---|---|---|---|
| No. | Emax (%) | EC50 (nM) | Emax (%) | EC50 (nM) |
| Ref-1-1 | 99.8 | 0.009 | 99.5 | 0.004 |
| 1-11 | 99.9 | 6.112 | 99.4 | 0.340 |
| 1-12 | 97.5 | 297.253 | 99.4 | 107.930 |
| 1-13 | 100.0 | 9.115 | 99.7 | 1.622 |

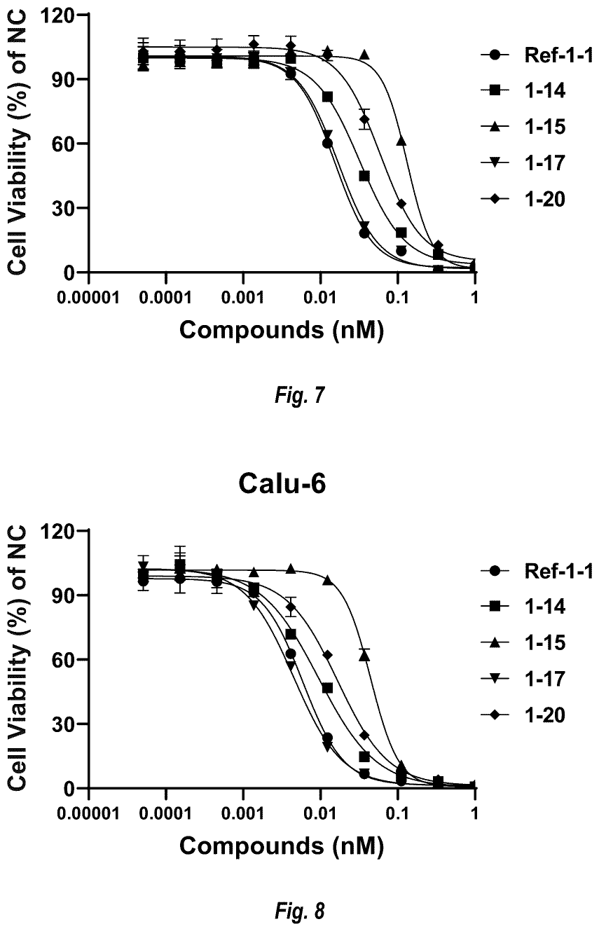

The cellular killing data of compounds 1-14, 1-15, 1-17, and 1-20 are presented in FIGS. 7 and 8 and in the following table:

| Compound | A375 | | Calu-6 | |
|---|---|---|---|---|
| No. | Emax (%) | EC50 (nM) | Emax (%) | EC50 (nM) |
| Ref-1-1 | 99.9 | 0.016 | 99.8 | 0.006 |
| 1-14 | 98.9 | 0.033 | 99.2 | 0.010 |
| 1-15 | 99.5 | 0.134 | 99.667 | 0.045 |
| 1-17 | 99.9 | 0.017 | 99.7 | 0.005 |
| 1-20 | 95.9 | 0.064 | 98.3 | 0.016 |

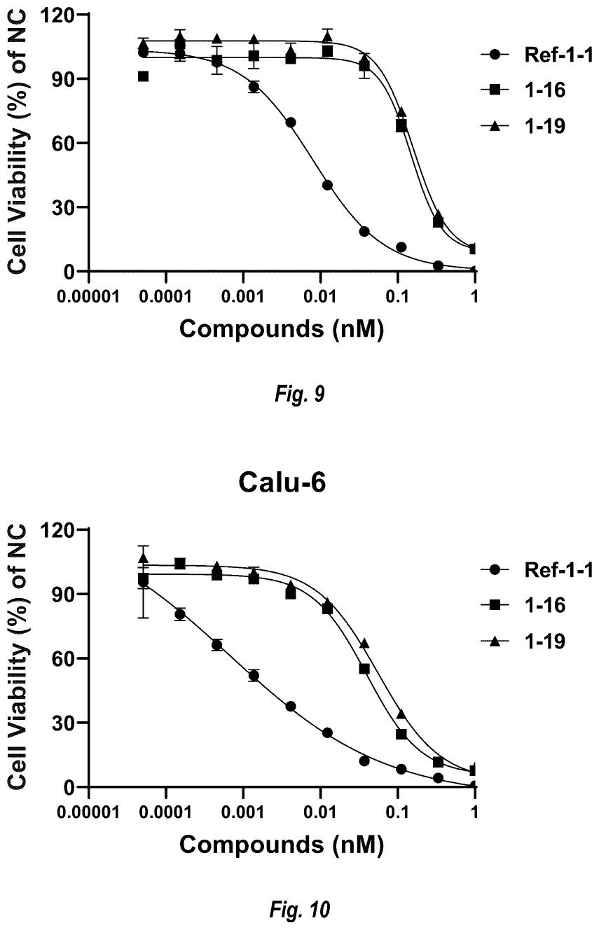

The cellular killing data of compounds 1-16 and 1-19 are presented in FIGS. 9 and 10 and in the following table:

| Compound | A375 | | Calu-6 | |
|---|---|---|---|---|
| No. | Emax (%) | EC50 (nM) | Emax (%) | EC50 (nM) |
| Ref-1-1 | 99.9 | 0.009 | 99.3 | 0.002 |
| 1-16 | 89.6 | 0.146 | 92.3 | 0.038 |
| 1-19 | 88.7 | 0.165 | 90.570 | 0.052 |

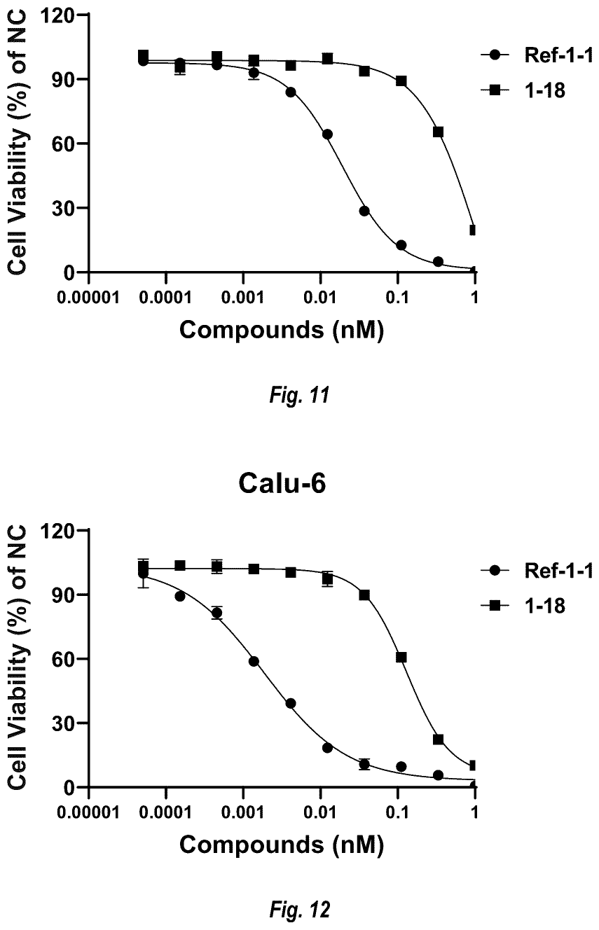

The cellular killing data of compound 1-18 are presented in FIGS. 11 and 12 and in the following table:

| Compound | A375 | | Calu-6 | |
|---|---|---|---|---|
| No. | Emax (%) | EC50 (nM) | Emax (%) | EC50 (nM) |
| Ref-1-1 | 99.6 | 0.019 | 99.3 | 0.002 |
| 1-18 | 80.3 | 0.398 | 89.8 | 0.126 |

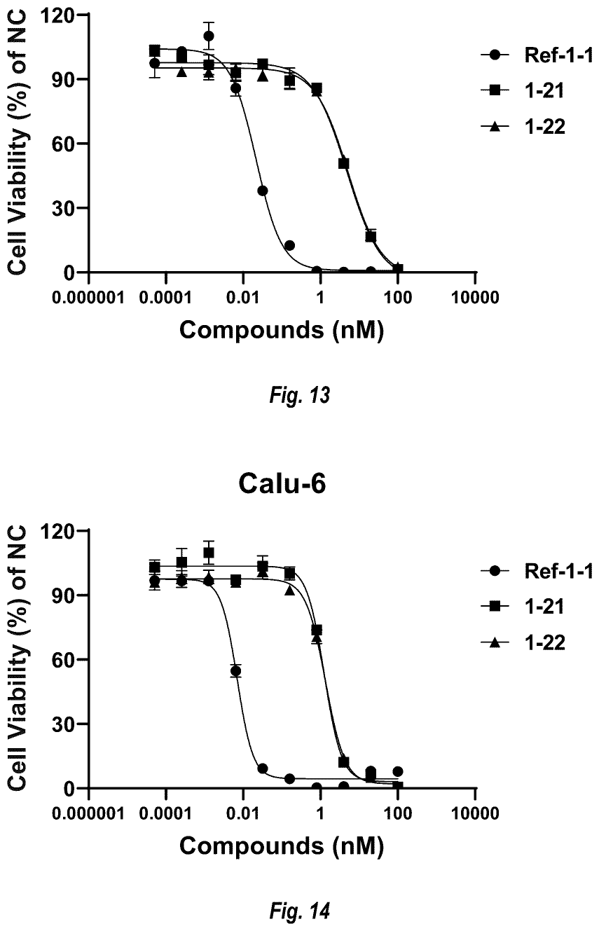

The cellular killing data of compounds 1-21 and 1-22 are presented in FIGS. 13 and 14 and in the following table:

| Compound | A375 | | Calu-6 | |
|---|---|---|---|---|
| No. | Emax (%) | EC50 (nM) | Emax (%) | EC50 (nM) |
| Ref-1-1 | 99.9 | 0.014 | 93.1 | 0.008 |
| 1-21 | 97.8 | 4.200 | 99.3 | 1.200 |
| 1-22 | 97.3 | 5.500 | 99.200 | 1.300 |

Although the foregoing disclosure has been presented in some detail by way of illustration and example for purposes of clarity of understanding, it is apparent to those skilled in the art that certain minor changes and modifications will be practiced. Therefore, the description and examples should not be construed as limiting.

It is to be understood that, if any prior art publication is referred to herein, such reference does not constitute an admission that the publication forms a part of the common general knowledge in the art in any country.

The disclosures of all non-patent publications, patents, patent applications, and published patent applications referred to herein by an identifying citation are hereby incorporated herein by reference in their entireties.

What is claimed is:

1. A compound of Formula (I): 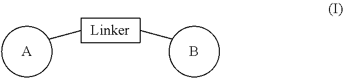
or a pharmaceutically acceptable salt, tautomer, solvate, or stereoisomer thereof,
wherein each of ring A and ring B is, independently, one of the following formulas: 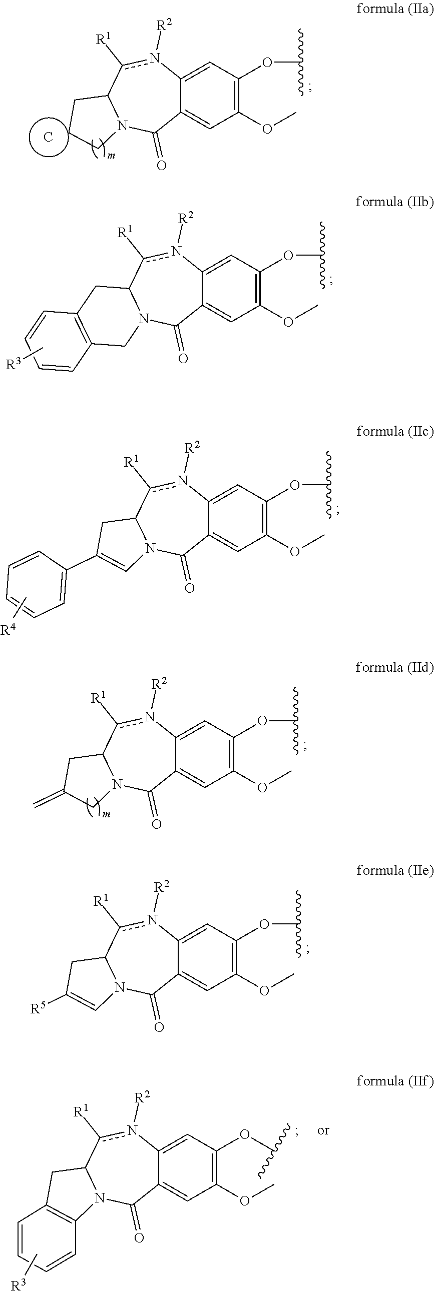 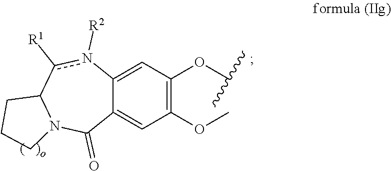 [symbol]
indicates the point of attachment to Linker;
Linker is —(CH₂)ᵣ—, —(CH₂)ₚ—X—(CH₂)_{q}—, or —(CH₂)ₚ—CH═CH—(CH₂)_{q}—;
X is NR⁶, NHC(═O), C(═O)NH, O, SO₂, a substituted or unsubstituted aryl ring, substituted or unsubstituted heteroaryl ring, substituted or unsubstituted heterocyclic ring, or substituted or unsubstituted cyclic ring;
ring C is a cyclopropyl ring or a cyclobutyl ring;
each of the dotted bonds [symbol] between —C(R¹)— and —N(R²)— is, independently, a single bond or a double bond;
when the dotted bond is a single bond, each R¹ is, independently, H or OH, and each R² is H;
when the dotted bond is a double bond, each R¹ is H, and each R² is absent;
each of R³ and R⁴ is, independently, H, NH₂, NR^{a}R^{b}, OH, C₁₋₄ alkyl, C₁₋₄ alkoxy, or aryl;
R^{a} and R^{b} are each independently H or C₁₋₄ alkyl;
R⁵ is H, C₁₋₄ alkyl, C₁₋₄ alkoxy, or aryl;
R⁶ is H, or C₁₋₄ alkyl;
each of m, n, and o is, independently, 1 or 2;
each of r, p, and q, is, independently, an integer from 1 to 8; and
the sum of p and q is an integer from 1 to 8.

2. The compound of claim 1, wherein when Linker is —(CH₂)ᵣ—, ring A is of formula (IIa) and ring B is of formula (IIb).

3. The compound of claim 1, wherein when Linker is —(CH₂)ₚ—X—(CH₂)_{q}— or —(CH₂)ₚ—CH═CH—(CH₂)_{q}—, ring A is of formula (IIa), m in ring A is 2, and ring B is one of formulas (IIa), (IIb), (IIc), (IId), (IIe), (IIf), and (JIg).

4. The compound of claim 1, wherein when Linker is —(CH₂)ₚ—X—(CH₂)_{q}— or —(CH₂)ₚ—CH═CH—(CH₂)_{q}—, ring A is of formula (IIa), m in ring A is 1, ring B is one of formulas (IIa), (IIb), (IIc), (IId), (IIe), (IIf), and (IIg), and formula (IIc) is the following formula (IIc3) or formula (IIc4):

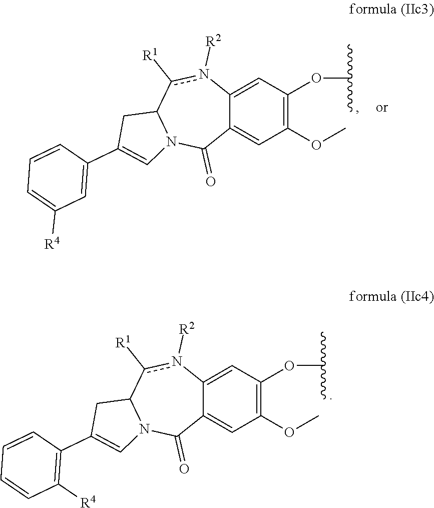

5. The compound of any one of claims 1, 3, and 4, wherein X is O, NR⁶, NHC(═O), -(m-C₆H₄)—,

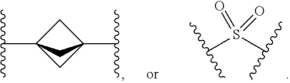 [SMILES: *[C@]12C[C@](*)(C1)C2 ; *S(*)(=O)=O]

6. The compound of claim 1, wherein Linker is —(CH₂)ᵣ—.

7. The compound of claim 1, 2, or 6, wherein r is 3 or 5.

8. The compound of claim 1 or 5, wherein Linker is —(CH₂)ₚ—O—(CH₂)_{q}— or —(CH₂)ₚ—NH—(CH₂)_{q}—.

9. The compound of any one of claims 1, 3-5, and 8, wherein the sum of p and q is 4.

10. The compound of any one of claims 1, 3, and 4, wherein Linker is

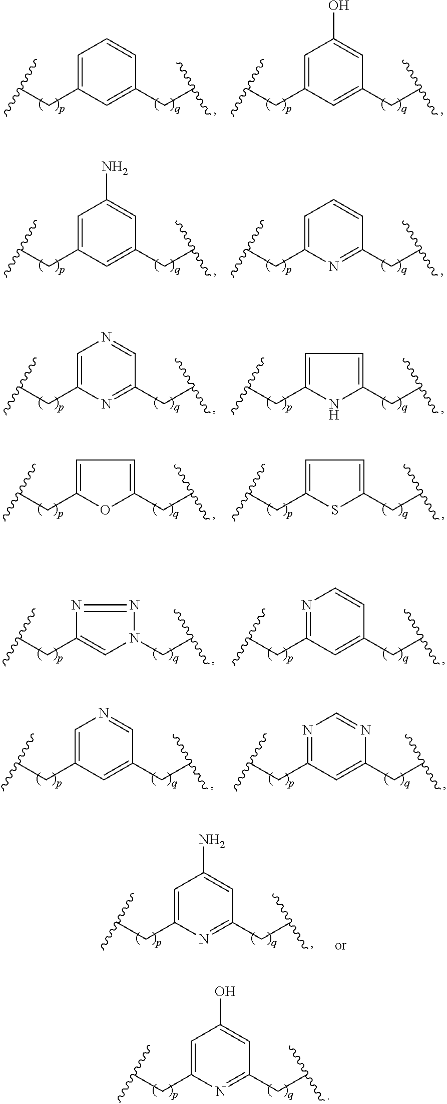

11. The compound of claim 10, wherein the sum of p and q is 2.

12. The compound of claim 5, wherein Linker is

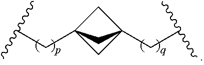

13. The compound of claim 12, wherein the sum of p and q is 2.

14. The compound of claim 1, 3, or 4, wherein Linker is —(CH₂)ₚ—CH═CH—(CH₂)_{q}—.

15. The compound of claim 14, wherein the sum of p and q is 3.

16. The compound of any one of claims 1 and 3-15, wherein ring B is of formula (IIa).

17. The compound of claim 16, wherein m in ring B is 1.

18. The compound of claim 16 or 17, wherein ring C in ring B is a cyclopropyl ring.

19. The compound of any one of claims 16-18, wherein the dotted bond in ring B is a single bond, R¹ is H or OH, and R² is H.

20. The compound of any one of claims 16-18, wherein the dotted bond in ring B is a double bond, R¹ is H, and R² is absent.

21. The compound of claim 1, wherein ring A is of formula (IIb).

22. The compound of claim 21, wherein ring A is of formula (IIb2):

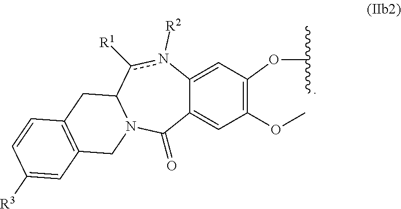

23. The compound of claim 21 or 22, wherein R³ is H.

24. The compound of any one of claims 21-23, wherein the dotted bond in ring A is a single bond, R¹ is H or OH, and R² is H.

25. The compound of any one of claims 21-23, wherein the dotted bond in ring A is a double bond, R¹ is H, and R² is absent.

26. The compound of claim 23, wherein the compound is 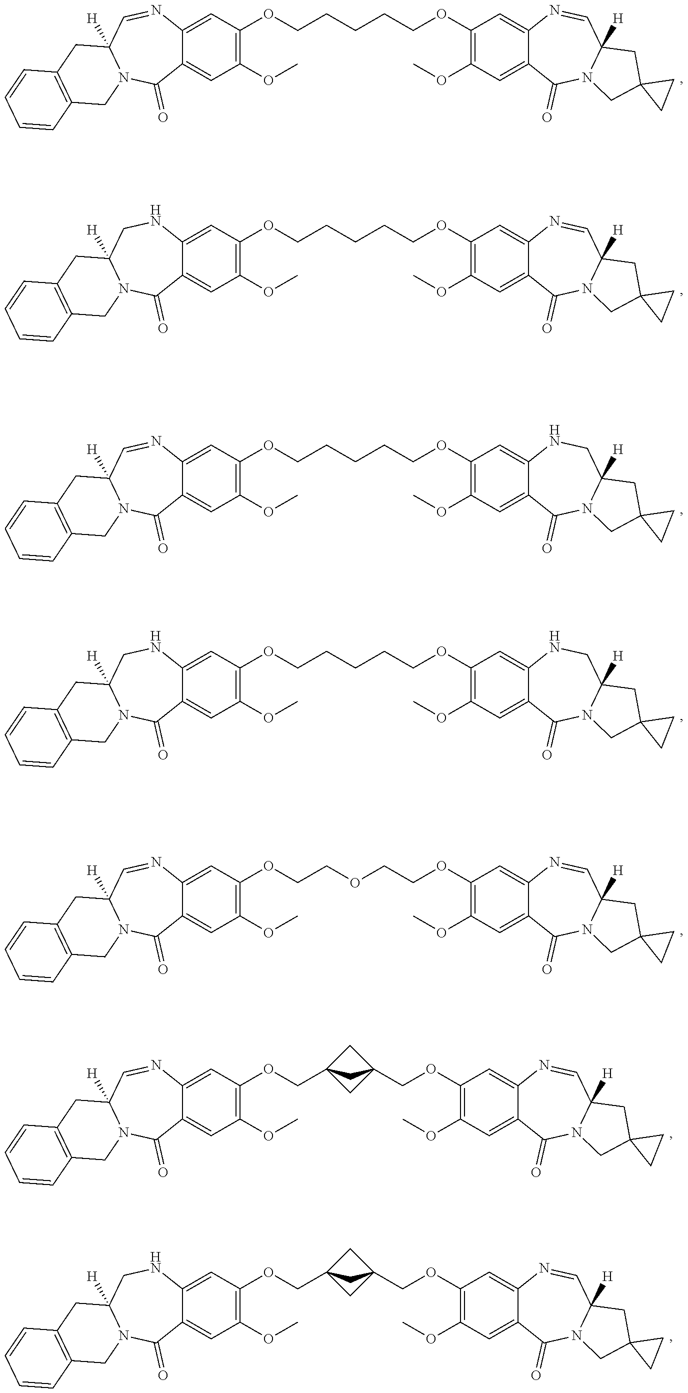 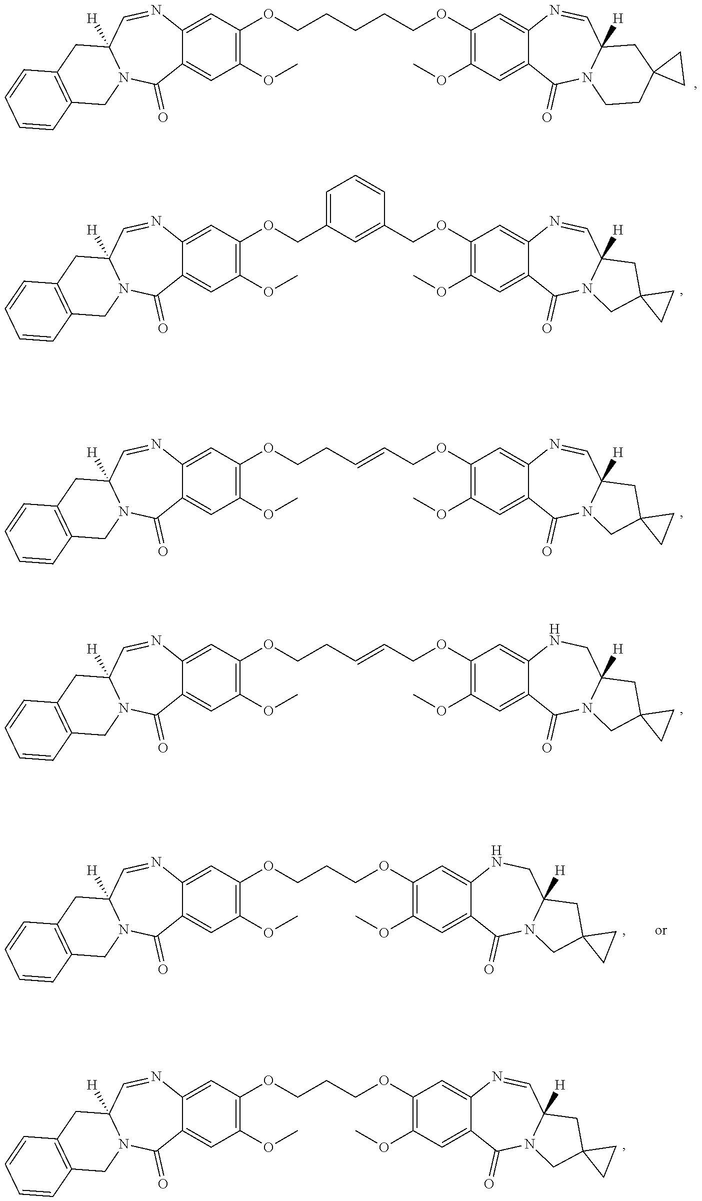
or a pharmaceutically acceptable salt, tautomer, solvate, or stereoisomer thereof.

27. The compound of claim 1, wherein ring A is of formula (IIg).

28. The compound of claim 27, wherein o in ring A is 2.

29. The compound of claim 27 or 28, wherein the dotted bond in ring A is a single bond, R¹ is H or OH, and R² is H.

30. The compound of claim 27 or 28, wherein the dotted bond in ring A is a double bond, R¹ is H, and R² is absent.

31. The compound of claim 28, wherein the compound is 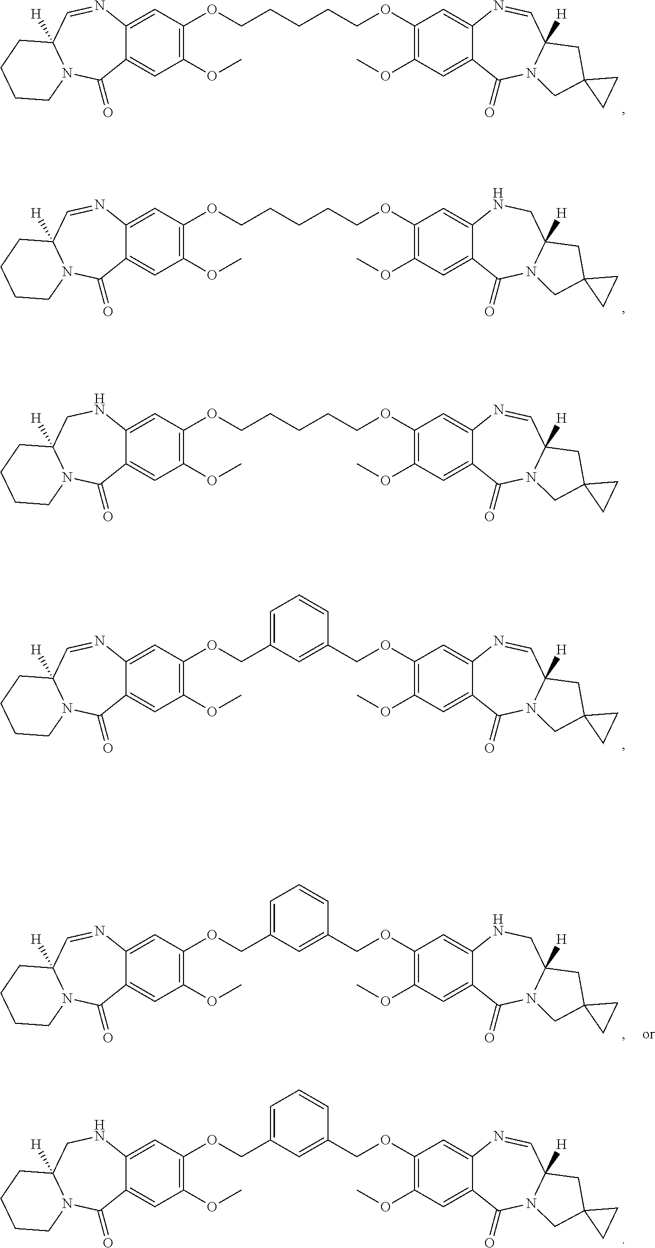
or a pharmaceutically acceptable salt, tautomer, solvate, or stereoisomer thereof.

32. The compound of claim 1, wherein ring A is of formula (IId).

33. The compound of claim 32, wherein n in ring A is 1.

34. The compound of claim 32 or 33, wherein the dotted bond in ring A is a single bond, R¹ is H or OH, and R² is H.

35. The compound of claim 32 or 33, wherein the dotted bond in ring A is a double bond, R¹ is H, and R² is absent.

36. The compound of claim 33, wherein the compound is 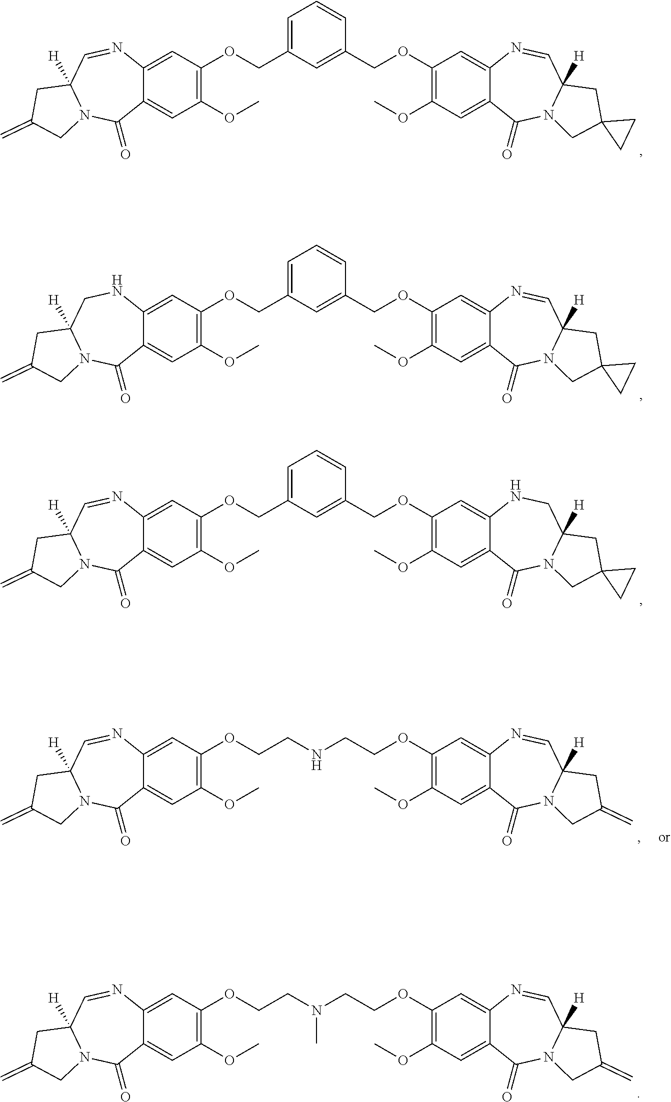
or a pharmaceutically acceptable salt, tautomer, solvate, or stereoisomer thereof.

37. The compound of claim 32, wherein n in ring A is 2.

38. The compound of claim 32 or 37, wherein the dotted bond in ring A is a single bond, R¹ is H or OH, and R² is H.

39. The compound of claim 32 or 37, wherein the dotted bond in ring A is a double bond, R¹ is H, and R² is absent.

40. The compound of claim 39, wherein the compound is 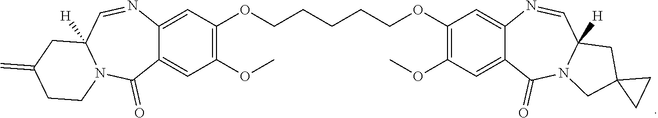 [SMILES: C=C1CCN2C(=O)c3cc(OC)c(OCCCCCOc4cc5c(cc4OC)C(=O)N4CC6(CC6)C[C@H]4C=N5)cc3N=C[C@@H]2C1]
or a pharmaceutically acceptable salt, tautomer, solvate, or stereoisomer thereof.

41. The compound of claim 1, wherein ring A is of formula (IIc).

42. The compound of claim 41, wherein ring A is formula (IIc2):

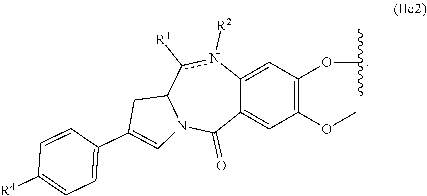

43. The compound of claim 41 or 42, wherein R⁴ is CH₃O—.

44. The compound of any one of claims 41-43, wherein the dotted bond in ring A is a single bond, R¹ is H or OH, and R² is H.

45. The compound of any one of claims 41-43, wherein the dotted bond in ring A is a double bond, R¹ is H, and R² is absent.

46. The compound of claim 42, wherein the compound is 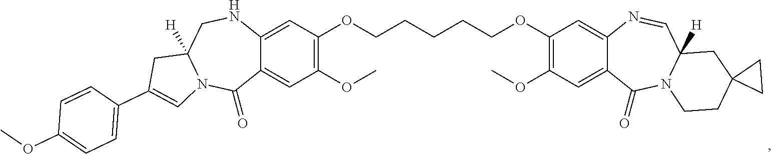 [SMILES: COc1ccc(C2=CN3C(=O)c4cc(OC)c(OCCCCCOc5cc6c(cc5OC)C(=O)N5CCC7(CC7)C[C@H]5C=N6)cc4NC[C@@H]3C2)cc1]
or a pharmaceutically acceptable salt, tautomer, solvate, or stereoisomer thereof.

47. The compound of claim 1, wherein ring A is of formula (IIe).

48. The compound of claim 47, wherein R⁵ is methyl.

49. The compound of claim 47 or 48, wherein the dotted bond in ring A is a single bond, R¹ is H or OH, and R² is H.

50. The compound of claim 47 or 48, wherein the dotted bond in ring A is a double bond, R¹ is H, and R² is absent.

51. The compound of claim 48, wherein the compound is 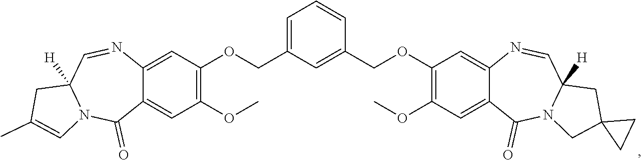 [SMILES: COc1cc2c(cc1OCc1cccc(COc3cc4c(cc3OC)C(=O)N3CC5(CC5)C[C@H]3C=N4)c1)N=C[C@@H]1CC(C)=CN1C2=O]
or a pharmaceutically acceptable salt, tautomer, solvate, or stereoisomer thereof.

52. The compound of claim 1, wherein each of ring A and ring B, independently, is of formula (IIa).

53. The compound of claim 52, wherein m is 1.

54. The compound of claim 52 or 53, wherein ring C is a cyclopropyl ring.

55. The compound of any one of claims 52-54, wherein the dotted bond in ring A is a single bond, R¹ is H or OH, and R² is H.

56. The compound of any one of claims 52-54, wherein the dotted bond in ring A is a double bond, R¹ is H, and R² is absent.

57. The compound of any one of claims 52-56, wherein the dotted bond in ring B is a single bond, R¹ is H or OH, and R² is H.

58. The compound of any one of claims 52-56, wherein the dotted bond in ring B is a double bond, R¹ is H, and R² is absent.

59. The compound of claim 54, wherein the compound is 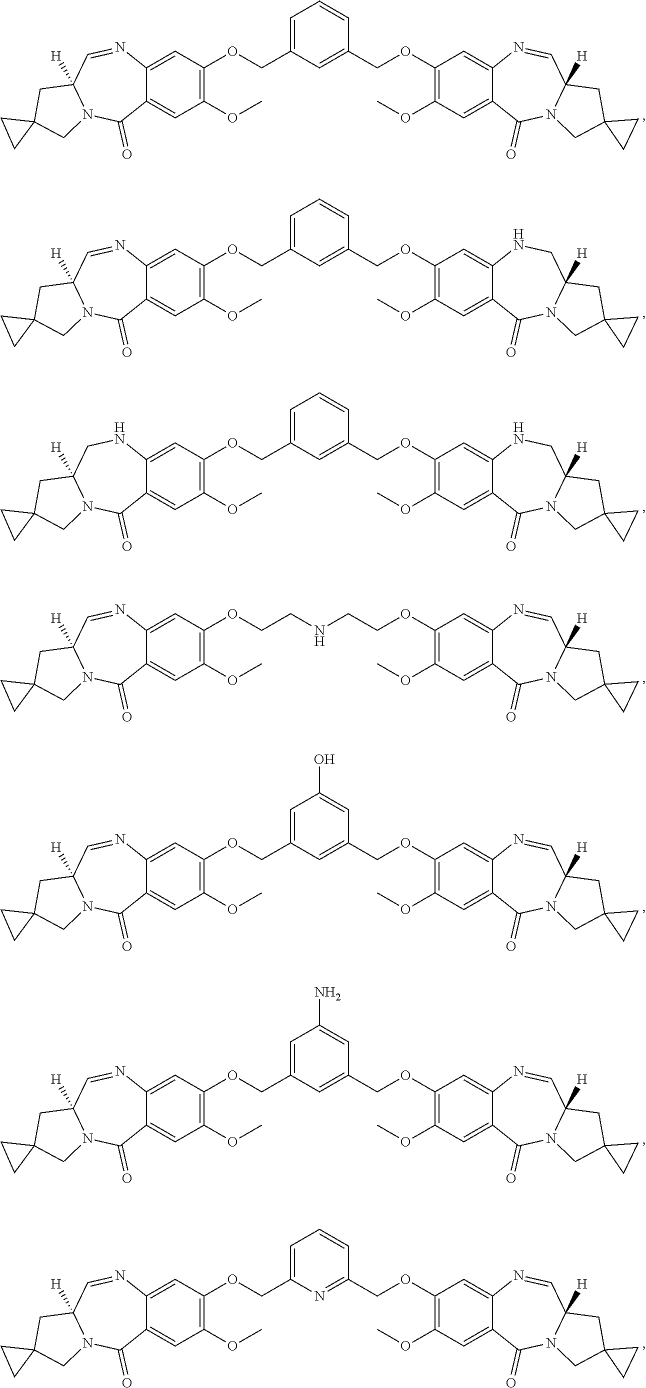 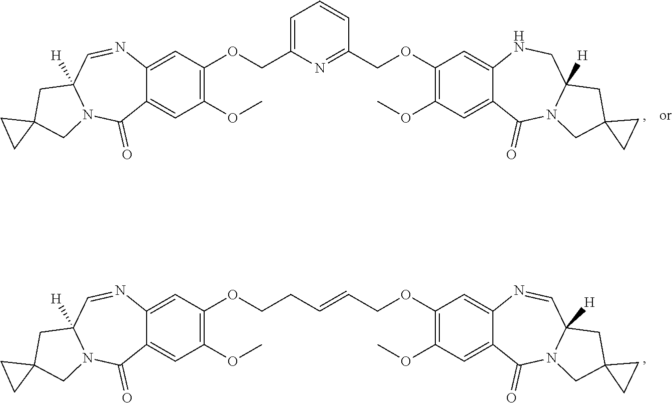 [SMILES: COc1cc2c(cc1OCc1cccc(COc3cc4c(cc3OC)C(=O)N3CC5(CC5)C[C@H]3CN4)n1)N=C[C@@H]1CC3(CC3)CN1C2=O ; COc1cc2c(cc1OC/C=C/CCOc1cc3c(cc1OC)C(=O)N1CC4(CC4)C[C@H]1C=N3)N=C[C@@H]1CC3(CC3)CN1C2=O]
or a pharmaceutically acceptable salt, tautomer, solvate, or stereoisomer thereof.

60. The compound of claim 1, wherein ring A and ring B are different.

61. A compound of Formula B(i) or B(ii): 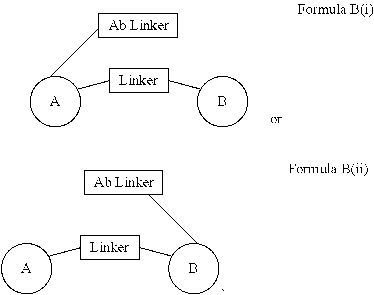
or a pharmaceutically acceptable salt, tautomer, solvate, or stereoisomer thereof, wherein:
each of ring A and ring B is, independently, one of the following formulas: 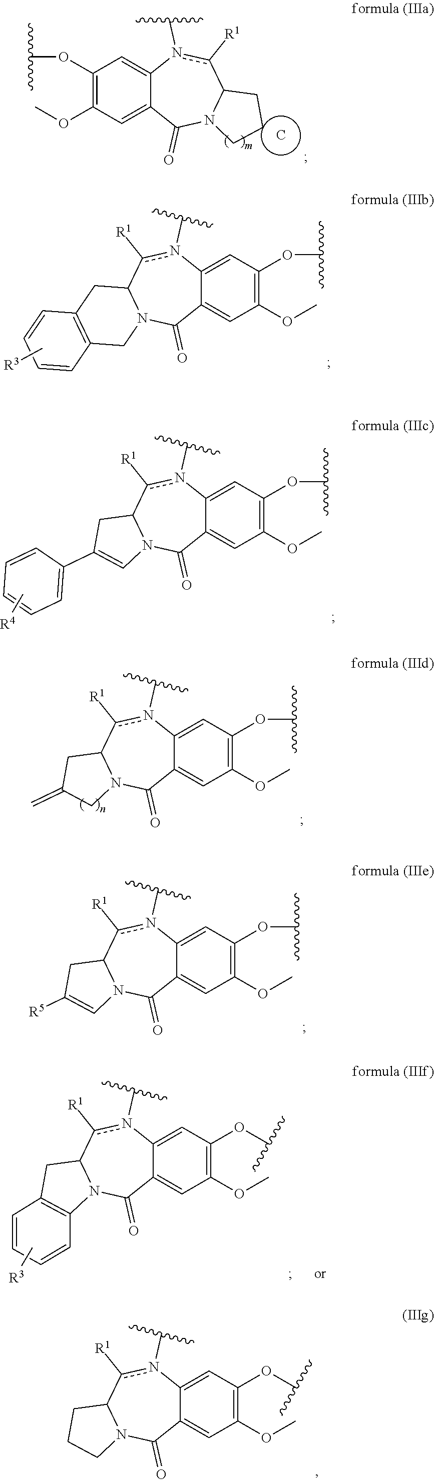 [symbol]
indicates the point of attachment to Linker or Ab Linker;
Linker is —(CH₂)ᵣ—, —(CH₂)ₚ—X—(CH₂)_{q}—, or —(CH₂)ₚ—CH═CH—(CH₂)_{q}—;
X is NR⁶, NHC(═O), C(═O)NH, O, SO₂, a substituted or unsubstituted aryl ring, substituted or unsubstituted heteroaryl ring, substituted or unsubstituted heterocyclic ring, or substituted or unsubstituted cyclic ring;
ring C is a cyclopropyl ring or a cyclobutyl ring;
each of the dotted bonds [symbol] between —C(R¹)— and —N(R²)— is, independently, a single bond or a double bond;
when the dotted bond is a single bond, each R¹ is, independently, H or OH, and each R² is H;
when the dotted bond is a double bond, each R¹ is, independently, H, and each R² is absent;
each of R³ and R⁴ is, independently, H, NH₂, N^{a}R^{b}, OH, C₁₋₄ alkyl, C₁₋₄ alkoxyl, or aryl;
R^{a} and R^{b} are each independently H or C₁₋₄ alkyl;
R⁵ is H, C₁₋₄ alkyl, C₁₋₄ alkoxyl, or aryl;
R⁶ is H or C₁₋₄ alkyl;
each of m, n, and o is, independently, 1 or 2;
each of r, p, and q, is, independently, an integer from 1 to 8;
the sum of p and q is an integer from 1 to 8; and
Ab Linker is a compound able to join ring A or ring B to a binding agent.

62. The compound of claim 61, wherein Ab Linker has the following formula: 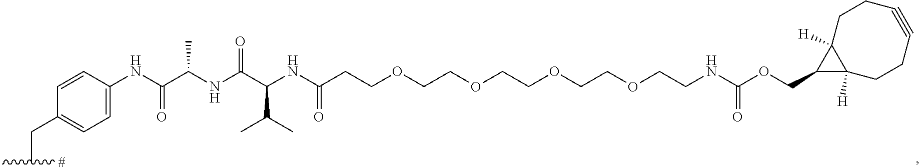
wherein [symbol] #indicates the point of attachment to ring A or ring B.

63. The compound of claim 61 or 62, wherein the compound has the following formula: 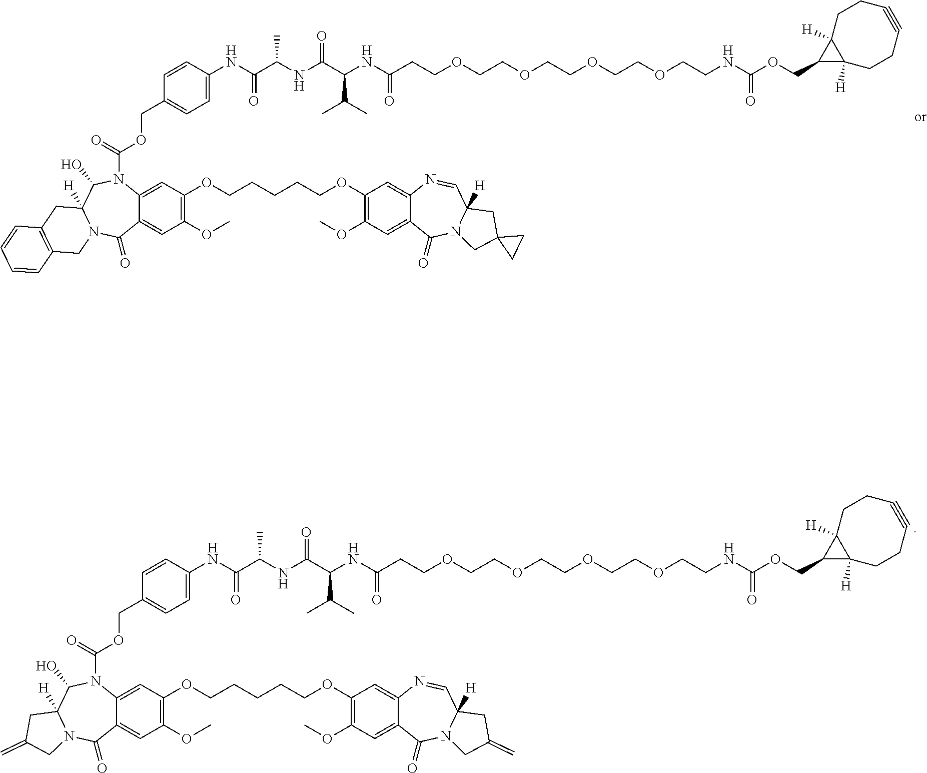 [SMILES: COc1cc2c(cc1OCCCCCOc1cc3c(cc1OC)C(=O)N1Cc4ccccc4C[C@H]1[C@H](O)N3C(=O)OCc1ccc(NC(=O)[C@H](C)NC(=O)[C@@H](NC(=O)CCOCCOCCOCCOCCNC(=O)OC[C@@H]3[C@@H]4CCC#CCC[C@@H]43)C(C)C)cc1)N=C[C@@H]1CC3(CC3)CN1C2=O ; C=C1C[C@H]2C=Nc3cc(OCCCCCOc4cc5c(cc4OC)C(=O)N4CC(=C)C[C@H]4[C@H](O)N5C(=O)OCc4ccc(NC(=O)[C@H](C)NC(=O)[C@@H](NC(=O)CCOCCOCCOCCOCCNC(=O)OC[C@@H]5[C@@H]6CCC#CCC[C@@H]65)C(C)C)cc4)c(OC)cc3C(=O)N2C1]
or a pharmaceutically acceptable salt, tautomer, solvate, or stereoisomer thereof.

64. A conjugate of Formula A(i) or A(ii): 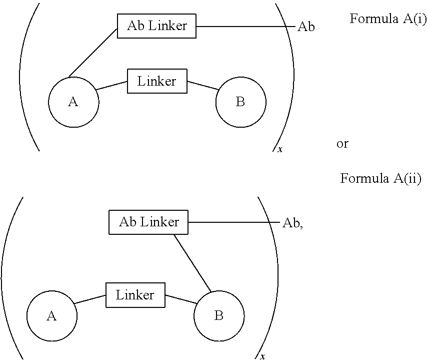
or a pharmaceutically acceptable salt, tautomer, solvate, or stereoisomer thereof, wherein:
each of ring A and ring B is, independently, one of the following formulas: 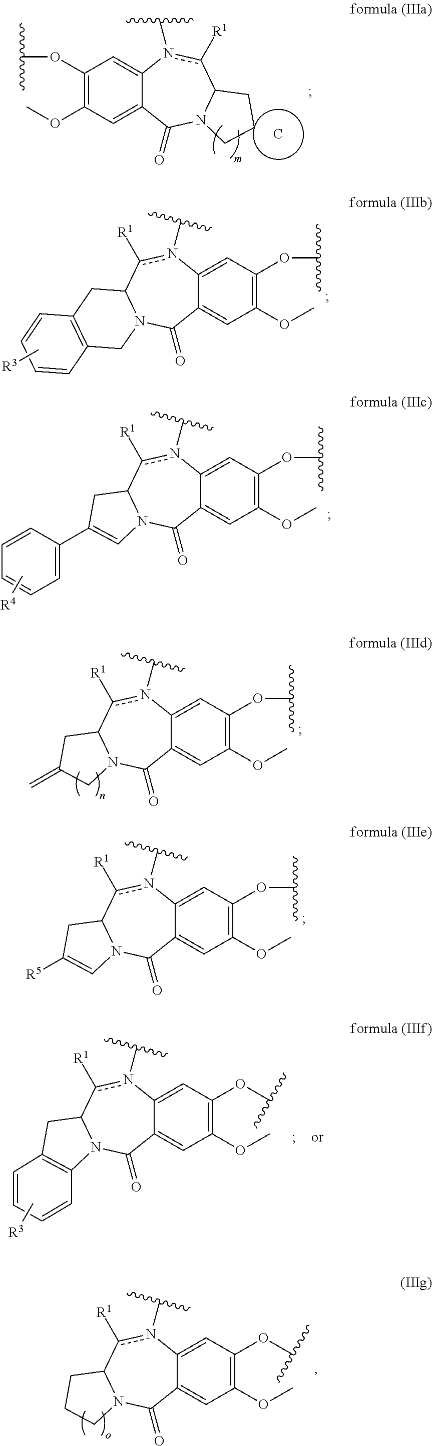 [symbol]
indicates the point of attachment to Linker or Ab Linker;
Linker is —(CH₂)ᵣ—, —(CH₂)ₚ—X—(CH₂)_{q}—, or —(CH₂)ₚ—CH═CH—(CH₂)_{q}—;
X is NR⁶, NHC(═O), C(═O)NH, O, SO₂, a substituted or unsubstituted aryl ring, substituted or unsubstituted heteroaryl ring, substituted or unsubstituted heterocyclic ring, or substituted or unsubstituted cyclic ring;
ring C is a cyclopropyl ring or a cyclobutyl ring;
each of the dotted bonds [symbol] between —C(R¹)— and —N(R²)— is, independently, a single bond or a double bond;
when the dotted bond is a single bond, each R¹ is, independently, H or OH, and each R² is H;
when the dotted bond is a double bond, each R¹ is, independently, H, and each R² is absent;
each of R³ and R⁴ is, independently, H, NH₂, NRaR^{b}, OH, C₁₋₄ alkyl, C₁₋₄ alkoxyl, or aryl;
R^{a} and R^{b} are each independently H or C₁₋₄ alkyl;
R⁵ is H, C₁₋₄ alkyl, C₁₋₄ alkoxyl, or aryl;
R⁶ is H or C₁₋₄ alkyl;
each of m, n, and o is, independently, 1 or 2;
each of r, p, and q, is, independently, an integer from 1 to 8;
the sum of p and q is an integer from 1 to 8;
Ab Linker is a compound that joins Ab to ring A or ring B;
Ab is a binding agent selected from a humanized, chimeric, or human antibody, or an antigen binding fragment thereof, and subscript x is from 1 to 15.

65. The conjugate of claim 64, wherein Ab Linker has the following formula: 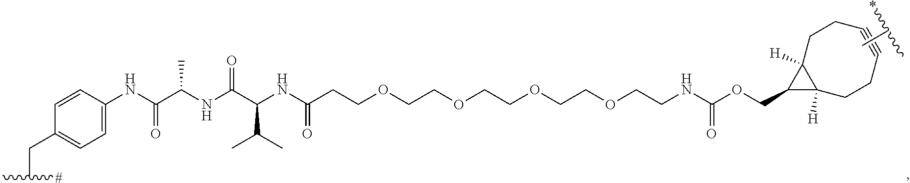
wherein [symbol] * indicates the point of attachment to Ab, and [symbol] #indicates the point of attachment to ring A or ring B.

66. The conjugate of claim 64 or 65, wherein the conjugate has the following formula: 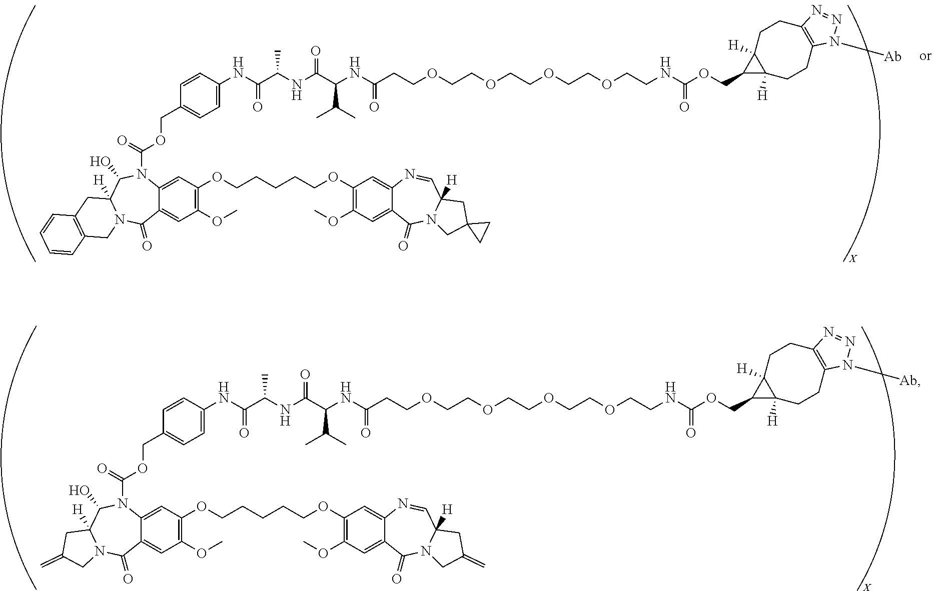
or a pharmaceutically acceptable salt, tautomer, solvate, or stereoisomer thereof.

67. The conjugate of claim 66, wherein subscript x is about 2.

68. The conjugate of any one of claims 64 to 67, wherein the conjugate has the following formula: 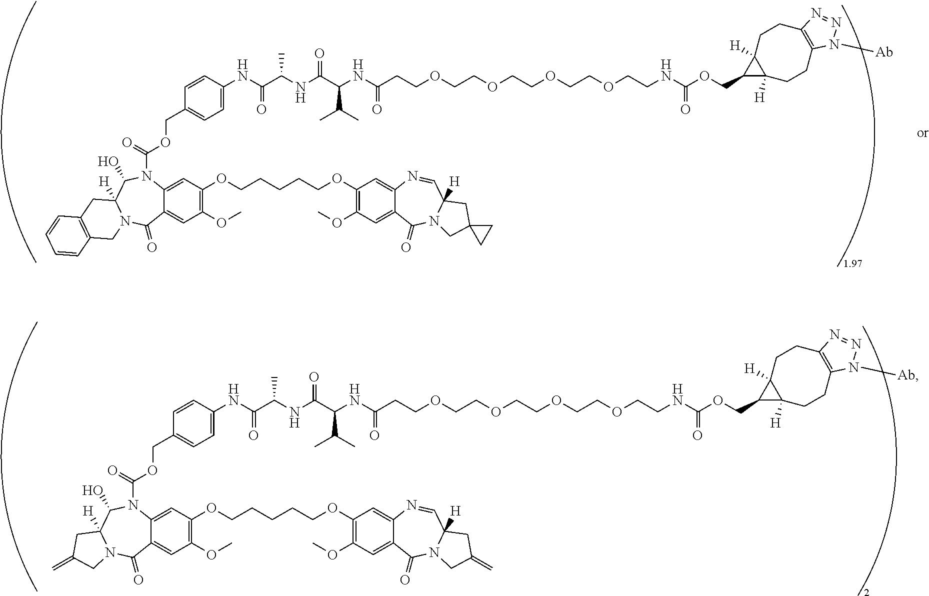
or a pharmaceutically acceptable salt, tautomer, solvate, or stereoisomer thereof.

69. A pharmaceutical composition comprising the conjugate of any one of claims 64 to 68, or a pharmaceutically acceptable salt, tautomer, solvate, or stereoisomer thereof, and a pharmaceutically acceptable excipient.
